(19) United States
(12) Patent Application Publication
(10) Pub. No.: US 2023/0417747 A1
(43) Pub. Date: Dec. 28, 2023

(54) METHODS AND COMPOSITIONS FOR DIAGNOSING AND TREATING VIRALLY-ASSOCIATED DISEASE

(71) Applicants: LA JOLLA INSTITUTE FOR IMMUNOLOGY, La Jolla, CA (US); THE UNIVERSITY OF LIVERPOOL, Liverpool, Merseyside (GB)

(72) Inventors: Pandurangan VIJAYANAND, La Jolla, CA (US); Christian OTTENSMEIER, La Jolla, CA (US)

(21) Appl. No.: 18/009,690

(22) PCT Filed: Jun. 11, 2021
(86) PCT No.: PCT/US21/37129

§ 371 Date: Dec. 9, 2022

Related U.S. Application Data
(60) Provisional application No. 63/038,121, filed on Jun. 11, 2020.

(51) Int. Cl. G01N 33/569; C12Q 1/6881

(52) U.S. Cl. CPC G01N 33/56983; C12Q 1/6881; C12Q 2600/158; G01N 2469/20

(57) ABSTRACT

Methods of treating viral diseases are disclosed herein. Certain methods include diagnostic methods that quantify levels of biological features associated with T_{FH} or CD4-CTL cells. Certain methods include treatment methods that affect the number, functionality, activity, or expression of T_{FH} or CD4-CTL cells or T_{REG} cells.

### CROSS-REFERENCE TO RELATED APPLICATION

This Application claims priority to, and the benefit of, U.S. Provisional Patent Application Ser. No. 63/038,121, entitled “Methods and Compositions for Diagnosing and Treating Virally-Associated Disease” and filed on Jun. 11, 2020, the entire contents of which are incorporated herein by reference in its entirety.

### STATEMENT REGARDING FEDERALLY-SPONSORED RESEARCH OR GOVERNMENT LICENSE RIGHTS

This invention was made with government support under grant number U19AI142742, U19AI118626, and R01HL114093 awarded by the National Institute of Health (NIH). The U.S. Government has certain rights in the invention.

### FIELD

The present disclosure relates to methods and compositions for diagnosing and treating viral diseases and disorders and, more particularly, to methods and compositions for treating and diagnosing diseases and disorders associated with elevated levels of cytotoxic CD4⁺ T cell expression or activity.

### BACKGROUND

Coronavirus disease 2019 (COVID-19) is causing substantial mortality, morbidity and economic losses and effective vaccines and therapeutics may take several months or years to become available. A substantial number of patients become life-threateningly ill, and the mechanisms responsible for causing severe respiratory distress syndrome (SARS) in COVID-19 are not well understood. Therefore, there is an urgent need to understand the key players driving protective and pathogenic immune responses in COVID-19. This knowledge may help devise better therapeutics and vaccines for tackling the current pandemic. CD4⁺ T cells are key orchestrators of anti-viral immune responses, either through direct killing of infected cells, or by enhancing the effector functions of other immune cell types like cytotoxic CD8⁺ T cells, NK cells and B cells. Recent studies in patients with COVID-19 have verified the presence of CD4⁺ T cells that are reactive to SARS-CoV-2 (see, for e.g.: Braun et al., 2020; Grifoni et al., 2020; Thieme et al., 2020). However, the nature and types of CD4⁺ T cell subsets that respond to SARS-CoV-2 and whether they play an important role in driving protective or pathogenic immune responses remain elusive. Here, the inventors have analyzed single-cell transcriptomes of virus-reactive CD4⁺ T cells to determine associations with severity of COVID-19 illness, and to compare the molecular properties of SARS-CoV2-reactive CD4⁺ T cells to other common respiratory virus-reactive CD4⁺ T cells from healthy control subjects.

### SUMMARY

This Summary is provided to introduce a selection of concepts in a simplified form that are further described below in the Detailed Description. This Summary is not intended to identify key aspects or essential aspects of the claimed subject matter.

All features of exemplary embodiments which are described in this disclosure and are not mutually exclusive can be combined with one another. Elements of one embodiment can be utilized in other embodiments without further mention. Other aspects and features of the present invention will become apparent to those ordinarily skilled in the art upon review of the following description of specific embodiments in conjunction with the accompanying Figures.

As embodied and broadly described herein, an aspect of the present disclosure relates to a method of diagnosing a viral infection in a subject, the method comprising obtaining a biological sample from the subject, quantifying a level of a biological feature associated with cytotoxic follicular helper (TFH) or cytotoxic CD4⁺ (CD4-CTL) cells from the biological sample; and comparing the level of the biological feature associated with the TFH or CD4-CTL cells against a quantifiable reference value, wherein when the level of the biological feature is higher than the quantifiable reference value, the viral infection is associated with SARS-CoV-2. In various embodiments, the quantifiable reference value comprises a biological feature associated with the activity or number of TFH or CD4-CTL cells isolated from a source infected with a non-SARS-CoV-2 virus. In various embodiments the quantifiable reference value comprises a biological feature associated with T_{FH} or CD4-CTL cells isolated from a source infected with an influenza virus. In various embodiments, the biological feature comprises the expression or activity of one or more genes set forth in Table 2 and/or Table 3, or one or more of the T-cell receptor (TCR) sequences set forth in Table 6, or a homolog, variant, subsequence, or derivative thereof. In various embodiments, the biological feature comprises expression or activity of one or more of CXCL13, IL21, CD200, BTLA, POU2AF1, PRF1, GZMB, GZMH, GNLY, or NKG7.

In another aspect, described herein is a method of diagnosing the severity of a virally-induced disease in a subject, the method comprising obtaining a biological sample from the subject; quantifying a level of a biological feature associated with T_{FH} cells from the biological sample; and comparing the level of the biological feature against a quantifiable reference value, wherein when the level of the biological feature is above the quantifiable reference value, the virally-induced disease is severe. In various embodiments, the quantifiable reference value comprises a biological feature associated with the number or activity of T_{FH} cells isolated from a second subject suffering from a non-severe case of the virally-induced disease. In various embodiments, the biological feature comprises expression or activity of one or more genes set forth in Table 3, or one or more of the TCR sequences set forth in Table 6, or a homolog, variant, subsequence, or derivative thereof. In various embodiments, the biological feature comprises expression or activity of one or more of ZBED2, ZBTB32, TIGIT, LAG3, TIM3, PD1, DUSP4, CD70, PRF1, or GZMB. In various embodiments, the virally-induced disease is COVID-19 or is associated with SARS-CoV-2.

In some embodiments, the virally-induced disease is the result of a viral infection. In some embodiments, the viral infection is caused by a virus selected from the group consisting of influenza virus, coronavirus, enterovirus (such as coxsackievirus and echovirus), cytomegalovirus, Zika virus, rabies virus, West Nile virus, rubella virus, polio virus, rotavirus, norovirus, herpes simplex virus, varicella-zoster virus, lymphocytic choriomeningitis virus, human immunodeficiency virus, Chikungunya virus, Crimean-Congo hemorrhagic fever virus, Japanese encephalitis virus, Rift Valley Fever virus, Ross River virus, and louping ill virus. In various embodiments, the virally-induced disease is COVID-19 or is associated with SARS-CoV-2.

In another aspect, described herein is a method of diagnosing the severity of a virally-induced disease in a subject, the method comprising obtaining a biological sample from the subject; quantifying a level of a biological feature associated with CD4-CTL cells from the biological sample; and comparing the level of the biological feature against a quantifiable reference value, wherein when the level of the biological feature is above the quantifiable reference value, the virally-induced disease is severe. In various embodiments, the quantifiable reference value comprises a biological feature associated with the number or activity of CD4-CTL cells isolated from a second subject suffering from a non-severe case of the virally-induced disease. In various embodiments, the biological feature comprises expression or activity of one or more genes set forth in Table 2 or Table 4, or one or more of the TCR sequences set forth in Table 6, or a homolog, variant, subsequence, or derivative thereof. In various embodiments, the biological feature comprises expression or activity of one or more of CD72, GPR18, HOPX, ZEB2, CCL3, CCL4, CCL5, CCR1, CCR3, CCR5, XCL1, or XCL2. In various embodiments, the virally-induced disease is COVID-19 or is associated with SARS-CoV-2.

In another aspect, described herein is a method of diagnosing severity of a virally-induced disease in a subject, the method comprising obtaining a biological sample from the subject; quantifying a level of a biological feature associated with T_{REG} cells from the biological sample; and comparing the level of the biological feature associated with T_{REG} against a quantifiable reference value, wherein when the level of the biological feature is below the quantifiable reference value, the virally-induced disease is severe. In various embodiments, the quantifiable reference value comprises a biological feature associated with the number or activity of T_{REG} cells isolated from a second subject suffering from a mild form of the virally-induced disease. In various embodiments, the biological feature comprises expression or activity of FOXP3, or one or more of the TCR sequences set forth in Table 7, or a homolog, variant, subsequence, or derivative thereof. In various embodiments, the virally-induced disease is COVID-19 or is associated with SARS-CoV-2. In various embodiments, the biological feature comprises the expression or activity of T-bet, IFN-γ, IL-2, TNF, IL-3, CSF2, IL-23A, or CCL20. In various embodiments, the virally-induced disease is COVID-19 or is associated with SARS-CoV-2.

In another aspect, described herein is a method of treating a coronavirus infection, treating a disease associated with coronavirus infection, or decreasing, reducing, inhibiting, suppressing, limiting or controlling an adverse symptom or disorder resulting from the coronavirus infection in a subject, the method comprising administering to the subject a therapeutically effective amount of T_{REG} cells.

In another aspect, described herein is a method of treating a coronavirus infection, treating a disease associated with coronavirus infection, or decreasing, reducing, inhibiting, suppressing, limiting or controlling an adverse symptom or disorder resulting from the coronavirus infection in a subject, the method comprising administering to the subject a therapeutic effective amount of an agent that can selectively increase T_{REG} cells in the subject.

In another aspect, described herein is a method of treating a coronavirus infection, treating a disease associated with coronavirus infection, or decreasing, reducing, inhibiting, suppressing, limiting or controlling an adverse symptom or disorder resulting from the coronavirus infection in a subject, the method comprising administering to the subject a therapeutic effective amount of an agent that can selectively reduce T_{FH} or CD4+ CTL cells in the subject. In various embodiments, the agent comprises an antibody that selectively binds to a protein expressed by T_{FH} or CD4+ CTL cells.

In another aspect, described herein is a method of treating a coronavirus infection, treating a disease associated with coronavirus infection, or decreasing, reducing, inhibiting, suppressing, limiting or controlling an adverse symptom or disorder resulting from the coronavirus infection in a subject, the method comprising administering to the subject an effective amount of a population of T-cells that exhibit higher than or lower than baseline expression of one or more genes set forth in Table 1, Table 2, Table 3, Table 4, Table 5, or that express a T-cell receptor (TCR) comprising at least one of the amino acid sequences set forth in Tables 6 and 7, or a homolog, variant, subsequence, or derivative thereof. In various embodiments, the method comprises administering a population of T-cells that exhibit higher than baseline expression of one or more genes set forth in Table 1 and Table 5, or that express a TCR comprising at least one of the amino acid sequences set forth in Table 7, or a homolog, variant, subsequence, or derivative thereof. In various embodiments, the T-cell is a T_{REG} cell. In various embodiments, the one or more genes are selected from the group of T-bet, IFN-γ, IL-2, TNF, IL-3, CSF2, IL-23A, CCL20, IL17A, FOXP3, and IL17F. In various embodiments, the at least one amino acid sequence is selected from Table 7. In various embodiments, the method comprises administering a population of T-cells that exhibit lower than baseline expression of one or more genes set forth in Table 2, Table 3, or Table 4, or that express a TCR comprising at least one of the amino acid sequences set forth in Table 6, or a homolog, variant, subsequence, or derivative thereof. In various embodiments, the one or more genes are selected from the group of ZBED2, ZBTB32, TIGIT, LAG3, TIM3, PD1, DUSP4, CD70, PRF1, and GZMB. In various embodiments, the T-cell is a T_{FH} cell. In various embodiments, the one or more genes are selected from the group of CD72, GPR18, HOPX, ZEB2, CCL3, CCL4, CCL5, CCR1, CCR3, CCR5, XCL1, and XCL2. In various embodiments, the T cell is a CD4-CTL T cell. In various embodiments, the at least one amino acid sequence is selected from Table 6.

In another aspect, described herein is a method of treating a coronavirus infection, treating a disease associated with coronavirus infection, or decreasing, reducing, inhibiting, suppressing, limiting or controlling an adverse symptom or disorder resulting from the coronavirus in a subject, the method comprising administering to the subject an effective amount of an agent that induces higher than or lower than baseline expression of one or more genes set forth in Table 1, Table 2, Table 3, Table 4, and/or Table 5 in T cells, or of a TCR of at least one of the amino acid sequences set forth in Tables 6 and 7, or a homolog, variant, subsequence, or derivative thereof.

In another aspect, described herein is a method of treating a coronavirus infection, treating a disease associated with coronavirus infection, or decreasing, reducing, inhibiting, suppressing, limiting or controlling an adverse symptom or disorder resulting from the coronavirus in a subject, the method comprising administering an effective amount of an agent that induces or inhibits T cell activity of one or more proteins encoded by one or more genes set forth in Table 1, Table 2, Table 3, Table 4, and/or Table 5, or that modulates expression of a T-cell receptor (TCR) comprising at least one of the amino acid sequences set forth in Tables 6 and 7, or a homolog, variant, subsequence, or derivative thereof. In various embodiments, the agent is an antibody, a small molecule, a protein, a peptide, a ligand mimetic or a nucleic acid. In various embodiments, the baseline expression is normalized mean gene expression. In various embodiments, higher than baseline expression is at least about a 2-fold increase in expression relative to baseline expression and/or lower than baseline expression is at least about a 2-fold decrease in expression relative to baseline expression.

In another aspect, described herein is a modified T-cell modified to exhibit higher than or lower than baseline expression of one or more genes set forth in Table 1, Table 2, Table 3, Table 4, and/or Table 5, or one or more T-cell receptor (TCR) comprising at least one of the amino acid sequences set forth in Tables 6 and 7, or a homolog, variant, subsequence, or derivative thereof. In various embodiments, the modified T cell exhibits higher than baseline expression of one or more genes set forth in Table 1 or Table 5, or expresses a TCR comprising at least one of the amino acid sequences set forth in Table 7, or a homolog, variant, subsequence, or derivative thereof. In various embodiments, the one or more genes are selected from the group of T-bet, IFN-γ, IL-2, TNF, IL-3, CSF2, IL-23A, CCL20, IL17A, FOXP3, and IL17F. In various embodiments, the at least one amino acid sequence is selected from Table 7. In various embodiments, the modified T cell is a T_{REG} cell. In various embodiments, the baseline expression is normalized mean gene expression. In various embodiments, higher than baseline expression is at least about a 2-fold increase in expression relative to baseline expression and/or lower than baseline expression is at least about a 2-fold decrease in expression relative to baseline expression. In various embodiments, the modified T-cell is genetically modified, optionally using one or more of gene editing, recombinant methods and/or a CRISPR/Cas system.

In various embodiments, the modified T-cell is further modified to express a protein that binds to a cytokine, chemokine, lymphokine, or a receptor each thereof. In various embodiments, the protein comprises an antibody or an antigen binding fragment thereof. In various embodiments, the antibody is an IgG, IgA, IgM, IgE or IgD, or a subclass thereof. In various embodiments, the antibody is an IgG selected from the group of IgG1, IgG2, IgG3 or IgG4. In various embodiments, the antigen binding fragment is selected from the group of a Fab, Fab′, F(ab′)2, Fv, Fd, single-chain Fvs (scFv), disulfide-linked Fvs (sdFv) or VL or VH In various embodiments, the modified T-cell comprises a chimeric antigen receptor (CAR). In various embodiments, the chimeric antigen receptor (CAR) comprises: (a) an antigen binding domain; (b) a hinge domain; (c) a transmembrane domain; (d) and an intracellular domain.

In various embodiments, the CAR further comprises one or more costimulatory signaling regions. In various embodiments, the antigen binding domain comprises an anti-CD19 antigen binding domain, the transmembrane domain comprises a CD28 or a CD8 α transmembrane domain, the one or more costimulatory regions selected from a CD28 costimulatory signaling region, a 4-1BB costimulatory signaling region, an ICOS costimulatory signaling region, and an OX40 costimulatory region or a CD3 zeta signaling domain. In various embodiments, the anti-CD19 binding domain comprises a single-chain variable fragment (scFv) that specifically recognizes a humanized anti-CD19 binding domain. In various embodiments, the anti-CD19 binding domain scFv of the CAR comprises a heavy chain variable region and a light chain variable region. In various embodiments, the anti-CD19 binding domain of the CAR further comprises a linker polypeptide located between the anti-CD19 binding domain scFv heavy chain variable region and the anti-CD19 binding domain scFv light chain variable region. In various embodiments, the linker polypeptide of the CAR comprises a polypeptide of the sequence (GGGGS)n wherein n is an integer from 1 to 6. In various embodiments, the CAR further comprises a detectable marker attached to the CAR. In various embodiments, the CAR further comprises a purification marker attached to the CAR. In various embodiments, the modified T-cell comprises a polynucleotide encoding the CAR, and optionally, wherein the polynucleotide encodes and anti-CD19 binding domain.

In various embodiments, the polynucleotide further comprises a promoter operatively linked to the polynucleotide to express the polynucleotide in the modified T-cell. In various embodiments, the polynucleotide further comprises a 2A self-cleaving peptide (T2A) encoding polynucleotide sequence located upstream of a polynucleotide encoding the anti-CD19 binding domain. In various embodiments, the polynucleotide further comprises a polynucleotide encoding a signal peptide located upstream of a polynucleotide encoding the anti-CD19 binding domain. In various embodiments, the polynucleotide further comprises a vector. In various embodiments, the vector is a plasmid. In various embodiments, the vector is a viral vector selected from the group of a retroviral vector, a lentiviral vector, an adenoviral vector, and an adeno-associated viral vector.

In another aspect, described herein is a composition comprising a population of modified T-cells as detailed herein.

In another aspect, described herein is a method of treating a coronavirus infection, treating a disease associated with coronavirus infection, or decreasing, reducing, inhibiting, suppressing, limiting or controlling an adverse symptom or disorder resulting from the coronavirus in a subject, the method comprising administering to the subject an effective amount of modified T-cells as detailed herein and/or a composition as detailed herein. In various embodiments, the coronavirus infection is SARS-CoV-2. In various embodiments, the disease associated with coronavirus infection is COVID-19. In various embodiments, the method comprises agonizing a population of or increasing the level, expression, or activity of T_{REG} cells in the subject. In various embodiments, the method comprises antagonizing a population of or decreasing or depleting the level, expression, or activity of T_{FH} or CD4-CTL cells in the subject.

In another aspect, described herein is a method of diagnosing a viral infection ex vivo, the method comprising quantifying, ex vivo, a level of a biological feature associated with T_{FH} or CD4-CTL cells from a biological sample; and comparing the level of the biological feature associated with the T_{FH} or CD4-CTL cells against a quantifiable reference value, wherein when the level of the biological feature is higher than the quantifiable reference value, the viral infection is associated with SARS-CoV-2. In various embodiments, the quantifiable reference value comprises a biological feature associated with the activity or number of T_{FH} or CD4-CTL cells isolated from a biological sample infected with a non-SARS-CoV-2 virus. In various embodiments, the quantifiable reference value comprises a biological feature associated with T_{FH} or CD4-CTL cells isolated from a biological sample infected with an influenza virus. In various embodiments, the biological feature comprises the expression or activity of one or more genes set forth in Table 2 and/or Table 3, or one or more of the T-cell receptor (TCR) sequences set forth in Table 6, or a homolog, variant, subsequence, or derivative thereof. In various embodiments, the biological feature comprises expression or activity of one or more of CXCL13, IL21, CD200, BTLA, POU2AF1, PRF1, GZMB, GZMH, GNLY, or NKG7.

In another aspect, described herein is a method of diagnosing the severity of a virally-induced disease ex vivo, the method comprising quantifying, ex vivo, a level of a biological feature associated with T_{FH} cells from the biological sample; and comparing the level of the biological feature against a quantifiable reference value, wherein when the level of the biological feature is above the quantifiable reference value, the virally-induced disease is severe. In various embodiments, the quantifiable reference value comprises a biological feature associated with the number or activity of T_{FH} cells isolated from a biological sample of a subject suffering from a non-severe case of the virally-induced disease. In various embodiments, the biological feature comprises expression or activity of one or more genes set forth in Table 3, or one or more of the TCR sequences set forth in Table 6, or a homolog, variant, subsequence, or derivative thereof. In various embodiments, the biological feature comprises expression or activity of one or more of ZBED2, ZBTB32, TIGIT, LAG3, TIM3, PD1, DUSP4, CD70, PRF1, or GZMB. In various embodiments, the virally-induced disease is COVID-19 or is associated with SARS-CoV-2.

In another aspect, described herein is a method of diagnosing the severity of a virally-induced disease ex vivo, the method comprising quantifying, ex vivo, a level of a biological feature associated with CD4-CTL cells from the biological sample; and comparing the level of the biological feature against a quantifiable reference value, wherein when the level of the biological feature is above the quantifiable reference value, the virally-induced disease is severe. In various embodiments, the quantifiable reference value comprises a biological feature associated with the number or activity of CD4-CTL cells isolated from a biological sample of a subject suffering from a non-severe case of the virally-induced disease. In various embodiments, the biological feature comprises expression or activity of one or more genes set forth in Table 2 or Table 4, or one or more of the TCR sequences set forth in Table 6, or a homolog, variant, subsequence, or derivative thereof. In various embodiments, the biological feature comprises expression or activity of one or more of CD72, GPR18, HOPX, ZEB2, CCL3, CCL4, CCL5, CCR1, CCR3, CCR5, XCL1, or XCL2. In various embodiments, the virally-induced disease is COVID-19 or is associated with SARS-CoV-2.

In another aspect, described herein is a method of diagnosing severity of a virally-induced disease ex vivo, the method comprising quantifying, ex vivo, a level of a biological feature associated with T_{REG} cells from the biological sample; and comparing the level of the biological feature associated with T_{REG} against a quantifiable reference value, wherein when the level of the biological feature is below the quantifiable reference value, the virally-induced disease is severe. In various embodiments, the quantifiable reference value comprises a biological feature associated with the number or activity of T_{REG} cells isolated from a biological sample of a subject suffering from the virally-induced disease. In various embodiments, the biological sample is isolated from a subject suffering from a mild form of the virally-induced disease. In various embodiments, the biological sample is isolated from a subject suffering from a severe form of the virally-induced disease. In various embodiments, the biological feature comprises expression or activity of FOXP3, or one or more of the TCR sequences set forth in Table 7, or a homolog, variant, subsequence, or derivative thereof. In various embodiments, the virally-induced disease is COVID-19 or is associated with SARS-CoV-2. In various embodiments, the biological feature comprises the expression or activity of T-bet, IFN-γ, IL-2, TNF, IL-3, CSF2, IL-23A, or CCL20. In various embodiments, the virally-induced disease is COVID-19 or is associated with SARS-CoV-2.

In another aspect, described herein is a method of treating a viral infection, treating a disease associated with the viral infection, or decreasing, reducing, inhibiting, suppressing, limiting or controlling an adverse symptom or disorder resulting from the viral infection in a subject, the method comprising administering to the subject a therapeutically effective amount of T_{REG} cells.

In another aspect, described herein is a method of treating a viral infection, treating a disease associated with viral infection, or decreasing, reducing, inhibiting, suppressing, limiting or controlling an adverse symptom or disorder resulting from the viral infection in a subject, the method comprising administering to the subject a therapeutic effective amount of an agent that can selectively increase T_{REG} cells in the subject.

In another aspect, described herein is a method of treating a viral infection, treating a disease associated with the viral infection, or decreasing, reducing, inhibiting, suppressing, limiting or controlling an adverse symptom or disorder resulting from the viral infection in a subject, the method comprising administering to the subject a therapeutic effective amount of an agent that can selectively reduce T_{FH} or CD4+ CTL cells in the subject. In various embodiments, the agent comprises an antibody that selectively binds to a protein expressed by T_{FH} or CD4+ CTL cells.

In another aspect, described herein is a method of treating a viral infection, treating a disease associated with the viral infection, or decreasing, reducing, inhibiting, suppressing, limiting or controlling an adverse symptom or disorder resulting from the viral infection in a subject, the method comprising administering to the subject an effective amount of a population of T-cells that exhibit higher than or lower than baseline expression of one or more genes set forth in Table 1, Table 2, Table 3, Table 4, and/or Table 5, or that express a T-cell receptor (TCR) comprising at least one of the amino acid sequences set forth in Tables 6 and 7, or a homolog, variant, subsequence, or derivative thereof. In various embodiments, the method comprises administering a population of T-cells that exhibit higher than baseline expression of one or more genes set forth in Table 1 or Table 5, or that express a TCR comprising at least one of the amino acid sequences set forth in Table 7, or a homolog, variant, subsequence, or derivative thereof. In various embodiments, the T-cell is a T_{REG} cell. In various embodiments, the one or more genes are selected from the group of T-bet, IFN-γ, IL-2, TNF, IL-3, CSF2, IL-23A, CCL20, IL17A, FOXP3, and IL17F. In various embodiments, the at least one amino acid sequence is selected from Table 7. In various embodiments, the method comprises administering a population of T-cells that exhibit lower than baseline expression of one or more genes set forth in Table 2, Table 3, or Table 4, or that express a TCR comprising at least one of the amino acid sequences set forth in Table 6, or a homolog, variant, subsequence, or derivative thereof. In various embodiments, the one or more genes are selected from the group of ZBED2, ZBTB32, TIGIT, LAG3, TIM3, PD1, DUSP4, CD70, PRF1, and GZMB. In various embodiments, the T-cell is a T_{FH} cell. In various embodiments, the one or more genes are selected from the group of CD72, GPR18, HOPX, ZEB2, CCL3, CCL4, CCL5, CCR1, CCR3, CCR5, XCL1, and XCL2. In various embodiments, the T cell is a CD4-CTL T cell. In various embodiments, the at least one amino acid sequence is selected from Table 6.

In another aspect, described herein is a method of treating a viral infection, treating a disease associated with the viral infection, or decreasing, reducing, inhibiting, suppressing, limiting or controlling an adverse symptom or disorder resulting from the viral infection in a subject, the method comprising administering to the subject an effective amount of an agent that induces higher than or lower than baseline expression of one or more genes set forth in Table 1, Table 2, Table 3, Table 4, and/or Table 5 in T cells, or of a TCR of at least one of the amino acid sequences set forth in Tables 6 and 7, or a homolog, variant, subsequence, or derivative thereof.

In another aspect, described herein is a method of treating a viral infection, treating a disease associated with the viral infection, or decreasing, reducing, inhibiting, suppressing, limiting or controlling an adverse symptom or disorder resulting from the viral infection in a subject, the method comprising administering an effective amount of an agent that induces or inhibits T cell activity of one or more proteins encoded by one or more genes set forth in Table 1, Table 2, Table 3, Table 4, and/or Table 5, or that modulates expression of a T-cell receptor (TCR) comprising at least one of the amino acid sequences set forth in Tables 6 and 7, or a homolog, variant, subsequence, or derivative thereof. In various embodiments, the agent is an antibody, a small molecule, a protein, a peptide, a ligand mimetic or a nucleic acid. In various embodiments, baseline expression is normalized mean gene expression. In various embodiments, higher than baseline expression is at least about a 2-fold increase in expression relative to baseline expression and/or lower than baseline expression is at least about a 2-fold decrease in expression relative to baseline expression.

In another aspect, described herein is a method of treating a viral infection, treating a disease associated with viral infection, or decreasing, reducing, inhibiting, suppressing, limiting or controlling an adverse symptom or disorder resulting from the viral infection in a subject, the method comprising administering to the subject an effective amount of modified T-cells as detailed herein and/or a composition as detailed herein. In various embodiments, the method further comprises agonizing a population of or increasing the level, expression, or activity of T_{REG} cells in the subject. In various embodiments, the method comprises antagonizing a population of or decreasing or depleting the level, expression, or activity of T_{FH} or CD4-CTL cells in the subject.

Disclosed herein is a large-scale single-cell transcriptomic analysis of viral antigen-reactive CD4+ T cells from COVID-19 patients. In patients with severe disease compared to mild disease, increased proportions of cytotoxic follicular helper (TFH) cells and cytotoxic T helper cells (CD4-CTLs) responding to SARS-CoV-2 were discovered, and, alternatively, reduced proportion of SARS-CoV-2 reactive regulatory T cells. The CD4-CTLs were highly enriched for the expression of transcripts encoding chemokines that are involved in the recruitment of myeloid cells and dendritic cells to the sites of viral infection. Polyfunctional T helper (TH)1 cells and TH17 cell subsets were underrepresented in the repertoire of SARS-CoV-2-reactive CD4+ T cells compared to influenza-reactive CD4+ T cells.

In an aspect, a method of diagnosing a viral infection in a subject is provided, the method comprising obtaining a biological sample from the subject; quantifying a level of a biological feature associated with Th1 cells or Th17 cells from the biological sample; and comparing the level of the biological feature against a quantifiable reference value, wherein when the level of the biological feature is below the quantifiable reference value, the viral infection is associated with SARS-CoV-2.

In some embodiments, the quantifiable reference value comprises a biological feature associated with Th1 cells or Th17 cells isolated from a source infected with a non-SARS-CoV-2 virus. In other embodiments, the quantifiable reference value comprises a biological feature associated with the activity or number of Th1 cells or Th17 cells isolated from a source infected with influenza. In certain embodiments, the biological feature comprises the expression or activity of one or more genes set forth in Table 1 and/or Table 5. In some embodiments, the biological feature comprises the expression or activity of T-bet, IFN-γ, IL-2, TNF, IL-3, CSF2, IL-23A, CCL20, IL17A, or IL17F.

In an aspect, a method of diagnosing a viral infection in a subject is provided, the method comprising: obtaining a biological sample from the subject; quantifying a level of a biological feature associated with Tfh or CD4-CTL cells from the biological sample; and comparing the level of the biological feature associated with the Tfh or CD4-CTL cells against a quantifiable reference value, wherein when the level of the biological feature is higher than the quantifiable reference value, the viral infection is associated with SARS-CoV-2.

In some embodiments, the quantifiable reference value comprises a biological feature associated with the activity or number of Tfh or CD4-CTL cells isolated from a source infected with a non-SARS-CoV-2 virus. In other embodiments, quantifiable reference value comprises a biological feature associated with Tfh or CD4-CTL cells isolated from a source infected with an influenza virus. In still other embodiments, the biological feature comprises the expression or activity of one or more genes set forth in Table 2 and/or Table 3, or one or more of the T-cell receptor (TCR) sequences set forth in Table 6, or a homolog, variant, subsequence, or derivative thereof. In certain embodiments, the biological feature comprises expression or activity of one or more of CXCL13, IL21, CD200, BTLA, POU2AF1, PRF1, GZMB, GZMH, GNLY, or NKG7.

In an aspect, a method of diagnosing the severity of a virally-induced disease in a subject is provided, the method comprising: obtaining a biological sample from the subject; quantifying a level of a biological feature associated with Tfh cells from the biological sample; and comparing the level of the biological feature against a quantifiable reference value, wherein when the level of the biological feature is above the quantifiable reference value, the virally-induced disease is severe.

In some embodiments the quantifiable reference value comprises a biological feature associated with the number or activity of Tfh cells isolated from a second subject suffering from a non-severe case of the virally-induced disease. In other embodiments, the biological feature comprises expression or activity of one or more genes set forth in Table 3, or one or more of the TCR sequences set forth in Table 6, or a homolog, variant, subsequence, or derivative thereof.

In some embodiments, the biological feature comprises expression or activity of one or more of ZBED2, ZBTB32, TIGIT, LAG3, TIM3, PD1, DUSP4, CD70, PRF1, or GZMB. In certain embodiments, the virally-induced disease is COVID-19 or is associated with SARS-CoV-2.

In an aspect, a method of diagnosing the severity of a virally-induced disease in a subject is provided, the method comprising: obtaining a biological sample from the subject; quantifying a level of a biological feature associated with CD4-CTL cells from the biological sample; and comparing the level of the biological feature against a quantifiable reference value, wherein when the level of the biological feature is above the quantifiable reference value, the virally-induced disease is severe.

In some embodiments, the quantifiable reference value comprises a biological feature associated with the number or activity of CD4-CTL cells isolated from a second subject suffering from a non-severe case of the virally-induced disease. In certain embodiments, the biological feature comprises expression or activity of one or more genes set forth in Table 2 or Table 4, or one or more of the TCR sequences set forth in Table 6, or a homolog, variant, subsequence, or derivative thereof. In still other embodiments, the biological feature comprises expression or activity of one or more of CD72, GPR18, HOPX, ZEB2, CCL3, CCL4, CCL5, CCR1, CCR3, CCR5, XCL1, or XCL2. In some embodiments, the virally-induced disease is COVID-19 or is associated with SARS-CoV-2.

In an aspect, a method of diagnosing severity of a virally-induced disease in a subject is provided, the method comprising: obtaining a biological sample from the subject; quantifying a level of a biological feature associated with T_{REG} cells from the biological sample; and comparing the level of the biological feature associated with T_{REG} against a quantifiable reference value, wherein when the level of the biological feature is below the quantifiable reference value, the virally-induced disease is severe.

In some embodiments, the quantifiable reference value comprises a biological feature associated with the number or activity of T_{REG} cells isolated from a second subject suffering from a mild form of the virally-induced disease. In certain embodiments, the biological feature comprises expression or activity of FOXP3, or one or more of the TCR sequences set forth in Table 6, or a homolog, variant, subsequence, or derivative thereof. In other embodiments, the virally-induced disease is COVID-19 or is associated with SARS-CoV-2.

In an aspect, a method of diagnosing severity of a virally-induced disease in a subject is provided, the method comprising: obtaining a biological sample from the subject; quantifying a level of a biological feature associated with Th1 cells from the biological sample; and comparing the level of the biological feature associated with Th1 cells against a quantifiable reference value, wherein when the level of the biological feature is below the quantifiable reference value, the virally-induced disease is severe.

In certain embodiments, the quantifiable reference value comprises a biological feature associated with the number or activity Th1 cells isolated from a second subject suffering from a mild form of the virally-induced disease. In certain embodiments, the biological feature comprises the expression or activity of T-bet, IFN-γ, IL-2, TNF, IL-3, CSF2, IL-23A, or CCL20. In some embodiments, the virally-induced disease is COVID-19 or is associated with SARS-CoV-2.

In an aspect, a method of treating a coronavirus infection, disease associated with coronavirus infection, or decreasing, reducing, inhibiting, suppressing, limiting or controlling an adverse symptom or disorder resulting from the coronavirus infection in a subject is provided, the method comprising: administering to the subject a therapeutically effective amount of T_{REG} or Th1 cells.

In an aspect, a method of treating a coronavirus infection, disease associated with coronavirus infection, or decreasing, reducing, inhibiting, suppressing, limiting or controlling an adverse symptom or disorder resulting from the coronavirus infection in a subject is provided, the method comprising: administering to the subject a therapeutic effective amount of an agent that can selectively reduce Tfh or CD4+ CTL cells in the subject. In certain aspects, the agent comprises an antibody that selectively binds to a protein expressed by Tfh or CD4+ CTL cells.

In an aspect, a method of treating a coronavirus infection, disease associated with coronavirus infection, or decreasing, reducing, inhibiting, suppressing, limiting or controlling an adverse symptom or disorder resulting from the coronavirus in a subject is provided, the method comprising administering to the subject an effective amount of a population of T-cells that exhibit higher than or lower than baseline expression of one or more genes set forth in Table 1, Table 2, Table 3, Table 4, and/or Table 5, or that express a T-cell receptor (TCR) comprising at least one of the amino acid sequences set forth in Tables 6 and 7, or a homolog, variant, subsequence, or derivative thereof. In certain embodiments, the method comprises administering a population of T-cells that exhibit higher than baseline expression of one or more genes set forth in Table 1 or Table 5, or that express a TCR comprising at least one of the amino acid sequences set forth in Table 7, or a homolog, variant, subsequence, or derivative thereof. In some embodiments, the T-cell is a Th1, Th17, or T_{REG} cell. In other embodiments, the one or more genes are selected from the group of T-bet, IFN-γ, IL-2, TNF, IL-3, CSF2, IL-23A, CCL20, IL17A, FOXP3, and IL17F. In certain particular embodiments, the at least one amino acid sequence is selected from Table 7.

In some embodiments, the method comprises administering a population of T-cells that exhibit lower than baseline expression of one or more genes set forth in Table 2, Table 3, or Table 4, or that express a TCR comprising at least one of the amino acid sequences set forth in Table 6, or a homolog, variant, subsequence, or derivative thereof. In other embodiments, the one or more genes are selected from the group of ZBED2, ZBTB32, TIGIT, LAG3, TIM3, PD1, DUSP4, CD70, PRF1, and GZMB. In certain embodiments, the T-cell is a Tfh cell. In other embodiments, the one or more genes are selected from the group of CD72, GPR18, HOPX, ZEB2, CCL3, CCL4, CCL5, CCR1, CCR3, CCR5, XCL1, and XCL2. In some embodiments, the T cell is a CD4-CTL T cell. In other embodiments, the at least one amino acid sequence is selected from Table 6.

In an aspect, a method of treating a coronavirus infection, disease associated with coronavirus infection, or decreasing, reducing, inhibiting, suppressing, limiting or controlling an adverse symptom or disorder resulting from the coronavirus in a subject is provided, the method comprising administering to the subject an effective amount of an agent that induces higher than or lower than baseline expression of one or more genes set forth in Table 1, Table 2, Table 3, Table 4, and/or Table 5 in T cells, or of a TCR of at least one of the amino acid sequences set forth in Tables 6 and 7, or a homolog, variant, subsequence, or derivative thereof.

In an aspect, a method of treating a coronavirus infection, disease associated with coronavirus infection, or decreasing, reducing, inhibiting, suppressing, limiting or controlling an adverse symptom or disorder resulting from the coronavirus in a subject is provided, the method comprising administering an effective amount of an agent that induces or inhibits T cell activity of one or more proteins encoded by one or more genes set forth in Table 1, Table 2, Table 3, Table 4, and/or Table 5, or that modulates expression of a T-cell receptor (TCR) comprising at least one of the amino acid sequences set forth in Tables 6 and 7, or a homolog, variant, subsequence, or derivative thereof.

In some embodiments, the agent is an antibody, a small molecule, a protein, a peptide, a ligand mimetic, or a nucleic acid. In other embodiments, the baseline expression is normalized mean gene expression. In certain embodiment, higher than baseline expression is at least about a 2-fold increase in expression relative to baseline expression and/or lower than baseline expression is at least about a 2-fold decrease in expression relative to baseline expression.

In an aspect, a modified T-cell is provided, wherein the T cell is modified to exhibit higher than or lower than baseline expression of one or more genes set forth in Table 1, Table 2, Table 3, Table 4, and/or Table 5, or one or more T-cell receptor (TCR) comprising at least one of the amino acid sequences set forth in Tables 6 and 7, or a homolog, variant, subsequence, or derivative thereof. In some embodiments, the modified T cell exhibits higher than baseline expression of one or more genes set forth in Table 1 or Table 5, or expresses a TCR comprising at least one of the amino acid sequences set forth in Table 7, or a homolog, variant, subsequence, or derivative thereof.

In certain embodiments, the one or more genes are selected from the group of T-bet, IFN-γ, IL-2, TNF, IL-3, CSF2, IL-23A, CCL20, IL17A, FOXP3, and IL17F. In some embodiments, the at least one amino acid sequence is selected from Table 7. In some embodiments, the modified T cell is a T_{REG}, Th1, or Th17 cell. In specific embodiments, the baseline expression is normalized mean gene expression. In some embodiments, higher than baseline expression is at least about a 2-fold increase in expression relative to baseline expression and/or lower than baseline expression is at least about a 2-fold decrease in expression relative to baseline expression.

In certain embodiments, the modified T-cell is genetically modified, optionally using one or more of gene editing, recombinant methods and/or a CRISPR/Cas system. In other embodiments, the modified T cell is further modified to express a protein that binds to a cytokine, chemokine, lymphokine, or a receptor each thereof. In particular embodiments, the protein comprises an antibody or an antigen binding fragment thereof. In some embodiments, the antibody is an IgG, IgA, IgM, IgE or IgD, or a subclass thereof. In certain embodiments, the antibody is an IgG selected from the group of IgG1, IgG2, IgG3 or IgG4. In other embodiments, the antigen binding fragment is selected from the group of a Fab, Fab′, F(ab′)2, Fv, Fd, single-chain Fvs (scFv), disulfide-linked Fvs (sdFv) or VL or VH.

In some embodiments, the modified T-cell comprises a chimeric antigen receptor (CAR). In other embodiments, the chimeric antigen receptor (CAR) comprises: (a) an antigen binding domain; (b) a hinge domain; (c) a transmembrane domain; (d) and an intracellular domain. In some embodiments, the CAR further comprises one or more costimulatory signaling regions.

In certain embodiments, the antigen binding domain comprises an anti-CD19 antigen binding domain, the transmembrane domain comprises a CD28 or a CD8 α transmembrane domain, the one or more costimulatory regions selected from a CD28 costimulatory signaling region, a 4-1BB costimulatory signaling region, an ICOS costimulatory signaling region, and an OX40 costimulatory region or a CD3 zeta signaling domain.

In some embodiments, the anti-CD19 binding domain comprises a single-chain variable fragment (scFv) that specifically recognizes a humanized anti-CD19 binding domain. In other embodiments, the anti-CD19 binding domain scFv of the CAR comprises a heavy chain variable region and a light chain variable region. In some embodiments, the anti-CD19 binding domain of the CAR further comprises a linker polypeptide located between the anti-CD19 binding domain scFv heavy chain variable region and the anti-CD19 binding domain scFv light chain variable region. In certain embodiments, the linker polypeptide of the CAR comprises a polypeptide of the sequence (GGGGS)n wherein n is an integer from 1 to 6.

In certain embodiments, the CAR further comprises a detectable marker attached to the CAR. In other embodiments, the CAR further comprises a purification marker attached to the CAR. In some embodiments, the modified T-cell comprises a polynucleotide encoding the CAR, and optionally, wherein the polynucleotide encodes and anti-CD19 binding domain. In certain specific embodiments, the polynucleotide further comprises a promoter operatively linked to the polynucleotide to express the polynucleotide in the modified T-cell.

In some embodiments, the polynucleotide further comprises a 2A self-cleaving peptide (T2A) encoding polynucleotide sequence located upstream of a polynucleotide encoding the anti-CD19 binding domain. In other embodiments, the polynucleotide further comprises a polynucleotide encoding a signal peptide located upstream of a polynucleotide encoding the anti-CD19 binding domain.

In certain embodiments, the polynucleotide further comprises a vector. In other embodiments, the vector is a plasmid. In some embodiments, the vector is a viral vector selected from the group of a retroviral vector, a lentiviral vector, an adenoviral vector, and an adeno-associated viral vector.

In an aspect, a composition is provided comprising a population of modified T-cells described herein.

In an aspect, a method of treating a viral infection, disease associated with viral infection, or decreasing, reducing, inhibiting, suppressing, limiting or controlling an adverse symptom or disorder resulting from the virus in a subject is provided, the method comprising administering to the subject an effective amount of the modified T-cells and/or the compositions described herein.

In certain embodiments, the viral infection may result from any of the following viral families: Arenaviridae, Arterivirus, Astroviridae, Baculoviridae, Badnavirus, Bamaviridae, Birnaviridae, Bromoviridae, Bunyaviridae, Caliciviridae, Capillovirus, Carlavirus, Caulimovirus, Circoviridae, Closterovirus, Comoviridae, Coronaviridae (e.g., Coronavirus, such as severe acute respiratory syndrome (SARS) virus), Corticoviridae, Cystoviridae, Deltavirus, Dianthovirus, Enamovirus, Filoviridae (e.g., Marburg vims and Ebola virus (e.g., Zaire, Reston, Ivory Coast, or Sudan strain)), Flaviviridae, (e.g., Hepatitis C vims, Dengue vims 1, Dengue vims 2, Dengue virus 3, and Dengue virus 4), Hepadnaviridae, Herpesviridae (e.g., Human herpesvirus 1, 3, 4, 5, and 6, and Cytomegalovirus), Hypoviridae, Iridoviridae, Leviviridae, Lipothrixviridae, Microviridae, Orthomyxoviridae (e.g., Influenzavirus A and B and C), Papovaviridae, Paramyxoviridae (e.g., measles, mumps, and human respiratory syncytial virus), Parvoviridae, Picomaviridae (e.g., poliovirus, rhinovirus, hepatovims, and aphthovirus), Poxviridae (e.g., vaccinia and smallpox vims), Reoviridae (e.g., rotavims), Retroviridae (e.g., lentivirus, such as human immunodeficiency vims (HIV) 1 and HIV 2), Rhabdoviridae (for example, rabies vims, measles virus, respiratory syncytial virus, etc.), Togaviridae (for example, mbella virus, dengue virus, etc.), and Totiviridae. Suitable viral antigens also include all or part of Dengue protein M, Dengue protein E, Dengue DiNS1, Dengue D1NS2, and Dengue D1NS3.

The viral infection or virus may be derived from a particular strain such as a papilloma vims, a herpes vims, e.g., herpes simplex 1 and 2; a hepatitis vims, for example, hepatitis A vims (HAV), hepatitis B vims (HBV), hepatitis C virus (HCV), the delta hepatitis D vims (HDV), hepatitis E virus (HEV) and hepatitis G vims (HGV), the tick-borne encephalitis viruses; parainfluenza, varicella-zoster, cytomeglavirus, Epstein-Barr, rotavirus, rhinovims, adenovims, coxsackieviruses, equine encephalitis, Japanese encephalitis, yellow fever, Rift Valley fever, and lymphocytic choriomeningitis.

In an aspect, a method of treating a coronavirus infection, disease associated with coronavirus infection, or decreasing, reducing, inhibiting, suppressing, limiting or controlling an adverse symptom or disorder resulting from the coronavirus in a subject is provided, the method comprising administering to the subject an effective amount of the modified T-cells and/or the compositions described herein.

In some embodiments, the coronavirus infection is SARS-CoV-2. In other embodiments, the disease associated with coronavirus infection is COVID-19.

In certain embodiments described herein, the methods and treatments described comprise agonizing a population of or increasing the level, expression, or activity of Th1, Th17, or T_{REG} cells in the subject.

In certain embodiments described herein, the methods and treatments described comprises antagonizing a population of or decreasing or depleting the level, expression, or activity of Tfh or CD4-CTL cells in the subject.

### BRIEF DESCRIPTION OF THE FIGURES

In the present Application:

FIGS. 1A-1C. FIG. 1A depicts a study overview of a screen of healthy subjects stimulated with viral peptides. FIG. 1B. provides representative FACS plots showing surface staining of CD154 (CD40L) and CD69 in memory CD4⁺ T cells stimulated for 6H with SARS-CoV-2 peptide pools, post-enrichment, in hospitalized and non-hospitalized infected individuals (left) and summary of number of cells sorted (right). FIG. 1C provides representative FACS plots (left) showing surface expression of CD137 (4-1BB) and HLA-DR in memory CD4⁺ T cells ex vivo and in CD154⁺ CD69⁺ memory CD4⁺ T cells following stimulation, post-enrichment and corresponding summary plots (right).

FIGS. 2A-2D. FIG. 2A depicts a gating strategy to sort, lymphocytes, single cells (Height vs Area forward scatter (FSC)), live, CD3⁺ CD4⁺ memory (CD45RA⁺ CCR7⁺ naïve cells excluded) activated CD154⁺ CD69⁺ T cells. Surface expression of activation markers was analyzed on memory CD4⁺ T cells. FIG. 2B depicts representative FACS plots (left) showing surface expression off PD-1 and CD38 in memory CD4⁺ T cells ex vivo and in CD154⁺ CD69⁺ memory CD4⁺ T cells following stimulation post-enrichment and summary of PD-1 and CD38 frequencies in CD154⁺ CD69⁺ memory CD4⁺ T cell following stimulation post-enrichment in hospitalized and non-hospitalized individuals (right). FIG. 2C depicts representative FACS plots showing surface staining of CD154 and CD69 in memory CD4⁺ T cells stimulated with individual virus megapools pre-enrichment (top) and post-enrichment (bottom) in healthy non-exposed donors. Summary of CD154⁺ CD69⁺ memory CD4⁺ T cell frequencies following stimulation with individual virus megapools without enrichment. FIG. 2D depicts representative FACS plots showing surface staining of CD154 in memory CD4⁺ T cells stimulated with Influenza megapool, post-enrichment, in healthy donors pre- and post-vaccination.

FIGS. 3A-3F: Transcriptome of CD4⁺ T cells responding to SARS-CoV-2. FIG. 3A depicts an analysis of 10× single-cell RNA-seq from sorted CD154⁺ CD69⁺ memory CD4⁺ T cells following 6H stimulation displayed by manifold approximation and projection (UMAP). Seurat clustering of 91,140 activated CD4⁺ T cells colored based on cluster type. FIG. 3B depicts UMAPs of sorted, activated memory CD4⁺ T cells for individual virus megapool stimulation (left) and normalized proportion per cluster (right). FIG. 3C depicts a heatmap comparing gene expression in all clusters. Transcripts that change expression >0.25 fold and adjusted P value of ≤0.05 are depicted. FIG. 3D depicts average expression and percent expression of selected marker genes in each cluster. FIG. 3E depicts violin plots comparing expression of T_{FH} (top), T_{H}1 (middle) and T_{H}17 (bottom) marker transcripts in designated clusters compared to an aggregation of remaining cells. FIG. 3F depicts a UMAP depicting mean expression of transcripts associated with T_{FH}, CD4-CTL, T_{H}17 and interferon (IFN) response gene signatures.

FIGS. 4A-4G. FIG. 4A depicts the number of genes recovered from all libraries sequenced. FIG. 4B depicts distribution of individual clusters in all batches of sorted cells. FIG. 4C depicts pie charts with proportion per cluster for individual virus stimulations. Notable clusters are referenced with numbers. FIG. 4D depicts violin plots showing gene signature score for T_{H}17, interferon (IFN) response, T_{FH}, and CD4-CTLs. The different shading indicates mean expression of genes. FIG. 4E depicts violin plots comparing expression of T_{H}1, T_{H}17, IFN response, T_{FH} and CD4-CTL marker transcripts in designated clusters compared to an aggregation of remaining cells. FIG. 4F depicts a scatter plot displaying co-expression of IL2 and TNF in IFNG-expressing, virus-reactive memory CD4⁺ T cells. FIG. 4G depicts a gene set enrichment analysis (GSEA) for T_{H}17, cell cycling, T_{FH} and CD4-CTL features in a given cluster compared to the rest of the dataset.

FIGS. 5A-5E: CTL and T_{F}H CD4⁺ T cell profiles enriched in SARS-CoV-2 infected individuals. FIG. 5A depicts UMAP of sorted, activated memory CD4⁺ T cells for non-hospitalized and hospitalized SARS-CoV-2 infected individuals and proportions per cluster (right). FIG. 5B depicts violin plots showing expression of ZBTB32 and ZBED2 (top) clusters 6,0,7 from SARS-CoV-2 infected individuals (top) and average expression and percent expression of selected genes in each cluster 6,0,7 (bottom). FIG. 5C depicts a scatter plot displaying co-expression of PRF1 and GZMB in in clusters 0,6,7 from SARS-CoV-2 infected individuals. Frequencies indicate percentage of cells inside each of the graph sections. FIG. 5D depicts violin plots comparing expression of HOPX and ZEB2, SLAMF7, CD72 and GPR18 in clusters 4,8 and an aggregate of remaining cells. FIG. 5E depicts a UMAP showing Seurat normalized expression of CCL3, CCL4, CCL5, XCL1 and XCL2.

FIGS. 6A-6G. FIG. 6A depicts frequencies of T_{FH} CD4⁺ T cells (clusters 0,6,7) as a proportion of the total CD4⁺ T cell pool in non-hospitalized and hospitalized SARS-CoV-2 infected individuals. Frequencies of cluster 6,0,7 as a proportion of all T_{FH} in non-hospitalized and hospitalized SARS-CoV-2 infected individuals. FIG. 6B depicts volcano plot showing differentially expressed genes between cluster 6 and 0 from SARS-CoV-2 infected individuals. FIG. 6C depicts violin plots showing expression of TIGIT, LAG3, HAVCR2, PDCD1, DUSP4, CD70 and DOK5 in clusters 6,0,7 (SARS-CoV-2 infected individuals). FIG. 6D depicts violin plots showing expression of PRF1 and GZMB in clusters 6,0,7 (SARS-CoV-2 infected individuals). FIG. 6E depicts an average expression and percent expression of selected genes in clusters 4, 8 and an aggregate of remaining cells. FIG. 6F depicts violin plots showing expression CCL3, CCL4, CCL5, XCL1 and XCL2 in clusters 4,8 and an aggregate of remaining cells. FIG. 6G depicts scatter plot displaying co-expression of XCL1 and XCL2 in in clusters 4,8,11 from SARS-CoV-2 infected individuals. Frequencies indicate percentage of cells inside each of the graph sections.

FIGS. 7A-7I: Clonotypic expansion and late activation in SARS-CoV-2 infected individuals. FIG. 7A shows a UMAP depicting clone size of sorted, activated memory CD4⁺ T cells from SARS-CoV-2 infected individuals following 6H stimulation (left). FIG. 7B depicts single-cell trajectory constructed using Monocle 3. FIG. 7C depicts TCR sharing between individual clusters. Bars indicate number of cells intersecting in indicated clusters. FIG. 7D depicts analysis of 10× single-cell RNA-seq from sorted CD137⁺ CD69⁺ memory CD4⁺ T cells displayed following 24H stimulation by UMAP. Seurat clustering of 31,341 activated CD4⁺ T cells colored based on cluster type. FIG. 7E depicts a heatmap comparing gene expression in all clusters. Transcripts that change expression >0.25 fold and adjusted P value of ≤0.05 are depicted. FIG. 7F depicts average expression and percent expression of selected marker genes in each cluster. FIG. 7G depicts a UMAP showing Seurat normalized expression of FOXP3 (left) and GSEA for T_{REG} features in cluster A (right). FIG. 7H depicts normalized proportions of analyzed CD4⁺ T cells from 24H dataset per cluster from non-hospitalized and hospitalized (red) SARS-CoV-2 infected individuals. FIG. 7I depicts pie charts with proportion per cluster in non-hospitalized and non-hospitalized SARS-CoV-2 infected individuals following 24H stimulation.

FIGS. 8A-8D. FIG. 8A depicts a proportion of expanded clonotypes (clone size ≥2) in hospitalized and non-hospitalized SARS-CoV-2 infected individuals following 6H stimulation. FIG. 8B depicts a representative FACS plots showing surface staining of CD137 and CD69 in memory CD4⁺ T cells stimulated for 24H with SARS-CoV-2 peptide pools, post-enrichment, in hospitalized and non-hospitalized individuals. Summary of number of cells sorted (right). FIG. 8C depicts GSEA for cytotoxicity, T_{FH} and T_{H}17 features in a given cluster compared to the rest of the 24H dataset. FIG. 8D depicts a UMAP depicting clone size of sorted, activated memory CD4⁺ T cells following 24H stimulation (left) and proportion of expanded clonotypes (clone size ≥2) in each cluster (right).

FIGS. 9A-9C. FIG. 9A depicts a study overview of a screen of healthy subjects stimulated with viral peptides. FIG. 9B depicts a representative FACS plots showing surface staining of CD154 (CD40L) and CD69 memory CD4+ T cells stimulated for 6 h with SARS-CoV-2 peptide pools, post-enrichment (CD154-based), in 22 hospitalized and 18 non-hospitalized COVID-19 patients (left), and summary of numbers of cells sorted (right); data are mean±SEM. FIG. 9C depicts a representative FACS plots (left) showing surface expression of CD137 (4-1BB) and HLA-DR in memory CD4+ T cells ex vivo (without in vitro stimulation) and in CD154+ CD69+ memory CD4+ T cells following stimulation, post-enrichment (CD154-based). (Right) Percentage of CD154+ CD69+ memory CD4+ T cells expressing CD137 (4-1BB) or HLA-DR in 17 hospitalized and 18 non-hospitalized COVID-19 patients; data are mean±SEM.

FIGS. 10A-10F: SARS-CoV-2-Reactive CD4+ T Cells Are Enriched for TFH Cells and CD4-CTLs. FIG. 10A depicts single-cell transcriptomes of sorted CD154+ CD69+ memory CD4+ T cells following 6 h stimulation with virus-specific peptide megapools are displayed by uniform manifold approximation and projection (UMAP). Seurat-based clustering of 102,230 cells colored based on cluster type. FIG. 10B depicts UMAPs showing memory CD4+ T cells for individual virus-specific megapool stimulation conditions (left), and normalized proportions of each virus-reactive cells per cluster is shown (right). FIG. 10C depicts a heatmap showing expression of the most significantly enriched transcripts in each cluster (see Table S2F). Seurat marker gene analysis (comparison of cluster of interest versus all other cells). The top 200 transcripts are shown based on adjusted P value <0.05, log 2 fold change >0.25 and >10% difference in the percentage of cells expressing selected transcript between two groups of cells compared. FIG. 10D depicts a plot that shows average expression (color scale) and percent of expressing cells (size scale) for selected marker gene transcripts in each cluster. FIG. 10E depicts violin plots showing normalized expression level (log 2(CPM+1)) of TFH (top), TH1 (middle), and TH17 (bottom) marker transcripts in designated clusters compared to an aggregation of remaining cells (Rest). Color indicates percentage of cells expressing indicated transcript. FIG. 10F depicts a UMAP showing TFH, CD4-CTL, TH17, and interferon (IFN) response signature scores for each cell.

FIGS. 11A-11H: SARS-CoV-2-Reactive CD4+ T Cell Subsets Associated with Disease Severity. FIG. 11A depicts unsupervised clustering of COVID-19 patients based on the proportions of SARS-CoV-2-reactive CD4+ T cells in different clusters following 6 h peptide stimulation. Clusters with fewer than 5% of the total dataset are not depicted. Gender and hospitalization status per patient are indicated by different color schemes above the heatmap. FIG. 11B depicts a percentage of TFH cells (clusters 0, 5, and 7) in the total SARS-CoV-2-reactive CD4+ T cell pool for non-hospitalized and hospitalized COVID-19 patients; dots indicate data from a single subject. Data are mean±SEM; significance for comparisons was computed using Mann-Whitney U test; ns, non-significant P value. FIG. 11C depicts a proportion of clusters 5 and 0 cells in SARS-CoV-2-reactive TFH cells (clusters 0, 5, and 7) in non-hospitalized and hospitalized COVID-19 patients. Data are mean±SEM; significance for comparisons was computed using Mann-Whitney U test; ****p<0.0001. FIG. 11D depicts violin plots showing normalized expression level (log 2(CPM+1)) of ZBTB32 and ZBED2 transcripts in SARS-CoV-2-reactive cells from clusters 0, 5, and 7 (top); color indicates percentage of cells expressing indicated transcript. Plots below show average expression and percent of cells expressing selected transcripts in indicated clusters. FIG. 11E depicts a scatterplot displaying normalized co-expression level (log 2(CPM+1)) between PRF1 and GZMB transcripts in SARS-CoV-2-reactive cells present in clusters 5 (left) and 0 (right). Numbers indicate percentage of cells in each quadrant. FIG. 11F depicts a correlation between percentage of SARS-CoV-2-reactive CD4+ TFH cells and S1/S2 antibody titers in 15 non-hospitalized (left) and 20 hospitalized (right) COVID-19 patients. Correlation coefficient r and the related P value were computed using Spearman correlation; *p<0.05. FIG. 11G depicts a correlation between percentage of SARS-CoV-2-reactive CD4+ TFH cells form cluster 5 as a frequency of total CD4+ TFH and S1/S2 antibody titers (left two plots) and interval between symptom onset and blood draw (right two plots) in 15 non-hospitalized and 20 hospitalized (left) COVID-19 patients. Correlation coefficient r and the related P value were computed using Spearman correlation; **p<0.01; ***p<0.001; ns, non-significant P value. FIG. 11H depicts a single-cell trajectory analysis of cells in cluster 5 and 0 showing pseudotime, expression of indicated genes, and IFN response signature score.

FIGS. 12A-12G: SARS-CoV-2-Reactive CD4-CTLs and Single-Cell TCR Sequence Analysis. FIG. 12A depicts UMAPs showing Seurat-normalized expression level of PRF1, GZMB, GNLY, and NKG7 transcripts in each virus-reactive cell. FIG. 12B depicts a percentage of CD4-CTLs (clusters 6 and 9) in the total SARS-CoV-2-reactive CD4+ T cell pool for non-hospitalized and hospitalized COVID-19 patients; dots indicate data from a single subject. Data are mean±SEM; significance for comparisons was computed using Mann-Whitney U test; ns, non-significant P value. FIG. 12C depicts violin plots showing normalized expression level (log 2(CPM+1)) of transcription factors HOPX and ZEB2 and effector molecules CD72, GPR18, and SLAMF7 transcripts in virus-reactive cells from designated clusters (6 and 9) compared to an aggregation of remaining cells (Rest). FIG. 12D depicts UMAPs showing Seurat-normalized expression of CCL3, CCL4, CCL5, XCL1, and XCL2 transcripts in each virus-reactive cell. FIG. 12E depicts a UMAP showing TCR clone size (log 2, color scale) of SARS-CoV-2-reactive cells from COVID-19 patients (6 h stimulation condition). FIG. 12F depicts a histogram bar graph (top) displaying single-cell TCR sequence analysis of SARS-CoV-2-reactive cells. Each bar shows the number of TCRs shared between cells from individual clusters (rows, connected by lines). Connected lines (bottom) indicates what clusters are sharing TCRs. Clusters 6 (green), 9 (blue), and 11 (pink), i.e., CD4-CTLs, are highlighted. FIG. 12G depicts a single-cell trajectory analysis showing relationship between cells in different clusters (line), constructed using Monocle 3. Only SARS-CoV-2-reactive cells from COVID-19 patients (6 h stimulation condition) are shown.

FIGS. 13A-13I: Analysis of SARS-CoV-2-Reactive CD4+ T Cells from 24 h Stimulation Condition. FIG. 13A depicts single-cell transcriptomes of sorted CD137+ CD69+ memory CD4+ T cells following 24 h stimulation with SARS-CoV-2-specific peptide megapools are displayed by UMAP. Seurat-based clustering of 38,519 cells colored based on cluster type. FIG. 13B depicts a heatmap showing expression of the most significantly enriched transcripts in each cluster (see Table S5C). Seurat marker gene analysis-comparison of cluster of interest versus all other cells-shown are top 200 transcripts with adjusted P value <0.05, log 2 fold change >0.25, and >10% difference in the percentage of cells expressing differentially expressed transcript between two groups compared. FIG. 13C depicts a plot showing average expression (color scale) and percent of expression (size scale) of selected marker gene transcripts in each cluster. FIG. 13D depicts a UMAP showing Seurat-normalized expression level of FOXP3 transcripts (left). Percentage of T_{REG} cells (cluster A) in the total SARS-CoV-2-reactive CD4+ T cell pool for non-hospitalized and hospitalized COVID-19 patients; dots indicate data from a single subject (right plot). Data are mean±SEM; significance for comparisons was computed using Mann-Whitney U test; ***p<0.001. FIG. 13E depicts average frequency of cells per cluster from hospitalized and non-hospitalized COVID-19 patients. FIG. 13F depicts a UMAP showing CD4-CTL signature score for each cell (left) and percentage of CD4-CTLs (clusters B and F) in the total SARS-CoV-2-reactive CD4+ T cell pool for non-hospitalized and hospitalized COVID-19 patients; dots indicate data from a single subject (left plot). Data are mean±SEM. Significance for comparisons was computed using Mann-Whitney U test; ns, non-significant P value.

FIG. 13G depicts a correlation between percentage of SARS-CoV-2-reactive CD4+T_{REG} and percentage of SARS-CoV-2-reactive CD4-CTLs in 13 non-hospitalized and 17 hospitalized (left) COVID-19 patients. Correlation coefficient r and the related P value were computed using Spearman correlation; ****p<0.0001. FIG. 13H UMAP showing Seurat-normalized expression level of IL1R2 transcripts (left) and percentage of TFR cells (IL1R2-expressing cells in cluster A) in the total SARS-CoV-2-reactive CD4+ T cell pool for non-hospitalized and hospitalized COVID-19 patients; dots indicate data from a single subject (left plot). Data are mean±SEM; significance for comparisons were computed using Mann-Whitney U test; ***p<0.001. (I) Correlation between percentage of SARS-CoV-2-reactive cytotoxic TFH cells (proportion of TFH cells in cluster 5, from 6 h stimulation dataset as in FIG. 3C) and percentage of TFR cells (IL1R2-expressing cells in cluster A) in 25 COVID-19 patients (left). Correlation coefficient r was computed using Spearman correlation; ns, non-significant P value.

FIGS. 14A-14E: CD4+ T Cell Responses in COVID-19 Illness (related to FIGS. 9A-9C): FIG. 14A depicts a gating strategy to sort: lymphocytes size-scatter gate, single cells (Height versus Area forward scatter (FSC)), live, CD3+ CD4+ memory (CD45RA+ CCR7+ naive cells excluded) activated CD154+ CD69+ cells. Surface expression of activation markers was analyzed on memory CD4+ T cells. FIG. 14B representative FACS plots (left) showing surface expression of PD-1 and CD38 in memory CD4+ T cells ex vivo and in CD154+ CD69+ memory CD4+ T cells following 6 h of stimulation, post-enrichment (CD154-based). (Middle) Plots depicting percentage of CD154+ CD69+ memory CD4+ T cells expressing PD-1 or CD38 following stimulation and post-enrichment (CD154-based) in 17 hospitalized and 18 non-hospitalized COVID-19 patients. (Right) Plot showing the total number of sorted CD154+ CD69+ memory CD4+ T cells per million PBMCs; data are mean±SEM. FIG. 14C depicts representative FACS plots showing surface staining of CD154 and CD69 in memory CD4+ T cells stimulated for 6 h with individual virus megapools, pre-enrichment (top) and post-enrichment (CD154-based) (bottom) in healthy non-exposed subjects. (Right) Percentage of memory CD4+ T cells co-expressing CD154 and CD69 following stimulation with individual virus megapools (pre-enrichment); data are mean±SEM. FIG. 14D depicts representative FACS plots (left) showing surface staining of CD154 in memory CD4+ T cells stimulated with Influenza megapool, pre-enrichment in healthy subjects pre and/or post-vaccination. (Right) Percentage of memory CD4+ T cells expressing CD154 following stimulation with Influenza megapool (pre-enrichment); data are mean±SEM. FIG. 14E depicts representative FACS plots showing surface staining of CD154 in memory CD4+ T cells stimulated with Influenza megapool, post-enrichment (CD154-based), in healthy subjects pre and/or post-vaccination.

FIGS. 15A-15G: SARS-CoV-2-Reactive CD4+ T Cells Are Enriched for TFH Cells and CD4-CTLs (related to FIGS. 10A-10F). FIG. 15A depicts the number of genes recovered for each 10× library sequenced. FIG. 15B depicts the proportion of cells in each cluster for the 6 batches of donors. FIG. 15C depicts donut charts show proportion of individual virus-reactive CD4+ T cells per cluster for different viruses. Notable clusters are highlighted. FIG. 15D depicts a violin plots showing enrichment patterns of TH17, IFN response, TFH, and CD4-CTLs gene signatures for each cluster. Color indicates mean signature score of cells within a cluster. FIG. 15E depicts violin plots showing normalized expression level (log 2(CPM+ 1)) of select TH1, TH17, IFN response, TFH and CD4-CTL marker transcripts in designated clusters compared to an aggregation of remaining cells (Rest). Color indicates the percentage of cells expressing indicated transcript. FIG. 15F depicts a scatterplot displaying co-expression level (log 2(CPM+1)) of IL2 and TNF transcripts in IFNG-expressing, virus-reactive memory CD4+ T cells in cluster 1. Numbers indicate percentage of cells in each quadrant. FIG. 15G depicts a gene set enrichment analysis (GSEA) for TH17, IFN response, cell cycling, TFH and CD4-CTL signature genes in a given cluster compared to the rest of the cells; *p<0.05; ***p<0.01; ***p<0.001.

FIGS. 16A-16K: SARS-CoV-2-Reactive CD4+ T Cell Subsets Associated with Disease Severity (related to FIGS. 11A-11H). FIG. 16A depicts average frequency of cells per cluster from hospitalized and non-hospitalized COVID-19 patients. FIG. 16B depicts the proportion of cluster 5 cells in SARS-CoV-2-reactive cytotoxic TFH cells (cluster 0, 5, and 7) in non-hospitalized and hospitalized COVID-19 patients who provided blood samples under 21 days (left) and over 21 days (right) after onset of symptoms. Data are mean±S.E.M; significance for comparisons was computed using Mann-Whitney U test; **p<0.01; ***p<0.001. FIG. 16C depicts the Proportion of cluster 7 cells in SARS-CoV-2-reactive TFH cells in non-hospitalized and hospitalized COVID-19 patients. Data are mean±SEM. Significance for comparisons was computed using Mann-Whitney U test; ns identifies non-significant P value. FIG. 16D depicts a volcano plot showing differentially expressed genes between SARS-CoV-2-reactive CD4+ T cells in cluster 5 versus cluster 0. FIG. 16E depicts violin plots showing expression level (log 2(CPM+ 1)) of PRF1 and GZMB transcripts in cells from clusters 0, 5 and 7. FIG. 16F depicts a scatterplot displaying co-expression level (log 2(CPM+ 1)) of PRF1 and GZMB transcripts in SARS-CoV-2-reactive cells present in cluster 7. Numbers indicate percentage of cells in each quadrant. FIG. 16G depicts the concentration of S1/S2 antibodies in the circulation of 22 hospitalized and 16 hospitalized non-hospitalized COVID-19 patients. Data are mean±S.E.M; significance for comparisons was computed using Mann-Whitney U test; *p<0.05. FIG. 16H depicts the correlation between percentage of SARS-CoV-2-reactive CD4+ TFH cells form cluster 0 as a frequency of total CD4+ TFH cells and S1/S2 antibody titers (left two plots) and interval between symptom onset and blood draw (right two plots) in 15 non-hospitalized and 20 hospitalized (left) COVID-19 patients. Correlation coefficient r and the related P value were computed using Spearman correlation; ***p<0.001. FIG. 16I depicts FACS plots showing S1/S2-specific B cells in 9 COVID-19 patients. Patient ID and proportion of SARS-CoV-2-reactive TFH cells in cluster 5 is specified. FIG. 16J depicts an ingenuity pathway analysis (IPA) of genes with increased expression (adjusted p<0.05 and log 2 fold change >1) between cells from cluster 5 versus cluster 0. Upstream regulatory network analysis of genes in IFN alpha pathway. FIG. 16K depicts a GSEA for IFN response signature genes in cluster 5 versus cluster 0; ***p<0.001.

FIGS. 17A-17H: Single-Cell TCR Sequence Analysis and Analysis of SARS-CoV-2-Reactive CD4+ T Cells from 24 h Stimulation and Ex Vivo Conditions (related to FIGS. 12A-12G). FIG. 17A depicts the average expression and percent expression of selected transcripts in indicated clusters. FIG. 17B depicts violin plots showing normalized expression level (log 2(CPM+1)) of CCL3, CCL4, CCL5, XCL1, and XCL2 transcripts in designated clusters (6 and 9) compared to an aggregation of remaining cells (Rest). FIG. 17C depicts scatterplots displaying co-expression level (log 2(CPM+1)) of XCL1 and XCL2 transcripts in SARS-CoV-2-reactive cells present in designated clusters. Numbers indicate percentage of cells in each quadrant. FIG. 17D depicts the proportion of expanded SARS-CoV-2-reactive CD4+ T cells (clone size >2) in hospitalized and non-hospitalized COVID-19 patients (6 h stimulation condition). Data are mean S.E.M; significance for comparisons were computed using Mann-Whitney U test; *p<0.05. FIG. 17E depicts single-cell transcriptomes of memory CD4+ T cells expressing activation markers (CD38, HLA-DR, PD-1) ex vivo (0 h; blue) and sorted CD154+CD69+ memory CD4+ T cells following 6 h stimulation with virus-specific peptide megapools (6 h; red) are displayed by UMAP. Seurat-based clustering of 122,292 cells. FIG. 17F depicts UMAP showing activation, TFH, and CD4-CTL signature scores for each cell. FIG. 17G depicts violin plots showing expression level (log 2(CPM+1)) of TNFRSF4, TNFRSF18, MIR155HG, CD200, IFNG, IL2, TNF, and POU2AF1 transcripts in 0- and 6 h time points. FIG. 17H depicts the number of cells from matched patients with shared (yellow) and unique (blue) TCRs between activation marker-positive cells sorted ex vivo (0 h) and 6 h peptide stimulated populations (left). Venn diagram illustrating the number of shared clones between activation marker-positive CD4+ T cells sorted ex vivo (0 h) and 6 h peptide stimulated populations.

FIGS. 18A-18F: Analysis of SARS-CoV-2-Reactive CD4+ T Cells from 24 h Stimulation Condition (related to FIGS. 13A-13I). FIG. 18A depicts representative FACS plots showing surface staining of CD137 and CD69 in memory CD4+ T cells stimulated for 24 h with SARS-CoV-2 peptide pools, post-enrichment (CD137-based), in hospitalized and non-hospitalized COVID-19 patients (left). Summary of number of cells sorted in 14 hospitalized and 17 non-hospitalized COVID-19 patients (right); data are mean±SEM. FIG. 18B depicts GSEA for T_{REG}, cytotoxicity, TFH and T_{H}17 signature genes in a given cluster compared to the rest of the cells; **p<0.01; ***p<0.001. FIG. 18C depicts unsupervised clustering of 17 hospitalized and 13 non-hospitalized COVID-19 patients based on the proportions of SARS CoV-2-reactive CD4+ T cells in different clusters following 24 h peptide stimulation. Clusters with fewer than 5% of the total dataset are not depicted. Hospitalization status (red versus green) and sex (pink versus blue) are indicated in the annotation rows immediately below the dendrogram. FIG. 18D depicts a UMAP showing TCR clone size (log 2, color scale) of SARS-CoV-2-reactive cells from COVID-19 patients (24 h stimulation condition). FIG. 18E depicts the proportion of clonally expanded (clone size >2) and non-expanded cells in each cluster (24 h stimulation condition). FIG. 18F depicts GSEA for TFH and TFR signature genes in IL1R2+ cells compared to IL1R2− cells in cluster A; *p<0.05; ***p<0.001.

### DETAILED DESCRIPTION

A detailed description of one or more embodiments of the disclosure is provided below along with any accompanying figures that illustrate the principles of the embodiments described herein. The disclosure is described in connection with such embodiments, but the disclosure is not limited to any embodiment. Numerous specific details are set forth in the following description in order to provide a thorough understanding of the disclosure. These details are provided for the purpose of non-limiting examples and the embodiments may be practiced according to the claims without some or all of these specific details. For the purpose of clarity, technical material that is known in the technical fields related to the disclosure has not been described in detail so that the disclosure is not unnecessarily obscured.

### Overview of the Disclosure

The present disclosure describes methods for the diagnosis and treatment of viral infections including viral infections associated with SARS-CoV-2. The disclosure describes methods of assessing and modulating the levels of TFH, CD4-CTL, and T_{REG} cells. The disclosure also describes modified T-cells for treating viral infections.

### Definitions and Interpretation

The terms “acceptable,” “effective,” or “sufficient”, if and as used herein, and when used to describe the selection of any components, ranges, dose forms, etc. as disclosed herein intend that said component, range, dose form, etc. is suitable for the disclosed purpose.

As used herein, the phrase “baseline expression”, in reference to a gene, refers to the expression of a gene in normal, untreated conditions.

As used herein, the phrase “CD4-CTL cells” refers to a subset of CD4⁺ T cells that have cytotoxic activity. “CD4-CTL cells” referenced herein include any type of CD4-CTL cells known in the art. “CD4-CTL cells” is synonymous with “CD4⁺-CTL cells.”

As used herein, the term “composition” typically but not always intends a combination of the active agent, e.g., an cell or an engineered immune cell, and a naturally-occurring or non-naturally-occurring carrier, inert (for example, a detectable agent or label) or active, such as an adjuvant, diluent, binder, stabilizer, buffers, salts, lipophilic solvents, preservative, adjuvant or the like and include pharmaceutically acceptable carriers. Carriers also include pharmaceutical excipients and additives proteins, peptides, amino acids, lipids, and carbohydrates (e.g., sugars, including monosaccharides, di-, tri-, tetra-oligosaccharides, and oligosaccharides; derivatized sugars such as alditols, aldonic acids, esterified sugars and the like; and polysaccharides or sugar polymers), which can be present singly or in combination, comprising alone or in combination 1-99.99% by weight or volume. Exemplary protein excipients include serum albumin such as human serum albumin (HSA), recombinant human albumin (rHA), gelatin, casein, and the like. Representative amino acid/antibody components, which can also function in a buffering capacity, include alanine, arginine, glycine, arginine, betaine, histidine, glutamic acid, aspartic acid, cysteine, lysine, leucine, isoleucine, valine, methionine, phenylalanine, aspartame, and the like. Carbohydrate excipients are also intended within the scope of this technology, examples of which include but are not limited to monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol) and myoinositol.

As used herein, the term “derivative”, in reference to an amino acid sequence, refers to an amino acid sequence in which at least one of an amino group or an acyl group has been modified.

An “effective amount” is an amount sufficient to effect beneficial or desired results. An effective amount can be administered in one or more administrations, applications or dosages. Such delivery is dependent on a number of variables including the time period for which the individual dosage unit is to be used, the bioavailability of the therapeutic agent, the route of administration, etc. It is understood, however, that specific dose levels of the therapeutic agents disclosed herein for any particular subject depends upon a variety of factors including the activity of the specific compound employed, bioavailability of the compound, the route of administration, the age of the animal and its body weight, general health, sex, the diet of the animal, the time of administration, the rate of excretion, the drug combination, and the severity of the particular disorder being treated and form of administration. In general, one will desire to administer an amount of the compound that is effective to achieve a serum level commensurate with the concentrations found to be effective in vivo. These considerations, as well as effective formulations and administration procedures are well known in the art and are described in standard textbooks.

In and as used herein, the term “expression level” refers to protein, RNA, or mRNA level of a particular gene of interest. Any methods known in the art can be utilized to determine the expression level of a particular gene of interest. Examples include, but are not limited to, reverse transcription and amplification assays (such as PCR, ligation RT-PCR or quantitative RT-PCT), hybridization assays, Northern blotting, dot blotting, in situ hybridization, gel electrophoresis, capillary electrophoresis, column chromatography, Western blotting, immunohistochemistry, immunostaining, or mass spectrometry. Assays can be performed directly on biological samples or on protein/nucleic acids isolated from the samples. It is routine practice in the relevant art to carry out these assays. For example, the detecting step in any method described herein includes contacting the nucleic acid sample from the biological sample obtained from the subject with one or more primers that specifically hybridize to the gene of interest presented herein. Alternatively, the detecting step of any method described herein includes contacting the protein sample from the biological sample obtained from the subject with one or more antibodies that bind to the gene product of the interest presented herein. In some embodiment, the level is an absolute amount or concentration of the protein, RNA, or mRNA level of a particular gene of interest in a cell. In some embodiments, the level is normalized to a control, such as a housekeeping gene.

As used herein, the term “homolog”, in reference to an amino acid sequence, refers to an amino acid sequence that shares similarity to a reference amino acid sequence due to having a common evolutionary origin.

The term “isolated” as used herein refers to molecules, biologicals, cellular materials, cells or biological samples being substantially free from other materials. In one aspect, the term “isolated” refers to nucleic acid, such as DNA or RNA, or protein or polypeptide (e.g., an antibody or derivative thereof), or cell or cellular organelle, or tissue or organ, separated from other DNAs or RNAs, or proteins or polypeptides, or cells or cellular organelles, or tissues or organs, respectively, that are present in the natural source. In some embodiments, the term “isolated” is used herein to refer to cells or tissues that are isolated from other cells or tissues and is meant to encompass both cultured and engineered cells or tissues.

As used herein, the term “isolated cell” generally refers to a cell that is substantially separated from other cells of a tissue.

As used herein, the phrase “ligand mimetic” refers to a composition that contains similar binding properties to ligands, such as the ability to bind receptors.

As used herein, the phrase “normalized mean gene expression” refers to the average intensity of expression of a gene measured on a given array.

As used herein, the term “subsequence”, in reference to an amino acid sequence, refers to a portion or a fragment of a larger amino acid sequence.

If and as used herein, “substantially” or “essentially” means nearly totally or completely, for instance, 95% or greater of some given quantity. In some embodiments, “substantially” or “essentially” means 95%, 96%, 97%, 98%, 99%, 99.5%, or 99.9%.

As used herein, the phrase “T-cell receptor (TCR)” refers to any receptor found on the surface of T cells that is capable of recognizing fragments of an antigen bound to major histocompatibility complex.

If and as used herein, “therapeutically effective amount” of a drug or an agent refers to an amount of the drug or the agent that is an amount sufficient to obtain a pharmacological response; or alternatively, is an amount of the drug or agent that, when administered to a patient with a specified disorder or disease, is sufficient to have the intended effect, e.g., treatment, alleviation, amelioration, palliation or elimination of one or more manifestations of the specified disorder or disease in the patient. A therapeutic effect does not necessarily occur by administration of one dose, and may occur only after administration of a series of doses. Thus, a therapeutically effective amount may be administered in one or more administrations.

As used here, the phrase “T_{FH} cells” refers to any type of follicular helper T cell known in the art.

As used herein, the phrase “T_{REG} cells” refers to any type of regulatory T cell known in the art.

As used herein, the term “variant” refers to an equivalent having a native polypeptide sequence and structure with one or more amino acid additions, substitutions (generally conservative in nature) or deletions, so long as the modifications do not destroy biological activity and which are substantially identical to the reference polypeptide. Variants generally include substitutions that are conservative in nature, i.e., those substitutions that take place within a family of amino acids that are related in their side chains. Specifically, amino acids are generally divided into four families: (1) acidic: aspartate and glutamate; (2) basic: lysine, arginine, histidine; (3) non-polar: alanine, valine, leucine, isoleucine, proline, phenylalanine, methionine, tryptophan; and (4) uncharged polar: glycine, asparagine, glutamine, cysteine, serine threonine, tyrosine. Phenylalanine, tryptophan, and tyrosine are sometimes classified as aromatic amino acids. For example, it is reasonably predictable that an isolated replacement of leucine with isoleucine or valine, an aspartate with a glutamate, a threonine with a serine, or a similar conservative replacement of an amino acid with a structurally related amino acid, will not have a major effect on the biological activity. For example, the polypeptide of interest can include up to about 5-10 conservative or non-conservative amino acid substitutions, or even up to about 15-25 conservative or non-conservative amino acid substitutions, or any integer between 5-25, so long as the desired function of the polypeptide remains intact. One of skill in the art can readily determine regions of the polypeptide of interest that can tolerate change by reference to Hopp/Woods and Kyte-Doolittle plots, well known in the art.

### DESCRIPTION OF ASPECTS AND EMBODIMENTS OF THE DISCLOSURE

As embodied and broadly described herein, an aspect of the present disclosure relates to a method of diagnosing a viral infection in a subject, the method comprising obtaining a biological sample from the subject, quantifying a level of a biological feature associated with TFH or CD4-CTL cells from the biological sample; and comparing the level of the biological feature associated with the TFH or CD4-CTL cells against a quantifiable reference value, wherein when the level of the biological feature is higher than the quantifiable reference value, the viral infection is associated with SARS-CoV-2. In various embodiments, the quantifiable reference value comprises a biological feature associated with the activity or number of TFH or CD4-CTL cells isolated from a source infected with a non-SARS-CoV-2 virus. In various embodiments the quantifiable reference value comprises a biological feature associated with TFH or CD4-CTL cells isolated from a source infected with an influenza virus. In various embodiments, the biological feature comprises the expression or activity of one or more genes set forth in Table 2 and/or Table 3, or one or more of the T-cell receptor (TCR) sequences set forth in Table 6, or a homolog, variant, subsequence, or derivative thereof. In various embodiments, the biological feature comprises expression or activity of one or more of CXCL13, IL21, CD200, BTLA, POU2AF1, PRF1, GZMB, GZMH, GNLY, or NKG7.

In another aspect, described herein is a method of diagnosing the severity of a virally-induced disease in a subject, the method comprising obtaining a biological sample from the subject; quantifying a level of a biological feature associated with T_{FH} cells from the biological sample; and comparing the level of the biological feature against a quantifiable reference value, wherein when the level of the biological feature is above the quantifiable reference value, the virally-induced disease is severe. In various embodiments, the quantifiable reference value comprises a biological feature associated with the number or activity of T_{FH} cells isolated from a second subject suffering from a non-severe case of the virally-induced disease. In various embodiments, the biological feature comprises expression or activity of one or more genes set forth in Table 3, or one or more of the TCR sequences set forth in Table 6, or a homolog, variant, subsequence, or derivative thereof. In various embodiments, the biological feature comprises expression or activity of one or more of ZBED2, ZBTB32, TIGIT, LAG3, TIM3, PD1, DUSP4, CD70, PRF1, or GZMB. In various embodiments, the virally-induced disease is COVID-19 or is associated with SARS-CoV-2.

In some embodiments, the virally-induced disease is the result of a viral infection. In some embodiments, the viral infection is caused by a virus selected from the group consisting of influenza virus, coronavirus, enterovirus (such as coxsackievirus and echovirus), cytomegalovirus, Zika virus, rabies virus, West Nile virus, rubella virus, polio virus, rotavirus, norovirus, herpes simplex virus, varicella-zoster virus, lymphocytic choriomeningitis virus, human immunodeficiency virus, Chikungunya virus, Crimean-Congo hemorrhagic fever virus, Japanese encephalitis virus, Rift Valley Fever virus, Ross River virus, and louping ill virus. In various embodiments, the virally-induced disease is COVID-19 or is associated with SARS-CoV-2.

In another aspect, described herein is a method of diagnosing the severity of a virally-induced disease in a subject, the method comprising obtaining a biological sample from the subject; quantifying a level of a biological feature associated with CD4-CTL cells from the biological sample; and comparing the level of the biological feature against a quantifiable reference value, wherein when the level of the biological feature is above the quantifiable reference value, the virally-induced disease is severe. In various embodiments, the quantifiable reference value comprises a biological feature associated with the number or activity of CD4-CTL cells isolated from a second subject suffering from a non-severe case of the virally-induced disease. In various embodiments, the biological feature comprises expression or activity of one or more genes set forth in Table 2 or Table 4, or one or more of the TCR sequences set forth in Table 6, or a homolog, variant, subsequence, or derivative thereof. In various embodiments, the biological feature comprises expression or activity of one or more of CD72, GPR18, HOPX, ZEB2, CCL3, CCL4, CCL5, CCR1, CCR3, CCR5, XCL1, or XCL2. In various embodiments, the virally-induced disease is COVID-19 or is associated with SARS-CoV-2.

In another aspect, described herein is a method of diagnosing severity of a virally-induced disease in a subject, the method comprising obtaining a biological sample from the subject; quantifying a level of a biological feature associated with T_{REG} cells from the biological sample; and comparing the level of the biological feature associated with T_{REG} against a quantifiable reference value, wherein when the level of the biological feature is below the quantifiable reference value, the virally-induced disease is severe. In various embodiments, the quantifiable reference value comprises a biological feature associated with the number or activity of T_{REG} cells isolated from a second subject suffering from a mild form of the virally-induced disease. In various embodiments, the biological feature comprises expression or activity of FOXP3, or one or more of the TCR sequences set forth in Table 7, or a homolog, variant, subsequence, or derivative thereof. In various embodiments, the virally-induced disease is COVID-19 or is associated with SARS-CoV-2. In various embodiments, the biological feature comprises the expression or activity of T-bet, IFN-γ, IL-2, TNF, IL-3, CSF2, IL-23A, or CCL20. In various embodiments, the virally-induced disease is COVID-19 or is associated with SARS-CoV-2.

In another aspect, described herein is a method of treating a coronavirus infection, treating a disease associated with coronavirus infection, or decreasing, reducing, inhibiting, suppressing, limiting or controlling an adverse symptom or disorder resulting from the coronavirus infection in a subject, the method comprising administering to the subject a therapeutically effective amount of T_{REG} cells.

In another aspect, described herein is a method of treating a coronavirus infection, treating a disease associated with coronavirus infection, or decreasing, reducing, inhibiting, suppressing, limiting or controlling an adverse symptom or disorder resulting from the coronavirus infection in a subject, the method comprising administering to the subject a therapeutic effective amount of an agent that can selectively increase T_{REG} cells in the subject.

In another aspect, described herein is a method of treating a coronavirus infection, treating a disease associated with coronavirus infection, or decreasing, reducing, inhibiting, suppressing, limiting or controlling an adverse symptom or disorder resulting from the coronavirus infection in a subject, the method comprising administering to the subject a therapeutic effective amount of an agent that can selectively reduce T_{FH} or CD4+ CTL cells in the subject. In various embodiments, the agent comprises an antibody that selectively binds to a protein expressed by T_{FH} or CD4+ CTL cells.

In another aspect, described herein is a method of treating a coronavirus infection, treating a disease associated with coronavirus infection, or decreasing, reducing, inhibiting, suppressing, limiting or controlling an adverse symptom or disorder resulting from the coronavirus infection in a subject, the method comprising administering to the subject an effective amount of a population of T-cells that exhibit higher than or lower than baseline expression of one or more genes set forth in Table 1, Table 2, Table 3, Table 4, Table 5, or that express a T-cell receptor (TCR) comprising at least one of the amino acid sequences set forth in Tables 6 and 7, or a homolog, variant, subsequence, or derivative thereof. In various embodiments, the method comprises administering a population of T-cells that exhibit higher than baseline expression of one or more genes set forth in Table 1 and Table 5, or that express a TCR comprising at least one of the amino acid sequences set forth in Table 7, or a homolog, variant, subsequence, or derivative thereof. In various embodiments, the T-cell is a T_{REG} cell. In various embodiments, the one or more genes are selected from the group of T-bet, IFN-γ, IL-2, TNF, IL-3, CSF2, IL-23A, CCL20, IL17A, FOXP3, and IL17F. In various embodiments, the at least one amino acid sequence is selected from Table 7. In various embodiments, the method comprises administering a population of T-cells that exhibit lower than baseline expression of one or more genes set forth in Table 2, Table 3, or Table 4, or that express a TCR comprising at least one of the amino acid sequences set forth in Table 6, or a homolog, variant, subsequence, or derivative thereof. In various embodiments, the one or more genes are selected from the group of ZBED2, ZBTB32, TIGIT, LAG3, TIM3, PD1, DUSP4, CD70, PRF1, and GZMB. In various embodiments, the T-cell is a T_{FH} cell. In various embodiments, the one or more genes are selected from the group of CD72, GPR18, HOPX, ZEB2, CCL3, CCL4, CCL5, CCR1, CCR3, CCR5, XCL1, and XCL2. In various embodiments, the T cell is a CD4-CTL T cell. In various embodiments, the at least one amino acid sequence is selected from Table 6.

In another aspect, described herein is a method of treating a coronavirus infection, treating a disease associated with coronavirus infection, or decreasing, reducing, inhibiting, suppressing, limiting or controlling an adverse symptom or disorder resulting from the coronavirus in a subject, the method comprising administering to the subject an effective amount of an agent that induces higher than or lower than baseline expression of one or more genes set forth in Table 1, Table 2, Table 3, Table 4, and/or Table 5 in T cells, or of a TCR of at least one of the amino acid sequences set forth in Tables 6 and 7, or a homolog, variant, subsequence, or derivative thereof.

In another aspect, described herein is a method of treating a coronavirus infection, treating a disease associated with coronavirus infection, or decreasing, reducing, inhibiting, suppressing, limiting or controlling an adverse symptom or disorder resulting from the coronavirus in a subject, the method comprising administering an effective amount of an agent that induces or inhibits T cell activity of one or more proteins encoded by one or more genes set forth in Table 1, Table 2, Table 3, Table 4, and/or Table 5, or that modulates expression of a T-cell receptor (TCR) comprising at least one of the amino acid sequences set forth in Tables 6 and 7, or a homolog, variant, subsequence, or derivative thereof. In various embodiments, the agent is an antibody, a small molecule, a protein, a peptide, a ligand mimetic, or a nucleic acid. In various embodiments, the baseline expression is normalized mean gene expression. In various embodiments, higher than baseline expression is at least about a 2-fold increase in expression relative to baseline expression and/or lower than baseline expression is at least about a 2-fold decrease in expression relative to baseline expression.

In another aspect, described herein is a modified T-cell modified to exhibit higher than or lower than baseline expression of one or more genes set forth in Table 1, Table 2, Table 3, Table 4, and/or Table 5, or one or more T-cell receptor (TCR) comprising at least one of the amino acid sequences set forth in Tables 6 and 7, or a homolog, variant, subsequence, or derivative thereof. In various embodiments, the modified T cell exhibits higher than baseline expression of one or more genes set forth in Table 1 or Table 5, or expresses a TCR comprising at least one of the amino acid sequences set forth in Table 7, or a homolog, variant, subsequence, or derivative thereof. In various embodiments, the one or more genes are selected from the group of T-bet, IFN-γ, IL-2, TNF, IL-3, CSF2, IL-23A, CCL20, IL17A, FOXP3, and IL17F. In various embodiments, the at least one amino acid sequence is selected from Table 7. In various embodiments, the modified T cell is a T_{REG} cell. In various embodiments, the baseline expression is normalized mean gene expression. In various embodiments, higher than baseline expression is at least about a 2-fold increase in expression relative to baseline expression and/or lower than baseline expression is at least about a 2-fold decrease in expression relative to baseline expression. In various embodiments, the modified T-cell is genetically modified, optionally using one or more of gene editing, recombinant methods and/or a CRISPR/Cas system.

In various embodiments, the modified T-cell is further modified to express a protein that binds to a cytokine, chemokine, lymphokine, or a receptor each thereof. In various embodiments, the protein comprises an antibody or an antigen binding fragment thereof. In various embodiments, the antibody is an IgG, IgA, IgM, IgE or IgD, or a subclass thereof. In various embodiments, the antibody is an IgG selected from the group of IgG1, IgG2, IgG3 or IgG4. In various embodiments, the antigen binding fragment is selected from the group of a Fab, Fab′, F(ab′)2, Fv, Fd, single-chain Fvs (scFv), disulfide-linked Fvs (sdFv) or VL or VH In various embodiments, the modified T-cell comprises a chimeric antigen receptor (CAR). In various embodiments, the chimeric antigen receptor (CAR) comprises: (a) an antigen binding domain; (b) a hinge domain; (c) a transmembrane domain; (d) and an intracellular domain.

In various embodiments, the CAR further comprises one or more costimulatory signaling regions. In various embodiments, the antigen binding domain comprises an anti-CD19 antigen binding domain, the transmembrane domain comprises a CD28 or a CD8 α transmembrane domain, the one or more costimulatory regions selected from a CD28 costimulatory signaling region, a 4-1BB costimulatory signaling region, an ICOS costimulatory signaling region, and an OX40 costimulatory region or a CD3 zeta signaling domain. In various embodiments, the anti-CD19 binding domain comprises a single-chain variable fragment (scFv) that specifically recognizes a humanized anti-CD19 binding domain. In various embodiments, the anti-CD19 binding domain scFv of the CAR comprises a heavy chain variable region and a light chain variable region. In various embodiments, the anti-CD19 binding domain of the CAR further comprises a linker polypeptide located between the anti-CD19 binding domain scFv heavy chain variable region and the anti-CD19 binding domain scFv light chain variable region. In various embodiments, the linker polypeptide of the CAR comprises a polypeptide of the sequence (GGGGS)n wherein n is an integer from 1 to 6. In various embodiments, the CAR further comprises a detectable marker attached to the CAR. In various embodiments, the CAR further comprises a purification marker attached to the CAR. In various embodiments, the modified T-cell comprises a polynucleotide encoding the CAR, and optionally, wherein the polynucleotide encodes and anti-CD19 binding domain.

In various embodiments, the polynucleotide further comprises a promoter operatively linked to the polynucleotide to express the polynucleotide in the modified T-cell. In various embodiments, the polynucleotide further comprises a 2A self-cleaving peptide (T2A) encoding polynucleotide sequence located upstream of a polynucleotide encoding the anti-CD19 binding domain. In various embodiments, the polynucleotide further comprises a polynucleotide encoding a signal peptide located upstream of a polynucleotide encoding the anti-CD19 binding domain. In various embodiments, the polynucleotide further comprises a vector. In various embodiments, the vector is a plasmid. In various embodiments, the vector is a viral vector selected from the group of a retroviral vector, a lentiviral vector, an adenoviral vector, and an adeno-associated viral vector.

In another aspect, described herein is a composition comprising a population of modified T-cells as detailed herein.

In an aspect, a method of treating a viral infection, disease associated with viral infection, or decreasing, reducing, inhibiting, suppressing, limiting or controlling an adverse symptom or disorder resulting from the virus in a subject is provided, the method comprising administering to the subject an effective amount of the modified T-cells and/or the compositions described herein.

In certain embodiments, the viral infection may result from any of the following viral families: Arenaviridae, Arterivirus, Astroviridae, Baculoviridae, Badnavirus, Barnaviridae, Birnaviridae, Bromoviridae, Bunyaviridae, Caliciviridae, Capillovirus, Carlavirus, Caulimovirus, Circoviridae, Closterovirus, Comoviridae, Coronaviridae (e.g., Coronavirus, such as severe acute respiratory syndrome (SARS) virus), Corticoviridae, Cystoviridae, Deltavirus, Dianthovirus, Enamovirus, Filoviridae (e.g., Marburg vims and Ebola virus (e.g., Zaire, Reston, Ivory Coast, or Sudan strain)), Flaviviridae, (e.g., Hepatitis C vims, Dengue vims 1, Dengue vims 2, Dengue virus 3, and Dengue virus 4), Hepadnaviridae, Herpesviridae (e.g., Human herpesvirus 1, 3, 4, 5, and 6, and Cytomegalovirus), Hypoviridae, Iridoviridae, Leviviridae, Lipothrixviridae, Microviridae, Orthomyxoviridae (e.g., Influenzavirus A and B and C), Papovaviridae, Paramyxoviridae (e.g., measles, mumps, and human respiratory syncytial virus), Parvoviridae, Picomaviridae (e.g., poliovirus, rhinovirus, hepatovims, and aphthovirus), Poxviridae (e.g., vaccinia and smallpox vims), Reoviridae (e.g., rotavims), Retroviridae (e.g., lentivirus, such as human immunodeficiency vims (HIV) 1 and HIV 2), Rhabdoviridae (for example, rabies vims, measles virus, respiratory syncytial virus, etc.), Togaviridae (for example, mbella virus, dengue virus, etc.), and Totiviridae. Suitable viral antigens also include all or part of Dengue protein M, Dengue protein E, Dengue DiNS1, Dengue D1NS2, and Dengue DINS3.

The viral infection or virus may be derived from a particular strain such as a papilloma vims, a herpes vims, e.g., herpes simplex 1 and 2; a hepatitis vims, for example, hepatitis A vims (HAV), hepatitis B vims (HBV), hepatitis C virus (HCV), the delta hepatitis D vims (HDV), hepatitis E virus (HEV) and hepatitis G vims (HGV), the tick-borne encephalitis viruses; parainfluenza, varicella-zoster, cytomeglavirus, Epstein-Barr, rotavirus, rhinovims, adenovims, coxsackieviruses, equine encephalitis, Japanese encephalitis, yellow fever, Rift Valley fever, and lymphocytic choriomeningitis.

In another aspect, described herein is a method of treating a coronavirus infection, treating a disease associated with coronavirus infection, or decreasing, reducing, inhibiting, suppressing, limiting or controlling an adverse symptom or disorder resulting from the coronavirus in a subject, the method comprising administering to the subject an effective amount of modified T-cells as detailed herein and/or a composition as detailed herein. In various embodiments, the coronavirus infection is SARS-CoV-2. In various embodiments, the disease associated with coronavirus infection is COVID-19. In various embodiments, the method comprises agonizing a population of or increasing the level, expression, or activity of T_{REG} cells in the subject. In various embodiments, the method comprises antagonizing a population of or decreasing or depleting the level, expression, or activity of T_{FH} or CD4-CTL cells in the subject.

In another aspect, described herein is a method of diagnosing a viral infection ex vivo, the method comprising quantifying, ex vivo, a level of a biological feature associated with T_{FH} or CD4-CTL cells from a biological sample; and comparing the level of the biological feature associated with the T_{FH} or CD4-CTL cells against a quantifiable reference value, wherein when the level of the biological feature is higher than the quantifiable reference value, the viral infection is associated with SARS-CoV-2. In various embodiments, the quantifiable reference value comprises a biological feature associated with the activity or number of T_{FH} or CD4-CTL cells isolated from a biological sample infected with a non-SARS-CoV-2 virus. In various embodiments, the quantifiable reference value comprises a biological feature associated with T_{FH} or CD4-CTL cells isolated from a biological sample infected with an influenza virus. In various embodiments, the biological feature comprises the expression or activity of one or more genes set forth in Table 2 and/or Table 3, or one or more of the T-cell receptor (TCR) sequences set forth in Table 6, or a homolog, variant, subsequence, or derivative thereof. In various embodiments, the biological feature comprises expression or activity of one or more of CXCL13, IL21, CD200, BTLA, POU2AF1, PRF1, GZMB, GZMH, GNLY, or NKG7.

In another aspect, described herein is a method of diagnosing the severity of a virally-induced disease ex vivo, the method comprising quantifying, ex vivo, a level of a biological feature associated with T_{FH} cells from the biological sample; and comparing the level of the biological feature against a quantifiable reference value, wherein when the level of the biological feature is above the quantifiable reference value, the virally-induced disease is severe. In various embodiments, the quantifiable reference value comprises a biological feature associated with the number or activity of T_{FH} cells isolated from a biological sample of a subject suffering from a non-severe case of the virally-induced disease. In various embodiments, the biological feature comprises expression or activity of one or more genes set forth in Table 3, or one or more of the TCR sequences set forth in Table 6, or a homolog, variant, subsequence, or derivative thereof. In various embodiments, the biological feature comprises expression or activity of one or more of ZBED2, ZBTB32, TIGIT, LAG3, TIM3, PD1, DUSP4, CD70, PRF1, or GZMB. In various embodiments, the virally-induced disease is COVID-19 or is associated with SARS-CoV-2.

In another aspect, described herein is a method of diagnosing the severity of a virally-induced disease ex vivo, the method comprising quantifying, ex vivo, a level of a biological feature associated with CD4-CTL cells from the biological sample; and comparing the level of the biological feature against a quantifiable reference value, wherein when the level of the biological feature is above the quantifiable reference value, the virally-induced disease is severe. In various embodiments, the quantifiable reference value comprises a biological feature associated with the number or activity of CD4-CTL cells isolated from a biological sample of a subject suffering from a non-severe case of the virally-induced disease. In various embodiments, the biological feature comprises expression or activity of one or more genes set forth in Table 2 or Table 4, or one or more of the TCR sequences set forth in Table 6, or a homolog, variant, subsequence, or derivative thereof. In various embodiments, the biological feature comprises expression or activity of one or more of CD72, GPR18, HOPX, ZEB2, CCL3, CCL4, CCL5, CCR1, CCR3, CCR5, XCL1, or XCL2. In various embodiments, the virally-induced disease is COVID-19 or is associated with SARS-CoV-2.

In another aspect, described herein is a method of diagnosing severity of a virally-induced disease ex vivo, the method comprising quantifying, ex vivo, a level of a biological feature associated with T_{REG} cells from the biological sample; and comparing the level of the biological feature associated with T_{REG} against a quantifiable reference value, wherein when the level of the biological feature is below the quantifiable reference value, the virally-induced disease is severe. In various embodiments, the quantifiable reference value comprises a biological feature associated with the number or activity of T_{REG} cells isolated from a biological sample of a subject suffering from the virally-induced disease. In various embodiments, the biological sample is isolated from a subject suffering from a mild form of the virally-induced disease. In various embodiments, the biological sample is isolated from a subject suffering from a severe form of the virally-induced disease. In various embodiments, the biological feature comprises expression or activity of FOXP3, or one or more of the TCR sequences set forth in Table 7, or a homolog, variant, subsequence, or derivative thereof. In various embodiments, the virally-induced disease is COVID-19 or is associated with SARS-CoV-2. In various embodiments, the biological feature comprises the expression or activity of T-bet, IFN-γ, IL-2, TNF, IL-3, CSF2, IL-23A, or CCL20. In various embodiments, the virally-induced disease is COVID-19 or is associated with SARS-CoV-2.

In another aspect, described herein is a method of treating a viral infection, treating a disease associated with the viral infection, or decreasing, reducing, inhibiting, suppressing, limiting or controlling an adverse symptom or disorder resulting from the viral infection in a subject, the method comprising administering to the subject a therapeutically effective amount of T_{REG} cells.

In another aspect, described herein is a method of treating a viral infection, treating a disease associated with viral infection, or decreasing, reducing, inhibiting, suppressing, limiting or controlling an adverse symptom or disorder resulting from the viral infection in a subject, the method comprising administering to the subject a therapeutic effective amount of an agent that can selectively increase T_{REG} cells in the subject.

In another aspect, described herein is a method of treating a viral infection, treating a disease associated with the viral infection, or decreasing, reducing, inhibiting, suppressing, limiting or controlling an adverse symptom or disorder resulting from the viral infection in a subject, the method comprising administering to the subject a therapeutic effective amount of an agent that can selectively reduce T_{FH} or CD4+ CTL cells in the subject. In various embodiments, the agent comprises an antibody that selectively binds to a protein expressed by T_{FH} or CD4+ CTL cells.

In another aspect, described herein is a method of treating a viral infection, treating a disease associated with the viral infection, or decreasing, reducing, inhibiting, suppressing, limiting or controlling an adverse symptom or disorder resulting from the viral infection in a subject, the method comprising administering to the subject an effective amount of a population of T-cells that exhibit higher than or lower than baseline expression of one or more genes set forth in Table 1, Table 2, Table 3, Table 4, and/or Table 5, or that express a T-cell receptor (TCR) comprising at least one of the amino acid sequences set forth in Tables 6 and 7, or a homolog, variant, subsequence, or derivative thereof. In various embodiments, the method comprises administering a population of T-cells that exhibit higher than baseline expression of one or more genes set forth in Table 1 or Table 5, or that express a TCR comprising at least one of the amino acid sequences set forth in Table 7, or a homolog, variant, subsequence, or derivative thereof. In various embodiments, the T-cell is a T_{REG} cellIn various embodiments, the one or more genes are selected from the group of T-bet, IFN-γ, IL-2, TNF, IL-3, CSF2, IL-23A, CCL20, IL17A, FOXP3, and IL17F. In various embodiments, the at least one amino acid sequence is selected from Table 7. In various embodiments, the method comprises administering a population of T-cells that exhibit lower than baseline expression of one or more genes set forth in Table 2, Table 3, or Table 4, or that express a TCR comprising at least one of the amino acid sequences set forth in Table 6, or a homolog, variant, subsequence, or derivative thereof. In various embodiments, the one or more genes are selected from the group of ZBED2, ZBTB32, TIGIT, LAG3, TIM3, PD1, DUSP4, CD70, PRF1, and GZMB. In various embodiments, the T-cell is a T_{FH} cell. In various embodiments, the one or more genes are selected from the group of CD72, GPR18, HOPX, ZEB2, CCL3, CCL4, CCL5, CCR1, CCR3, CCR5, XCL1, and XCL2. In various embodiments, the T cell is a CD4-CTL T cell. In various embodiments, the at least one amino acid sequence is selected from Table 6.

In another aspect, described herein is a method of treating a viral infection, treating a disease associated with the viral infection, or decreasing, reducing, inhibiting, suppressing, limiting or controlling an adverse symptom or disorder resulting from the viral infection in a subject, the method comprising administering to the subject an effective amount of an agent that induces higher than or lower than baseline expression of one or more genes set forth in Table 1, Table 2, Table 3, Table 4, and/or Table 5 in T cells, or of a TCR of at least one of the amino acid sequences set forth in Tables 6 and 7, or a homolog, variant, subsequence, or derivative thereof.

In another aspect, described herein is a method of treating a viral infection, treating a disease associated with the viral infection, or decreasing, reducing, inhibiting, suppressing, limiting or controlling an adverse symptom or disorder resulting from the viral infection in a subject, the method comprising administering an effective amount of an agent that induces or inhibits T cell activity of one or more proteins encoded by one or more genes set forth in Table 1, Table 2, Table 3, Table 4, and/or Table 5, or that modulates expression of a T-cell receptor (TCR) comprising at least one of the amino acid sequences set forth in Tables 6 and 7, or a homolog, variant, subsequence, or derivative thereof. In various embodiments, the agent is an antibody, a small molecule, a protein, a peptide, a ligand mimetic or a nucleic acid. In various embodiments, baseline expression is normalized mean gene expression. In various embodiments, higher than baseline expression is at least about a 2-fold increase in expression relative to baseline expression and/or lower than baseline expression is at least about a 2-fold decrease in expression relative to baseline expression.

In another aspect, described herein is a method of treating a viral infection, treating a disease associated with viral infection, or decreasing, reducing, inhibiting, suppressing, limiting or controlling an adverse symptom or disorder resulting from the viral infection in a subject, the method comprising administering to the subject an effective amount of modified T-cells as detailed herein and/or a composition as detailed herein. In various embodiments, the method further comprises agonizing a population of or increasing the level, expression, or activity of T_{REG} cells in the subject. In various embodiments, the method comprises antagonizing a population of or decreasing or depleting the level, expression, or activity of T_{FH} or CD4-CTL cells in the subject.

### Methods of Isolating and Detecting CD4-CTLs

Numerous methods can be used to isolate CD4-CTL cells. In an aspect, CD4-CTL cells are detected using an Interferon-Gamma Release Assay. In embodiments, peripheral blood mononuclear cells (PBMCs) are isolated from a patient and the level of Interferon-Gamma in the PBMCs are detected. In embodiments, high levels of Interferon-Gamma would be indicative of the patient having high levels of CD4-CTL cells. In embodiments, the high levels of CD4-CTL cells would indicate that the patient is suffering from a viral disease described herein.

In an aspect, CD4-CTL cells are detected using flow cytometry. In embodiments a sample is derived from a patient. In embodiments, the sample is PBMCs. In embodiments, the sample is assayed for gene expression of a specific gene subset. In embodiments, the specific gene subset is correlated to CD4-CTL cell expression or activity.

### Viral Infections

In an aspect, the methods and compositions described herein can be used to diagnose and treat SARS-CoV-2.

Coronaviruses is a family of single-stranded, positive-strand RNA viruses characterized with crown-like spikes on their surface. The coronaviruses belong to the Coronaviridae family, Nidovirales order. There are four sub-groupings or categories of CoVs, alpha, beta, gamma, and delta. The CoVs are the largest known RNA viruses, comprising 16 non-structural proteins and 4 structural proteins which include spike (S) protein, envelope (E) protein, membrane (M) protein, and nucleocapsid (N) protein.

There are seven species of coronaviruses that are known to cause respiratory and intestinal infections in humans. The seven species are 229E (or α-type HCoV-229E), NL63 (or α-type HCoV-NL63), OC43 (or β-type HCoV-OC43), HKU1 (or 3-type HCoV-HKU1), MERS-CoV (the β-type HCoV that causes Middle East Respiratory Syndrome or MERS), SARS-CoV (the β-type HCoV that causes severe acute respiratory syndrome or SARS), and SARS-CoV2 (the β-type HCoV that causes the coronavirus disease of 2019, COVID-19, or 2019-nCoV).

In some embodiments, the CoVs are also classified based on their pathogenicity. In some instances, the mild pathogenic CoVs include HCoV-229E, HCoV-OC43, HCoV-NL63, and HCoV-HKU1. In some instances, the highly pathogenic CoVs include SARS-CoV, MERS-CoV, and SARS-CoV2. In some cases, the mild pathogens infect the upper respiratory tract and causes seasonal, mild to moderate cold-like respiratory diseases in the subject. In some cases, the highly pathogenic CoVs infect the lower respiratory tract and cause severe pneumonia, leading, in some cases, to fatal acute lung injury (ALI) and/or acute respiratory distress syndrome (ARDS).

In an aspect, the methods and compositions described herein can be used to diagnose and treat viral infections that result from viruses other than SARS-CoV-2. In embodiments, the methods and compositions described herein can be used to treat viral infections that result from any of the following viral families: Arenaviridae, Arterivirus, Astroviridae, Baculoviridae, Badnavirus, Bamaviridae, Birnaviridae, Bromoviridae, Bunyaviridae, Caliciviridae, Capillovirus, Carlavirus, Caulimovirus, Circoviridae, Closterovirus, Comoviridae, Coronaviridae (e.g., Coronavirus, such as severe acute respiratory syndrome (SARS) virus), Corticoviridae, Cystoviridae, Deltavirus, Dianthovirus, Enamovirus, Filoviridae (e.g., Marburg vims and Ebola virus (e.g., Zaire, Reston, Ivory Coast, or Sudan strain)), Flaviviridae, (e.g., Hepatitis C vims, Dengue vims 1, Dengue vims 2, Dengue virus 3, and Dengue virus 4), Hepadnaviridae, Herpesviridae (e.g., Human herpesvirus 1, 3, 4, 5, and 6, and Cytomegalovirus), Hypoviridae, Iridoviridae, Leviviridae, Lipothrixviridae, Microviridae, Orthomyxoviridae (e.g., Influenzavirus A and B and C), Papovaviridae, Paramyxoviridae (e.g., measles, mumps, and human respiratory syncytial virus), Parvoviridae, Picornaviridae (e.g., poliovirus, rhinovirus, hepatovims, and aphthovirus), Poxviridae (e.g., vaccinia and smallpox vims), Reoviridae (e.g., rotavims), Retroviridae (e.g., lentivirus, such as human immunodeficiency vims (HIV) 1 and HIV 2), Rhabdoviridae (for example, rabies vims, measles virus, respiratory syncytial virus, etc.), Togaviridae (for example, mbella virus, dengue virus, etc.), and Totiviridae. Suitable viral antigens also include all or part of Dengue protein M, Dengue protein E, Dengue DiNS1, Dengue D1NS2, and Dengue D1NS3.

In an aspect, the technology described herein may be used to diagnose and treat viral infections that preferentially upregulate the levels, expression, or activity of TFH or CD4-CTL cells and/or downregulate the levels, expression, or activity of T_{REG} cells.

### Compositions

In compositions used in accordance with the disclosure, including cells, treatments, therapies, agents, drugs and pharmaceutical formulations can be packaged in dosage unit form for ease of administration and uniformity of dosage. The term “unit dose” or “dosage” refers to physically discrete units suitable for use in a subject, each unit containing a predetermined quantity of the composition calculated to produce the desired responses in association with its administration, i.e., the appropriate route and regimen. The quantity to be administered, both according to number of treatments and unit dose, depends on the result and/or protection desired. Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition. Upon formulation, solutions will be administered in a manner compatible with the dosage formulation and in such amount as is therapeutically or prophylactically effective. The formulations are easily administered in a variety of dosage forms, such as the type of injectable solutions described herein.

In some embodiments, the compositions disclosed herein are administered to a subject by multiple administration routes, including but not limited to, parenteral, oral, buccal, rectal, sublingual, or transdermal administration routes. In some cases, parenteral administration comprises intravenous, subcutaneous, intramuscular, intracerebral, intranasal, intra-arterial, intra-articular, intradermal, intravitreal, intraosseous infusion, intraperitoneal, or intratechal administration. In some instances, the composition (e.g., pharmaceutical composition) is formulated for local administration. In other instances, the composition (e.g., pharmaceutical composition) is formulated for systemic administration.

In some embodiments, the compositions (e.g., pharmaceutical composition or formulations) include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations (e.g., nanoparticle formulations), and mixed immediate and controlled release formulations.

In some embodiments, the compositions (e.g., pharmaceutical composition or formulations) include a carrier or carrier materials selected on the basis of compatibility with the composition disclosed herein, and the release profile properties of the desired dosage form. Exemplary carrier materials include, e.g., binders, suspending agents, disintegration agents, filling agents, surfactants, solubilizers, stabilizers, lubricants, wetting agents, diluents, and the like.

In some instances, the compositions (e.g., pharmaceutical composition or formulations) further include pH adjusting agents or buffering agents. In some instances, the compositions (e.g., pharmaceutical composition or formulations) includes one or more salts in an amount required to bring osmolality of the composition into an acceptable range.

In some embodiments, the compositions (e.g., pharmaceutical composition or formulations) include, but are not limited to, sugars or salts and/or other agents such as heparin to increase the solubility and in vivo stability of polypeptides.

In some instances, the compositions (e.g., pharmaceutical composition or formulations) further include diluent which are used to stabilize compounds because they can provide a more stable environment. In some cases, the compositions (e.g., pharmaceutical composition or formulations) include disintegration agents or disintegrants to facilitate the breakup or disintegration of a substance.

As it would be understood by one of skill in the art, any embodiments, instances, aspects, examples, or cases can be combined or substituted with any other embodiments, instances, aspects, examples, or cases as disclosed herein, no matter where the embodiments, instances, aspects, examples or cases are provided in this disclosure.

### Tables

As referred to herein, Tables 1 and 5 generally depict transcriptome analysis of various genes in T_{REG} cells. As referred to herein, Tables 2 and 4 generally depict transcriptome analysis of various genes in CD4-CTLs. As referred to herein, Table 3 generally depicts transcriptome analysis of various genes in Tfh cells. As referred to herein, Table 6 generally depicts CD4-CTL-related TCR sequences. As referred to herein, Table 7 generally depicts T_{REG}-related TCR sequences.

As referred herein, Table 1 depicts as follows:

TABLE 1

| | | | | Test statistics | | |
|---|---|---|---|---|---|---|
| | | Fraction of | | Average | | |
| | | expressing cells | | logged | | |
| | | Cluster- | Other | Fold | | Adjusted |
| Gene ID | Cluster | specific | cells | Change | P-value | P-value |
| CXCL10 | 2 | 0.28 | 0.02 | 2.08 | 0 | 0 |
| LTB | 2 | 0.99 | 0.63 | 1.92 | 0 | 0 |
| S100A4 | 2 | 0.93 | 0.49 | 1.53 | 0 | 0 |
| LGALS3 | 2 | 0.88 | 0.22 | 1.53 | 0 | 0 |
| S100A6 | 2 | 0.99 | 0.68 | 1.41 | 0 | 0 |
| IFIT3 | 2 | 0.70 | 0.16 | 1.36 | 0 | 0 |
| CORO1A | 2 | 0.97 | 0.56 | 1.29 | 0 | 0 |
| GBP5 | 2 | 0.85 | 0.41 | 1.27 | 0 | 0 |
| IL32 | 2 | 1.00 | 0.87 | 1.25 | 0 | 0 |
| CISH | 2 | 0.81 | 0.15 | 1.23 | 0 | 0 |
| GBP1 | 2 | 0.91 | 0.42 | 1.22 | 0 | 0 |
| IL4I1 | 2 | 0.76 | 0.14 | 1.20 | 0 | 0 |
| LY6E | 2 | 0.94 | 0.66 | 1.20 | 0 | 0 |
| CYTIP | 2 | 0.94 | 0.49 | 1.18 | 0 | 0 |
| TYMP | 2 | 0.88 | 0.37 | 1.18 | 0 | 0 |
| GBP4 | 2 | 0.80 | 0.25 | 1.16 | 0 | 0 |
| PTPRCAP | 2 | 0.89 | 0.37 | 1.15 | 0 | 0 |
| IFI6 | 2 | 0.83 | 0.48 | 1.15 | 0 | 0 |
| RGS1 | 2 | 0.55 | 0.15 | 1.14 | 0 | 0 |
| TMSB10 | 2 | 1.00 | 0.95 | 1.13 | 0 | 0 |
| STAT1 | 2 | 0.96 | 0.62 | 1.13 | 0 | 0 |
| MYL12A | 2 | 0.99 | 0.85 | 1.13 | 0 | 0 |
| SAMHD1 | 2 | 0.82 | 0.17 | 1.12 | 0 | 0 |
| S100A11 | 2 | 0.97 | 0.68 | 1.11 | 0 | 0 |
| ALOX5AP | 2 | 0.79 | 0.29 | 1.07 | 0 | 0 |
| FLT3LG | 2 | 0.85 | 0.18 | 1.07 | 0 | 0 |
| OSM | 2 | 0.47 | 0.05 | 1.06 | 0 | 0 |
| ISG15 | 2 | 0.87 | 0.48 | 1.05 | 0 | 0 |
| MT2A | 2 | 0.58 | 0.29 | 1.05 | 0 | 0 |
| LGALS1 | 2 | 0.74 | 0.38 | 1.05 | 0 | 0 |
| TMSB4X | 2 | 1.00 | 0.96 | 1.03 | 0 | 0 |
| BST2 | 2 | 0.92 | 0.57 | 1.03 | 0 | 0 |
| IL22 | 2 | 0.15 | 0.02 | 1.02 | 0 | 0 |
| CMTM6 | 2 | 0.90 | 0.50 | 1.00 | 0 | 0 |
| SAMD9L | 2 | 0.73 | 0.15 | 0.99 | 0 | 0 |
| VIM | 2 | 0.99 | 0.82 | 0.99 | 0 | 0 |
| OAS1 | 2 | 0.64 | 0.18 | 0.99 | 0 | 0 |
| GIMAP7 | 2 | 0.71 | 0.24 | 0.99 | 0 | 0 |
| RSAD2 | 2 | 0.47 | 0.06 | 0.98 | 0 | 0 |
| IFI35 | 2 | 0.83 | 0.36 | 0.96 | 0 | 0 |
| RNF213 | 2 | 0.86 | 0.34 | 0.96 | 0 | 0 |
| CTSH | 2 | 0.62 | 0.11 | 0.94 | 0 | 0 |
| MX1 | 2 | 0.76 | 0.35 | 0.91 | 0 | 0 |
| PSME1 | 2 | 0.99 | 0.83 | 0.91 | 0 | 0 |
| IFITM1 | 2 | 0.97 | 0.78 | 0.90 | 0 | 0 |
| TRADD | 2 | 0.71 | 0.12 | 0.86 | 0 | 0 |
| IFIT1 | 2 | 0.43 | 0.10 | 0.86 | 0 | 0 |
| OAS3 | 2 | 0.63 | 0.12 | 0.86 | 0 | 0 |
| PLP2 | 2 | 0.88 | 0.48 | 0.86 | 0 | 0 |
| OSTF1 | 2 | 0.80 | 0.26 | 0.85 | 0 | 0 |
| ISG20 | 2 | 0.89 | 0.64 | 0.85 | 0 | 0 |
| FAS | 2 | 0.71 | 0.17 | 0.85 | 0 | 0 |
| ARHGDIB | 2 | 0.94 | 0.52 | 0.84 | 0 | 0 |
| PSMB9 | 2 | 0.98 | 0.73 | 0.84 | 0 | 0 |
| ANXA2 | 2 | 0.92 | 0.55 | 0.84 | 0 | 0 |
| PIM2 | 2 | 0.64 | 0.32 | 0.84 | 0 | 0 |
| IRF1 | 2 | 0.93 | 0.60 | 0.83 | 0 | 0 |
| TNFSF13B | 2 | 0.43 | 0.06 | 0.83 | 0 | 0 |
| KLF6 | 2 | 0.99 | 0.76 | 0.83 | 0 | 0 |
| DPP4 | 2 | 0.63 | 0.08 | 0.83 | 0 | 0 |
| CASP1 | 2 | 0.67 | 0.15 | 0.82 | 0 | 0 |
| PSMB10 | 2 | 0.94 | 0.60 | 0.82 | 0 | 0 |
| CLDND1 | 2 | 0.82 | 0.51 | 0.82 | 0 | 0 |
| SOCS1 | 2 | 0.66 | 0.27 | 0.81 | 0 | 0 |
| XAF1 | 2 | 0.77 | 0.27 | 0.81 | 0 | 0 |
| DUSP1 | 2 | 0.61 | 0.30 | 0.80 | 0 | 0 |
| HSPA1A | 2 | 0.59 | 0.26 | 0.78 | 0 | 0 |
| SAT1 | 2 | 0.87 | 0.64 | 0.77 | 0 | 0 |
| GIMAP4 | 2 | 0.59 | 0.19 | 0.76 | 0 | 0 |
| OPTN | 2 | 0.75 | 0.24 | 0.76 | 0 | 0 |
| IFI44L | 2 | 0.74 | 0.30 | 0.75 | 0 | 0 |
| PIM1 | 2 | 0.78 | 0.33 | 0.74 | 0 | 0 |
| GPSM3 | 2 | 0.88 | 0.42 | 0.73 | 0 | 0 |
| ARHGAP15 | 2 | 0.76 | 0.31 | 0.72 | 0 | 0 |
| HAPLN3 | 2 | 0.64 | 0.20 | 0.72 | 0 | 0 |
| PSME2 | 2 | 0.99 | 0.90 | 0.72 | 0 | 0 |
| IRF7 | 2 | 0.71 | 0.31 | 0.72 | 0 | 0 |
| CARD16 | 2 | 0.67 | 0.16 | 0.72 | 0 | 0 |
| GSDMD | 2 | 0.67 | 0.19 | 0.72 | 0 | 0 |
| TPM4 | 2 | 0.78 | 0.35 | 0.71 | 0 | 0 |
| MVP | 2 | 0.78 | 0.35 | 0.71 | 0 | 0 |
| TUBA1A | 2 | 0.60 | 0.17 | 0.70 | 0 | 0 |
| EMP3 | 2 | 1.00 | 0.89 | 0.70 | 0 | 0 |
| CDKN1A | 2 | 0.52 | 0.20 | 0.69 | 0 | 0 |
| SQSTM1 | 2 | 0.82 | 0.50 | 0.69 | 0 | 0 |
| CD47 | 2 | 0.86 | 0.45 | 0.69 | 0 | 0 |
| ANKRD12 | 2 | 0.89 | 0.52 | 0.68 | 0 | 0 |
| ARPC1B | 2 | 0.97 | 0.71 | 0.68 | 0 | 0 |
| DDX58 | 2 | 0.49 | 0.10 | 0.68 | 0 | 0 |
| CAST | 2 | 0.79 | 0.35 | 0.67 | 0 | 0 |
| TUBB | 2 | 0.95 | 0.76 | 0.67 | 0 | 0 |
| PPM1K | 2 | 0.55 | 0.16 | 0.67 | 0 | 0 |
| CAPN2 | 2 | 0.67 | 0.24 | 0.67 | 0 | 0 |
| PARP9 | 2 | 0.71 | 0.24 | 0.67 | 0 | 0 |
| GSTK1 | 2 | 0.88 | 0.55 | 0.67 | 0 | 0 |
| MAL | 2 | 0.50 | 0.11 | 0.66 | 0 | 0 |
| FOXP3 | 2 | 0.18 | 0.03 | 0.65 | 0 | 0 |
| OASL | 2 | 0.49 | 0.18 | 0.65 | 0 | 0 |
| LIMDZ | 2 | 0.88 | 0.54 | 0.65 | 0 | 0 |
| CCR6 | 2 | 0.48 | 0.07 | 0.65 | 0 | 0 |
| IFIT2 | 2 | 0.31 | 0.05 | 0.64 | 0 | 0 |
| CXCR4 | 2 | 0.51 | 0.16 | 0.64 | 0 | 0 |
| IFI44 | 2 | 0.57 | 0.16 | 0.64 | 0 | 0 |
| UBE2L6 | 2 | 0.90 | 0.59 | 0.63 | 0 | 0 |
| EIF2AK2 | 2 | 0.69 | 0.33 | 0.63 | 0 | 0 |
| SAMD9 | 2 | 0.59 | 0.19 | 0.62 | 0 | 0 |
| IL10RA | 2 | 0.56 | 0.11 | 0.62 | 0 | 0 |
| ETV7 | 2 | 0.47 | 0.07 | 0.62 | 0 | 0 |
| VAMP8 | 2 | 0.80 | 0.40 | 0.62 | 0 | 0 |
| ACAT2 | 2 | 0.58 | 0.18 | 0.62 | 0 | 0 |
| GIMAP5 | 2 | 0.70 | 0.38 | 0.62 | 0 | 0 |
| PLSCR1 | 2 | 0.58 | 0.26 | 0.61 | 0 | 0 |
| IFITM2 | 2 | 0.90 | 0.70 | 0.61 | 0 | 0 |
| TAPBP | 2 | 0.92 | 0.65 | 0.61 | 0 | 0 |
| ARL6IP5 | 2 | 0.92 | 0.60 | 0.61 | 0 | 0 |
| MYL6 | 2 | 1.00 | 0.94 | 0.61 | 0 | 0 |
| PSMB8 | 2 | 0.96 | 0.78 | 0.61 | 0 | 0 |
| SOS1 | 2 | 0.45 | 0.06 | 0.60 | 0 | 0 |
| APOL2 | 2 | 0.54 | 0.13 | 0.60 | 0 | 0 |
| APOL3 | 2 | 0.49 | 0.07 | 0.60 | 0 | 0 |
| ANXA1 | 2 | 0.89 | 0.58 | 0.60 | 0 | 0 |
| APOL6 | 2 | 0.73 | 0.33 | 0.59 | 0 | 0 |
| DRAP1 | 2 | 0.89 | 0.62 | 0.59 | 0 | 0 |
| SQRDL | 2 | 0.65 | 0.21 | 0.58 | 0 | 0 |
| CMPK2 | 2 | 0.41 | 0.06 | 0.58 | 0 | 0 |
| ILK | 2 | 0.64 | 0.22 | 0.58 | 0 | 0 |
| IFITM3 | 2 | 0.25 | 0.11 | 0.58 | 0 | 0 |
| MX2 | 2 | 0.50 | 0.17 | 0.57 | 0 | 0 |
| AHNAK | 2 | 0.63 | 0.25 | 0.57 | 0 | 0 |
| LCP2 | 2 | 0.78 | 0.45 | 0.57 | 0 | 0 |
| HSPB1 | 2 | 0.71 | 0.38 | 0.57 | 0 | 0 |
| GLRX | 2 | 0.46 | 0.08 | 0.57 | 0 | 0 |
| CD74 | 2 | 0.97 | 0.74 | 0.57 | 0 | 0 |
| TNFSF10 | 2 | 0.79 | 0.58 | 0.57 | 0 | 0 |
| TNFRSF14 | 2 | 0.67 | 0.25 | 0.56 | 0 | 0 |
| CAPG | 2 | 0.41 | 0.07 | 0.56 | 0 | 0 |
| ACAP1 | 2 | 0.56 | 0.15 | 0.56 | 0 | 0 |
| HERC5 | 2 | 0.39 | 0.09 | 0.56 | 0 | 0 |
| CDK2AP2 | 2 | 0.74 | 0.43 | 0.56 | 0 | 0 |
| TAP1 | 2 | 0.97 | 0.77 | 0.55 | 0 | 0 |
| EPSTI1 | 2 | 0.72 | 0.37 | 0.55 | 0 | 0 |
| TXN | 2 | 0.96 | 0.84 | 0.55 | 0 | 0 |
| RTN4 | 2 | 0.67 | 0.35 | 0.55 | 0 | 0 |
| LAPTM5 | 2 | 0.87 | 0.51 | 0.55 | 0 | 0 |
| C10orf128 | 2 | 0.44 | 0.10 | 0.55 | 0 | 0 |
| RAC2 | 2 | 0.97 | 0.79 | 0.55 | 0 | 0 |
| SP100 | 2 | 0.84 | 0.57 | 0.55 | 0 | 0 |
| PFN1 | 2 | 1.00 | 0.98 | 0.54 | 0 | 0 |
| STK17B | 2 | 0.82 | 0.52 | 0.54 | 0 | 0 |
| LGALS9 | 2 | 0.39 | 0.08 | 0.54 | 0 | 0 |
| RARRES3 | 2 | 0.56 | 0.23 | 0.54 | 0 | 0 |
| KLRB1 | 2 | 0.58 | 0.24 | 0.54 | 0 | 0 |
| FLNA | 2 | 0.74 | 0.41 | 0.54 | 0 | 0 |
| ZFP36 | 2 | 0.49 | 0.22 | 0.54 | 0 | 0 |
| DTX3L | 2 | 0.58 | 0.19 | 0.54 | 0 | 0 |
| ACTB | 2 | 1.00 | 0.99 | 0.53 | 0 | 0 |
| SNX10 | 2 | 0.50 | 0.12 | 0.53 | 0 | 0 |
| CLEC2B | 2 | 0.53 | 0.20 | 0.53 | 0 | 0 |
| EML4 | 2 | 0.74 | 0.39 | 0.53 | 0 | 0 |
| CYB5A | 2 | 0.51 | 0.13 | 0.53 | 0 | 0 |
| TBCB | 2 | 0.75 | 0.42 | 0.52 | 0 | 0 |
| ERAP2 | 2 | 0.45 | 0.11 | 0.52 | 0 | 0 |
| ACTG1 | 2 | 0.99 | 0.96 | 0.52 | 0 | 0 |
| IFIH1 | 2 | 0.48 | 0.12 | 0.51 | 0 | 0 |
| GNB2 | 2 | 0.73 | 0.43 | 0.51 | 0 | 0 |
| SELPLG | 2 | 0.44 | 0.09 | 0.51 | 0 | 0 |
| CFL1 | 2 | 1.00 | 0.96 | 0.51 | 0 | 0 |
| ITM2B | 2 | 0.97 | 0.79 | 0.51 | 0 | 0 |
| AHR | 2 | 0.63 | 0.29 | 0.51 | 0 | 0 |
| HERC6 | 2 | 0.42 | 0.08 | 0.50 | 0 | 0 |
| SERPINB1 | 2 | 0.59 | 0.22 | 0.50 | 0 | 0 |
| OAS2 | 2 | 0.57 | 0.23 | 0.50 | 0 | 0 |
| NFKB2 | 2 | 0.71 | 0.42 | 0.50 | 0 | 0 |
| DYNLT1 | 2 | 0.64 | 0.33 | 0.50 | 0 | 0 |
| PARP14 | 2 | 0.64 | 0.30 | 0.50 | 0 | 0 |
| IL2RA | 2 | 0.73 | 0.48 | 0.50 | 0 | 0 |
| PDE4B | 2 | 0.52 | 0.18 | 0.49 | 0 | 0 |
| PAG1 | 2 | 0.56 | 0.20 | 0.49 | 0 | 0 |
| PARP12 | 2 | 0.48 | 0.12 | 0.49 | 0 | 0 |
| UNC119 | 2 | 0.44 | 0.10 | 0.49 | 0 | 0 |
| IL15RA | 2 | 0.52 | 0.19 | 0.49 | 0 | 0 |
| DBI | 2 | 0.88 | 0.68 | 0.49 | 0 | 0 |
| CASP4 | 2 | 0.60 | 0.26 | 0.49 | 0 | 0 |
| CALM1 | 2 | 0.99 | 0.92 | 0.49 | 0 | 0 |
| TANK | 2 | 0.72 | 0.39 | 0.49 | 0 | 0 |
| LMO4 | 2 | 0.46 | 0.11 | 0.49 | 0 | 0 |
| XRN1 | 2 | 0.59 | 0.27 | 0.49 | 0 | 0 |
| MGST3 | 2 | 0.61 | 0.23 | 0.48 | 0 | 0 |
| KIAA1551 | 2 | 0.62 | 0.32 | 0.48 | 0 | 0 |
| BHLHE40 | 2 | 0.77 | 0.46 | 0.48 | 0 | 0 |
| DDX60 | 2 | 0.48 | 0.13 | 0.48 | 0 | 0 |
| LPXN | 2 | 0.72 | 0.43 | 0.48 | 0 | 0 |
| CNN2 | 2 | 0.46 | 0.16 | 0.48 | 0 | 0 |
| CD63 | 2 | 0.74 | 0.47 | 0.48 | 0 | 0 |
| TIFA | 2 | 0.47 | 0.18 | 0.48 | 0 | 0 |
| FAM6SB | 2 | 0.41 | 0.08 | 0.47 | 0 | 0 |
| ARID5A | 2 | 0.56 | 0.26 | 0.47 | 0 | 0 |
| ICAM1 | 2 | 0.42 | 0.12 | 0.47 | 0 | 0 |
| IL2RB | 2 | 0.63 | 0.32 | 0.47 | 0 | 0 |
| GABARAP | 2 | 0.94 | 0.71 | 0.47 | 0 | 0 |
| SNX6 | 2 | 0.75 | 0.47 | 0.47 | 0 | 0 |
| CCSER2 | 2 | 0.49 | 0.14 | 0.47 | 0 | 0 |
| TSPO | 2 | 0.73 | 0.45 | 0.46 | 0 | 0 |
| IRF2 | 2 | 0.50 | 0.15 | 0.46 | 0 | 0 |
| BIN2 | 2 | 0.49 | 0.16 | 0.46 | 0 | 0 |
| NFKBIA | 2 | 0.96 | 0.86 | 0.46 | 0 | 0 |
| EBP | 2 | 0.50 | 0.16 | 0.46 | 0 | 0 |
| IFIT5 | 2 | 0.51 | 0.19 | 0.46 | 0 | 0 |
| JAK1 | 2 | 0.85 | 0.56 | 0.46 | 0 | 0 |
| PARP10 | 2 | 0.40 | 0.07 | 0.46 | 0 | 0 |
| SH3BP5 | 2 | 0.46 | 0.13 | 0.46 | 0 | 0 |
| RSU1 | 2 | 0.51 | 0.16 | 0.46 | 0 | 0 |
| ACTR3 | 2 | 0.96 | 0.83 | 0.46 | 0 | 0 |
| HUWE1 | 2 | 0.61 | 0.34 | 0.46 | 0 | 0 |
| ARL4C | 2 | 0.51 | 0.20 | 0.46 | 0 | 0 |
| PRMT2 | 2 | 0.54 | 0.18 | 0.46 | 0 | 0 |
| NDUFV2 | 2 | 0.96 | 0.82 | 0.46 | 0 | 0 |
| JUNB | 2 | 0.83 | 0.65 | 0.46 | 0 | 0 |
| DDIT4 | 2 | 0.50 | 0.28 | 0.45 | 0 | 0 |
| WIPF1 | 2 | 0.72 | 0.41 | 0.45 | 0 | 0 |
| CALCOCO2 | 2 | 0.66 | 0.33 | 0.45 | 0 | 0 |
| UPP1 | 2 | 0.47 | 0.14 | 0.45 | 0 | 0 |
| SP110 | 2 | 0.57 | 0.26 | 0.45 | 0 | 0 |
| CSTB | 2 | 0.88 | 0.66 | 0.45 | 0 | 0 |
| PDE4D | 2 | 0.48 | 0.16 | 0.45 | 0 | 0 |
| SLFN5 | 2 | 0.43 | 0.11 | 0.45 | 0 | 0 |
| DHRS7 | 2 | 0.66 | 0.31 | 0.45 | 0 | 0 |
| KDSR | 2 | 0.64 | 0.38 | 0.45 | 0 | 0 |
| NECAP2 | 2 | 0.61 | 0.29 | 0.44 | 0 | 0 |
| KCNA3 | 2 | 0.42 | 0.09 | 0.44 | 0 | 0 |
| PHF11 | 2 | 0.74 | 0.48 | 0.44 | 0 | 0 |
| ARHGDIA | 2 | 0.93 | 0.77 | 0.44 | 0 | 0 |
| SOCS2 | 2 | 0.37 | 0.08 | 0.44 | 0 | 0 |
| USP18 | 2 | 0.36 | 0.10 | 0.44 | 0 | 0 |
| CTSS | 2 | 0.53 | 0.21 | 0.44 | 0 | 0 |
| NUB1 | 2 | 0.51 | 0.22 | 0.43 | 0 | 0 |
| SMCHD1 | 2 | 0.74 | 0.48 | 0.43 | 0 | 0 |
| C19orf66 | 2 | 0.75 | 0.49 | 0.43 | 0 | 0 |
| CDC42SE2 | 2 | 0.73 | 0.44 | 0.43 | 0 | 0 |
| TAGLN2 | 2 | 0.91 | 0.76 | 0.43 | 0 | 0 |
| DDX60L | 2 | 0.41 | 0.12 | 0.43 | 0 | 0 |
| ARHGAP30 | 2 | 0.53 | 0.21 | 0.43 | 0 | 0 |
| STAT2 | 2 | 0.43 | 0.13 | 0.42 | 0 | 0 |
| LCP1 | 2 | 0.94 | 0.75 | 0.42 | 0 | 0 |
| CD53 | 2 | 0.94 | 0.77 | 0.42 | 0 | 0 |
| MYO1G | 2 | 0.43 | 0.12 | 0.42 | 0 | 0 |
| NAPA | 2 | 0.75 | 0.50 | 0.42 | 0 | 0 |
| KIFZA | 2 | 0.67 | 0.40 | 0.42 | 0 | 0 |
| PML | 2 | 0.45 | 0.17 | 0.42 | 0 | 0 |
| GLTSCR2 | 2 | 0.89 | 0.74 | 0.42 | 0 | 0 |
| MAGED2 | 2 | 0.50 | 0.19 | 0.41 | 0 | 0 |
| RABAC1 | 2 | 0.86 | 0.62 | 0.41 | 0 | 0 |
| ITGB7 | 2 | 0.44 | 0.15 | 0.41 | 0 | 0 |
| CYBA | 2 | 0.87 | 0.73 | 0.41 | 0 | 0 |
| CYTH1 | 2 | 0.47 | 0.16 | 0.41 | 0 | 0 |
| TREX1 | 2 | 0.42 | 0.15 | 0.41 | 0 | 0 |
| ARL6IP6 | 2 | 0.46 | 0.15 | 0.41 | 0 | 0 |
| RBMS1 | 2 | 0.51 | 0.20 | 0.40 | 0 | 0 |
| CCND3 | 2 | 0.75 | 0.49 | 0.40 | 0 | 0 |
| NMI | 2 | 0.61 | 0.35 | 0.40 | 0 | 0 |
| BIN1 | 2 | 0.41 | 0.10 | 0.40 | 0 | 0 |
| AES | 2 | 0.64 | 0.33 | 0.40 | 0 | 0 |
| NMRK1 | 2 | 0.41 | 0.11 | 0.40 | 0 | 0 |
| EVL | 2 | 0.71 | 0.38 | 0.40 | 0 | 0 |
| ETS1 | 2 | 0.43 | 0.13 | 0.40 | 0 | 0 |
| RAB11FIP1 | 2 | 0.56 | 0.27 | 0.40 | 0 | 0 |
| ODF2L | 2 | 0.39 | 0.09 | 0.40 | 0 | 0 |
| AC017002.1 | 2 | 0.37 | 0.21 | 0.40 | 0 | 0 |
| ZC3HAV1 | 2 | 0.56 | 0.27 | 0.40 | 0 | 0 |
| ICAM3 | 2 | 0.63 | 0.34 | 0.40 | 0 | 0 |
| PPP1CA | 2 | 0.89 | 0.69 | 0.40 | 0 | 0 |
| ARPC5 | 2 | 0.83 | 0.59 | 0.40 | 0 | 0 |
| LAP3 | 2 | 0.67 | 0.50 | 0.39 | 0 | 0 |
| RPS27L | 2 | 0.67 | 0.50 | 0.39 | 0 | 0 |
| GIMAP1 | 2 | 0.40 | 0.13 | 0.39 | 0 | 0 |
| S100A10 | 2 | 0.97 | 0.79 | 0.39 | 0 | 0 |
| YWHAH | 2 | 0.49 | 0.23 | 0.39 | 0 | 0 |
| MAT2B | 2 | 0.67 | 0.41 | 0.39 | 0 | 0 |
| VAMP5 | 2 | 0.40 | 0.14 | 0.39 | 0 | 0 |
| SOCS3 | 2 | 0.32 | 0.09 | 0.39 | 0 | 0 |
| TRAT1 | 2 | 0.59 | 0.28 | 0.39 | 0 | 0 |
| ITGA4 | 2 | 0.52 | 0.30 | 0.39 | 0 | 0 |
| MYL12B | 2 | 0.99 | 0.93 | 0.39 | 0 | 0 |
| NAGK | 2 | 0.47 | 0.18 | 0.38 | 0 | 0 |
| SHISA5 | 2 | 0.64 | 0.39 | 0.38 | 0 | 0 |
| TMEM123 | 2 | 0.81 | 0.60 | 0.38 | 0 | 0 |
| FDPS | 2 | 0.66 | 0.45 | 0.38 | 0 | 0 |
| AQP3 | 2 | 0.35 | 0.13 | 0.38 | 0 | 0 |
| HLA-C | 2 | 1.00 | 1.00 | 0.38 | 0 | 0 |
| HSP90AA1 | 2 | 1.00 | 0.96 | 0.38 | 0 | 0 |
| LSP1 | 2 | 0.66 | 0.35 | 0.38 | 0 | 0 |
| MYD88 | 2 | 0.48 | 0.21 | 0.38 | 0 | 0 |
| UGP2 | 2 | 0.48 | 0.20 | 0.38 | 0 | 0 |
| ADAM8 | 2 | 0.36 | 0.09 | 0.38 | 0 | 0 |
| TRIM21 | 2 | 0.48 | 0.20 | 0.38 | 0 | 0 |
| TALDO1 | 2 | 0.77 | 0.54 | 0.38 | 0 | 0 |
| FTH1 | 2 | 1.00 | 0.96 | 0.38 | 0 | 0 |
| PIGER2 | 2 | 0.45 | 0.20 | 0.38 | 0 | 0 |
| NUCB1 | 2 | 0.54 | 0.25 | 0.38 | 0 | 0 |
| TMEM50A | 2 | 0.91 | 0.74 | 0.38 | 0 | 0 |
| PPDPF | 2 | 0.92 | 0.77 | 0.37 | 0 | 0 |
| RPS6KAS | 2 | 0.36 | 0.08 | 0.37 | 0 | 0 |
| MYH9 | 2 | 0.77 | 0.56 | 0.37 | 0 | 0 |
| CLIP1 | 2 | 0.42 | 0.16 | 0.37 | 0 | 0 |
| RPL10 | 2 | 1.00 | 1.00 | 0.37 | 0 | 0 |
| CLIC1 | 2 | 0.98 | 0.93 | 0.37 | 0 | 0 |
| LDLR | 2 | 0.41 | 0.14 | 0.37 | 0 | 0 |
| SGK1 | 2 | 0.46 | 0.22 | 0.37 | 0 | 0 |
| GNA15 | 2 | 0.48 | 0.20 | 0.37 | 0 | 0 |
| SPOCK2 | 2 | 0.67 | 0.34 | 0.37 | 0 | 0 |
| KIAA0040 | 2 | 0.34 | 0.07 | 0.37 | 0 | 0 |
| TPM3 | 2 | 0.98 | 0.89 | 0.37 | 0 | 0 |
| FAM177A1 | 2 | 0.56 | 0.29 | 0.37 | 0 | 0 |
| GRAP2 | 2 | 0.41 | 0.14 | 0.37 | 0 | 0 |
| ADAR | 2 | 0.76 | 0.56 | 0.37 | 0 | 0 |
| ACAP2 | 2 | 0.50 | 0.21 | 0.37 | 0 | 0 |
| RALB | 2 | 0.34 | 0.06 | 0.36 | 0 | 0 |
| HELZ2 | 2 | 0.35 | 0.09 | 0.36 | 0 | 0 |
| TBC1D10C | 2 | 0.37 | 0.10 | 0.36 | 0 | 0 |
| C5orf56 | 2 | 0.40 | 0.12 | 0.36 | 0 | 0 |
| TRPV2 | 2 | 0.35 | 0.07 | 0.36 | 0 | 0 |
| PRDX5 | 2 | 0.79 | 0.57 | 0.36 | 0 | 0 |
| TXNIP | 2 | 0.32 | 0.10 | 0.36 | 0 | 0 |
| ANXA2R | 2 | 0.31 | 0.06 | 0.36 | 0 | 0 |
| TRAFD1 | 2 | 0.43 | 0.17 | 0.36 | 0 | 0 |
| SYNE2 | 2 | 0.80 | 0.56 | 0.36 | 0 | 0 |
| MB21D1 | 2 | 0.44 | 0.19 | 0.36 | 0 | 0 |
| COX17 | 2 | 0.72 | 0.50 | 0.35 | 0 | 0 |
| ZNF267 | 2 | 0.56 | 0.28 | 0.35 | 0 | 0 |
| RPL41 | 2 | 0.99 | 0.97 | 0.35 | 0 | 0 |
| TRAPPC1 | 2 | 0.73 | 0.49 | 0.35 | 0 | 0 |
| PPP1R15A | 2 | 0.74 | 0.59 | 0.35 | 0 | 0 |
| TMEM219 | 2 | 0.48 | 0.20 | 0.35 | 0 | 0 |
| CCR2 | 2 | 0.21 | 0.01 | 0.35 | 0 | 0 |
| TUBB4B | 2 | 0.81 | 0.67 | 0.35 | 0 | 0 |
| RNASEK | 2 | 0.89 | 0.72 | 0.35 | 0 | 0 |
| ANXA6 | 2 | 0.71 | 0.44 | 0.34 | 0 | 0 |
| CSF1 | 2 | 0.29 | 0.08 | 0.34 | 0 | 0 |
| TMEM50B | 2 | 0.40 | 0.13 | 0.34 | 0 | 0 |
| GUK1 | 2 | 0.93 | 0.78 | 0.34 | 0 | 0 |
| TUBA1B | 2 | 0.92 | 0.83 | 0.34 | 0 | 0 |
| MGAT4A | 2 | 0.42 | 0.14 | 0.34 | 0 | 0 |
| HMHA1 | 2 | 0.33 | 0.07 | 0.34 | 0 | 0 |
| LIF | 2 | 0.24 | 0.08 | 0.34 | 0 | 0 |
| RP11- | 2 | 0.26 | 0.09 | 0.34 | 0 | 0 |
| 124N14.3 | | | | | | |
| TMEM230 | 2 | 0.59 | 0.33 | 0.34 | 0 | 0 |
| CYLD | 2 | 0.65 | 0.38 | 0.34 | 0 | 0 |
| PHTF2 | 2 | 0.40 | 0.14 | 0.34 | 0 | 0 |
| MAP4 | 2 | 0.53 | 0.28 | 0.33 | 0 | 0 |
| SEPW1 | 2 | 0.82 | 0.62 | 0.33 | 0 | 0 |
| FDFT1 | 2 | 0.78 | 0.61 | 0.33 | 0 | 0 |
| PMVK | 2 | 0.53 | 0.30 | 0.33 | 0 | 0 |
| ANXA11 | 2 | 0.71 | 0.46 | 0.33 | 0 | 0 |
| IDH2 | 2 | 0.35 | 0.11 | 0.33 | 0 | 0 |
| P2RY8 | 2 | 0.28 | 0.04 | 0.33 | 0 | 0 |
| CYB5R3 | 2 | 0.51 | 0.25 | 0.33 | 0 | 0 |
| SATB1 | 2 | 0.50 | 0.28 | 0.33 | 0 | 0 |
| GLIPRZ | 2 | 0.39 | 0.14 | 0.33 | 0 | 0 |
| C9orf142 | 2 | 0.69 | 0.47 | 0.33 | 0 | 0 |
| LYSMD2 | 2 | 0.57 | 0.33 | 0.33 | 0 | 0 |
| LAMP3 | 2 | 0.32 | 0.11 | 0.32 | 0 | 0 |
| GNAI2 | 2 | 0.39 | 0.14 | 0.32 | 0 | 0 |
| RPL28 | 2 | 1.00 | 0.99 | 0.32 | 0 | 0 |
| DCTN2 | 2 | 0.55 | 0.30 | 0.32 | 0 | 0 |
| VPS28 | 2 | 0.74 | 0.53 | 0.32 | 0 | 0 |
| CAPN1 | 2 | 0.38 | 0.13 | 0.32 | 0 | 0 |
| IKBKE | 2 | 0.29 | 0.06 | 0.32 | 0 | 0 |
| DCK | 2 | 0.37 | 0.13 | 0.32 | 0 | 0 |
| FYB | 2 | 0.59 | 0.37 | 0.32 | 0 | 0 |
| CD37 | 2 | 0.82 | 0.61 | 0.32 | 0 | 0 |
| RPS12 | 2 | 1.00 | 1.00 | 0.32 | 0 | 0 |
| HLA-F | 2 | 0.87 | 0.67 | 0.32 | 0 | 0 |
| FBXW5 | 2 | 0.44 | 0.19 | 0.32 | 0 | 0 |
| RGS19 | 2 | 0.45 | 0.24 | 0.32 | 0 | 0 |
| FURIN | 2 | 0.37 | 0.18 | 0.32 | 0 | 0 |
| EMB | 2 | 0.46 | 0.21 | 0.32 | 0 | 0 |
| PRKX | 2 | 0.35 | 0.15 | 0.31 | 0 | 0 |
| CHST12 | 2 | 0.34 | 0.13 | 0.31 | 0 | 0 |
| FXYD5 | 2 | 0.97 | 0.89 | 0.31 | 0 | 0 |
| SELK | 2 | 0.84 | 0.66 | 0.31 | 0 | 0 |
| MB21D2 | 2 | 0.28 | 0.04 | 0.31 | 0 | 0 |
| WAS | 2 | 0.49 | 0.24 | 0.31 | 0 | 0 |
| RCSD1 | 2 | 0.44 | 0.20 | 0.31 | 0 | 0 |
| VPS29 | 2 | 0.62 | 0.40 | 0.31 | 0 | 0 |
| S1PR1 | 2 | 0.33 | 0.13 | 0.31 | 0 | 0 |
| CRYZ | 2 | 0.31 | 0.08 | 0.31 | 0 | 0 |
| SLC4A10 | 2 | 0.20 | 0.02 | 0.31 | 0 | 0 |
| LGALS3BP | 2 | 0.28 | 0.12 | 0.31 | 0 | 0 |
| RORA | 2 | 0.63 | 0.33 | 0.31 | 0 | 0 |
| ATP5H | 2 | 0.77 | 0.57 | 0.31 | 0 | 0 |
| CD247 | 2 | 0.76 | 0.51 | 0.31 | 0 | 0 |
| CAP1 | 2 | 0.88 | 0.73 | 0.31 | 0 | 0 |
| PGLS | 2 | 0.57 | 0.34 | 0.31 | 0 | 0 |
| PARP8 | 2 | 0.39 | 0.15 | 0.31 | 0 | 0 |
| ETHE1 | 2 | 0.40 | 0.17 | 0.31 | 0 | 0 |
| C19orf60 | 2 | 0.59 | 0.36 | 0.30 | 0 | 0 |
| ACAA2 | 2 | 0.45 | 0.20 | 0.30 | 0 | 0 |
| EHD4 | 2 | 0.57 | 0.35 | 0.30 | 0 | 0 |
| OST4 | 2 | 0.93 | 0.81 | 0.30 | 0 | 0 |
| COMMD6 | 2 | 0.84 | 0.66 | 0.30 | 0 | 0 |
| CPNE3 | 2 | 0.40 | 0.17 | 0.30 | 0 | 0 |
| C4orf3 | 2 | 0.83 | 0.64 | 0.30 | 0 | 0 |
| DCTN3 | 2 | 0.46 | 0.21 | 0.30 | 0 | 0 |
| MSC | 2 | 0.23 | 0.07 | 0.30 | 0 | 0 |
| TMEM59 | 2 | 0.77 | 0.56 | 0.30 | 0 | 0 |
| RGS14 | 2 | 0.26 | 0.05 | 0.30 | 0 | 0 |
| RPL13 | 2 | 1.00 | 1.00 | 0.30 | 0 | 0 |
| FKBP2 | 2 | 0.80 | 0.64 | 0.29 | 0 | 0 |
| RCAN3 | 2 | 0.33 | 0.11 | 0.29 | 0 | 0 |
| RBX1 | 2 | 0.84 | 0.69 | 0.29 | 0 | 0 |
| ELOVL1 | 2 | 0.54 | 0.31 | 0.29 | 0 | 0 |
| C6orf1 | 2 | 0.32 | 0.09 | 0.29 | 0 | 0 |
| DYNLRB1 | 2 | 0.73 | 0.53 | 0.29 | 0 | 0 |
| RASAL3 | 2 | 0.54 | 0.30 | 0.29 | 0 | 0 |
| VPS13C | 2 | 0.44 | 0.21 | 0.29 | 0 | 0 |
| PRDM1 | 2 | 0.41 | 0.17 | 0.29 | 0 | 0 |
| APOL1 | 2 | 0.27 | 0.05 | 0.29 | 0 | 0 |
| YWHAZ | 2 | 0.99 | 0.94 | 0.29 | 0 | 0 |
| IQGAP1 | 2 | 0.59 | 0.34 | 0.29 | 0 | 0 |
| PSIP1 | 2 | 0.44 | 0.21 | 0.29 | 0 | 0 |
| HLA-B | 2 | 1.00 | 1.00 | 0.29 | 0 | 0 |
| FLOT1 | 2 | 0.45 | 0.27 | 0.29 | 0 | 0 |
| GMFG | 2 | 0.83 | 0.62 | 0.29 | 0 | 0 |
| C14orf1 | 2 | 0.56 | 0.35 | 0.29 | 0 | 0 |
| IKZF1 | 2 | 0.64 | 0.39 | 0.29 | 0 | 0 |
| COMMD7 | 2 | 0.37 | 0.14 | 0.29 | 0 | 0 |
| IFI16 | 2 | 0.69 | 0.51 | 0.29 | 0 | 0 |
| TMEM173 | 2 | 0.35 | 0.16 | 0.29 | 0 | 0 |
| LMF2 | 2 | 0.39 | 0.15 | 0.29 | 0 | 0 |
| GNG2 | 2 | 0.77 | 0.53 | 0.29 | 0 | 0 |
| RAB11A | 2 | 0.70 | 0.55 | 0.29 | 0 | 0 |
| LST1 | 2 | 0.22 | 0.03 | 0.28 | 0 | 0 |
| NFKBIZ | 2 | 0.46 | 0.23 | 0.28 | 0 | 0 |
| RPS4X | 2 | 1.00 | 0.99 | 0.28 | 0 | 0 |
| TNIP1 | 2 | 0.71 | 0.48 | 0.28 | 0 | 0 |
| ECH1 | 2 | 0.51 | 0.28 | 0.28 | 0 | 0 |
| SMAP | 2 | 0.55 | 0.33 | 0.28 | 0 | 0 |
| SUMO3 | 2 | 0.57 | 0.36 | 0.28 | 0 | 0 |
| DOCK8 | 2 | 0.51 | 0.28 | 0.28 | 0 | 0 |
| SPINT2 | 2 | 0.24 | 0.07 | 0.28 | 0 | 0 |
| SLC25A24 | 2 | 0.26 | 0.05 | 0.28 | 0 | 0 |
| RAB1B | 2 | 0.72 | 0.53 | 0.28 | 0 | 0 |
| LRP10 | 2 | 0.49 | 0.25 | 0.28 | 0 | 0 |
| GLO1 | 2 | 0.50 | 0.29 | 0.28 | 0 | 0 |
| STK17A | 2 | 0.49 | 0.28 | 0.28 | 0 | 0 |
| SPG20 | 2 | 0.33 | 0.11 | 0.28 | 0 | 0 |
| CAMK4 | 2 | 0.48 | 0.24 | 0.27 | 0 | 0 |
| B2M | 2 | 1.00 | 1.00 | 0.27 | 0 | 0 |
| RAB7L1 | 2 | 0.37 | 0.16 | 0.27 | 0 | 0 |
| NME3 | 2 | 0.38 | 0.20 | 0.27 | 0 | 0 |
| GPR65 | 2 | 0.44 | 0.23 | 0.27 | 0 | 0 |
| CRELD2 | 2 | 0.46 | 0.25 | 0.27 | 0 | 0 |
| MANF | 2 | 0.76 | 0.60 | 0.27 | 0 | 0 |
| GPR137 | 2 | 0.34 | 0.13 | 0.27 | 0 | 0 |
| ARL2BP | 2 | 0.42 | 0.21 | 0.27 | 0 | 0 |
| MITD1 | 2 | 0.43 | 0.23 | 0.27 | 0 | 0 |
| ANXA5 | 2 | 0.62 | 0.35 | 0.27 | 0 | 0 |
| C19orf70 | 2 | 0.75 | 0.58 | 0.27 | 0 | 0 |
| GDI1 | 2 | 0.55 | 0.36 | 0.27 | 0 | 0 |
| ITSN2 | 2 | 0.51 | 0.29 | 0.27 | 0 | 0 |
| ATOX1 | 2 | 0.58 | 0.41 | 0.27 | 0 | 0 |
| BCL3 | 2 | 0.23 | 0.04 | 0.27 | 0 | 0 |
| PNRC1 | 2 | 0.66 | 0.44 | 0.27 | 0 | 0 |
| HBEGF | 2 | 0.14 | 0.03 | 0.27 | 0 | 0 |
| MAPKAPK3 | 2 | 0.48 | 0.31 | 0.27 | 0 | 0 |
| RTP4 | 2 | 0.24 | 0.05 | 0.26 | 0 | 0 |
| CHMP4A | 2 | 0.52 | 0.33 | 0.26 | 0 | 0 |
| STK10 | 2 | 0.33 | 0.12 | 0.26 | 0 | 0 |
| BLVRA | 2 | 0.39 | 0.19 | 0.26 | 0 | 0 |
| PSENEN | 2 | 0.54 | 0.33 | 0.26 | 0 | 0 |
| HMGN3 | 2 | 0.39 | 0.18 | 0.26 | 0 | 0 |
| PYCARD | 2 | 0.24 | 0.06 | 0.26 | 0 | 0 |
| KMT2A | 2 | 0.35 | 0.16 | 0.26 | 0 | 0 |
| GCH1 | 2 | 0.31 | 0.12 | 0.26 | 0 | 0 |
| REEP5 | 2 | 0.76 | 0.57 | 0.26 | 0 | 0 |
| HINT1 | 2 | 0.99 | 0.96 | 0.26 | 0 | 0 |
| CIGALT1 | 2 | 0.52 | 0.32 | 0.26 | 0 | 0 |
| RASA2 | 2 | 0.33 | 0.13 | 0.26 | 0 | 0 |
| FAM46C | 2 | 0.28 | 0.08 | 0.26 | 0 | 0 |
| SNX3 | 2 | 0.60 | 0.42 | 0.26 | 0 | 0 |
| TMEM256- | 2 | 0.35 | 0.16 | 0.26 | 0 | 0 |
| PLSCR3 | | | | | | |
| STOM | 2 | 0.42 | 0.21 | 0.26 | 0 | 0 |
| JAK3 | 2 | 0.42 | 0.22 | 0.26 | 0 | 0 |
| SPATS2L | 2 | 0.24 | 0.05 | 0.26 | 0 | 0 |
| NDUFB7 | 2 | 0.71 | 0.55 | 0.25 | 0 | 0 |
| LAMTOR4 | 2 | 0.68 | 0.50 | 0.25 | 0 | 0 |
| LNPEP | 2 | 0.37 | 0.15 | 0.25 | 0 | 0 |
| UQCRB | 2 | 0.89 | 0.76 | 0.25 | 0 | 0 |
| SLC39A8 | 2 | 0.32 | 0.13 | 0.25 | 0 | 0 |
| C1orf86 | 2 | 0.40 | 0.21 | 0.25 | 0 | 0 |
| FBXO6 | 2 | 0.27 | 0.09 | 0.25 | 0 | 0 |
| PHF1 | 2 | 0.42 | 0.20 | 0.25 | 0 | 0 |
| SEPT1 | 2 | 0.66 | 0.47 | 0.26 | 4.41813263137076e−319 | 6.12220638729047e−315 |
| GSTP1 | 2 | 0.85 | 0.73 | 0.27 | 3.67359914106818e−314 | 5.09050632977817e−310 |
| CKLF | 2 | 0.55 | 0.40 | 0.30 | 8.6472877116679e−311 | 1.20E−306 |
| GPX1 | 2 | 0.60 | 0.40 | 0.29 | 8.37E−298 | 1.16E−293 |
| CTSC | 2 | 0.68 | 0.50 | 0.30 | 3.58E−294 | 4.97E−290 |
| SOD2 | 2 | 0.52 | 0.36 | 0.26 | 2.16E−257 | 2.99E−253 |
| CCR7 | 2 | 0.34 | 0.20 | 0.34 | 2.13E−222 | 2.95E−218 |
| FOS | 2 | 0.43 | 0.41 | 0.35 | 3.30E−192 | 4.58E−188 |
| JUN | 2 | 0.49 | 0.38 | 0.37 | 8.10E−191 | 1.12E−186 |
| GZMA | 2 | 0.15 | 0.08 | 0.28 | 2.03E−142 | 2.82E−138 |
| FTL | 2 | 0.99 | 0.96 | 0.25 | 1.07E−123 | 1.48E−119 |

As referred to herein, Table 2 depicts as follows:

TABLE 2

| | | | | Test statistics | | |
|---|---|---|---|---|---|---|
| | | Fraction of | | Average | | |
| | | expressing cells | | logged | | |
| | | Cluster- | Other | fold | | Adjusted |
| Gene ID | Cluster | specific | cells | Change | P-value | P-value |
| CCL4 | 4 | 0.97 | 0.30 | 2.99 | 0 | 0 |
| XCL1 | 4 | 0.81 | 0.13 | 2.99 | 0 | 0 |
| XCL2 | 4 | 0.78 | 0.10 | 2.95 | 0 | 0 |
| GZMB | 4 | 0.96 | 0.19 | 2.44 | 0 | 0 |
| CCL3 | 4 | 0.77 | 0.11 | 2.43 | 0 | 0 |
| PRF1 | 4 | 0.94 | 0.20 | 1.99 | 0 | 0 |
| CCL4L2 | 4 | 0.57 | 0.04 | 1.84 | 0 | 0 |
| CCL5 | 4 | 0.93 | 0.23 | 1.71 | 0 | 0 |
| PLEK | 4 | 0.86 | 0.12 | 1.68 | 0 | 0 |
| NKG7 | 4 | 0.89 | 0.18 | 1.61 | 0 | 0 |
| CCL4L1 | 4 | 0.47 | 0.06 | 1.60 | 0 | 0 |
| GZMH | 4 | 0.69 | 0.06 | 1.54 | 0 | 0 |
| GNLY | 4 | 0.64 | 0.10 | 1.51 | 0 | 0 |
| CRTAM | 4 | 0.40 | 0.05 | 1.46 | 0 | 0 |
| SLAMF7 | 4 | 0.67 | 0.08 | 1.14 | 0 | 0 |
| HOPX | 4 | 0.78 | 0.20 | 1.14 | 0 | 0 |
| CD72 | 4 | 0.53 | 0.08 | 1.03 | 0 | 0 |
| CST7 | 4 | 0.91 | 0.52 | 0.92 | 0 | 0 |
| FASLG | 4 | 0.80 | 0.41 | 0.91 | 0 | 0 |
| EGR2 | 4 | 0.76 | 0.40 | 0.86 | 0 | 0 |
| ZEB2 | 4 | 0.70 | 0.30 | 0.83 | 0 | 0 |
| PCID2 | 4 | 0.55 | 0.41 | 0.75 | 0 | 0 |
| IQCG | 4 | 0.29 | 0.07 | 0.75 | 0 | 0 |
| PPP1R2 | 4 | 0.92 | 0.77 | 0.73 | 0 | 0 |
| ZFP36L1 | 4 | 0.93 | 0.83 | 0.69 | 0 | 0 |
| TNFRSF9 | 4 | 0.83 | 0.53 | 0.69 | 0 | 0 |
| BTG1 | 4 | 0.99 | 0.95 | 0.67 | 0 | 0 |
| TRIM22 | 4 | 0.86 | 0.66 | 0.66 | 0 | 0 |
| CD160 | 4 | 0.20 | 0.02 | 0.66 | 0 | 0 |
| LITAF | 4 | 0.70 | 0.47 | 0.65 | 0 | 0 |
| APOBEC3G | 4 | 0.83 | 0.58 | 0.65 | 0 | 0 |
| TAGAP | 4 | 0.92 | 0.76 | 0.62 | 0 | 0 |
| CFLAR | 4 | 0.89 | 0.79 | 0.59 | 0 | 0 |
| GPR18 | 4 | 0.40 | 0.13 | 0.59 | 0 | 0 |
| TGIF1 | 4 | 0.69 | 0.52 | 0.59 | 0 | 0 |
| CBLB | 4 | 0.79 | 0.59 | 0.58 | 0 | 0 |
| EVI2A | 4 | 0.72 | 0.53 | 0.58 | 0 | 0 |
| TMBIM1 | 4 | 0.63 | 0.47 | 0.53 | 0 | 0 |
| IL18RAP | 4 | 0.39 | 0.15 | 0.53 | 0 | 0 |
| LTBP4 | 4 | 0.73 | 0.55 | 0.53 | 0 | 0 |
| TNFSF9 | 4 | 0.48 | 0.19 | 0.53 | 0 | 0 |
| CX3CR1 | 4 | 0.25 | 0.03 | 0.52 | 0 | 0 |
| APOBEC3C | 4 | 0.55 | 0.37 | 0.51 | 0 | 0 |
| CD84 | 4 | 0.50 | 0.29 | 0.51 | 0 | 0 |
| CD97 | 4 | 0.74 | 0.59 | 0.50 | 0 | 0 |
| LYST | 4 | 0.61 | 0.46 | 0.50 | 0 | 0 |
| CD58 | 4 | 0.67 | 0.51 | 0.50 | 0 | 0 |
| NUCB2 | 4 | 0.38 | 0.27 | 0.50 | 0 | 0 |
| TNFAIP8 | 4 | 0.90 | 0.83 | 0.49 | 0 | 0 |
| PAM | 4 | 0.69 | 0.50 | 0.48 | 0 | 0 |
| VCL | 4 | 0.32 | 0.11 | 0.47 | 0 | 0 |
| THEMIS | 4 | 0.34 | 0.18 | 0.47 | 0 | 0 |
| CCDC107 | 4 | 0.53 | 0.38 | 0.46 | 0 | 0 |
| SRGN | 4 | 1.00 | 0.99 | 0.45 | 0 | 0 |
| HMGB1 | 4 | 0.89 | 0.86 | 0.45 | 0 | 0 |
| DUSP18 | 4 | 0.39 | 0.23 | 0.45 | 0 | 0 |
| RHOG | 4 | 0.94 | 0.86 | 0.45 | 0 | 0 |
| SLAMF6 | 4 | 0.48 | 0.31 | 0.44 | 0 | 0 |
| STAT5A | 4 | 0.58 | 0.47 | 0.44 | 0 | 0 |
| CD6 | 4 | 0.74 | 0.62 | 0.43 | 0 | 0 |
| DNAJB9 | 4 | 0.47 | 0.32 | 0.43 | 0 | 0 |
| ARL6IP1 | 4 | 0.77 | 0.74 | 0.42 | 0 | 0 |
| CCL3L1 | 4 | 0.11 | 0.01 | 0.42 | 0 | 0 |
| UCP2 | 4 | 0.62 | 0.49 | 0.42 | 0 | 0 |
| UBB | 4 | 0.96 | 0.93 | 0.41 | 0 | 0 |
| PRKCH | 4 | 0.82 | 0.70 | 0.41 | 0 | 0 |
| XIRP1 | 4 | 0.19 | 0.08 | 0.41 | 0 | 0 |
| PDHA1 | 4 | 0.60 | 0.51 | 0.41 | 0 | 0 |
| CD82 | 4 | 0.98 | 0.91 | 0.40 | 0 | 0 |
| BCL2A1 | 4 | 0.86 | 0.68 | 0.40 | 0 | 0 |
| TUBA1B | 4 | 0.90 | 0.84 | 0.40 | 0 | 0 |
| PGAM1 | 4 | 1.00 | 0.97 | 0.39 | 0 | 0 |
| PRSS23 | 4 | 0.17 | 0.04 | 0.39 | 0 | 0 |
| SSR2 | 4 | 0.84 | 0.84 | 0.39 | 0 | 0 |
| RINS | 4 | 0.26 | 0.09 | 0.39 | 0 | 0 |
| CHMP4B | 4 | 0.62 | 0.51 | 0.39 | 0 | 0 |
| YWHAQ | 4 | 0.88 | 0.85 | 0.38 | 0 | 0 |
| SEC61B | 4 | 0.97 | 0.94 | 0.38 | 0 | 0 |
| H3F3B | 4 | 1.00 | 0.99 | 0.38 | 0 | 0 |
| MIR4435-1HG | 4 | 0.64 | 0.42 | 0.38 | 0 | 0 |
| ARF4 | 4 | 0.82 | 0.78 | 0.38 | 0 | 0 |
| RP11- | 4 | 0.49 | 0.41 | 0.38 | 0 | 0 |
| 773D16.1 | | | | | | |
| GLUD1 | 4 | 0.71 | 0.62 | 0.38 | 0 | 0 |
| PPM1B | 4 | 0.36 | 0.23 | 0.36 | 0 | 0 |
| HLA-DPB1 | 4 | 0.34 | 0.16 | 0.36 | 0 | 0 |
| ETS2 | 4 | 0.32 | 0.22 | 0.36 | 0 | 0 |
| HECTD2 | 4 | 0.32 | 0.18 | 0.36 | 0 | 0 |
| TPS12 | 4 | 0.40 | 0.30 | 0.36 | 0 | 0 |
| PIGT | 4 | 0.51 | 0.42 | 0.35 | 0 | 0 |
| IL12RB2 | 4 | 0.27 | 0.16 | 0.35 | 0 | 0 |
| RAP1B | 4 | 0.85 | 0.80 | 0.35 | 0 | 0 |
| SSR3 | 4 | 0.68 | 0.65 | 0.35 | 0 | 0 |
| SEC61A1 | 4 | 0.61 | 0.55 | 0.35 | 0 | 0 |
| BCL2L1 | 4 | 0.69 | 0.62 | 0.35 | 0 | 0 |
| MAST3 | 4 | 0.23 | 0.10 | 0.35 | 0 | 0 |
| OSTC | 4 | 0.83 | 0.81 | 0.34 | 0 | 0 |
| BCL2L11 | 4 | 0.31 | 0.21 | 0.34 | 0 | 0 |
| SERP1 | 4 | 0.89 | 0.89 | 0.34 | 0 | 0 |
| ATP1B3 | 4 | 0.82 | 0.76 | 0.34 | 0 | 0 |
| MIR155HG | 4 | 0.99 | 0.94 | 0.34 | 0 | 0 |
| PKM | 4 | 1.00 | 0.98 | 0.33 | 0 | 0 |
| PTTG1 | 4 | 0.35 | 0.26 | 0.33 | 0 | 0 |
| SPCS2 | 4 | 0.85 | 0.84 | 0.33 | 0 | 0 |
| KDELR2 | 4 | 0.73 | 0.71 | 0.33 | 0 | 0 |
| UBE2B | 4 | 0.68 | 0.64 | 0.32 | 0 | 0 |
| ATP1B1 | 4 | 0.19 | 0.07 | 0.32 | 0 | 0 |
| AGO2 | 4 | 0.47 | 0.37 | 0.32 | 0 | 0 |
| TROVE2 | 4 | 0.44 | 0.38 | 0.32 | 0 | 0 |
| RHOB | 4 | 0.24 | 0.12 | 0.31 | 0 | 0 |
| LRRFIP2 | 4 | 0.36 | 0.29 | 0.31 | 0 | 0 |
| GORASP2 | 4 | 0.56 | 0.53 | 0.31 | 0 | 0 |
| C10orf54 | 4 | 0.91 | 0.75 | 0.31 | 0 | 0 |
| SEC61G | 4 | 0.84 | 0.84 | 0.31 | 0 | 0 |
| GSTO1 | 4 | 0.53 | 0.51 | 0.29 | 0 | 0 |
| IFNG | 4 | 0.95 | 0.62 | 0.29 | 0 | 0 |
| CCND3 | 4 | 0.59 | 0.51 | 0.28 | 0 | 0 |
| TMEM167A | 4 | 0.57 | 0.56 | 0.28 | 0 | 0 |
| C19orf10 | 4 | 0.80 | 0.80 | 0.28 | 0 | 0 |
| MAP2K3 | 4 | 0.87 | 0.75 | 0.27 | 0 | 0 |
| INPP1 | 4 | 0.20 | 0.11 | 0.27 | 0 | 0 |
| RAB27A | 4 | 0.68 | 0.58 | 0.27 | 0 | 0 |
| RGCC | 4 | 0.90 | 0.74 | 0.27 | 0 | 0 |
| ARF1 | 4 | 0.92 | 0.92 | 0.27 | 0 | 0 |
| HMGN2 | 4 | 0.72 | 0.74 | 0.26 | 0 | 0 |
| TMED2 | 4 | 0.77 | 0.79 | 0.25 | 0 | 0 |
| ZNF706 | 4 | 0.80 | 0.78 | 0.25 | 0 | 0 |
| CREB3L2 | 4 | 0.19 | 0.10 | 0.25 | 0 | 0 |
| SLAMF1 | 4 | 0.87 | 0.76 | 0.40 | 1.88733076711356e−321 | 2.61527424398926e−317 |
| PPP1R18 | 4 | 0.63 | 0.57 | 0.34 | 3.08626504790714e−318 | 4.27663747688492e−314 |
| EXOC2 | 4 | 0.36 | 0.27 | 0.35 | 2.40955222598001e−317 | 3.3389165195405e−313 |
| HLA-DPA1 | 4 | 0.35 | 0.22 | 0.26 | 3.41094275739994e−317 | 4.7265433789291e−313 |
| HLA-B | 4 | 1.00 | 1.00 | 0.31 | 1.45823270332801e−315 | 2.02067305700162e−311 |
| HCST | 4 | 0.77 | 0.71 | 0.40 | 2.13866733213076e−312 | 2.96E−308 |
| PAIP2 | 4 | 0.61 | 0.59 | 0.27 | 4.82158305607204e−310 | 6.68E−306 |
| TESK1 | 4 | 0.21 | 0.08 | 0.27 | 1.29E−305 | 1.78E−301 |
| CINNA1 | 4 | 0.44 | 0.32 | 0.38 | 2.14E−305 | 2.96E−301 |
| CLCF1 | 4 | 0.17 | 0.08 | 0.25 | 3.74E−302 | 5.18E−298 |
| STARD4 | 4 | 0.43 | 0.34 | 0.31 | 6.32E−302 | 8.76E−298 |
| HLA-E | 4 | 1.00 | 1.00 | 0.27 | 1.79E−301 | 2.48E−297 |
| QPCT | 4 | 0.20 | 0.09 | 0.30 | 3.38E−301 | 4.68E−297 |
| CTSC | 4 | 0.60 | 0.52 | 0.33 | 5.06E−299 | 7.01E−295 |
| TMED10 | 4 | 0.76 | 0.75 | 0.31 | 3.14E−297 | 4.35E−293 |
| BIRC3 | 4 | 0.80 | 0.69 | 0.59 | 1.52E−295 | 2.11E−291 |
| SSR1 | 4 | 0.57 | 0.56 | 0.25 | 5.61E−293 | 7.78E−289 |
| GPR137B | 4 | 0.24 | 0.15 | 0.29 | 1.87E−289 | 2.60E−285 |
| IGF2R | 4 | 0.31 | 0.18 | 0.33 | 2.16E−285 | 2.99E−281 |
| ARMCX3 | 4 | 0.39 | 0.32 | 0.31 | 9.66E−280 | 1.34E−275 |
| PRR13 | 4 | 0.67 | 0.67 | 0.27 | 1.66E−274 | 2.30E−270 |
| ATP2B4 | 4 | 0.33 | 0.21 | 0.34 | 3.27E−273 | 4.53E−269 |
| SERPINE2 | 4 | 0.26 | 0.18 | 0.34 | 1.56E−272 | 2.17E−268 |
| ANKRD28 | 4 | 0.34 | 0.18 | 0.37 | 1.84E−269 | 2.55E−265 |
| TUBA1C | 4 | 0.78 | 0.78 | 0.25 | 5.71E−268 | 7.91E−264 |
| NR3C1 | 4 | 0.63 | 0.54 | 0.40 | 9.96E−267 | 1.38E−262 |
| ZYX | 4 | 0.58 | 0.52 | 0.32 | 2.41E−264 | 3.34E−260 |
| VASP | 4 | 0.72 | 0.70 | 0.27 | 6.99E−260 | 9.68E−256 |
| TNFRSF1B | 4 | 0.76 | 0.72 | 0.28 | 1.69E−252 | 2.34E−248 |
| SDCBP | 4 | 0.70 | 0.65 | 0.40 | 6.48E−246 | 8.98E−242 |
| MDFIC | 4 | 0.51 | 0.43 | 0.32 | 1.69E−243 | 2.35E−239 |
| CHSY1 | 4 | 0.24 | 0.14 | 0.27 | 3.49E−242 | 4.84E−238 |
| TNFRSF1A | 4 | 0.31 | 0.20 | 0.31 | 2.44E−239 | 3.38E−235 |
| GLUL | 4 | 0.33 | 0.22 | 0.31 | 5.30E−239 | 7.35E−235 |
| TIGIT | 4 | 0.34 | 0.26 | 0.35 | 1.23E−237 | 1.70E−233 |
| HBP1 | 4 | 0.30 | 0.19 | 0.33 | 1.67E−232 | 2.31E−228 |
| IQGAP2 | 4 | 0.32 | 0.24 | 0.28 | 1.68E−232 | 2.32E−228 |
| KIF21A | 4 | 0.33 | 0.26 | 0.29 | 1.36E−231 | 1.89E−227 |
| MAP3K8 | 4 | 0.47 | 0.35 | 0.33 | 3.45E−227 | 4.78E−223 |
| DYNLT3 | 4 | 0.50 | 0.41 | 0.37 | 1.93E−225 | 2.67E−221 |
| NFATC3 | 4 | 0.29 | 0.22 | 0.26 | 1.60E−224 | 2.21E−220 |
| STX5 | 4 | 0.35 | 0.30 | 0.25 | 1.96E−221 | 2.71E−217 |
| TNFAIP3 | 4 | 0.74 | 0.66 | 0.40 | 5.43E−220 | 7.53E−216 |
| CDC42EP3 | 4 | 0.59 | 0.45 | 0.42 | 6.46E−219 | 8.96E−215 |
| SLC29A1 | 4 | 0.36 | 0.36 | 0.27 | 1.12E−213 | 1.56E−209 |
| NR4A2 | 4 | 0.58 | 0.43 | 0.38 | 1.83E−213 | 2.54E−209 |
| MAP1LC3A | 4 | 0.34 | 0.24 | 0.32 | 9.57E−213 | 1.33E−208 |
| TMC6 | 4 | 0.35 | 0.26 | 0.32 | 2.14E−212 | 2.96E−208 |
| CREB3 | 4 | 0.36 | 0.31 | 0.26 | 5.57E−209 | 7.72E−205 |
| JARID2 | 4 | 0.49 | 0.41 | 0.33 | 3.89E−205 | 5.38E−201 |
| EIF1B | 4 | 0.69 | 0.69 | 0.25 | 6.51E−204 | 9.02E−200 |
| CD83 | 4 | 0.50 | 0.41 | 0.51 | 7.44E−203 | 1.03E−198 |
| TNFSF10 | 4 | 0.73 | 0.59 | 0.28 | 4.11E−202 | 5.70E−198 |
| N4BP2L1 | 4 | 0.25 | 0.17 | 0.27 | 9.24E−199 | 1.28E−194 |
| HERPUD2 | 4 | 0.24 | 0.18 | 0.26 | 6.84E−196 | 9.48E−192 |
| HOXB2 | 4 | 0.26 | 0.17 | 0.26 | 1.22E−193 | 1.69E−189 |
| GNAS | 4 | 0.55 | 0.54 | 0.26 | 3.34E−192 | 4.63E−188 |
| EPS15 | 4 | 0.34 | 0.28 | 0.25 | 8.72E−191 | 1.21E−186 |
| TLN1 | 4 | 0.42 | 0.36 | 0.28 | 1.15E−181 | 1.60E−177 |
| PIM1 | 4 | 0.48 | 0.38 | 0.31 | 2.52E−181 | 3.49E−177 |
| FYN | 4 | 0.74 | 0.66 | 0.31 | 3.41E−180 | 4.73E−176 |
| PRNP | 4 | 0.82 | 0.74 | 0.46 | 4.41E−180 | 6.11E−176 |
| RLF | 4 | 0.41 | 0.32 | 0.30 | 5.49E−179 | 7.61E−175 |
| BATF3 | 4 | 0.17 | 0.12 | 0.26 | 1.05E−174 | 1.45E−170 |
| LBH | 4 | 0.57 | 0.53 | 0.28 | 1.24E−164 | 1.72E−160 |
| SLA | 4 | 0.69 | 0.60 | 0.35 | 1.73E−160 | 2.40E−156 |
| SLC4A7 | 4 | 0.34 | 0.24 | 0.30 | 7.47E−160 | 1.04E−155 |
| TRAF1 | 4 | 0.72 | 0.66 | 0.32 | 5.74E−153 | 7.95E−149 |
| N4BP2L2 | 4 | 0.59 | 0.60 | 0.25 | 2.93E−151 | 4.06E−147 |
| RASSF5 | 4 | 0.70 | 0.66 | 0.29 | 1.09E−148 | 1.52E−144 |
| SIT1 | 4 | 0.31 | 0.24 | 0.27 | 6.04E−145 | 8.37E−141 |
| CD48 | 4 | 0.89 | 0.88 | 0.26 | 3.64E−135 | 5.04E−131 |
| SLC20A1 | 4 | 0.40 | 0.34 | 0.29 | 9.68E−134 | 1.34E−129 |
| IRF4 | 4 | 0.59 | 0.51 | 0.29 | 1.62E−130 | 2.24E−126 |
| TM2D3 | 4 | 0.65 | 0.60 | 0.25 | 8.24E−129 | 1.14E−124 |
| PIGER2 | 4 | 0.36 | 0.22 | 0.31 | 2.95E−121 | 4.08E−117 |
| BCL2 | 4 | 0.43 | 0.36 | 0.28 | 1.60E−118 | 2.21E−114 |
| IL7R | 4 | 0.91 | 0.79 | 0.41 | 1.91E−116 | 2.65E−112 |
| AC006369.2 | 4 | 0.26 | 0.15 | 0.31 | 1.02E−111 | 1.41E−107 |
| KLF10 | 4 | 0.50 | 0.43 | 0.30 | 4.43E−110 | 6.14E−106 |
| MAML2 | 4 | 0.42 | 0.33 | 0.26 | 8.95E−108 | 1.24E−103 |
| KMT2E | 4 | 0.70 | 0.68 | 0.28 | 2.92E−107 | 4.05E−103 |
| CTLA4 | 4 | 0.42 | 0.41 | 0.25 | 1.06E−105 | 1.46E−101 |
| XBP1 | 4 | 0.74 | 0.70 | 0.29 | 1.65E−103 | 2.28E−99 |
| KDM6B | 4 | 0.62 | 0.53 | 0.30 | 5.01E−103 | 6.95E−99 |
| ITK | 4 | 0.65 | 0.62 | 0.26 | 2.88E−102 | 3.99E−98 |
| LGALS1 | 4 | 0.50 | 0.42 | 0.27 | 6.13E−97 | 8.49E−93 |
| PHLDA1 | 4 | 0.61 | 0.57 | 0.27 | 8.32E−81 | 1.15E−76 |
| PIGER4 | 4 | 0.56 | 0.47 | 0.32 | 1.52E−71 | 2.11E−67 |
| BIG2 | 4 | 0.55 | 0.47 | 0.38 | 1.04E−68 | 1.45E−64 |
| NR4A3 | 4 | 0.42 | 0.35 | 0.32 | 1.03E−56 | 1.42E−52 |

As referred to herein, Table 3 depicts as follows:

TABLE 3

| | | | | Test statistics | | |
|---|---|---|---|---|---|---|
| | | Fraction of | | Average | | |
| | | expressing cells | | logged | | |
| | | Cluster- | Other | Fold | | Adjusted |
| Gene ID | Cluster | specific | cells | Change | P-value | P-value |
| AC006129.4 | 6 | 0.72 | 0.10 | 1.25 | 0 | 0 |
| DOK5 | 6 | 0.41 | 0.04 | 1.23 | 0 | 0 |
| NMB | 6 | 0.58 | 0.18 | 1.07 | 0 | 0 |
| FABPS | 6 | 1.00 | 0.67 | 1.06 | 0 | 0 |
| ZBED2 | 6 | 0.59 | 0.15 | 0.99 | 0 | 0 |
| HLA-DRA | 6 | 0.58 | 0.08 | 0.91 | 0 | 0 |
| POUZAF1 | 6 | 0.89 | 0.28 | 0.90 | 0 | 0 |
| FKBP1A | 6 | 0.99 | 0.84 | 0.87 | 0 | 0 |
| CD70 | 6 | 0.51 | 0.16 | 0.86 | 0 | 0 |
| SLC27A2 | 6 | 0.91 | 0.28 | 0.84 | 0 | 0 |
| DYNLL1 | 6 | 0.95 | 0.61 | 0.82 | 0 | 0 |
| DUSP4 | 6 | 0.93 | 0.44 | 0.79 | 0 | 0 |
| EID1 | 6 | 0.96 | 0.72 | 0.78 | 0 | 0 |
| MIR4435-1HG | 6 | 0.94 | 0.40 | 0.76 | 0 | 0 |
| ITM2A | 6 | 0.90 | 0.55 | 0.75 | 0 | 0 |
| GNG4 | 6 | 0.77 | 0.22 | 0.70 | 0 | 0 |
| C16orf45 | 6 | 0.59 | 0.13 | 0.68 | 0 | 0 |
| RAB27A | 6 | 0.94 | 0.56 | 0.65 | 0 | 0 |
| REXO2 | 6 | 0.95 | 0.62 | 0.64 | 0 | 0 |
| ANXA5 | 6 | 0.75 | 0.35 | 0.64 | 0 | 0 |
| LAT | 6 | 0.90 | 0.58 | 0.64 | 0 | 0 |
| CCND3 | 6 | 0.80 | 0.50 | 0.63 | 0 | 0 |
| PGAM1 | 6 | 1.00 | 0.97 | 0.62 | 0 | 0 |
| ZBTB32 | 6 | 0.68 | 0.18 | 0.61 | 0 | 0 |
| HLA-DRB1 | 6 | 0.58 | 0.17 | 0.60 | 0 | 0 |
| GALM | 6 | 0.75 | 0.29 | 0.60 | 0 | 0 |
| LAG3 | 6 | 0.68 | 0.27 | 0.59 | 0 | 0 |
| AHI1 | 6 | 0.85 | 0.40 | 0.59 | 0 | 0 |
| AGK | 6 | 0.81 | 0.34 | 0.58 | 0 | 0 |
| TRAF3IP3 | 6 | 0.66 | 0.24 | 0.57 | 0 | 0 |
| CD200 | 6 | 0.93 | 0.50 | 0.57 | 0 | 0 |
| ANKH | 6 | 0.62 | 0.26 | 0.57 | 0 | 0 |
| ATP5G3 | 6 | 0.99 | 0.89 | 0.56 | 0 | 0 |
| SOD1 | 6 | 1.00 | 0.91 | 0.56 | 0 | 0 |
| RPS6KA1 | 6 | 0.75 | 0.27 | 0.56 | 0 | 0 |
| TBC1D4 | 6 | 0.84 | 0.35 | 0.55 | 0 | 0 |
| PPP1CC | 6 | 0.95 | 0.65 | 0.54 | 0 | 0 |
| TIMMDC1 | 6 | 0.75 | 0.28 | 0.53 | 0 | 0 |
| ARMC9 | 6 | 0.46 | 0.07 | 0.52 | 0 | 0 |
| RGCC | 6 | 0.98 | 0.73 | 0.52 | 0 | 0 |
| COTL1 | 6 | 0.99 | 0.78 | 0.51 | 0 | 0 |
| CNIH1 | 6 | 0.95 | 0.68 | 0.50 | 0 | 0 |
| C7orf73 | 6 | 0.85 | 0.42 | 0.50 | 0 | 0 |
| C1QBP | 6 | 0.99 | 0.88 | 0.49 | 0 | 0 |
| DSTN | 6 | 0.75 | 0.40 | 0.49 | 0 | 0 |
| IFNG | 6 | 0.93 | 0.62 | 0.49 | 0 | 0 |
| TIMM13 | 6 | 0.94 | 0.62 | 0.48 | 0 | 0 |
| PDCD1 | 6 | 0.81 | 0.40 | 0.48 | 0 | 0 |
| PRDX3 | 6 | 0.93 | 0.62 | 0.48 | 0 | 0 |
| LINC00152 | 6 | 0.96 | 0.62 | 0.48 | 0 | 0 |
| PFDN4 | 6 | 0.80 | 0.42 | 0.48 | 0 | 0 |
| ADSS | 6 | 0.89 | 0.53 | 0.47 | 0 | 0 |
| PARVB | 6 | 0.66 | 0.18 | 0.47 | 0 | 0 |
| SMS | 6 | 0.89 | 0.56 | 0.47 | 0 | 0 |
| LDHA | 6 | 1.00 | 0.96 | 0.47 | 0 | 0 |
| FERMT3 | 6 | 0.90 | 0.54 | 0.47 | 0 | 0 |
| TIGIT | 6 | 0.54 | 0.24 | 0.47 | 0 | 0 |
| SEC11A | 6 | 0.94 | 0.64 | 0.46 | 0 | 0 |
| UBASH3B | 6 | 0.62 | 0.16 | 0.46 | 0 | 0 |
| GEM | 6 | 0.61 | 0.17 | 0.46 | 0 | 0 |
| SDC4 | 6 | 0.75 | 0.37 | 0.46 | 0 | 0 |
| COA6 | 6 | 0.86 | 0.47 | 0.46 | 0 | 0 |
| PARK7 | 6 | 1.00 | 0.94 | 0.46 | 0 | 0 |
| GLRX3 | 6 | 0.94 | 0.62 | 0.45 | 0 | 0 |
| TMED3 | 6 | 0.75 | 0.32 | 0.45 | 0 | 0 |
| MRPS34 | 6 | 0.89 | 0.55 | 0.45 | 0 | 0 |
| MDH2 | 6 | 0.95 | 0.68 | 0.45 | 0 | 0 |
| PLEKHF1 | 6 | 0.42 | 0.08 | 0.44 | 0 | 0 |
| HLA-DRB5 | 6 | 0.47 | 0.13 | 0.44 | 0 | 0 |
| GALNT2 | 6 | 0.59 | 0.16 | 0.42 | 0 | 0 |
| INPP5F | 6 | 0.47 | 0.10 | 0.42 | 0 | 0 |
| C12orf10 | 6 | 0.73 | 0.31 | 0.42 | 0 | 0 |
| TMEM173 | 6 | 0.51 | 0.16 | 0.42 | 0 | 0 |
| XIRP1 | 6 | 0.31 | 0.07 | 0.42 | 0 | 0 |
| CCDC50 | 6 | 0.35 | 0.10 | 0.42 | 0 | 0 |
| MYO1E | 6 | 0.36 | 0.02 | 0.42 | 0 | 0 |
| C16orf87 | 6 | 0.63 | 0.20 | 0.42 | 0 | 0 |
| GRAMD1A | 6 | 0.54 | 0.19 | 0.42 | 0 | 0 |
| ANAPC1 | 6 | 0.43 | 0.21 | 0.41 | 0 | 0 |
| SMOX | 6 | 0.48 | 0.13 | 0.41 | 0 | 0 |
| PPP1R2 | 6 | 0.98 | 0.77 | 0.41 | 0 | 0 |
| NUCB2 | 6 | 0.58 | 0.25 | 0.41 | 0 | 0 |
| CXCR3 | 6 | 0.81 | 0.38 | 0.41 | 0 | 0 |
| CD109 | 6 | 0.56 | 0.13 | 0.40 | 0 | 0 |
| GTF3C6 | 6 | 0.93 | 0.67 | 0.40 | 0 | 0 |
| GPI | 6 | 0.94 | 0.67 | 0.40 | 0 | 0 |
| FAM3C | 6 | 0.48 | 0.17 | 0.40 | 0 | 0 |
| POU2F2 | 6 | 0.91 | 0.58 | 0.40 | 0 | 0 |
| TSHZ2 | 6 | 0.70 | 0.25 | 0.39 | 0 | 0 |
| YWHAE | 6 | 0.92 | 0.62 | 0.39 | 0 | 0 |
| MYL6B | 6 | 0.49 | 0.08 | 0.39 | 0 | 0 |
| APOBEC3C | 6 | 0.75 | 0.36 | 0.38 | 0 | 0 |
| PSMA1 | 6 | 0.98 | 0.82 | 0.38 | 0 | 0 |
| CD74 | 6 | 0.94 | 0.75 | 0.38 | 0 | 0 |
| TIMM17A | 6 | 0.89 | 0.58 | 0.38 | 0 | 0 |
| ATP1B3 | 6 | 0.98 | 0.74 | 0.38 | 0 | 0 |
| RDH10 | 6 | 0.42 | 0.08 | 0.38 | 0 | 0 |
| SNX8 | 6 | 0.56 | 0.17 | 0.38 | 0 | 0 |
| ENOPH1 | 6 | 0.79 | 0.43 | 0.38 | 0 | 0 |
| C12orf75 | 6 | 0.42 | 0.12 | 0.38 | 0 | 0 |
| LEPROTL1 | 6 | 0.87 | 0.51 | 0.38 | 0 | 0 |
| CTPS1 | 6 | 0.88 | 0.50 | 0.38 | 0 | 0 |
| APOBEC3G | 6 | 0.87 | 0.58 | 0.38 | 0 | 0 |
| PHB2 | 6 | 0.97 | 0.79 | 0.37 | 0 | 0 |
| CLTA | 6 | 0.94 | 0.69 | 0.37 | 0 | 0 |
| RCC1 | 6 | 0.92 | 0.61 | 0.37 | 0 | 0 |
| POMP | 6 | 0.99 | 0.87 | 0.37 | 0 | 0 |
| NDUFS8 | 6 | 0.86 | 0.53 | 0.37 | 0 | 0 |
| PRKCDBP | 6 | 0.21 | 0.01 | 0.37 | 0 | 0 |
| SFT2D1 | 6 | 0.72 | 0.31 | 0.37 | 0 | 0 |
| UBE2N | 6 | 0.96 | 0.80 | 0.37 | 0 | 0 |
| CRTAM | 6 | 0.23 | 0.07 | 0.37 | 0 | 0 |
| PDCD6 | 6 | 0.91 | 0.62 | 0.37 | 0 | 0 |
| PFKP | 6 | 0.93 | 0.63 | 0.37 | 0 | 0 |
| ATP5J | 6 | 0.95 | 0.75 | 0.36 | 0 | 0 |
| AC006129.2 | 6 | 0.52 | 0.17 | 0.36 | 0 | 0 |
| C1orf43 | 6 | 0.92 | 0.64 | 0.36 | 0 | 0 |
| PSMBS | 6 | 0.83 | 0.50 | 0.36 | 0 | 0 |
| PSMA4 | 6 | 0.93 | 0.66 | 0.36 | 0 | 0 |
| STAMBP | 6 | 0.53 | 0.17 | 0.36 | 0 | 0 |
| MDH1 | 6 | 0.92 | 0.67 | 0.36 | 0 | 0 |
| HSBP1 | 6 | 0.82 | 0.46 | 0.36 | 0 | 0 |
| CD58 | 6 | 0.86 | 0.49 | 0.36 | 0 | 0 |
| MICAL2 | 6 | 0.43 | 0.10 | 0.36 | 0 | 0 |
| ATP5C1 | 6 | 0.96 | 0.76 | 0.35 | 0 | 0 |
| TXNDC17 | 6 | 0.90 | 0.58 | 0.35 | 0 | 0 |
| IFNAR2 | 6 | 0.79 | 0.37 | 0.35 | 0 | 0 |
| HMGB1 | 6 | 0.98 | 0.85 | 0.35 | 0 | 0 |
| HDDC2 | 6 | 0.77 | 0.39 | 0.35 | 0 | 0 |
| DESI1 | 6 | 0.80 | 0.45 | 0.35 | 0 | 0 |
| VDAC2 | 6 | 0.95 | 0.75 | 0.35 | 0 | 0 |
| ITPA | 6 | 0.79 | 0.41 | 0.35 | 0 | 0 |
| SHFM1 | 6 | 0.96 | 0.74 | 0.35 | 0 | 0 |
| ELMO1 | 6 | 0.87 | 0.47 | 0.35 | 0 | 0 |
| UBE2V1 | 6 | 0.85 | 0.54 | 0.35 | 0 | 0 |
| ATP5A1 | 6 | 0.93 | 0.69 | 0.35 | 0 | 0 |
| MPG | 6 | 0.68 | 0.28 | 0.35 | 0 | 0 |
| ATP5G2 | 6 | 0.99 | 0.90 | 0.34 | 0 | 0 |
| EXOSC7 | 6 | 0.76 | 0.39 | 0.34 | 0 | 0 |
| ARL5A | 6 | 0.90 | 0.61 | 0.34 | 0 | 0 |
| MMADHC | 6 | 0.90 | 0.61 | 0.34 | 0 | 0 |
| ASF1A | 6 | 0.63 | 0.28 | 0.34 | 0 | 0 |
| MRPL51 | 6 | 0.89 | 0.60 | 0.34 | 0 | 0 |
| UBE2V2 | 6 | 0.79 | 0.42 | 0.33 | 0 | 0 |
| PTPN11 | 6 | 0.76 | 0.41 | 0.33 | 0 | 0 |
| WDR1 | 6 | 0.96 | 0.77 | 0.33 | 0 | 0 |
| HTATIP2 | 6 | 0.68 | 0.30 | 0.33 | 0 | 0 |
| SNX9 | 6 | 0.75 | 0.45 | 0.33 | 0 | 0 |
| NTSC | 6 | 0.77 | 0.38 | 0.33 | 0 | 0 |
| ETFA | 6 | 0.79 | 0.45 | 0.33 | 0 | 0 |
| ZNF706 | 6 | 0.97 | 0.77 | 0.33 | 0 | 0 |
| NUDT21 | 6 | 0.85 | 0.52 | 0.33 | 0 | 0 |
| MRPS23 | 6 | 0.84 | 0.51 | 0.33 | 0 | 0 |
| PSTPIP1 | 6 | 0.52 | 0.16 | 0.33 | 0 | 0 |
| CD99 | 6 | 0.98 | 0.85 | 0.33 | 0 | 0 |
| NDUFS3 | 6 | 0.80 | 0.45 | 0.32 | 0 | 0 |
| ETFB | 6 | 0.58 | 0.24 | 0.32 | 0 | 0 |
| PSMB2 | 6 | 0.95 | 0.74 | 0.32 | 0 | 0 |
| PSMD8 | 6 | 0.98 | 0.85 | 0.32 | 0 | 0 |
| PSMD11 | 6 | 0.95 | 0.74 | 0.32 | 0 | 0 |
| PSMB1 | 6 | 0.99 | 0.88 | 0.32 | 0 | 0 |
| LAMTOR5 | 6 | 0.95 | 0.73 | 0.31 | 0 | 0 |
| MRPL42 | 6 | 0.73 | 0.37 | 0.31 | 0 | 0 |
| LBH | 6 | 0.81 | 0.51 | 0.31 | 0 | 0 |
| HAVCR2 | 6 | 0.26 | 0.04 | 0.31 | 0 | 0 |
| CMC2 | 6 | 0.90 | 0.62 | 0.31 | 0 | 0 |
| TMEM167A | 6 | 0.86 | 0.54 | 0.31 | 0 | 0 |
| PAM | 6 | 0.89 | 0.49 | 0.31 | 0 | 0 |
| SH2D2A | 6 | 0.97 | 0.83 | 0.31 | 0 | 0 |
| UBE2L3 | 6 | 0.92 | 0.68 | 0.31 | 0 | 0 |
| NUDT5 | 6 | 0.82 | 0.49 | 0.31 | 0 | 0 |
| PCED1B | 6 | 0.76 | 0.42 | 0.30 | 0 | 0 |
| MRPL34 | 6 | 0.77 | 0.44 | 0.30 | 0 | 0 |
| HLA-DPA1 | 6 | 0.53 | 0.20 | 0.30 | 0 | 0 |
| ATP6V1B2 | 6 | 0.66 | 0.31 | 0.30 | 0 | 0 |
| UQCRFS1 | 6 | 0.96 | 0.77 | 0.30 | 0 | 0 |
| SRGN | 6 | 1.00 | 0.99 | 0.29 | 0 | 0 |
| C11orf31 | 6 | 0.97 | 0.81 | 0.29 | 0 | 0 |
| MBOAT7 | 6 | 0.55 | 0.23 | 0.29 | 0 | 0 |
| SNRNP35 | 6 | 0.61 | 0.27 | 0.29 | 0 | 0 |
| SLC6A6 | 6 | 0.49 | 0.15 | 0.29 | 0 | 0 |
| C11orf58 | 6 | 0.95 | 0.76 | 0.29 | 0 | 0 |
| LAMTOR1 | 6 | 0.83 | 0.50 | 0.29 | 0 | 0 |
| EIF4E | 6 | 0.92 | 0.67 | 0.29 | 0 | 0 |
| ARL1 | 6 | 0.70 | 0.34 | 0.28 | 0 | 0 |
| RAC1 | 6 | 0.89 | 0.62 | 0.28 | 0 | 0 |
| MAP2K3 | 6 | 0.98 | 0.74 | 0.27 | 0 | 0 |
| H3F3A | 6 | 1.00 | 0.96 | 0.27 | 0 | 0 |
| GSTO1 | 6 | 0.79 | 0.49 | 0.27 | 0 | 0 |
| AIP | 6 | 0.83 | 0.53 | 0.27 | 0 | 0 |
| CAPZB | 6 | 0.96 | 0.81 | 0.27 | 0 | 0 |
| TNFRSF9 | 6 | 0.87 | 0.53 | 0.27 | 0 | 0 |
| TERF2IP | 6 | 0.88 | 0.60 | 0.27 | 0 | 0 |
| MEAF6 | 6 | 0.85 | 0.54 | 0.26 | 0 | 0 |
| CSNK2B | 6 | 0.96 | 0.78 | 0.26 | 0 | 0 |
| GBP2 | 6 | 0.98 | 0.84 | 0.26 | 0 | 0 |
| GCNT1 | 6 | 0.44 | 0.14 | 0.26 | 0 | 0 |
| SLA2 | 6 | 0.40 | 0.10 | 0.26 | 0 | 0 |
| STX10 | 6 | 0.59 | 0.25 | 0.25 | 0 | 0 |
| PRSS23 | 6 | 0.22 | 0.04 | 0.25 | 0 | 0 |
| EWSR1 | 6 | 0.96 | 0.77 | 0.28 | 1.23516411460312e−322 | 1.71156691360554e−318 |
| SERPINE2 | 6 | 0.46 | 0.17 | 0.26 | 5.51031414806742e−320 | 7.63564231497703e−316 |
| TXNL1 | 6 | 0.90 | 0.64 | 0.27 | 6.18421968899488e−320 | 8.56947322304021e−316 |
| CDC123 | 6 | 0.90 | 0.61 | 0.30 | 3.68330879631108e−319 | 5.10396099904826e−315 |
| CLNS1A | 6 | 0.87 | 0.54 | 0.34 | 8.30761502169139e−319 | 1.15118621355578e−314 |
| MRPL36 | 6 | 0.88 | 0.57 | 0.32 | 1.71572694634352e−318 | 2.37748282954822e−314 |
| LYPLA1 | 6 | 0.84 | 0.50 | 0.34 | 2.70025007661444e−316 | 3.74173653116462e−312 |
| GAPDH | 6 | 1.00 | 0.99 | 0.43 | 3.93110653166459e−316 | 5.44733432092762e−312 |
| NTRK1 | 6 | 0.34 | 0.09 | 0.33 | 7.56614835544762e−315 | 1.04844117761438e−310 |
| COX5A | 6 | 0.98 | 0.86 | 0.37 | 2.87544253875645e−314 | 3.98450072595482e−310 |
| FAM96A | 6 | 0.70 | 0.35 | 0.29 | 1.44787776470577e−313 | 0.00E+00 |
| SNRPD3 | 6 | 0.97 | 0.78 | 0.32 | 2.50204094853181e−313 | 0.00E+00 |
| COMT | 6 | 0.65 | 0.29 | 0.30 | 3.03144507962767e−312 | 4.20E−308 |
| PSMB7 | 6 | 0.92 | 0.64 | 0.35 | 4.08084880701111e−311 | 5.65E−307 |
| UBA5 | 6 | 0.66 | 0.30 | 0.29 | 0.00E+00 | 1.41E−305 |
| BTLA | 6 | 0.76 | 0.34 | 0.38 | 6.86E−307 | 9.50E−303 |
| PRDX4 | 6 | 0.83 | 0.56 | 0.35 | 2.00E−305 | 2.77E−301 |
| SNX5 | 6 | 0.77 | 0.43 | 0.27 | 1.00E−301 | 1.39E−297 |
| ABHD2 | 6 | 0.43 | 0.13 | 0.31 | 5.74E−301 | 7.95E−297 |
| MINOS1 | 6 | 0.82 | 0.51 | 0.26 | 2.31E−299 | 3.20E−295 |
| C5orf15 | 6 | 0.59 | 0.27 | 0.27 | 1.08E−298 | 1.50E−294 |
| UCHL3 | 6 | 0.81 | 0.45 | 0.42 | 2.95E−295 | 4.09E−291 |
| NDUFA11 | 6 | 0.88 | 0.59 | 0.28 | 3.28E−294 | 4.54E−290 |
| MRPL17 | 6 | 0.87 | 0.59 | 0.29 | 3.67E−294 | 5.08E−290 |
| TCEB1 | 6 | 0.96 | 0.78 | 0.26 | 1.27E−293 | 1.76E−289 |
| MTCH2 | 6 | 0.81 | 0.47 | 0.30 | 5.79E−292 | 8.02E−288 |
| VDAC1 | 6 | 0.96 | 0.76 | 0.42 | 2.59E−291 | 3.59E−287 |
| HLA-DQB1 | 6 | 0.39 | 0.14 | 0.31 | 8.05E−291 | 1.12E−286 |
| SH3BGRL3 | 6 | 1.00 | 0.96 | 0.28 | 2.81E−288 | 3.90E−284 |
| NDUFB9 | 6 | 0.94 | 0.75 | 0.27 | 2.45E−286 | 3.40E−282 |
| POLR2K | 6 | 0.94 | 0.72 | 0.26 | 1.64E−285 | 2.27E−281 |
| LYRM4 | 6 | 0.69 | 0.32 | 0.34 | 3.03E−284 | 4.19E−280 |
| RBBP8 | 6 | 0.64 | 0.27 | 0.32 | 3.58E−284 | 4.97E−280 |
| MRPS7 | 6 | 0.91 | 0.68 | 0.28 | 3.96E−283 | 5.49E−279 |
| UCP2 | 6 | 0.80 | 0.48 | 0.42 | 5.45E−283 | 7.55E−279 |
| AIMP2 | 6 | 0.79 | 0.43 | 0.33 | 1.10E−282 | 1.53E−278 |
| PARL | 6 | 0.78 | 0.45 | 0.26 | 3.03E−281 | 4.20E−277 |
| LIMA1 | 6 | 0.43 | 0.12 | 0.31 | 1.73E−280 | 2.39E−276 |
| SUPT4H1 | 6 | 0.87 | 0.59 | 0.26 | 3.20E−277 | 4.44E−273 |
| EIF1AY | 6 | 0.56 | 0.24 | 0.33 | 7.96E−277 | 1.10E−272 |
| CTLA4 | 6 | 0.69 | 0.39 | 0.25 | 6.12E−276 | 8.49E−272 |
| DNPH1 | 6 | 0.80 | 0.45 | 0.34 | 8.80E−276 | 1.22E−271 |
| NPM3 | 6 | 0.80 | 0.38 | 0.42 | 1.39E−275 | 1.93E−271 |
| MRPL27 | 6 | 0.71 | 0.37 | 0.26 | 8.54E−275 | 1.18E−270 |
| ERH | 6 | 0.98 | 0.83 | 0.33 | 2.75E−274 | 3.81E−270 |
| GRSF1 | 6 | 0.86 | 0.58 | 0.26 | 7.54E−272 | 1.05E−267 |
| MFF | 6 | 0.77 | 0.44 | 0.28 | 4.62E−269 | 6.41E−265 |
| MRPL3 | 6 | 0.92 | 0.66 | 0.33 | 7.17E−268 | 9.94E−264 |
| RUVBL1 | 6 | 0.70 | 0.31 | 0.36 | 1.89E−267 | 2.61E−263 |
| WDR18 | 6 | 0.74 | 0.37 | 0.33 | 1.01E−266 | 1.39E−262 |
| FAM207A | 6 | 0.72 | 0.38 | 0.29 | 1.02E−266 | 1.42E−262 |
| FADD | 6 | 0.49 | 0.16 | 0.25 | 5.01E−266 | 6.95E−262 |
| TMEM70 | 6 | 0.84 | 0.49 | 0.30 | 5.63E−266 | 7.80E−262 |
| MRPS25 | 6 | 0.72 | 0.37 | 0.27 | 2.60E−264 | 3.60E−260 |
| STRAP | 6 | 0.96 | 0.76 | 0.27 | 4.81E−264 | 6.66E−260 |
| EIF4E2 | 6 | 0.72 | 0.37 | 0.28 | 1.79E−263 | 2.48E−259 |
| GPX1 | 6 | 0.78 | 0.39 | 0.44 | 2.61E−263 | 3.61E−259 |
| LSM2 | 6 | 0.86 | 0.56 | 0.29 | 4.65E−263 | 6.44E−259 |
| HN1 | 6 | 0.88 | 0.57 | 0.42 | 8.45E−263 | 1.17E−258 |
| GTPBP4 | 6 | 0.92 | 0.66 | 0.30 | 1.16E−261 | 1.61E−257 |
| PSMD13 | 6 | 0.95 | 0.75 | 0.30 | 1.25E−260 | 1.74E−256 |
| NUDCD2 | 6 | 0.76 | 0.43 | 0.27 | 2.11E−260 | 2.92E−256 |
| OLA1 | 6 | 0.88 | 0.57 | 0.30 | 4.50E−260 | 6.24E−256 |
| URM1 | 6 | 0.79 | 0.45 | 0.27 | 5.70E−260 | 7.89E−256 |
| PDHB | 6 | 0.66 | 0.30 | 0.28 | 1.10E−258 | 1.52E−254 |
| FHL3 | 6 | 0.43 | 0.11 | 0.28 | 1.35E−258 | 1.87E−254 |
| RBPJ | 6 | 0.93 | 0.68 | 0.29 | 6.56E−256 | 9.09E−252 |
| BZW2 | 6 | 0.91 | 0.62 | 0.33 | 7.42E−256 | 1.03E−251 |
| CINP | 6 | 0.55 | 0.22 | 0.26 | 2.46E−253 | 3.41E−249 |
| SMIM11 | 6 | 0.59 | 0.28 | 0.25 | 8.82E−253 | 1.22E−248 |
| PPA2 | 6 | 0.62 | 0.28 | 0.27 | 2.25E−252 | 3.12E−248 |
| NME1 | 6 | 0.88 | 0.52 | 0.36 | 1.65E−251 | 2.29E−247 |
| NAA10 | 6 | 0.88 | 0.59 | 0.29 | 7.14E−247 | 9.89E−243 |
| HPCAL1 | 6 | 0.65 | 0.32 | 0.30 | 3.25E−245 | 4.51E−241 |
| TOMM34 | 6 | 0.69 | 0.35 | 0.26 | 1.54E−243 | 2.13E−239 |
| CISD1 | 6 | 0.67 | 0.29 | 0.34 | 1.82E−243 | 2.52E−239 |
| EXOSC5 | 6 | 0.72 | 0.34 | 0.31 | 4.67E−241 | 6.48E−237 |
| ARMCX6 | 6 | 0.50 | 0.20 | 0.26 | 9.10E−239 | 1.26E−234 |
| NDFIP2 | 6 | 0.88 | 0.60 | 0.26 | 5.91E−238 | 8.19E−234 |
| C19orf24 | 6 | 0.86 | 0.54 | 0.32 | 7.25E−238 | 1.00E−233 |
| PPM1G | 6 | 0.93 | 0.72 | 0.25 | 9.91E−238 | 1.37E−233 |
| EEF1E1 | 6 | 0.89 | 0.61 | 0.28 | 2.57E−237 | 3.56E−233 |
| TXN2 | 6 | 0.68 | 0.35 | 0.25 | 3.64E−235 | 5.04E−231 |
| TIMP1 | 6 | 0.60 | 0.39 | 0.63 | 9.82E−234 | 1.36E−229 |
| C14orf166 | 6 | 0.96 | 0.79 | 0.26 | 3.39E−233 | 4.70E−229 |
| MRPL15 | 6 | 0.77 | 0.40 | 0.35 | 5.83E−232 | 8.09E−228 |
| PHF6 | 6 | 0.73 | 0.37 | 0.26 | 2.27E−231 | 3.14E−227 |
| GNG5 | 6 | 0.98 | 0.85 | 0.30 | 6.92E−230 | 9.58E−226 |
| NDUFB10 | 6 | 0.78 | 0.47 | 0.26 | 8.97E−229 | 1.24E−224 |
| ATP5G1 | 6 | 0.89 | 0.61 | 0.27 | 9.77E−229 | 1.35E−224 |
| BNIP3 | 6 | 0.41 | 0.23 | 0.31 | 2.63E−228 | 3.64E−224 |
| EED | 6 | 0.65 | 0.33 | 0.33 | 1.89E−226 | 2.63E−222 |
| ATIC | 6 | 0.89 | 0.56 | 0.35 | 1.87E−225 | 2.59E−221 |
| RANBP1 | 6 | 0.97 | 0.80 | 0.36 | 1.27E−223 | 1.76E−219 |
| CXCL13 | 6 | 0.16 | 0.02 | 1.54 | 1.75E−223 | 2.42E−219 |
| SSNA1 | 6 | 0.84 | 0.51 | 0.29 | 3.00E−223 | 4.15E−219 |
| FAM216A | 6 | 0.53 | 0.18 | 0.31 | 5.88E−223 | 8.15E−219 |
| BAG2 | 6 | 0.45 | 0.12 | 0.28 | 1.17E−222 | 1.63E−218 |
| TRAP1 | 6 | 0.82 | 0.47 | 0.36 | 2.47E−216 | 3.42E−212 |
| LDHB | 6 | 0.99 | 0.92 | 0.45 | 3.57E−215 | 4.95E−211 |
| MRPS26 | 6 | 0.74 | 0.41 | 0.28 | 6.39E−213 | 8.85E−209 |
| SNRPC | 6 | 0.93 | 0.67 | 0.29 | 1.10E−211 | 1.52E−207 |
| EXOSC4 | 6 | 0.69 | 0.34 | 0.28 | 1.84E−210 | 2.55E−206 |
| VDAC3 | 6 | 0.75 | 0.43 | 0.27 | 1.36E−208 | 1.89E−204 |
| NDUFAB1 | 6 | 0.97 | 0.81 | 0.31 | 5.70E−208 | 7.89E−204 |
| COA4 | 6 | 0.84 | 0.56 | 0.26 | 1.04E−205 | 1.44E−201 |
| GGCT | 6 | 0.69 | 0.33 | 0.30 | 1.06E−204 | 1.47E−200 |
| PRDX1 | 6 | 1.00 | 0.97 | 0.34 | 2.75E−204 | 3.81E−200 |
| LTV1 | 6 | 0.81 | 0.48 | 0.30 | 3.61E−203 | 5.00E−199 |
| CYC1 | 6 | 0.87 | 0.60 | 0.26 | 8.73E−201 | 1.21E−196 |
| TMEM121 | 6 | 0.38 | 0.10 | 0.25 | 1.22E−197 | 1.68E−193 |
| STOML2 | 6 | 0.89 | 0.61 | 0.31 | 2.20E−197 | 3.05E−193 |
| PFDN2 | 6 | 0.95 | 0.76 | 0.27 | 3.70E−197 | 5.13E−193 |
| ANXA2 | 6 | 0.87 | 0.57 | 0.42 | 5.17E−197 | 7.16E−193 |
| GK | 6 | 0.54 | 0.20 | 0.43 | 9.44E−197 | 1.31E−192 |
| CRIP1 | 6 | 0.84 | 0.52 | 0.38 | 7.91E−196 | 1.10E−191 |
| DCUN1D5 | 6 | 0.90 | 0.60 | 0.32 | 5.51E−195 | 7.64E−191 |
| UQCRC2 | 6 | 0.88 | 0.62 | 0.29 | 1.61E−187 | 2.23E−183 |
| AKZ | 6 | 0.91 | 0.59 | 0.36 | 1.33E−184 | 1.84E−180 |
| MRPL4 | 6 | 0.92 | 0.67 | 0.30 | 3.16E−178 | 4.38E−174 |
| OCIAD2 | 6 | 0.70 | 0.40 | 0.27 | 2.65E−177 | 3.67E−173 |
| SNRPD1 | 6 | 0.98 | 0.82 | 0.29 | 5.53E−177 | 7.66E−173 |
| FARSA | 6 | 0.88 | 0.58 | 0.30 | 5.93E−177 | 8.21E−173 |
| TOMM22 | 6 | 0.96 | 0.79 | 0.25 | 2.95E−171 | 4.09E−167 |
| ANP32A | 6 | 0.90 | 0.67 | 0.26 | 1.49E−170 | 2.07E−166 |
| IFI27 | 6 | 0.29 | 0.09 | 0.82 | 3.17E−170 | 4.39E−166 |
| UCK2 | 6 | 0.66 | 0.32 | 0.29 | 9.87E−170 | 1.37E−165 |
| PAICS | 6 | 0.94 | 0.68 | 0.29 | 3.50E−167 | 4.85E−163 |
| KIAA1217 | 6 | 0.29 | 0.07 | 0.26 | 1.08E−166 | 1.50E−162 |
| SLC25A3 | 6 | 0.99 | 0.92 | 0.28 | 3.26E−166 | 4.51E−162 |
| PLS3 | 6 | 0.12 | 0.01 | 0.33 | 3.83E−163 | 5.31E−159 |
| SRSF2 | 6 | 0.98 | 0.87 | 0.30 | 7.16E−161 | 9.93E−157 |
| SLC38A5 | 6 | 0.74 | 0.40 | 0.29 | 1.61E−158 | 2.23E−154 |
| EJF6 | 6 | 0.95 | 0.76 | 0.25 | 3.32E−157 | 4.61E−153 |
| APEX1 | 6 | 0.89 | 0.62 | 0.26 | 8.58E−157 | 1.19E−152 |
| PEBP1 | 6 | 0.91 | 0.69 | 0.29 | 6.73E−156 | 9.33E−152 |
| AGFG1 | 6 | 0.54 | 0.21 | 0.25 | 1.32E−154 | 1.83E−150 |
| CRADD | 6 | 0.34 | 0.11 | 0.28 | 5.01E−152 | 6.94E−148 |
| F5 | 6 | 0.66 | 0.28 | 0.27 | 3.46E−150 | 4.79E−146 |
| TIMM8B | 6 | 0.80 | 0.49 | 0.27 | 1.11E−145 | 1.53E−141 |
| RRP1 | 6 | 0.78 | 0.45 | 0.26 | 3.81E−145 | 5.28E−141 |
| IGFBP4 | 6 | 0.37 | 0.11 | 0.36 | 4.79E−145 | 6.63E−141 |
| TPI1 | 6 | 1.00 | 0.97 | 0.27 | 5.16E−141 | 7.15E−137 |
| TPM4 | 6 | 0.70 | 0.38 | 0.27 | 1.27E−140 | 1.76E−136 |
| HMGA1 | 6 | 0.81 | 0.50 | 0.36 | 1.03E−138 | 1.42E−134 |
| IRF8 | 6 | 0.66 | 0.33 | 0.30 | 1.66E−137 | 2.31E−133 |
| ATP5B | 6 | 0.99 | 0.92 | 0.32 | 1.00E−135 | 1.39E−131 |
| CPM | 6 | 0.46 | 0.12 | 0.41 | 2.34E−132 | 3.24E−128 |
| G0S2 | 6 | 0.20 | 0.03 | 0.83 | 2.85E−125 | 3.95E−121 |
| HSPD1 | 6 | 0.99 | 0.89 | 0.33 | 5.13E−124 | 7.12E−120 |
| PPIF | 6 | 0.71 | 0.41 | 0.31 | 6.88E−123 | 9.53E−119 |
| CD27 | 6 | 0.69 | 0.40 | 0.37 | 7.42E−121 | 1.03E−116 |
| POLR3K | 6 | 0.57 | 0.25 | 0.26 | 1.98E−118 | 2.75E−114 |
| ANKRD10 | 6 | 0.52 | 0.24 | 0.30 | 3.45E−110 | 4.78E−106 |
| CCT8 | 6 | 0.97 | 0.82 | 0.25 | 8.72E−108 | 1.21E−103 |
| RAN | 6 | 1.00 | 0.98 | 0.33 | 5.95E−107 | 8.24E−103 |
| CCDC86 | 6 | 0.78 | 0.45 | 0.28 | 5.59E−105 | 7.75E−101 |
| IER3 | 6 | 0.66 | 0.47 | 0.30 | 1.22E−104 | 1.69E−100 |
| PSMA2.1 | 6 | 0.90 | 0.65 | 0.28 | 2.32E−101 | 3.22E−97 |
| TXN | 6 | 0.99 | 0.85 | 0.29 | 1.36E−99 | 1.88E−95 |
| SORD | 6 | 0.46 | 0.15 | 0.26 | 3.97E−98 | 5.51E−94 |
| THAP4 | 6 | 0.49 | 0.23 | 0.25 | 8.64E−97 | 1.20E−92 |
| BOP1 | 6 | 0.71 | 0.38 | 0.26 | 3.35E−96 | 4.64E−92 |
| AHCY | 6 | 0.77 | 0.42 | 0.30 | 1.87E−95 | 2.60E−91 |
| TNFSF11 | 6 | 0.41 | 0.16 | 0.31 | 9.84E−95 | 1.36E−90 |
| TALDO1 | 6 | 0.84 | 0.55 | 0.26 | 4.41E−94 | 6.11E−90 |
| TKT | 6 | 0.91 | 0.66 | 0.30 | 8.79E−90 | 1.22E−85 |
| GYPC | 6 | 0.79 | 0.48 | 0.30 | 4.96E−78 | 6.87E−74 |
| SRSF3 | 6 | 0.97 | 0.84 | 0.28 | 2.72E−76 | 3.77E−72 |
| CCT3 | 6 | 0.98 | 0.86 | 0.27 | 1.96E−70 | 2.71E−66 |
| EBNA1BP2 | 6 | 0.92 | 0.65 | 0.27 | 6.28E−60 | 8.71E−56 |
| PPA1 | 6 | 0.98 | 0.84 | 0.29 | 3.16E−58 | 4.38E−54 |
| BCAT1 | 6 | 0.42 | 0.14 | 0.25 | 4.73E−42 | 6.56E−38 |
| CACYBP | 6 | 0.90 | 0.64 | 0.26 | 4.08E−39 | 5.66E−35 |
| NPM1 | 6 | 1.00 | 0.99 | 0.27 | 1.64E−13 | 2.28E−09 |

As referred to herein, Table 4 depicts as follows:

TABLE 4

| | | | | Test statistics | | |
|---|---|---|---|---|---|---|
| | | Fraction of | | Average | | |
| | | expressing cells | | logged | | |
| | | Cluster- | Other | Fold | | Adjusted |
| Gene ID | Cluster | specific | cells | Change | P-value | P-value |
| CCL4L1 | 8 | 0.72 | 0.06 | 2.62 | 0 | 0 |
| NKG7 | 8 | 0.99 | 0.21 | 2.58 | 0 | 0 |
| GNLY | 8 | 0.85 | 0.11 | 2.47 | 0 | 0 |
| CCLS | 8 | 0.96 | 0.26 | 1.93 | 0 | 0 |
| GZMB | 8 | 0.98 | 0.22 | 1.75 | 0 | 0 |
| HOPX | 8 | 0.91 | 0.22 | 1.74 | 0 | 0 |
| CCL3 | 8 | 0.75 | 0.14 | 1.72 | 0 | 0 |
| CCL4 | 8 | 0.98 | 0.33 | 1.71 | 0 | 0 |
| PLEK | 8 | 0.91 | 0.15 | 1.67 | 0 | 0 |
| GZMH | 8 | 0.81 | 0.08 | 1.57 | 0 | 0 |
| CST7 | 8 | 0.98 | 0.53 | 1.36 | 0 | 0 |
| HLA-DRB5 | 8 | 0.67 | 0.13 | 1.28 | 0 | 0 |
| PRF1 | 8 | 0.88 | 0.23 | 1.20 | 0 | 0 |
| CCL4L2 | 8 | 0.42 | 0.07 | 1.13 | 0 | 0 |
| KLRG1 | 8 | 0.58 | 0.10 | 1.04 | 0 | 0 |
| ARIDZ | 8 | 0.65 | 0.17 | 1.04 | 0 | 0 |
| HLA-DPB1 | 8 | 0.65 | 0.15 | 1.03 | 0 | 0 |
| SIT1 | 8 | 0.63 | 0.22 | 1.02 | 0 | 0 |
| CD74 | 8 | 0.94 | 0.76 | 0.97 | 0 | 0 |
| KLRB1 | 8 | 0.72 | 0.26 | 0.96 | 0 | 0 |
| CCDC107 | 8 | 0.76 | 0.37 | 0.95 | 0 | 0 |
| LAIR2 | 8 | 0.41 | 0.04 | 0.94 | 0 | 0 |
| LAG3 | 8 | 0.66 | 0.28 | 0.93 | 0 | 0 |
| CX3CR1 | 8 | 0.46 | 0.02 | 0.92 | 0 | 0 |
| CD72 | 8 | 0.52 | 0.10 | 0.91 | 0 | 0 |
| TAGAP | 8 | 0.94 | 0.76 | 0.88 | 0 | 0 |
| HLA-DPA1 | 8 | 0.66 | 0.21 | 0.88 | 0 | 0 |
| GADD45B | 8 | 0.72 | 0.55 | 0.86 | 0 | 0 |
| ITGB2 | 8 | 0.69 | 0.43 | 0.86 | 0 | 0 |
| ZEB2 | 8 | 0.73 | 0.31 | 0.85 | 0 | 0 |
| CD52 | 8 | 0.95 | 0.76 | 0.85 | 0 | 0 |
| HCST | 8 | 0.89 | 0.70 | 0.78 | 0 | 0 |
| HLA-DRB1 | 8 | 0.58 | 0.19 | 0.77 | 0 | 0 |
| LITAF | 8 | 0.80 | 0.47 | 0.77 | 0 | 0 |
| SLAMF7 | 8 | 0.53 | 0.11 | 0.75 | 0 | 0 |
| CD97 | 8 | 0.82 | 0.60 | 0.73 | 0 | 0 |
| HLA-F | 8 | 0.90 | 0.69 | 0.73 | 0 | 0 |
| SLA | 8 | 0.82 | 0.59 | 0.72 | 0 | 0 |
| EGR2 | 8 | 0.71 | 0.41 | 0.70 | 0 | 0 |
| FGFBP2 | 8 | 0.30 | 0.01 | 0.70 | 0 | 0 |
| GZMA | 8 | 0.38 | 0.08 | 0.69 | 0 | 0 |
| APOBEC3C | 8 | 0.65 | 0.37 | 0.69 | 0 | 0 |
| FEZ1 | 8 | 0.32 | 0.05 | 0.68 | 0 | 0 |
| GNG2 | 8 | 0.77 | 0.55 | 0.68 | 0 | 0 |
| HLA-B | 8 | 1.00 | 1.00 | 0.66 | 0 | 0 |
| APOBEC3G | 8 | 0.85 | 0.59 | 0.66 | 0 | 0 |
| PNRC1 | 8 | 0.74 | 0.45 | 0.66 | 0 | 0 |
| UCP2 | 8 | 0.60 | 0.50 | 0.65 | 0 | 0 |
| KMT2E | 8 | 0.86 | 0.67 | 0.65 | 0 | 0 |
| ABI3 | 8 | 0.39 | 0.07 | 0.64 | 0 | 0 |
| HLA-C | 8 | 1.00 | 1.00 | 0.64 | 0 | 0 |
| TNFRSF9 | 8 | 0.72 | 0.55 | 0.63 | 0 | 0 |
| ITGA4 | 8 | 0.58 | 0.32 | 0.62 | 0 | 0 |
| IL2RG | 8 | 0.99 | 0.98 | 0.61 | 0 | 0 |
| PTGER4 | 8 | 0.71 | 0.46 | 0.61 | 0 | 0 |
| AKAP13 | 8 | 0.68 | 0.43 | 0.60 | 0 | 0 |
| SAMD3 | 8 | 0.35 | 0.04 | 0.59 | 0 | 0 |
| UTS2 | 8 | 0.31 | 0.01 | 0.59 | 0 | 0 |
| GLIPR1 | 8 | 0.52 | 0.23 | 0.59 | 0 | 0 |
| ARL6IP5 | 8 | 0.83 | 0.63 | 0.59 | 0 | 0 |
| LINC00152 | 8 | 0.87 | 0.63 | 0.58 | 0 | 0 |
| LCP1 | 8 | 0.91 | 0.77 | 0.58 | 0 | 0 |
| HLA-E | 8 | 1.00 | 1.00 | 0.58 | 0 | 0 |
| PTPRC | 8 | 0.98 | 0.93 | 0.58 | 0 | 0 |
| CISD3 | 8 | 0.69 | 0.48 | 0.58 | 0 | 0 |
| NR3C1 | 8 | 0.73 | 0.54 | 0.57 | 0 | 0 |
| ANXA1 | 8 | 0.84 | 0.61 | 0.57 | 0 | 0 |
| RORA | 8 | 0.63 | 0.36 | 0.56 | 0 | 0 |
| CTSC | 8 | 0.74 | 0.51 | 0.56 | 0 | 0 |
| RAP1B | 8 | 0.90 | 0.80 | 0.55 | 0 | 0 |
| GPSM3 | 8 | 0.75 | 0.46 | 0.55 | 0 | 0 |
| TRIM22 | 8 | 0.84 | 0.67 | 0.55 | 0 | 0 |
| ID2 | 8 | 0.73 | 0.52 | 0.55 | 0 | 0 |
| ARHGDIB | 8 | 0.85 | 0.55 | 0.55 | 0 | 0 |
| LYST | 8 | 0.68 | 0.47 | 0.55 | 0 | 0 |
| RAMP1 | 8 | 0.23 | 0.01 | 0.55 | 0 | 0 |
| FLNA | 8 | 0.67 | 0.44 | 0.54 | 0 | 0 |
| AC017002.1 | 8 | 0.46 | 0.21 | 0.54 | 0 | 0 |
| ATP284 | 8 | 0.47 | 0.21 | 0.54 | 0 | 0 |
| BTG1 | 8 | 0.98 | 0.95 | 0.54 | 0 | 0 |
| SH3BGRL3 | 8 | 1.00 | 0.96 | 0.53 | 0 | 0 |
| PYHIN1 | 8 | 0.36 | 0.11 | 0.53 | 0 | 0 |
| SRGN | 8 | 1.00 | 0.99 | 0.53 | 0 | 0 |
| TNFRSF1A | 8 | 0.45 | 0.20 | 0.52 | 0 | 0 |
| CD3G | 8 | 0.71 | 0.51 | 0.52 | 0 | 0 |
| THEMIS | 8 | 0.45 | 0.18 | 0.52 | 0 | 0 |
| HLA-DQA1 | 8 | 0.25 | 0.03 | 0.52 | 0 | 0 |
| IKZF3 | 8 | 0.44 | 0.20 | 0.52 | 0 | 0 |
| NEAT1 | 8 | 0.66 | 0.42 | 0.52 | 0 | 0 |
| HOXB2 | 8 | 0.42 | 0.16 | 0.52 | 0 | 0 |
| TUBA4A | 8 | 0.62 | 0.40 | 0.51 | 0 | 0 |
| TGFBR3 | 8 | 0.38 | 0.13 | 0.51 | 0 | 0 |
| ITM2C | 8 | 0.32 | 0.12 | 0.50 | 0 | 0 |
| IGF2R | 8 | 0.43 | 0.18 | 0.50 | 0 | 0 |
| ALOX5AP | 8 | 0.63 | 0.34 | 0.49 | 0 | 0 |
| MT-ND4 | 8 | 0.96 | 0.93 | 0.49 | 0 | 0 |
| CD53 | 8 | 0.90 | 0.78 | 0.49 | 0 | 0 |
| ANP32E | 8 | 0.76 | 0.63 | 0.49 | 0 | 0 |
| IL18RAP | 8 | 0.41 | 0.16 | 0.49 | 0 | 0 |
| CLIC1 | 8 | 0.99 | 0.93 | 0.49 | 0 | 0 |
| HLA-A | 8 | 1.00 | 1.00 | 0.49 | 0 | 0 |
| RASSF5 | 8 | 0.79 | 0.66 | 0.48 | 0 | 0 |
| TNFRSF1B | 8 | 0.84 | 0.72 | 0.48 | 0 | 0 |
| IER2 | 8 | 0.61 | 0.47 | 0.48 | 0 | 0 |
| TLN1 | 8 | 0.54 | 0.36 | 0.48 | 0 | 0 |
| RASAL3 | 8 | 0.54 | 0.32 | 0.47 | 0 | 0 |
| CTSW | 8 | 0.29 | 0.05 | 0.47 | 0 | 0 |
| SEPT7 | 8 | 0.89 | 0.81 | 0.46 | 0 | 0 |
| BCL2L11 | 8 | 0.40 | 0.21 | 0.46 | 0 | 0 |
| CD99 | 8 | 0.95 | 0.86 | 0.46 | 0 | 0 |
| GZMM | 8 | 0.53 | 0.32 | 0.46 | 0 | 0 |
| HLA-DRA | 8 | 0.26 | 0.11 | 0.46 | 0 | 0 |
| MSN | 8 | 0.90 | 0.83 | 0.45 | 0 | 0 |
| PTMS | 8 | 0.34 | 0.15 | 0.45 | 0 | 0 |
| HLA-DMA | 8 | 0.29 | 0.08 | 0.45 | 0 | 0 |
| GPR137B | 8 | 0.33 | 0.15 | 0.45 | 0 | 0 |
| MALAT1 | 8 | 1.00 | 1.00 | 0.45 | 0 | 0 |
| ARHGAP25 | 8 | 0.42 | 0.19 | 0.44 | 0 | 0 |
| BTN3A2 | 8 | 0.41 | 0.19 | 0.44 | 0 | 0 |
| NFAT5 | 8 | 0.65 | 0.49 | 0.43 | 0 | 0 |
| PTPN7 | 8 | 0.79 | 0.67 | 0.42 | 0 | 0 |
| AC092580.4 | 8 | 0.24 | 0.05 | 0.42 | 0 | 0 |
| RHOC | 8 | 0.32 | 0.12 | 0.42 | 0 | 0 |
| JAK1 | 8 | 0.75 | 0.59 | 0.41 | 0 | 0 |
| B2M | 8 | 1.00 | 1.00 | 0.41 | 0 | 0 |
| MYL6 | 8 | 0.99 | 0.94 | 0.41 | 0 | 0 |
| ACTB | 8 | 1.00 | 0.99 | 0.41 | 0 | 0 |
| CCNI | 8 | 0.94 | 0.89 | 0.40 | 0 | 0 |
| CD3D | 8 | 0.99 | 0.96 | 0.40 | 0 | 0 |
| RAC2 | 8 | 0.91 | 0.81 | 0.40 | 0 | 0 |
| GABARAP | 8 | 0.86 | 0.73 | 0.40 | 0 | 0 |
| SH2D2A | 8 | 0.91 | 0.83 | 0.40 | 0 | 0 |
| RP11-94L15.2 | 8 | 0.33 | 0.13 | 0.39 | 0 | 0 |
| MT-ATP8 | 8 | 0.93 | 0.91 | 0.38 | 0 | 0 |
| CAP1 | 8 | 0.82 | 0.75 | 0.38 | 0 | 0 |
| ARPC2 | 8 | 0.99 | 0.96 | 0.38 | 0 | 0 |
| ARPC5L | 8 | 0.75 | 0.68 | 0.37 | 0 | 0 |
| PKM | 8 | 0.99 | 0.99 | 0.37 | 0 | 0 |
| C9orf16 | 8 | 0.84 | 0.82 | 0.37 | 0 | 0 |
| RP11-81H14.2 | 8 | 0.20 | 0.02 | 0.37 | 0 | 0 |
| SRSF5 | 8 | 0.88 | 0.84 | 0.36 | 0 | 0 |
| ARPC1B | 8 | 0.85 | 0.74 | 0.36 | 0 | 0 |
| UBB | 8 | 0.96 | 0.93 | 0.34 | 0 | 0 |
| HLA-DQA2 | 8 | 0.16 | 0.01 | 0.34 | 0 | 0 |
| RP11 | 8 | 0.21 | 0.05 | 0.34 | 0 | 0 |
| 325F22.2 | | | | | | |
| C1orf21 | 8 | 0.16 | 0.01 | 0.33 | 0 | 0 |
| DAZAP2 | 8 | 0.93 | 0.90 | 0.33 | 0 | 0 |
| WDR1 | 8 | 0.85 | 0.78 | 0.32 | 0 | 0 |
| MT-CO1 | 8 | 1.00 | 1.00 | 0.31 | 0 | 0 |
| LINC00938 | 8 | 0.20 | 0.05 | 0.30 | 0 | 0 |
| FCRL3 | 8 | 0.15 | 0.01 | 0.30 | 0 | 0 |
| MT-CO2 | 8 | 1.00 | 1.00 | 0.30 | 0 | 0 |
| HAVCR2 | 8 | 0.19 | 0.05 | 0.30 | 0 | 0 |
| GPR56 | 8 | 0.12 | 0.00 | 0.26 | 0 | 0 |
| H3F3A | 8 | 0.99 | 0.96 | 0.26 | 0 | 0 |
| EVL | 8 | 0.65 | 0.41 | 0.49 | 1.16599492418534e−321 | 1.61571916644363e−317 |
| CDC42EP3 | 8 | 0.65 | 0.45 | 0.50 | 7.94704591335645e−320 | 1.1012221522138e−315 |
| YWHAQ | 8 | 0.89 | 0.85 | 0.35 | 9.15996719258379e−318 | 1.26929665387634e−313 |
| MATK | 8 | 0.31 | 0.15 | 0.40 | 4.78308060498768e−314 | 6.62791479433142e−310 |
| RGS3 | 8 | 0.24 | 0.09 | 0.32 | 1.08782628591787e−311 | 1.51E−307 |
| TSC22D4 | 8 | 0.50 | 0.35 | 0.36 | 9.25382851542122e−311 | 1.28E−306 |
| SYTL2 | 8 | 0.18 | 0.04 | 0.29 | 2.31980791762278e−310 | 3.21E−306 |
| ZFP36L1 | 8 | 0.93 | 0.83 | 0.46 | 0.00E+00 | 2.48E−305 |
| GMFG | 8 | 0.78 | 0.64 | 0.36 | 6.57E−308 | 9.10E−304 |
| VCL | 8 | 0.31 | 0.11 | 0.37 | 4.08E−305 | 5.66E−301 |
| IL12RB1 | 8 | 0.32 | 0.15 | 0.35 | 1.21E−303 | 1.67E−299 |
| RPA3 | 8 | 0.54 | 0.44 | 0.34 | 7.08E−303 | 9.81E−299 |
| ARHGAP9 | 8 | 0.39 | 0.21 | 0.39 | 4.97E−300 | 6.89E−296 |
| RNF19A | 8 | 0.92 | 0.81 | 0.42 | 2.02E−299 | 2.80E−295 |
| PCED1B | 8 | 0.62 | 0.43 | 0.41 | 2.45E−298 | 3.39E−294 |
| CREB3 | 8 | 0.44 | 0.30 | 0.36 | 6.05E−298 | 8.39E−294 |
| ZYX | 8 | 0.65 | 0.52 | 0.41 | 8.41E−298 | 1.17E−293 |
| ZBTB38 | 8 | 0.26 | 0.10 | 0.34 | 7.03E−297 | 9.74E−293 |
| RAP1A | 8 | 0.83 | 0.76 | 0.35 | 3.77E−296 | 5.23E−292 |
| SPN | 8 | 0.51 | 0.32 | 0.43 | 7.41E−296 | 1.03E−291 |
| CALM1 | 8 | 0.96 | 0.93 | 0.31 | 4.05E−294 | 5.62E−290 |
| RHBDD2 | 8 | 0.50 | 0.35 | 0.40 | 1.31E−293 | 1.81E−289 |
| TAX1BP1 | 8 | 0.75 | 0.68 | 0.33 | 6.44E−293 | 8.93E−289 |
| SP140 | 8 | 0.58 | 0.43 | 0.41 | 7.71E−292 | 1.07E−287 |
| CD4 | 8 | 0.41 | 0.24 | 0.39 | 8.24E−292 | 1.14E−287 |
| FGL2 | 8 | 0.16 | 0.03 | 0.31 | 9.91E−288 | 1.37E−283 |
| ADRB2 | 8 | 0.14 | 0.02 | 0.26 | 3.39E−287 | 4.70E−283 |
| MACF1 | 8 | 0.52 | 0.30 | 0.45 | 9.67E−285 | 1.34E−280 |
| CTDSP1 | 8 | 0.34 | 0.17 | 0.37 | 7.30E−284 | 1.01E−279 |
| FTH1 | 8 | 0.99 | 0.97 | 0.36 | 1.48E−283 | 2.06E−279 |
| TNFAIP3 | 8 | 0.77 | 0.66 | 0.47 | 6.83E−283 | 9.47E−279 |
| ADO | 8 | 0.33 | 0.18 | 0.34 | 6.92E−279 | 9.58E−275 |
| C4orf3 | 8 | 0.74 | 0.66 | 0.34 | 7.55E−276 | 1.05E−271 |
| KMT2E−AS1 | 8 | 0.24 | 0.10 | 0.31 | 5.82E−275 | 8.06E−271 |
| GOLGA7 | 8 | 0.52 | 0.44 | 0.31 | 1.06E−273 | 1.47E−269 |
| PSMVB9 | 8 | 0.85 | 0.75 | 0.35 | 1.74E−272 | 2.41E−268 |
| ARID4B | 8 | 0.67 | 0.57 | 0.36 | 1.72E−271 | 2.39E−267 |
| SYNE1 | 8 | 0.21 | 0.07 | 0.31 | 6.50E−270 | 9.01E−266 |
| NFATC3 | 8 | 0.36 | 0.22 | 0.33 | 7.47E−268 | 1.04E−263 |
| CD247 | 8 | 0.66 | 0.53 | 0.36 | 3.54E−265 | 4.90E−261 |
| PRR5L | 8 | 0.20 | 0.05 | 0.30 | 2.74E−262 | 3.80E−258 |
| DHRS7 | 8 | 0.54 | 0.34 | 0.38 | 3.25E−262 | 4.50E−258 |
| MAST3 | 8 | 0.25 | 0.10 | 0.31 | 7.92E−261 | 1.10E−256 |
| TPP1 | 8 | 0.37 | 0.21 | 0.35 | 5.38E−259 | 7.46E−255 |
| MIR142 | 8 | 0.38 | 0.25 | 0.37 | 2.49E−258 | 3.45E−254 |
| CD84 | 8 | 0.50 | 0.30 | 0.44 | 4.95E−258 | 6.86E−254 |
| GNPTAB | 8 | 0.24 | 0.09 | 0.31 | 2.79E−256 | 3.86E−252 |
| RIN3 | 8 | 0.25 | 0.10 | 0.34 | 2.19E−255 | 3.04E−251 |
| ANXA6 | 8 | 0.60 | 0.47 | 0.36 | 1.33E−252 | 1.84E−248 |
| PTGER2 | 8 | 0.45 | 0.22 | 0.53 | 2.87E−252 | 3.98E−248 |
| CTB-58E17.1 | 8 | 0.28 | 0.15 | 0.30 | 2.75E−251 | 3.81E−247 |
| BNIP3L | 8 | 0.38 | 0.20 | 0.38 | 7.71E−250 | 1.07E−245 |
| CD3E | 8 | 0.98 | 0.97 | 0.28 | 1.82E−245 | 2.53E−241 |
| C10orf128 | 8 | 0.32 | 0.13 | 0.33 | 2.63E−245 | 3.64E−241 |
| TACC1 | 8 | 0.30 | 0.17 | 0.29 | 4.15E−238 | 5.75E−234 |
| PTPN6 | 8 | 0.57 | 0.45 | 0.35 | 9.73E−238 | 1.35E−233 |
| ARHGEF2 | 8 | 0.51 | 0.39 | 0.36 | 4.53E−237 | 6.27E−233 |
| IL12RB2 | 8 | 0.32 | 0.17 | 0.34 | 1.91E−236 | 2.64E−232 |
| LCP2 | 8 | 0.66 | 0.49 | 0.40 | 8.87E−236 | 1.23E−231 |
| TESK1 | 8 | 0.23 | 0.09 | 0.29 | 1.12E−235 | 1.56E−231 |
| GPX4 | 8 | 0.79 | 0.67 | 0.36 | 4.15E−235 | 5.75E−231 |
| TMEM66 | 8 | 0.98 | 0.95 | 0.30 | 5.58E−233 | 7.73E−229 |
| ST8SIA4 | 8 | 0.42 | 0.25 | 0.37 | 1.34E−232 | 1.86E−228 |
| IGFLR1 | 8 | 0.44 | 0.28 | 0.36 | 5.52E−231 | 7.65E−227 |
| SDCBP | 8 | 0.75 | 0.65 | 0.44 | 9.44E−231 | 1.31E−226 |
| ITGB1 | 8 | 0.60 | 0.43 | 0.36 | 1.66E−230 | 2.30E−226 |
| EDARADD | 8 | 0.22 | 0.08 | 0.36 | 1.78E−227 | 2.47E−223 |
| EFHD2 | 8 | 0.33 | 0.22 | 0.27 | 1.25E−225 | 1.74E−221 |
| MBNL1 | 8 | 0.85 | 0.79 | 0.35 | 1.53E−225 | 2.13E−221 |
| NFKBIA | 8 | 0.91 | 0.87 | 0.43 | 6.34E−219 | 8.79E−215 |
| TSC22D3 | 8 | 0.42 | 0.26 | 0.54 | 1.29E−218 | 1.79E−214 |
| C19orf66 | 8 | 0.64 | 0.52 | 0.34 | 4.62E−215 | 6.40E−211 |
| TAPBPL | 8 | 0.30 | 0.16 | 0.28 | 1.05E−213 | 1.46E−209 |
| VASP | 8 | 0.75 | 0.70 | 0.31 | 1.38E−211 | 1.91E−207 |
| FMNL1 | 8 | 0.32 | 0.18 | 0.29 | 7.78E−211 | 1.08E−206 |
| SH3BP1 | 8 | 0.26 | 0.15 | 0.27 | 4.47E−210 | 6.19E−206 |
| TRIM5 | 8 | 0.32 | 0.19 | 0.33 | 1.23E−209 | 1.71E−205 |
| S100A10 | 8 | 0.91 | 0.81 | 0.26 | 6.98E−209 | 9.68E−205 |
| GSTP1 | 8 | 0.80 | 0.74 | 0.25 | 1.13E−208 | 1.57E−204 |
| NUCB2 | 8 | 0.42 | 0.27 | 0.38 | 2.36E−207 | 3.27E−203 |
| LINC00861 | 8 | 0.40 | 0.23 | 0.40 | 8.81E−207 | 1.22E−202 |
| LGALS1 | 8 | 0.61 | 0.41 | 0.45 | 7.93E−206 | 1.10E−201 |
| LASP1 | 8 | 0.33 | 0.21 | 0.28 | 1.26E−205 | 1.75E−201 |
| CBLB | 8 | 0.75 | 0.60 | 0.39 | 3.09E−205 | 4.28E−201 |
| TOMM7 | 8 | 0.86 | 0.80 | 0.30 | 1.15E−204 | 1.60E−200 |
| UBE2E3 | 8 | 0.40 | 0.29 | 0.29 | 7.97E−204 | 1.10E−199 |
| TOB1 | 8 | 0.25 | 0.14 | 0.27 | 2.09E−203 | 2.89E−199 |
| PPP1R18 | 8 | 0.67 | 0.57 | 0.33 | 2.01E−201 | 2.79E−197 |
| LY9 | 8 | 0.25 | 0.10 | 0.29 | 5.12E−201 | 7.10E−197 |
| UPP1 | 8 | 0.34 | 0.18 | 0.34 | 1.37E−200 | 1.90E−196 |
| AHNAK | 8 | 0.47 | 0.29 | 0.36 | 2.77E−199 | 3.84E−195 |
| JUND | 8 | 0.41 | 0.28 | 0.33 | 2.78E−197 | 3.85E−193 |
| DECR1 | 8 | 0.38 | 0.28 | 0.27 | 1.53E−196 | 2.12E−192 |
| LBH | 8 | 0.66 | 0.53 | 0.38 | 2.22E−196 | 3.08E−192 |
| MAP3K8 | 8 | 0.50 | 0.36 | 0.43 | 2.92E−195 | 4.05E−191 |
| MAP1LC3A | 8 | 0.40 | 0.24 | 0.38 | 7.97E−195 | 1.10E−190 |
| TRIM69 | 8 | 0.52 | 0.41 | 0.31 | 1.13E−194 | 1.57E−190 |
| IQGAP1 | 8 | 0.50 | 0.37 | 0.31 | 1.48E−194 | 2.05E−190 |
| TAPSAR1 | 8 | 0.37 | 0.26 | 0.29 | 6.20E−194 | 8.59E−190 |
| OASL | 8 | 0.36 | 0.21 | 0.31 | 1.52E−193 | 2.10E−189 |
| WIPF1 | 8 | 0.58 | 0.44 | 0.30 | 2.98E−193 | 4.13E−189 |
| SH3KBP1 | 8 | 0.53 | 0.42 | 0.31 | 6.34E−193 | 8.79E−189 |
| STAT4 | 8 | 0.56 | 0.42 | 0.33 | 4.32E−192 | 5.98E−188 |
| GPR108 | 8 | 0.55 | 0.47 | 0.30 | 4.15E−191 | 5.75E−187 |
| PHLDA1 | 8 | 0.70 | 0.56 | 0.40 | 1.39E−190 | 1.92E−186 |
| BZW1 | 8 | 0.92 | 0.91 | 0.27 | 3.38E−189 | 4.68E−185 |
| ANKRD28 | 8 | 0.36 | 0.19 | 0.35 | 1.84E−188 | 2.55E−184 |
| TNIP1 | 8 | 0.60 | 0.50 | 0.28 | 3.77E−188 | 5.22E−184 |
| SYTL3 | 8 | 0.26 | 0.13 | 0.31 | 1.80E−186 | 2.50E−182 |
| LSP1 | 8 | 0.52 | 0.38 | 0.33 | 2.05E−186 | 2.84E−182 |
| ISCU | 8 | 0.77 | 0.73 | 0.26 | 3.51E−186 | 4.86E−182 |
| ITM2B | 8 | 0.91 | 0.81 | 0.30 | 6.11E−186 | 8.46E−182 |
| CHD4 | 8 | 0.60 | 0.52 | 0.31 | 3.24E−184 | 4.50E−180 |
| HMGN2 | 8 | 0.76 | 0.73 | 0.25 | 4.95E−184 | 6.86E−180 |
| GLIPR2 | 8 | 0.29 | 0.16 | 0.28 | 1.54E−182 | 2.13E−178 |
| PRKCH | 8 | 0.80 | 0.70 | 0.33 | 7.19E−181 | 9.96E−177 |
| NAB2 | 8 | 0.45 | 0.33 | 0.34 | 1.20E−180 | 1.66E−176 |
| GMIP | 8 | 0.29 | 0.18 | 0.25 | 6.68E−180 | 9.25E−176 |
| ARID5B | 8 | 0.76 | 0.65 | 0.41 | 2.52E−179 | 3.49E−175 |
| UQCRB | 8 | 0.81 | 0.78 | 0.26 | 2.99E−179 | 4.14E−175 |
| AFTPH | 8 | 0.44 | 0.34 | 0.30 | 1.62E−178 | 2.25E−174 |
| LRRFIP1 | 8 | 0.81 | 0.80 | 0.26 | 7.07E−178 | 9.80E−174 |
| TGIF1 | 8 | 0.61 | 0.53 | 0.42 | 1.65E−177 | 2.29E−173 |
| MYO1G | 8 | 0.29 | 0.15 | 0.29 | 3.55E−177 | 4.92E−173 |
| RILPL2 | 8 | 0.76 | 0.75 | 0.38 | 5.50E−177 | 7.62E−173 |
| BHLHE40 | 8 | 0.63 | 0.49 | 0.37 | 2.27E−176 | 3.14E−172 |
| TMC6 | 8 | 0.40 | 0.26 | 0.33 | 6.02E−175 | 8.34E−171 |
| PTPN22 | 8 | 0.61 | 0.49 | 0.31 | 5.78E−173 | 8.01E−169 |
| GIMAP7 | 8 | 0.45 | 0.29 | 0.27 | 3.42E−170 | 4.73E−166 |
| PAIP2 | 8 | 0.64 | 0.59 | 0.25 | 1.23E−169 | 1.70E−165 |
| ZNFX1 | 8 | 0.57 | 0.51 | 0.28 | 2.43E−169 | 3.37E−165 |
| ZBTB1 | 8 | 0.31 | 0.21 | 0.26 | 5.09E−169 | 7.05E−165 |
| SYNE2 | 8 | 0.73 | 0.58 | 0.31 | 1.74E−168 | 2.42E−164 |
| WSB2 | 8 | 0.27 | 0.17 | 0.26 | 1.23E−167 | 1.70E−163 |
| SH3BGRL | 8 | 0.44 | 0.33 | 0.26 | 1.53E−167 | 2.12E−163 |
| RARRES3 | 8 | 0.41 | 0.27 | 0.33 | 1.54E−167 | 2.14E−163 |
| CLEC2B | 8 | 0.38 | 0.23 | 0.30 | 8.47E−167 | 1.17E−162 |
| KLF10 | 8 | 0.55 | 0.43 | 0.41 | 1.85E−164 | 2.56E−160 |
| LAPTM5 | 8 | 0.67 | 0.55 | 0.31 | 9.45E−164 | 1.31E−159 |
| BCL2 | 8 | 0.47 | 0.36 | 0.32 | 6.97E−162 | 9.66E−158 |
| ZFP36L2 | 8 | 0.48 | 0.37 | 0.32 | 4.17E−159 | 5.78E−155 |
| CD6 | 8 | 0.73 | 0.62 | 0.28 | 2.04E−156 | 2.83E−152 |
| RARG | 8 | 0.30 | 0.17 | 0.26 | 3.78E−156 | 5.23E−152 |
| UTRN | 8 | 0.29 | 0.15 | 0.28 | 1.10E−154 | 1.53E−150 |
| CTSB | 8 | 0.54 | 0.43 | 0.31 | 2.03E−154 | 2.81E−150 |
| EPS15 | 8 | 0.38 | 0.28 | 0.27 | 2.93E−154 | 4.07E−150 |
| CD96 | 8 | 0.65 | 0.50 | 0.32 | 2.14E−152 | 2.96E−148 |
| ACADVL | 8 | 0.47 | 0.39 | 0.26 | 2.79E−152 | 3.87E−148 |
| YPEL5 | 8 | 0.39 | 0.28 | 0.27 | 3.04E−152 | 4.21E−148 |
| SAMSN1 | 8 | 0.61 | 0.51 | 0.28 | 3.43E−152 | 4.76E−148 |
| CD58 | 8 | 0.62 | 0.52 | 0.25 | 1.40E−151 | 1.94E−147 |
| CREM | 8 | 0.67 | 0.59 | 0.40 | 3.58E−151 | 4.97E−147 |
| GPR18 | 8 | 0.28 | 0.14 | 0.31 | 7.93E−151 | 1.10E−146 |
| MYH9 | 8 | 0.65 | 0.59 | 0.29 | 2.57E−150 | 3.56E−146 |
| FASLG | 8 | 0.63 | 0.44 | 0.37 | 5.87E−150 | 8.13E−146 |
| SQRDL | 8 | 0.38 | 0.26 | 0.25 | 1.49E−149 | 2.07E−145 |
| AD000671.6 | 8 | 0.35 | 0.23 | 0.28 | 2.04E−146 | 2.83E−142 |
| EVI2A | 8 | 0.68 | 0.54 | 0.48 | 6.02E−145 | 8.34E−141 |
| IDS | 8 | 0.57 | 0.47 | 0.29 | 5.66E−142 | 7.84E−138 |
| EIF1B | 8 | 0.70 | 0.69 | 0.26 | 2.49E−141 | 3.46E−137 |
| ITK | 8 | 0.69 | 0.62 | 0.29 | 4.57E−139 | 6.33E−135 |
| HSPB1 | 8 | 0.46 | 0.42 | 0.27 | 1.99E−138 | 2.76E−134 |
| IVNS1ABP | 8 | 0.42 | 0.39 | 0.27 | 5.28E−136 | 7.32E−132 |
| PBX4 | 8 | 0.39 | 0.26 | 0.27 | 3.60E−135 | 4.99E−131 |
| PRNP | 8 | 0.76 | 0.75 | 0.43 | 1.70E−133 | 2.36E−129 |
| DDX3Y | 8 | 0.41 | 0.30 | 0.28 | 2.13E−133 | 2.95E−129 |
| ARAP2 | 8 | 0.46 | 0.35 | 0.30 | 6.00E−132 | 8.32E−128 |
| MBP | 8 | 0.41 | 0.29 | 0.29 | 1.16E−131 | 1.60E−127 |
| BTG2 | 8 | 0.60 | 0.47 | 0.39 | 3.44E−131 | 4.76E−127 |
| SPPL2A | 8 | 0.54 | 0.44 | 0.26 | 4.39E−131 | 6.08E−127 |
| DOCK8 | 8 | 0.41 | 0.30 | 0.26 | 1.22E−130 | 1.69E−126 |
| RHOF | 8 | 0.54 | 0.48 | 0.25 | 1.57E−130 | 2.18E−126 |
| SLC44A2 | 8 | 0.29 | 0.18 | 0.25 | 4.14E−125 | 5.74E−121 |
| LINC00944 | 8 | 0.23 | 0.10 | 0.26 | 7.19E−125 | 9.96E−121 |
| FBXO34 | 8 | 0.29 | 0.20 | 0.26 | 3.52E−123 | 4.88E−119 |
| CRY1 | 8 | 0.49 | 0.41 | 0.30 | 5.70E−123 | 7.90E−119 |
| TRAF1 | 8 | 0.74 | 0.66 | 0.30 | 1.99E−119 | 2.76E−115 |
| HBP1 | 8 | 0.30 | 0.19 | 0.25 | 3.77E−119 | 5.22E−115 |
| SAMD9 | 8 | 0.36 | 0.24 | 0.27 | 7.17E−117 | 9.94E−113 |
| GIMAPS | 8 | 0.52 | 0.41 | 0.29 | 2.85E−116 | 3.95E−112 |
| TBX21 | 8 | 0.39 | 0.28 | 0.31 | 7.76E−114 | 1.08E−109 |
| IFITM2 | 8 | 0.75 | 0.73 | 0.36 | 6.07E−113 | 8.42E−109 |
| PHF1 | 8 | 0.35 | 0.22 | 0.26 | 2.02E−112 | 2.80E−108 |
| ANKRD44 | 8 | 0.45 | 0.35 | 0.28 | 2.74E−110 | 3.80E−106 |
| RGS16 | 8 | 0.59 | 0.51 | 0.34 | 8.74E−109 | 1.21E−104 |
| CRTAM | 8 | 0.16 | 0.07 | 0.57 | 5.09E−107 | 7.05E−103 |
| PBXIP1 | 8 | 0.25 | 0.14 | 0.27 | 8.89E−107 | 1.23E−102 |
| XAF1 | 8 | 0.44 | 0.33 | 0.26 | 1.03E−102 | 1.43E−98 |
| LTBP4 | 8 | 0.63 | 0.56 | 0.26 | 1.50E−102 | 2.07E−98 |
| SLC20A1 | 8 | 0.42 | 0.34 | 0.29 | 9.44E−101 | 1.31E−96 |
| RAB8B | 8 | 0.66 | 0.60 | 0.26 | 5.69E−97 | 7.89E−93 |
| TMBIM1 | 8 | 0.56 | 0.48 | 0.25 | 3.74E−81 | 5.18E−77 |
| SLC2A3 | 8 | 0.58 | 0.48 | 0.26 | 9.69E−76 | 1.34E−71 |
| KLF6 | 8 | 0.83 | 0.78 | 0.27 | 5.50E−70 | 7.62E−66 |
| CD83 | 8 | 0.48 | 0.41 | 0.26 | 5.77E−55 | 8.00E−51 |
| NR4A3 | 8 | 0.40 | 0.35 | 0.30 | 2.60E−28 | 3.61E−24 |

As referred to herein, Table 5 depicts as follows:

TABLE 5

| | | | | Test statistics | | |
|---|---|---|---|---|---|---|
| | | Fraction of | | Average | | |
| | | expressing cells | | logged | | |
| | | Cluster- | Other | Fold | | Adjusted |
| Gene ID | Cluster | specific | cells | Change | P-value | P-value |
| IL17F | 9 | 0.36 | 0.02 | 3.91 | 0 | 0 |
| IL17A | 9 | 0.39 | 0.02 | 3.45 | 0 | 0 |
| CTSH | 9 | 0.73 | 0.15 | 1.24 | 0 | 0 |
| LGALS3 | 9 | 0.87 | 0.28 | 1.24 | 0 | 0 |
| S100A4 | 9 | 0.88 | 0.53 | 1.16 | 0 | 0 |
| CCR6 | 9 | 0.68 | 0.10 | 1.14 | 0 | 0 |
| MSC | 9 | 0.58 | 0.06 | 1.12 | 0 | 0 |
| CCL20 | 9 | 0.67 | 0.33 | 1.10 | 0 | 0 |
| LTB | 9 | 0.94 | 0.67 | 0.99 | 0 | 0 |
| IL4I1 | 9 | 0.73 | 0.19 | 0.96 | 0 | 0 |
| IL32 | 9 | 0.99 | 0.88 | 0.93 | 0 | 0 |
| CORO1A | 9 | 0.93 | 0.60 | 0.91 | 0 | 0 |
| S100A6 | 9 | 0.96 | 0.71 | 0.89 | 0 | 0 |
| OSTF1 | 9 | 0.76 | 0.30 | 0.89 | 0 | 0 |
| IL2RA | 9 | 0.84 | 0.50 | 0.88 | 0 | 0 |
| CD74 | 9 | 0.97 | 0.76 | 0.88 | 0 | 0 |
| LGALS1 | 9 | 0.72 | 0.41 | 0.84 | 0 | 0 |
| NTRK2 | 9 | 0.43 | 0.04 | 0.81 | 0 | 0 |
| PTP4A3 | 9 | 0.37 | 0.14 | 0.80 | 0 | 0 |
| TMSB4X | 9 | 0.99 | 0.96 | 0.79 | 0 | 0 |
| CXCR6 | 9 | 0.36 | 0.07 | 0.78 | 0 | 0 |
| KLRBP | 9 | 0.62 | 0.27 | 0.77 | 0 | 0 |
| TYMP | 9 | 0.83 | 0.41 | 0.77 | 0 | 0 |
| ARHGDIB | 9 | 0.86 | 0.55 | 0.77 | 0 | 0 |
| TMSB10 | 9 | 1.00 | 0.95 | 0.76 | 0 | 0 |
| PTPRCAP | 9 | 0.76 | 0.42 | 0.74 | 0 | 0 |
| TNFRSF4 | 9 | 0.96 | 0.85 | 0.73 | 0 | 0 |
| GNA15 | 9 | 0.66 | 0.21 | 0.70 | 0 | 0 |
| CCR4 | 9 | 0.57 | 0.21 | 0.70 | 0 | 0 |
| TXN | 9 | 0.98 | 0.85 | 0.70 | 0 | 0 |
| ARPC1B | 9 | 0.94 | 0.74 | 0.69 | 0 | 0 |
| TNFRSF18 | 9 | 0.96 | 0.81 | 0.69 | 0 | 0 |
| VIM | 9 | 0.97 | 0.83 | 0.69 | 0 | 0 |
| TPM4 | 9 | 0.78 | 0.39 | 0.69 | 0 | 0 |
| ANXA2 | 9 | 0.90 | 0.58 | 0.64 | 0 | 0 |
| RGS1 | 9 | 0.45 | 0.19 | 0.64 | 0 | 0 |
| LAPTM5 | 9 | 0.82 | 0.54 | 0.63 | 0 | 0 |
| PIM2 | 9 | 0.68 | 0.34 | 0.63 | 0 | 0 |
| SPOCK2 | 9 | 0.72 | 0.36 | 0.62 | 0 | 0 |
| NFKBIA | 9 | 0.97 | 0.87 | 0.61 | 0 | 0 |
| CYTIP | 9 | 0.88 | 0.53 | 0.60 | 0 | 0 |
| ANKRD12 | 9 | 0.87 | 0.56 | 0.60 | 0 | 0 |
| LSP1 | 9 | 0.67 | 0.38 | 0.60 | 0 | 0 |
| FLT3LG | 9 | 0.72 | 0.25 | 0.60 | 0 | 0 |
| ACTG1 | 9 | 0.99 | 0.96 | 0.58 | 0 | 0 |
| FTH1 | 9 | 1.00 | 0.97 | 0.58 | 0 | 0 |
| BATF | 9 | 0.84 | 0.60 | 0.57 | 0 | 0 |
| CMTM6 | 9 | 0.84 | 0.54 | 0.57 | 0 | 0 |
| ARL6IPS | 9 | 0.89 | 0.63 | 0.57 | 0 | 0 |
| MYO1G | 9 | 0.54 | 0.14 | 0.56 | 0 | 0 |
| RORA | 9 | 0.76 | 0.35 | 0.55 | 0 | 0 |
| KLF6 | 9 | 0.96 | 0.78 | 0.55 | 0 | 0 |
| MYL6 | 9 | 0.99 | 0.94 | 0.55 | 0 | 0 |
| SQSTM1 | 9 | 0.82 | 0.53 | 0.55 | 0 | 0 |
| GBP5 | 9 | 0.75 | 0.45 | 0.54 | 0 | 0 |
| ACTB | 9 | 1.00 | 0.99 | 0.54 | 0 | 0 |
| FLNA | 9 | 0.75 | 0.44 | 0.54 | 0 | 0 |
| RNF213 | 9 | 0.74 | 0.39 | 0.54 | 0 | 0 |
| CTSC | 9 | 0.77 | 0.51 | 0.54 | 0 | 0 |
| GPX1 | 9 | 0.69 | 0.41 | 0.53 | 0 | 0 |
| SAMHD1 | 9 | 0.61 | 0.23 | 0.53 | 0 | 0 |
| KIF2A | 9 | 0.72 | 0.42 | 0.53 | 0 | 0 |
| TNFRSF25 | 9 | 0.82 | 0.56 | 0.53 | 0 | 0 |
| LCP1 | 9 | 0.96 | 0.77 | 0.53 | 0 | 0 |
| OPTN | 9 | 0.66 | 0.29 | 0.53 | 0 | 0 |
| LMO4 | 9 | 0.45 | 0.14 | 0.52 | 0 | 0 |
| EML4 | 9 | 0.76 | 0.42 | 0.52 | 0 | 0 |
| GPSM3 | 9 | 0.77 | 0.46 | 0.51 | 0 | 0 |
| EMP3 | 9 | 0.99 | 0.90 | 0.51 | 0 | 0 |
| CAMK4 | 9 | 0.63 | 0.25 | 0.51 | 0 | 0 |
| IL2RB | 9 | 0.68 | 0.35 | 0.50 | 0 | 0 |
| PTPN13 | 9 | 0.35 | 0.05 | 0.50 | 0 | 0 |
| CAPG | 9 | 0.37 | 0.10 | 0.50 | 0 | 0 |
| RORC | 9 | 0.41 | 0.11 | 0.49 | 0 | 0 |
| FAS | 9 | 0.60 | 0.22 | 0.49 | 0 | 0 |
| ENTPD1 | 9 | 0.25 | 0.03 | 0.49 | 0 | 0 |
| STK17B | 9 | 0.83 | 0.55 | 0.49 | 0 | 0 |
| PLP2 | 9 | 0.79 | 0.52 | 0.49 | 0 | 0 |
| ANXAS | 9 | 0.69 | 0.37 | 0.49 | 0 | 0 |
| LPXN | 9 | 0.77 | 0.45 | 0.48 | 0 | 0 |
| NFKB2 | 9 | 0.74 | 0.44 | 0.47 | 0 | 0 |
| GPR183 | 9 | 0.63 | 0.30 | 0.47 | 0 | 0 |
| PSME1 | 9 | 0.98 | 0.84 | 0.47 | 0 | 0 |
| TNFRSF14 | 9 | 0.64 | 0.29 | 0.47 | 0 | 0 |
| PHTF2 | 9 | 0.48 | 0.15 | 0.46 | 0 | 0 |
| PFN1 | 9 | 1.00 | 0.98 | 0.46 | 0 | 0 |
| TSPO | 9 | 0.75 | 0.48 | 0.45 | 0 | 0 |
| SH3BP5 | 9 | 0.47 | 0.16 | 0.45 | 0 | 0 |
| FURIN | 9 | 0.48 | 0.19 | 0.44 | 0 | 0 |
| NMRK1 | 9 | 0.42 | 0.13 | 0.44 | 0 | 0 |
| TNIP1 | 9 | 0.78 | 0.49 | 0.44 | 0 | 0 |
| RAC2 | 9 | 0.96 | 0.81 | 0.43 | 0 | 0 |
| PIM1 | 9 | 0.67 | 0.37 | 0.43 | 0 | 0 |
| JAK1 | 9 | 0.86 | 0.58 | 0.43 | 0 | 0 |
| TANK | 9 | 0.74 | 0.42 | 0.42 | 0 | 0 |
| NDUFV2 | 9 | 0.96 | 0.84 | 0.42 | 0 | 0 |
| GNG2 | 9 | 0.80 | 0.54 | 0.42 | 0 | 0 |
| TRADD | 9 | 0.52 | 0.17 | 0.42 | 0 | 0 |
| GSDMD | 9 | 0.55 | 0.23 | 0.42 | 0 | 0 |
| AHR | 9 | 0.61 | 0.32 | 0.41 | 0 | 0 |
| CISH | 9 | 0.54 | 0.21 | 0.41 | 0 | 0 |
| SQRDL | 9 | 0.58 | 0.25 | 0.41 | 0 | 0 |
| RAP1B | 9 | 0.92 | 0.80 | 0.41 | 0 | 0 |
| ACTR3 | 9 | 0.96 | 0.84 | 0.40 | 0 | 0 |
| SYTL3 | 9 | 0.39 | 0.12 | 0.40 | 0 | 0 |
| CUTA | 9 | 0.74 | 0.49 | 0.40 | 0 | 0 |
| UNC119 | 9 | 0.38 | 0.13 | 0.39 | 0 | 0 |
| DPP4 | 9 | 0.41 | 0.14 | 0.39 | 0 | 0 |
| CD80 | 9 | 0.25 | 0.03 | 0.38 | 0 | 0 |
| GPR65 | 9 | 0.53 | 0.24 | 0.38 | 0 | 0 |
| TAPBP | 9 | 0.90 | 0.67 | 0.38 | 0 | 0 |
| SOCS2 | 9 | 0.37 | 0.10 | 0.38 | 0 | 0 |
| PRDM1 | 9 | 0.48 | 0.19 | 0.37 | 0 | 0 |
| CFL1 | 9 | 1.00 | 0.97 | 0.37 | 0 | 0 |
| MGAT4A | 9 | 0.46 | 0.16 | 0.37 | 0 | 0 |
| IL12RB1 | 9 | 0.43 | 0.15 | 0.37 | 0 | 0 |
| RSU1 | 9 | 0.48 | 0.19 | 0.35 | 0 | 0 |
| PBX4 | 9 | 0.53 | 0.25 | 0.34 | 0 | 0 |
| KCNA3 | 9 | 0.39 | 0.12 | 0.33 | 0 | 0 |
| CCNG2 | 9 | 0.18 | 0.02 | 0.31 | 0 | 0 |
| IL26 | 9 | 0.20 | 0.03 | 0.30 | 0 | 0 |
| IL2RG | 9 | 1.00 | 0.98 | 0.28 | 0 | 0 |
| RP11- | 9 | 0.19 | 0.02 | 0.27 | 0 | 0 |
| 316P17.2 | | | | | | |
| MAL | 9 | 0.36 | 0.15 | 0.55 | 3.45845952088873e−323 | 4.79238735809551e−319 |
| ACAT2 | 9 | 0.51 | 0.21 | 0.42 | 1.43279037293961e−322 | 1.98541761978242e−318 |
| PSMB10 | 9 | 0.87 | 0.63 | 0.42 | 2.42092166462211e−322 | 3.35467115066686e−318 |
| PPARG | 9 | 0.20 | 0.03 | 0.29 | 2.69142260572019e−319 | 3.72950430474647e−315 |
| PBXIP1 | 9 | 0.37 | 0.13 | 0.34 | 1.12350527864299e−318 | 1.5568412646156e−314 |
| ADAM8 | 9 | 0.35 | 0.12 | 0.33 | 1.7049069086996e−318 | 2.36248950338503e−314 |
| AC017002.1 | 9 | 0.46 | 0.21 | 0.51 | 5.31755596794596e−316 | 7.36853730478272e−312 |
| CD47 | 9 | 0.79 | 0.49 | 0.35 | 6.56168624755142e−315 | 9.09252863323201e−311 |
| ALOX5AP | 9 | 0.63 | 0.34 | 0.47 | 1.84421290228056e−314 | 2.55552581869017e−310 |
| DSE | 9 | 0.28 | 0.07 | 0.27 | 2.12807192584966e−312 | 2.95E−308 |
| PLIN2 | 9 | 0.46 | 0.23 | 0.44 | 2.68658046365998e−310 | 3.72E−306 |
| SELPLG | 9 | 0.37 | 0.13 | 0.39 | 1.28E−307 | 1.77E−303 |
| CAST | 9 | 0.68 | 0.40 | 0.35 | 4.77E−305 | 6.61E−301 |
| CD247 | 9 | 0.79 | 0.53 | 0.40 | 8.61E−305 | 1.19E−300 |
| BHLHE40 | 9 | 0.74 | 0.48 | 0.40 | 2.18E−304 | 3.03E−300 |
| BLM | 9 | 0.25 | 0.06 | 0.28 | 3.95E−304 | 5.47E−300 |
| S1PR1 | 9 | 0.38 | 0.14 | 0.32 | 1.81E−303 | 2.51E−299 |
| PDE4D | 9 | 0.45 | 0.19 | 0.40 | 4.10E−301 | 5.68E−297 |
| FTL | 9 | 0.99 | 0.96 | 0.65 | 1.94E−299 | 2.68E−295 |
| MVP | 9 | 0.68 | 0.39 | 0.41 | 1.18E−295 | 1.63E−291 |
| PSME2 | 9 | 0.99 | 0.91 | 0.35 | 3.77E−295 | 5.22E−291 |
| C10orf128 | 9 | 0.36 | 0.13 | 0.36 | 7.38E−294 | 1.02E−289 |
| EVL | 9 | 0.66 | 0.41 | 0.42 | 6.62E−293 | 9.18E−289 |
| CNN2 | 9 | 0.42 | 0.18 | 0.44 | 9.63E−293 | 1.33E−288 |
| MYL12A | 9 | 0.94 | 0.86 | 0.45 | 3.32E−287 | 4.60E−283 |
| CARD16 | 9 | 0.49 | 0.21 | 0.33 | 1.71E−285 | 2.37E−281 |
| TNFRSF1B | 9 | 0.86 | 0.72 | 0.43 | 9.33E−283 | 1.29E−278 |
| PTPN4 | 9 | 0.33 | 0.11 | 0.27 | 2.60E−282 | 3.60E−278 |
| LCP2 | 9 | 0.73 | 0.48 | 0.40 | 1.70E−281 | 2.36E−277 |
| AHNAK | 9 | 0.55 | 0.28 | 0.40 | 1.56E−280 | 2.17E−276 |
| GPR25 | 9 | 0.16 | 0.03 | 0.35 | 2.84E−279 | 3.94E−275 |
| TBC1D10C | 9 | 0.35 | 0.13 | 0.33 | 3.12E−279 | 4.33E−275 |
| GBP1 | 9 | 0.75 | 0.47 | 0.47 | 2.35E−275 | 3.25E−271 |
| CALM1 | 9 | 0.98 | 0.93 | 0.30 | 1.36E−273 | 1.89E−269 |
| STAT1 | 9 | 0.86 | 0.66 | 0.47 | 2.94E−272 | 4.08E−268 |
| CYTH1 | 9 | 0.44 | 0.19 | 0.32 | 8.39E−271 | 1.16E−266 |
| ACAP1 | 9 | 0.43 | 0.19 | 0.35 | 6.68E−270 | 9.26E−266 |
| HUWE1 | 9 | 0.61 | 0.37 | 0.37 | 1.75E−268 | 2.42E−264 |
| DNPH1 | 9 | 0.73 | 0.47 | 0.41 | 1.76E−268 | 2.44E−264 |
| DBI | 9 | 0.88 | 0.70 | 0.39 | 1.85E−268 | 2.56E−264 |
| IL22 | 9 | 0.17 | 0.03 | 2.17 | 1.09E−266 | 1.51E−262 |
| TUBA1A | 9 | 0.48 | 0.21 | 0.35 | 1.13E−266 | 1.57E−262 |
| CCNI | 9 | 0.97 | 0.89 | 0.28 | 1.53E−266 | 2.12E−262 |
| ICAM1 | 9 | 0.38 | 0.15 | 0.38 | 1.59E−265 | 2.21E−261 |
| ITGAL | 9 | 0.32 | 0.10 | 0.28 | 5.69E−265 | 7.88E−261 |
| CALCOCO2 | 9 | 0.64 | 0.36 | 0.33 | 4.60E−262 | 6.37E−258 |
| LY6E | 9 | 0.91 | 0.69 | 0.42 | 4.66E−262 | 6.46E−258 |
| JUNB | 9 | 0.85 | 0.66 | 0.40 | 1.07E−259 | 1.48E−255 |
| FAM129A | 9 | 0.51 | 0.25 | 0.36 | 1.53E−257 | 2.12E−253 |
| ARHGAP15 | 9 | 0.62 | 0.35 | 0.32 | 7.29E−257 | 1.01E−252 |
| APOL3 | 9 | 0.34 | 0.11 | 0.28 | 5.18E−256 | 7.17E−252 |
| MAF | 9 | 0.48 | 0.22 | 0.39 | 3.32E−255 | 4.59E−251 |
| RAB11FIP1 | 9 | 0.55 | 0.30 | 0.41 | 9.09E−255 | 1.26E−250 |
| EED | 9 | 0.58 | 0.35 | 0.43 | 3.88E−252 | 5.38E−248 |
| VPS13C | 9 | 0.48 | 0.22 | 0.30 | 2.08E−251 | 2.89E−247 |
| FAM46C | 9 | 0.30 | 0.10 | 0.26 | 2.91E−247 | 4.03E−243 |
| CLDND1 | 9 | 0.76 | 0.54 | 0.41 | 5.16E−246 | 7.15E−242 |
| EBP | 9 | 0.44 | 0.19 | 0.31 | 1.85E−245 | 2.56E−241 |
| RAB9A | 9 | 0.43 | 0.24 | 0.35 | 2.52E−245 | 3.49E−241 |
| MAN2B1 | 9 | 0.39 | 0.16 | 0.28 | 6.35E−245 | 8.79E−241 |
| MVD | 9 | 0.37 | 0.14 | 0.28 | 1.98E−244 | 2.74E−240 |
| MAST4 | 9 | 0.31 | 0.11 | 0.27 | 1.25E−243 | 1.73E−239 |
| HLA-DQB1 | 9 | 0.36 | 0.15 | 0.47 | 6.72E−243 | 9.31E−239 |
| LIMD2 | 9 | 0.79 | 0.57 | 0.44 | 6.98E−243 | 9.67E−239 |
| XAF1 | 9 | 0.59 | 0.32 | 0.29 | 1.47E−238 | 2.03E−234 |
| PMVK | 9 | 0.58 | 0.32 | 0.34 | 1.65E−237 | 2.29E−233 |
| SEPT9 | 9 | 0.48 | 0.23 | 0.32 | 2.57E−235 | 3.56E−231 |
| CYB5A | 9 | 0.41 | 0.17 | 0.30 | 1.59E−234 | 2.20E−230 |
| FDPS | 9 | 0.67 | 0.47 | 0.37 | 4.79E−234 | 6.64E−230 |
| TPM3 | 9 | 0.97 | 0.90 | 0.30 | 5.25E−234 | 7.28E−230 |
| IL1R2 | 9 | 0.12 | 0.02 | 0.33 | 3.46E−233 | 4.80E−229 |
| CAPN2 | 9 | 0.55 | 0.28 | 0.36 | 9.55E−231 | 1.32E−226 |
| CD4 | 9 | 0.46 | 0.23 | 0.36 | 5.60E−228 | 7.77E−224 |
| GBP4 | 9 | 0.58 | 0.31 | 0.38 | 7.23E−228 | 1.00E−223 |
| ILK | 9 | 0.52 | 0.26 | 0.31 | 2.92E−227 | 4.04E−223 |
| MT2A | 9 | 0.57 | 0.31 | 0.55 | 8.78E−224 | 1.22E−219 |
| OSM | 9 | 0.29 | 0.10 | 0.45 | 8.66E−223 | 1.20E−218 |
| SASH3 | 9 | 0.38 | 0.16 | 0.30 | 9.58E−223 | 1.33E−218 |
| ARHGDIA | 9 | 0.92 | 0.78 | 0.32 | 1.85E−222 | 2.57E−218 |
| CDC42 | 9 | 0.95 | 0.86 | 0.27 | 4.88E−222 | 6.77E−218 |
| WIPF1 | 9 | 0.66 | 0.44 | 0.36 | 2.68E−221 | 3.71E−217 |
| AC092580.4 | 9 | 0.20 | 0.05 | 0.28 | 4.53E−221 | 6.27E−217 |
| TBCB | 9 | 0.69 | 0.46 | 0.33 | 1.60E−219 | 2.21E−215 |
| PLEC | 9 | 0.26 | 0.08 | 0.27 | 1.17E−218 | 1.62E−214 |
| PRMT2 | 9 | 0.46 | 0.21 | 0.26 | 1.18E−218 | 1.64E−214 |
| GABARAP | 9 | 0.88 | 0.73 | 0.27 | 1.91E−218 | 2.65E−214 |
| ISG15 | 9 | 0.80 | 0.51 | 0.40 | 3.42E−218 | 4.74E−214 |
| NFKBIZ | 9 | 0.50 | 0.25 | 0.34 | 1.03E−217 | 1.43E−213 |
| DUSP1 | 9 | 0.56 | 0.33 | 0.40 | 1.83E−216 | 2.53E−212 |
| SYTL1 | 9 | 0.31 | 0.12 | 0.26 | 1.84E−216 | 2.55E−212 |
| ACTN4 | 9 | 0.64 | 0.41 | 0.30 | 8.08E−216 | 1.12E−211 |
| IFI35 | 9 | 0.68 | 0.40 | 0.32 | 3.25E−214 | 4.50E−210 |
| BIN1 | 9 | 0.33 | 0.13 | 0.26 | 1.52E−213 | 2.10E−209 |
| CAP1 | 9 | 0.88 | 0.74 | 0.30 | 2.07E−213 | 2.86E−209 |
| PSMB9 | 9 | 0.92 | 0.75 | 0.32 | 4.82E−212 | 6.68E−208 |
| IRF1 | 9 | 0.84 | 0.63 | 0.34 | 3.19E−210 | 4.42E−206 |
| FNBP1 | 9 | 0.72 | 0.51 | 0.29 | 8.74E−209 | 1.21E−204 |
| GRINA | 9 | 0.40 | 0.19 | 0.29 | 1.84E−206 | 2.56E−202 |
| ICAM3 | 9 | 0.60 | 0.36 | 0.32 | 7.29E−206 | 1.01E−201 |
| SYNGR2 | 9 | 0.89 | 0.76 | 0.27 | 2.02E−205 | 2.80E−201 |
| HSPB1 | 9 | 0.64 | 0.41 | 0.37 | 1.52E−201 | 2.10E−197 |
| DDIT4 | 9 | 0.53 | 0.30 | 0.41 | 1.74E−201 | 2.41E−197 |
| ELOVL5 | 9 | 0.74 | 0.52 | 0.29 | 2.19E−201 | 3.04E−197 |
| NECAP2 | 9 | 0.54 | 0.32 | 0.28 | 1.95E−200 | 2.70E−196 |
| ANXA6 | 9 | 0.70 | 0.47 | 0.35 | 1.05E−197 | 1.46E−193 |
| FOXP3 | 9 | 0.17 | 0.04 | 0.47 | 6.98E−196 | 9.68E−192 |
| PPDPF | 9 | 0.91 | 0.78 | 0.28 | 1.07E−195 | 1.49E−191 |
| CDKZAP2 | 9 | 0.67 | 0.46 | 0.37 | 1.76E−195 | 2.44E−191 |
| PPP1CA | 9 | 0.87 | 0.71 | 0.31 | 2.40E−195 | 3.32E−191 |
| RDX | 9 | 0.34 | 0.14 | 0.29 | 9.71E−193 | 1.35E−188 |
| ARHGAP30 | 9 | 0.47 | 0.24 | 0.29 | 2.03E−192 | 2.81E−188 |
| DHCR7 | 9 | 0.33 | 0.13 | 0.25 | 2.26E−190 | 3.13E−186 |
| TUBB | 9 | 0.90 | 0.78 | 0.39 | 5.41E−190 | 7.50E−186 |
| HLA-DQA1 | 9 | 0.16 | 0.04 | 0.38 | 1.38E−183 | 1.92E−179 |
| AD000671.6 | 9 | 0.43 | 0.23 | 0.28 | 1.29E−182 | 1.79E−178 |
| IGFLR1 | 9 | 0.49 | 0.28 | 0.29 | 6.50E−182 | 9.01E−178 |
| SNX10 | 9 | 0.35 | 0.16 | 0.28 | 8.31E−182 | 1.15E−177 |
| GMFG | 9 | 0.81 | 0.64 | 0.33 | 1.49E−181 | 2.07E−177 |
| MIIP | 9 | 0.37 | 0.17 | 0.26 | 1.23E−180 | 1.71E−176 |
| INSIG1 | 9 | 0.60 | 0.38 | 0.57 | 4.10E−180 | 5.68E−176 |
| ID2 | 9 | 0.71 | 0.53 | 0.38 | 4.95E−180 | 6.86E−176 |
| ZFP36L1 | 9 | 0.91 | 0.83 | 0.35 | 8.91E−177 | 1.23E−172 |
| IFI6 | 9 | 0.76 | 0.51 | 0.25 | 2.45E−176 | 3.40E−172 |
| ARPC2 | 9 | 0.99 | 0.96 | 0.25 | 1.68E−173 | 2.32E−169 |
| TOX | 9 | 0.30 | 0.11 | 0.32 | 4.63E−172 | 6.41E−168 |
| ECH1 | 9 | 0.52 | 0.30 | 0.27 | 1.44E−171 | 2.00E−167 |
| ITGB1 | 9 | 0.59 | 0.43 | 0.26 | 2.67E−169 | 3.70E−165 |
| APOL2 | 9 | 0.37 | 0.17 | 0.25 | 2.77E−166 | 3.84E−162 |
| RPS4Y1 | 9 | 0.78 | 0.55 | 0.28 | 7.66E−163 | 1.06E−158 |
| MAP4 | 9 | 0.53 | 0.30 | 0.26 | 1.71E−162 | 2.36E−158 |
| CTSL | 9 | 0.12 | 0.09 | 0.51 | 4.13E−161 | 5.72E−157 |
| DBNL | 9 | 0.63 | 0.42 | 0.27 | 2.57E−159 | 3.57E−155 |
| S100A11 | 9 | 0.84 | 0.71 | 0.36 | 6.52E−153 | 9.03E−149 |
| RCSD1 | 9 | 0.43 | 0.22 | 0.26 | 1.06E−152 | 1.47E−148 |
| GSTK1 | 9 | 0.77 | 0.58 | 0.31 | 2.02E−152 | 2.80E−148 |
| MYH9 | 9 | 0.77 | 0.58 | 0.29 | 3.25E−151 | 4.51E−147 |
| HLA-DRB1 | 9 | 0.35 | 0.20 | 0.56 | 1.71E−148 | 2.38E−144 |
| VAMP8 | 9 | 0.65 | 0.44 | 0.25 | 4.34E−147 | 6.02E−143 |
| RTN4 | 9 | 0.58 | 0.38 | 0.27 | 2.86E−146 | 3.97E−142 |
| BST2 | 9 | 0.82 | 0.61 | 0.30 | 3.07E−146 | 4.25E−142 |
| CYP51A1 | 9 | 0.59 | 0.38 | 0.27 | 1.08E−145 | 1.49E−141 |
| ELOVL1 | 9 | 0.55 | 0.32 | 0.26 | 2.94E−144 | 4.08E−140 |
| ATFS | 9 | 0.29 | 0.14 | 0.28 | 3.46E−143 | 4.79E−139 |
| TIFA | 9 | 0.39 | 0.20 | 0.27 | 2.88E−142 | 3.99E−138 |
| TMEM173 | 9 | 0.36 | 0.18 | 0.35 | 4.98E−140 | 6.90E−136 |
| WDR1 | 9 | 0.91 | 0.78 | 0.26 | 2.21E−139 | 3.06E−135 |
| AQP3 | 9 | 0.31 | 0.15 | 0.27 | 1.54E−137 | 2.13E−133 |
| IL1R1 | 9 | 0.25 | 0.10 | 0.27 | 2.50E−129 | 3.46E−125 |
| TRAPPC1 | 9 | 0.71 | 0.51 | 0.27 | 4.77E−127 | 6.60E−123 |
| ITGA4 | 9 | 0.50 | 0.32 | 0.30 | 1.01E−126 | 1.40E−122 |
| LTA | 9 | 0.80 | 0.64 | 0.39 | 8.43E−126 | 1.17E−121 |
| CHCHD10 | 9 | 0.49 | 0.30 | 0.27 | 2.93E−124 | 4.06E−120 |
| ZBTB32 | 9 | 0.36 | 0.21 | 0.40 | 7.14E−121 | 9.90E−117 |
| CSTB | 9 | 0.82 | 0.68 | 0.27 | 1.81E−120 | 2.51E−116 |
| HLA-DRB5 | 9 | 0.30 | 0.15 | 0.42 | 1.47E−118 | 2.04E−114 |
| TAGLN2 | 9 | 0.90 | 0.77 | 0.30 | 2.08E−117 | 2.88E−113 |
| EPSTI1 | 9 | 0.61 | 0.41 | 0.29 | 2.59E−115 | 3.59E−111 |
| HLA-DPA1 | 9 | 0.38 | 0.22 | 0.31 | 5.73E−113 | 7.94E−109 |
| TALDO1 | 9 | 0.75 | 0.56 | 0.28 | 8.52E−105 | 1.18E−100 |
| ARPCS | 9 | 0.76 | 0.62 | 0.26 | 1.12E−104 | 1.55E−100 |
| GZMA | 9 | 0.15 | 0.09 | 0.41 | 2.37E−99 | 3.28E−95 |
| CTLA4 | 9 | 0.56 | 0.41 | 0.39 | 8.31E−96 | 1.15E−91 |
| LCK | 9 | 0.75 | 0.60 | 0.25 | 2.15E−74 | 2.98E−70 |
| LMNA | 9 | 0.56 | 0.44 | 0.29 | 1.25E−51 | 1.74E−47 |
| HLA-DRA | 9 | 0.21 | 0.12 | 0.37 | 1.51E−47 | 2.09E−43 |
| CD70 | 9 | 0.31 | 0.18 | 0.28 | 5.66E−45 | 7.84E−41 |

As referred to herein, Table 6 depicts as follows:

TABLE 6

| | | Virus- | |
|---|---|---|---|
| Clonotype ID | CD R3 Amino Acid Sequences | reactivity | Clone Size |
| clonotype32211 | TRA: CAVDPILTGGGNKLTF (SEQ ID NO: 1); | CV | 1871 |
| | TRB: CASSLSRDTYNEQFF (SEQ ID NO: 2) | | |
| clonotype20067 | TRA: CAMREVNTGNQFYF; (SEQ ID NO: 3) | CV | 1714 |
| | TRB: CASSPR DSAQSWYGYTF(SEQ ID NO: | | |
| | 4) | | |
| clonotype20068 | TRA: CAVSDGIQGAQKLVF; (SEQ ID NO: 5) | CV | 1175 |
| | TRB: CSVDQGLNYGYTF(SEQ ID NO: 6) | | |
| clonotype20069 | TRA: CAPLGAGGFKTIF; (SEQ ID NO: 7) | CV | 670 |
| | TRB: CASSEALSGGAFGGELFF(SEQ ID NO: | | |
| | 8) | | |
| clonotype20070 | TRA: CAESWAGGGADGLTF; (SEQ ID NO: 9) | CV | 622 |
| | TRB: CASNRPGQGINEQFF(SEQ ID NO: 10) | | |
| clonotype50222 | TRA: CAVDPILTGGGNKLTF; (SEQ ID NO: | CV | 155 |
| | 11) | | |
| | TRB: CSLSGTAATNYGYTF(SEQ ID NO: 12) | | |
| clonotype50223 | TRA: CALSSPNFGNEKLTF; (SEQ ID NO: 13) | CV | 118 |
| | TRA: CAVDSRGGATNKLIF; (SEQ ID NO: 14) | | |
| | TRB: CASSGGAATTNEKLFF(SEQ ID NO: 15) | | |
| clonotype20071 | TRB: CASSPRDSAQSWYGYTF(SEQ ID NO: | CV | 107 |
| | 16) | | |
| clonotype50224 | TRA: CALSSPNFGNEKLTF; (SEQ ID NO: 17) | CV | 95 |
| | TRB: CASSGGAATTNEKLFF(SEQ ID NO: 18) | | |
| clonotype50225 | TRA: CAARGTGTASKLTF; (SEQ ID NO: 19) | CV | 85 |
| | TRA: CAPDNYGGSQGNLIF; (SEQ ID NO: 20) | | |
| | TRB: CASTGAEAATNEKLFF(SEQ ID NO: 21) | | |
| clonotype20073 | TRA: CAPLGAGGFKTIF(SEQ ID NO: 22) | CV | 83 |
| clonotype25395 | TRA: CAMSDILTGGGNKLTF; (SEQ ID NO: | CV | 125 |
| | 23) | | |
| | TRB: CASSQVDRTEAFF(SEQ ID NO: 24) | | |
| clonotype32218 | TRA: CAFYASGGSYIPTF; (SEQ ID NO: 25) | CV | 68 |
| | TRB: CASSLAEGAYEQYF(SEQ ID NO: 26) | | |
| clonotype32213 | TRA: CAVEDRDGGATNKLIF; (SEQ ID NO: | CV | 99 |
| | 27) | | |
| | TRB: CASSLAQGAAGELFF(SEQ ID NO: 28) | | |
| clonotype20074 | TRB: CSVDQGLNYGYTF(SEQ ID NO: 29) | CV | 63 |
| clonotype50227 | TRA: CAARGTGTASKLTF; (SEQ ID NO: 30) | CV | 58 |
| | TRA: CAPDNYGGSQGNLIF(SEQ ID NO: 31) | | |
| clonotype32219 | TRA: CAVDPILTGGGNKLTF(SEQ ID NO: 32) | CV | 51 |
| clonotype32215 | TRA: CAENRLNYQLIW; (SEQ ID NO: 33) | CV | 75 |
| | TRB: CASSRAGMGRTEAFF(SEQ ID NO: 34) | | |
| clonotype50228 | TRA: CALSLSGYALNF; (SEQ ID NO: 35) | CV | 49 |
| | TRB: CASSEGIGQNQETQYF(SEQ ID NO: 36) | | |
| clonotype25398 | TRA: CAASNYGQNFVF; (SEQ ID NO: 37) | CV | 169 |
| | TRB: CASSPIAAYNEQFF(SEQ ID NO: 38) | | |
| clonotype38510 | TRA: CAMRGFNTNAGKSTF; (SEQ ID NO: 39) | CV | 41 |
| | TRB: CASTTGAAPYNEQFF(SEQ ID NO: 40) | | |
| clonotype32216 | TRA: CAVVAPQTGANNLFF; (SEQ ID NO: 41) | CV | 81 |
| | TRB: CASSTGAGSSYNEQFF(SEQ ID NO: 42) | | |
| clonotype20081 | TRA: CAVSDGIQGAQKLVF(SEQ ID NO: 43) | CV | 64 |
| clonotype38513 | TRA: CAMRPWNTGNQFYF; (SEQ ID NO: 44) | CV | 35 |
| | TRB: CASSQEEAGGIDTQYF(SEQ ID NO: 45) | | |
| clonotype50229 | TRA: CALSLSGYALNF(SEQ ID NO: 46) | CV | 34 |
| clonotype32221 | TRB: CASSLSRDTYNEQFF(SEQ ID NO: 47) | CV | 34 |
| clonotype25402 | TRA: CATPAGGYNKLIF; (SEQ ID NO: 48) | CV | 399 |
| | TRB: CASRGLSTDTQYF(SEQ ID NO: 49) | | |
| clonotype20132 | TRA: CAMREVNTGNQFYF; (SEQ ID NO: 50) | CV | 23 |
| | TRA: CAPLGAGGFKTIF; (SEQ ID NO: 51) | | |
| | TRB: CASSEALSGGAFGGELFF; (SEQ ID NO: | | |
| | 52) | | |
| | TRB: CASSPRDSAQSWYGYTF(SEQ ID NO: | | |
| | 53) | | |
| clonotype32225 | TRA: CAENRLNYQLIW; (SEQ ID NO: 54) | CV | 43 |
| | TRA: CAVYLNRDDKIIF; (SEQ ID NO: 55) | | |
| | TRB: CASSRAGMGRTEAFF(SEQ ID NO: 56) | | |
| clonotype25399 | TRA: CAMSPYSSASKIIF; (SEQ ID NO: 57) | CV | 85 |
| | TRB: CASSPSGLVQETQYF(SEQ ID NO: 58) | | |
| clonotype20105 | TRA: CAESWAGGGADGLTF(SEQ ID NO: 59) | CV | 22 |
| clonotype20072 | TRA: CAVRVAGGSYIPTF; (SEQ ID NO: 60) | CV | 159 |
| | TRB: CASSLRVETQYF(SEQ ID NO: 61) | | |
| clonotype25400 | TRA: CAYFPQGGSEKLVF; (SEQ ID NO: 62) | CV | 159 |
| | TRB: CASSPWGGSNQPQHF(SEQ ID NO: 63) | | |
| clonotype32217 | TRA: CALLNTNAGKSTF; (SEQ ID NO: 64) | CV | 50 |
| | TRB: CSARVAGGVYNEQFF(SEQ ID NO: 65) | | |
| clonotype32227 | TRB: CASSLAQGAAGELFF(SEQ ID NO: 66) | CV | 20 |
| clonotype20198 | TRA: CAMREVNTGNQFYF(SEQ ID NO: 67) | CV | 16 |
| clonotype32248 | TRA: CAMRETNQGGKLIF; (SEQ ID NO: 68) | CV | 18 |
| | TRB: CASSYGDRGFPDEKLFF(SEQ ID NO: | | |
| | 69) | | |
| clonotype50237 | TRA: CAASIVSDYKLSF; (SEQ ID NO: 70) | CV | 15 |
| | TRB: CASSPGATGGSTNYGYTF(SEQ ID NO: | | |
| | 71) | | |
| clonotype20171 | TRA: CAMREVNTGNQFYF; (SEQ ID NO: 72) | CV | 14 |
| | TRA: CAPLGAGGFKTIF; (SEQ ID NO: 73) | | |
| | TRB: CASSPRDSAQSWYGYTF(SEQ ID NO: | | |
| | 74) | | |
| clonotype50248 | TRA: CILRVDMRF; (SEQ ID NO: 75) | CV | 12 |
| | TRB: CASSEALVVASQPNQPQHF(SEQ ID | | |
| | NO: 76) | | |
| clonotype20186 | TRB: CASNRPGQGINEQFF(SEQ ID NO: 77) | CV | 11 |
| clonotype32251 | TRB: CASSLAEGAYEQYF(SEQ ID NO: 78) | CV | 11 |
| clonotype25406 | TRA: CVVSAASNKLIF; (SEQ ID NO: 79) | CV | 101 |
| | TRB: CASSLGYGLSTPDTQYF(SEQ ID NO: | | |
| | 80) | | |
| clonotype50256 | TRA: CAVSAPLQGGSEKLVF; (SEQ ID NO: | CV | 12 |
| | 81) | | |
| | TRB: CASSEFGTGFTEAFF(SEQ ID NO: 82) | | |
| clonotype50261 | TRA: CAVDSRGGATNKLIF; (SEQ ID NO: 83) | CV | 11 |
| | TRB: CASSGGAATTNEKLFF(SEQ ID NO: 84) | | |
| clonotype50253 | TRA: CAVTKGFGNVLHC; (SEQ ID NO: 85) | CV | 9 |
| | TRB: CARTSGFYNEQFF(SEQ ID NO: 86) | | |
| clonotype50292 | TRA: CAAILTGGGNKLTF; (SEQ ID NO: 87) | CV | 9 |
| | TRB: CASSPGQASGANVLTF(SEQ ID NO: 88) | | |
| clonotype32253 | TRA: CAASARAQGGSEKLVF; (SEQ ID NO: | CV | 23 |
| | 89) | | |
| | TRB: CASSHRTGVNEKLFF(SEQ ID NO: 90) | | |
| clonotype38533 | TRA: CAAIFQGGSEKLVF; (SEQ ID NO: 91) | CV | 17 |
| | TRB: CASSIVEAVAHNEQFF(SEQ ID NO: 92) | | |
| clonotype36501 | TRA: CAVQALNNDMRF; (SEQ ID NO: 93) | CV | 13 |
| | TRB: CASSYNHEQYF(SEQ ID NO: 94) | | |
| clonotype20203 | TRB: CASSEALSGGAFGGELFF(SEQ ID NO: | CV | 8 |
| | 95) | | |
| elonotype50274 | TRA: CAVSLWNTGNQFYF(SEQ ID NO: 96) | CV | 8 |
| clonotype50309 | TRA: CAVSAYSSASKIIF; (SEQ ID NO: 97) | CV | 8 |
| | TRB: CASSQGSAPATGELFF(SEQ ID NO: 98) | | |
| clonotype32223 | TRA: CAATQWNTGNQFYF; (SEQ ID NO: 99) | CV | 35 |
| | TRB: CASSRPGQGSTEAFF(SEQ ID NO: 100) | | |
| clonotype32518 | TRA: CAAAGVYTGNQFYF; (SEQ ID NO: 101) | CV | 13 |
| | TRA: CAAVRNNNNDMRF; (SEQ ID NO: 102) | | |
| | TRB: CASSQGGDTQYF(SEQ ID NO: 103) | | |
| clonotype38860 | TRA: CAVNPFTSGTYKYIF; (SEQ ID NO: 104) | CV | 8 |
| | TRB: CASSQNSLGYTYEQYF(SEQ ID NO: | | |
| | 105) | | |
| clonotype20225 | TRA: CAPLGAGGFKTIF; (SEQ ID NO: 106) | CV | 7 |
| | TRB: CASSEALSGGAFGGELFF; (SEQ ID NO: | | |
| | 107) | | |
| | TRB: CASSPRDSAQSWYGYTF(SEQ ID NO: | | |
| | 108) | | |
| clonotype20249 | TRA: CAMSAFGQGGSEKLVF; (SEQ ID NO: | CV | 7 |
| | 109) | | |
| | TRB: CASSSNSGNTIYF(SEQ ID NO: 110) | | |
| clonotype50252 | TRB: CSLSGTAATNYGYTF(SEQ ID NO: 111) | CV | 7 |
| clonotype50555 | TRA: CAVSLWNTGNQFYF; (SEQ ID NO: 112) | CV | 7 |
| | TRB: CASSFPGQGYTEAFF(SEQ ID NO: 113) | | |
| clonotype32220 | TRA: CAVDSILTGGGNKLTF; (SEQ ID NO: | CV | 71 |
| | 114) | | |
| | TRB: CASSLGGSVWSPLHF(SEQ ID NO: 115) | | |
| clonotype25474 | TRA: CAMRVLGGYQKVTF; (SEQ ID NO: | CV | 45 |
| | 116) | | |
| | TRB: CSATRLNADTQYF(SEQ ID NO: 117) | | |
| clonotype32237 | TRA: CAVSDSGGGADGLTF; (SEQ ID NO: | CV | 19 |
| | 118) | | |
| | TRB: CASSRAGFANYGYTF(SEQ ID NO: 119) | | |
| clonotype50287 | TRA: CAVDPILTGGGNKLTF; (SEQ ID NO: | CV | 7 |
| | 120) | | |
| | TRB: CASSFNRDTYNEQFF(SEQ ID NO: 121) | | |
| clonotype50270 | TRA: CAPDNYGGSQGNLIF; (SEQ ID NO: | CV | 6 |
| | 122) | | |
| | TRB: CASTGAEAATNEKLFF(SEQ ID NO: | | |
| | 123) | | |
| clonotype50442 | TRA: CAPDNYGGSQGNLIF(SEQ ID NO: 124) | CV | 6 |
| clonotype20383 | TRA: CAGPHASGGSYIPTF; (SEQ ID NO: 125) | CV | 9 |
| | TRB: CASSQRDPYNEQFF(SEQ ID NO: 126) | | |
| clonotype20072 | TRA: CAVRVAGGSYIPTF; (SEQ ID NO: 127) | CV | 159 |
| | TRB: CASSLRVETQYF(SEQ ID NO: 128) | | |
| clonotype25398 | TRA: CAASNYGQNFVF; (SEQ ID NO: 129) | CV | 169 |
| | TRB: CASSPIAAYNEQFF(SEQ ID NO: 130) | | |
| clonotype25402 | TRA: CATPAGGYNKLIF; (SEQ ID NO: 131) | CV | 399 |
| | TRB: CASRGLSTDTQYF(SEQ ID NO: 132) | | |
| clonotype25400 | TRA: CAYFPQGGSEKLVF; (SEQ ID NO: 133) | CV | 159 |
| | TRB: CASSPWGGSNQPQHF(SEQ ID NO: 134) | | |
| clonotype32211 | TRA: CAVDPILTGGGNKLTF; (SEQ ID NO: | CV | 1871 |
| | 135) | | |
| | TRB: CASSLSRDTYNEQFF(SEQ ID NO: 136) | | |
| clonotype25399 | TRA: CAMSPYSSASKIIF; (SEQ ID NO: 137) | CV | 85 |
| | TRB: CASSPSGLVQETQYF(SEQ ID NO: 138) | | |
| clonotype20067 | TRA: CAMREVNTGNQFYF; (SEQ ID NO: 139) | CV | 1714 |
| | TRB: CASSPRDSAQSWYGYTF(SEQ ID NO: | | |
| | 140) | | |
| clonotype20069 | TRA: CAPLGAGGFKTIF; (SEQ ID NO: 141) | CV | 670 |
| | TRB: CASSEALSGGAFGGELFF(SEQ ID NO: | | |
| | 142) | | |
| clonotype32213 | TRA: CAVEDRDGGATNKLIF; (SEQ ID NO: | CV | 99 |
| | 143) | | |
| | TRB: CASSLAQGAAGELFF(SEQ ID NO: 144) | | |
| clonotype32217 | TRA: CALLNTNAGKSTF; (SEQ ID NO: 145) | CV | 50 |
| | TRB: CSARVAGGVYNEQFF(SEQ ID NO: 146) | | |
| clonotype20070 | TRA: CAESWAGGGADGLTF; (SEQ ID NO: | CV | 622 |
| | 147) | | |
| | TRB: CASNRPGQGINEQFF(SEQ ID NO: 148) | | |
| clonotype25406 | TRA: CVVSAASNKLIF; (SEQ ID NO: 149) | CV | 101 |
| | TRB: CASSLGYGLSTPDTQYF(SEQ ID NO: | | |
| | 150) | | |
| clonotype20068 | TRA: CAVSDGIQGAQKLVF; (SEQ ID NO: | CV | 1175 |
| | 151) | | |
| | TRB: CSVDQGLNYGYTF(SEQ ID NO: 152) | | |
| clonotype25395 | TRA: CAMSDILTGGGNKLTF; (SEQ ID NO: | CV | 125 |
| | 153) | | |
| | TRB: CASSQVDRTEAFF(SEQ ID NO: 154) | | |
| clonotype32237 | TRA: CAVSDSGGGADGLTF; (SEQ ID NO: 155) | CV | 19 |
| | TRB: CASSRAGFANYGYTF(SEQ ID NO: 156) | | |
| clonotype20182 | TRA: CAVTSGAGSYQLTF; (SEQ ID NO: 157) | CV | 19 |
| | TRB: CASSYSLSSYNSPLHF(SEQ ID NO: 158) | | |
| clonotype25474 | TRA: CAMRVLGGYQKVTF; (SEQ ID NO: 159) | CV | 45 |
| | TRB: CSATRLNADTQYF(SEQ ID NO: 160) | | |
| clonotype39213 | TRA: CAVNPQGGSEKLVF; (SEQ ID NO: 161) | CV | 240 |
| | TRB: CSATSQGFSNQPQHF(SEQ ID NO: 162) | | |
| clonotype50222 | TRA: CAVDPILTGGGNKLTF; (SEQ ID NO: | CV | 155 |
| | 163) | | |
| | TRB: CSLSGTAATNYGYTF(SEQ ID NO: 164) | | |
| clonotype38529 | TRA: CVVSEPNYGQNFVF; (SEQ ID NO: 165) | CV | 14 |
| | TRB: CASSLRTGGTDTQYF(SEQ ID NO: 166) | | |
| clonotype38690 | TRA: CAFIRAGNMLTF; (SEQ ID NO: 167) | CV | 382 |
| | TRB: CASSADRDLEAFF(SEQ ID NO: 168) | | |
| clonotype48823 | TRA: CAVPGSQGGSEKLVF; (SEQ ID NO: | CV | 158 |
| | 169) | | |
| | TRB: CASNQAEAGELFF(SEQ ID NO: 170) | | |
| clonotype36501 | TRA: CAVQALNNDMRF; (SEQ ID NO: 171) | CV | 13 |
| | TRB: CASSYNHEQYF(SEQ ID NO: 172) | | |
| clonotype20433 | TRA: CAVRVAGGSYIPTF; (SEQ ID NO: 173) | CV | 6 |
| | TRB: CASSLRVETQYF(SEQ ID NO: 174) | | |
| clonotype50223 | TRA: CALSSPNFGNEKLTF; (SEQ ID NO: 175) | CV | 118 |
| | TRA: CAVDSRGGATNKLIF; (SEQ ID NO: | | |
| | 176) | | |
| | TRB: CASSGGAATTNEKLFF(SEQ ID NO: | | |
| | 177) | | |
| clonotype32215 | TRA: CAENRLNYQLIW; (SEQ ID NO: 178) | CV | 75 |
| | TRB: CASSRAGMGRTEAFF(SEQ ID NO: 179) | | |
| clonotype32227 | TRB: CASSLAQGAAGELFF(SEQ ID NO: 180) | CV | 20 |
| clonotype32518 | TRA: CAAAGVYTGNQFYF; (SEQ ID NO: 181) | CV | 13 |
| | TRA: CAAVRNNNNDMRF; (SEQ ID NO: 182) | | |
| | TRB: CASSQGGDTQYF(SEQ ID NO: 183) | | |
| clonotype26177 | TRB: CASSPWGGSNQPQHF(SEQ ID NO: 184) | CV | 7 |
| clonotype20074 | TRB: CSVDQGLNYGYTF(SEQ ID NO: 185) | CV | 63 |
| clonotype38634 | TRA: CAVEDGYGGATNKLIF; (SEQ ID NO: | CV | 25 |
| | 186) | | |
| | TRB: CASSLALGMGGETQYF(SEQ ID NO: | | |
| | 187) | | |
| clonotype38848 | TRA: CALSRNSGGSNYKLTF; (SEQ ID NO: | CV | 19 |
| | 188) | | |
| | TRB: CASRTGLRSGTEAFF(SEQ ID NO: 189) | | |
| clonotype36505 | TRA: CAVQALNNDMRF; (SEQ ID NO: 190) | CV | 8 |
| | TRB: CASSRPRVEQGKYEQYF; (SEQ ID NO: | | |
| | 191) | | |
| | TRB: CASSYNHEQYF(SEQ ID NO: 192) | | |
| clonotype25702 | TRA: CAMSTYSSASKIIF; (SEQ ID NO: 193) | CV | 7 |
| | TRB: CASSPSGLAYEQYF(SEQ ID NO: 194) | | |
| clonotype20275 | TRB: CASSLRVETQYF(SEQ ID NO: 195) | CV | 5 |
| clonotype38987 | TRA: CAVSAPNYGQNFVF; (SEQ ID NO: 196) | CV | 5 |
| | TRB: CASRPGTGGNQPQHF(SEQ ID NO: 197) | | |
| clonotype20684 | TRA: CAVREAGGSYIPTF; (SEQ ID NO: 198) | CV | 4 |
| | TRB: CASSLRVETQYF(SEQ ID NO: 199) | | |
| clonotype32511 | TRB: CSARVAGGVYNEQFF(SEQ ID NO: 200) | CV | 3 |
| clonotype39198 | TRA: CAASSHSGAGSYQLTF; (SEQ ID NO: | CV | 369 |
| | 201) | | |
| | TRB: CASSLVTDTQYF(SEQ ID NO: 202) | | |
| clonotype30274 | TRA: CAFTQEAGNTPLVF; (SEQ ID NO: 203) | CV | 77 |
| | TRB: CASRRGSPTDTQYF(SEQ ID NO: 204) | | |
| clonotype50228 | TRA: CALSLSGYALNF; (SEQ ID NO: 205) | CV | 49 |
| | TRB: CASSEGIGQNQETQYF(SEQ ID NO: 206) | | |
| clonotype32225 | TRA: CAENRLNYQLIW; (SEQ ID NO: 207) | CV | 43 |
| | TRA: CAVYLNRDDKIIF; (SEQ ID NO: 208) | | |
| | TRB: CASSRAGMGRTEAFF(SEQ ID NO: 209) | | |
| clonotype38510 | TRA: CAMRGENTNAGKSTF; (SEQ ID NO: | CV | 41 |
| | 210) | | |
| | TRB: CASTTGAAPYNEQFF(SEQ ID NO: 211) | | |
| clonotype40376 | TRA: CAVSDLYGGATNKLIF; (SEQ ID NO: | CV | 22 |
| | 212) | | |
| | TRB: CASSDGLAGYNEQFF(SEQ ID NO: 213) | | |
| clonotype38781 | TRA: CVVNMGTSYDKVIF; (SEQ ID NO: 214) | CV | 18 |
| | TRB: CASSLASYDNEQFF(SEQ ID NO: 215) | | |
| clonotype20462 | TRA: CVVSDQGNAGKSTF; (SEQ ID NO: 216) | CV | 15 |
| | TRB: CSASHLKETQYF(SEQ ID NO: 217) | | |
| clonotype50261 | TRA: CAVDSRGGATNKLIF; (SEQ ID NO: | CV | 11 |
| | 218) | | |
| | TRB: CASSGGAATTNEKLFF(SEQ ID NO: | | |
| | 219) | | |
| clonotype48858 | TRB: CSATSQGFSNQPQHF(SEQ ID NO: 220) | CV | 8 |
| clonotype20706 | TRA: CALSDPGGTYKYIF; (SEQ ID NO: 221) | CV | 7 |
| | TRB: CASSPGGGNTEAFF(SEQ ID NO: 222) | | |
| clonotype32913 | TRA: CAVGRGSTLGRLYF; (SEQ ID NO: 223) | CV | 6 |
| | TRB: CASSGDSRGGYNNEQFF(SEQ ID NO: | | |
| | 224) | | |
| clonotype25918 | TRA: CAARSLYNFNKFYF; (SEQ ID NO: 225) | CV | 6 |
| | TRB: CASSQDGGSGWETQYF(SEQ ID NO: | | |
| | 226) | | |
| clonotype26248 | TRA: CAVGVNNNDMRF; (SEQ ID NO: 227) | CV | 6 |
| | TRB: CSVPGPYYNEQFF(SEQ ID NO: 228) | | |
| clonotype25647 | TRA: CAASEVKVTSGSRLTF; (SEQ ID NO: | CV | 4 |
| | 229) | | |
| | TRB: CASSFGGLATQPQHF(SEQ ID NO: 230) | | |
| clonotype50406 | TRA: CAYKTSYDKVIF; (SEQ ID NO: 231) | CV | 3 |
| | TRB: CASSIEGTVSFYEQYF(SEQ ID NO: 232) | | |
| clonotype29234 | TRA: CAMTSYSSASKIIF; (SEQ ID NO: 233) | CV | 2 |
| | TRB: CASSPNGAYNEQFF(SEQ ID NO: 234) | | |
| clonotype36123 | TRA: CASLVEYGNKLVF; (SEQ ID NO: 235) | CV | 2 |
| | TRA: CATNTDKLIF; (SEQ ID NO: 236) | | |
| | TRB: CASRQGLDDTQYF(SEQ ID NO: 237) | | |
| clonotype41193 | TRA: CVVTYSGGYQKVTF; (SEQ ID NO: 238) | CV | 2 |
| | TRB: CASSPTGDDGYTF(SEQ ID NO: 239) | | |

As referred to herein, Table 7 depicts as follows:

TABLE 7

| | | Virus- | Clone |
|---|---|---|---|
| Clonotype ID | CD R3 Amino Acid Sequences | reactivity | Size |
| clonotype57836 | TRA: CAMKDSGYSTLTF; (SEQ ID NO: 240) | CV | 30 |
| | TRB: CASSFEGGDTEAFF(SEQ ID NO: 241) | | |
| clonotype57835 | TRA: CALSDLIGTASKLTF; (SEQ ID NO: | CV | 26 |
| | 242) | | |
| | TRB: CSARAGARNTGELFF(SEQ ID NO: | | |
| | 243) | | |
| clonotype57833 | TRA: CAASRVEAGTYKYIF; (SEQ ID NO: | CV | 21 |
| | 244) | | |
| | TRB: CSVEDGQWDTGELFF(SEQ ID NO: | | |
| | 245) | | |
| clonotype57837 | TRA: CAMSQNRDDKIIF; (SEQ ID NO: | CV | 21 |
| | 246) | | |
| | TRB: CASRYRGRENTEAFF(SEQ ID NO: | | |
| | 247) | | |
| clonotype57839 | TRA: CILRDRTGANNLFF; (SEQ ID NO: | CV | 18 |
| | 248) | | |
| | TRB: CSARGTGGRNTEAFF(SEQ ID NO: | | |
| | 249) | | |
| clonotype57840 | TRA: CALSVFVDDMRF; (SEQ ID NO: 250) | CV | 18 |
| | TRB: CASSYGGNQPQHF(SEQ ID NO: 251) | | |
| clonotype57842 | TRA: CAMSAYASNYQLIW; (SEQ ID NO: 252) | CV | 17 |
| | TRB: CASSGGLALALQETQYF(SEQ ID NO: | | |
| | 253) | | |
| clonotype57857 | TRA: CGTVRSNDYKLSF; (SEQ ID NO: 254) | CV | 15 |
| | TRB: CASSEAGGTGDTHSNQPQHF(SEQ ID | | |
| | NO: 255) | | |
| clonotype57859 | TRA: CAVISGYSTLTF; (SEQ ID NO: 256) | CV | 15 |
| | TRB: CASSFVSGGGTGELFF(SEQ ID NO: | | |
| | 257) | | |
| clonotype57887 | TRA: CAASRDRLMF; (SEQ ID NO: 258) | CV | 15 |
| | TRB: CASSLEGAEQYF(SEQ ID NO: 259) | | |
| clonotypes7846 | TRA: CAVSTILSGGYNKLIF; (SEQ ID NO: 260) | CV | 14 |
| | TRB: CASSPPSGGAYEQYF(SEQ ID NO: | | |
| | 261) | | |
| clonotype58345 | TRA: CAMSGNGNAGNMLTF; (SEQ ID NO: 262) | CV | 14 |
| | TRB: CATSRDPGGTDTQYF(SEQ ID NO: | | |
| | 263) | | |
| clonotype73522 | TRA: CASLGAGNMLTF; (SEQ ID NO: 264) | CV | 14 |
| | TRB: CASSLPLGAGGRDEQFF(SEQ ID NO: | | |
| | 265) | | |
| clonotype57841 | TRA: CAVQGAQKLVF; (SEQ ID NO: 266) | CV | 13 |
| | TRB: CASSTGTYYEQYF(SEQ ID NO: 267) | | |
| clonotype57855 | TRA: CALSDYGGSQGNLIF; (SEQ ID NO: 268) | CV | 13 |
| | TRB: CASSSGQGQTQYF(SEQ ID NO: 269) | | |
| clonotype57843 | TRA: CALIIQGAQKLVF; (SEQ ID NO: 270) | CV | 12 |
| | TRB: CASSSRTSGIFDTQYF(SEQ ID NO: | | |
| | 271) | | |
| clonotype57856 | TRA: CAVQGGSQGNLIF; (SEQ ID NO: 272) | CV | 12 |
| | TRB: CASSFIKNTEAFF(SEQ ID NO: 273) | | |
| clonotype73524 | TRA: CAVTGYAGNMLTF; (SEQ ID NO: 274) | CV | 12 |
| | TRB: CAWSPGLGSYEQYF(SEQ ID NO: 275) | | |
| clonotype57853 | TRA: CALTASRGSNYKLTF; (SEQ ID NO: 276) | CV | 12 |
| | TRB: CASSQVGTRDTEAFF(SEQ ID NO: | | |
| | 277) | | |
| clonotype73523 | TRA: CAMRRGGAQKLVF; (SEQ ID NO: 278) | CV | 12 |
| | TRB: CASSLEGQAGELFF(SEQ ID NO: 279) | | |
| clonotype57854 | TRA: CAMRGNTGKLIF; (SEQ ID NO: 280) | CV | 11 |
| | TRB: CASSGRTGANEKLFF(SEQ ID NO: | | |
| | 281) | | |
| clonotype57861 | TRA: CAVPTGNQFYF; (SEQ ID NO: 282) | CV | 11 |
| | TRB: CASSAPGLPGNEQFF(SEQ ID NO: | | |
| | 283) | | |
| clonotype57866 | TRA: CAFWGQGAQKLVF; (SEQ ID NO: | CV | 11 |
| | 284) | | |
| | TRB: CAISESPGQGNEQYF(SEQ ID NO: 285) | | |
| clonotype57867 | TRA: CIATNSGGYQKVTF; (SEQ ID NO: 286) | CV | 11 |
| | TRB: CATSRLTGATEQFF(SEQ ID NO: 287) | | |
| clonotype57882 | TRA: CAASISNAGGTSYGKLTF; (SEQ ID NO: 288) | CV | 11 |
| | TRB: CASRAQGRETQYF(SEQ ID NO: 289) | | |
| clonotype57885 | TRA: CAASGFGNVLHC; (SEQ ID NO: 290) | CV | 11 |
| | TRB: CASSLGRGVSAGELFF(SEQ ID NO: | | |
| | 291) | | |
| clonotype57888 | TRA: CAVRDSTGGFKTIF; (SEQ ID NO: 292) | CV | 11 |
| | TRB: CASIFSSGGQYEQYF(SEQ ID NO: 293) | | |
| clonotype57939 | TRA: CALTSGSRLTF; (SEQ ID NO: 294) | CV | 11 |
| | TRB: CATSDLGTGSRTGELFF(SEQ ID NO: | | |
| | 295) | | |
| clonotype57868 | TRA: CALSGNTPLVF; (SEQ ID NO: 296) | CV | 10 |
| | TRB: CASSQDSQRGNIQYF(SEQ ID NO: | | |
| | 297) | | |
| clonotype57895 | TRA: CIVRSITSGTYKYIF(SEQ ID NO: 298) | CV | 10 |
| clonotype57919 | TRA: CAAFSGTYKYIF; (SEQ ID NO: 299) | CV | 10 |
| | TRB: CATLFKAPYEQYF(SEQ ID NO: 300) | | |
| clonotype73526 | TRA: CAVERDDKIIF; (SEQ ID NO: 301) | CV | 10 |
| | TRB: CASSLDRGRDEQYF(SEQ ID NO: 302) | | |
| clonotype57844 | TRA: CAVNGYSSASKIIF; (SEQ ID NO: 303) | CV | 9 |
| | TRB: CSARERDDSPLHF(SEQ ID NO: 304) | | |
| clonotype57870 | TRA: CAVLMNTGFQKLVF; (SEQ ID NO: 305) | CV | 9 |
| | TRB: CASSGPGATNEKLFF(SEQ ID NO: | | |
| | 306) | | |
| clonotype57871 | TRA: CAMKDSGYSTLTF(SEQ ID NO: 307) | CV | 9 |
| clonotype57880 | TRA: CAARAPGRRALTF; (SEQ ID NO: 308) | CV | 9 |
| | TRA: CAVGKLIF; (SEQ ID NO: 309) | | |
| | TRB: CASSQEGPSNEQFF(SEQ ID NO: 310) | | |
| clonotype57894 | TRA: CAVRTGGSYIPTF; (SEQ ID NO: 311) | CV | 9 |
| | TRB: CAWSSGHTGELFF(SEQ ID NO: 312) | | |
| clonotype57924 | TRA: CATVPTTSGTYKYIF; (SEQ ID NO: 313) | CV | 9 |
| | TRB: CASSLLTGWAFF(SEQ ID NO: 314) | | |
| clonotype57947 | TRA: CAEKGGNNRLAF; (SEQ ID NO: 315) | CV | 9 |
| | TRB: CASSVDRDYEQYF(SEQ ID NO: 316) | | |
| clonotype57998 | TRA: CALLNTGGFKTIF; (SEQ ID NO: 317) | CV | 9 |
| | TRB: CAWSELGQGRGANVLTF(SEQ ID | | |
| | NO: 318) | | |
| clonotype58510 | TRA: CAMREYSSASKIIF; (SEQ ID NO: 319) | CV | 9 |
| | TRB: CASNDRREEAKNIQYF(SEQ ID NO: | | |
| | 320) | | |
| clonotype73525 | TRA: CALSDRAGGTSYGKLTF; (SEQ ID NO: 321) | CV | 9 |
| | TRB: CASSHGTDNSPLHF(SEQ ID NO: 322) | | |
| clonotype57848 | TRA: CAQRGFGNEKLTF; (SEQ ID NO: 323) | CV | 8 |
| | TRB: CASSSGIGGTSYEQYF(SEQ ID NO: | | |
| | 324) | | |
| clonotype57852 | TRA: CILSPVYSGTYKYIF; (SEQ ID NO: 325) | CV | 8 |
| | TRB: CSARKLAASSYNEQFF(SEQ ID NO: | | |
| | 326) | | |
| clonotypes7862 | TRA: CALQEAGGFKTIF; (SEQ ID NO: 327) | CV | 8 |
| | TRB: CATSRGDLLVNEQFF(SEQ ID NO: | | |
| | 328) | | |
| clonotype57879 | TRA: CAVRDTGFQKLVF; (SEQ ID NO: 329) | CV | 8 |
| | TRB: CASSVTRYEQYF(SEQ ID NO: 330) | | |
| clonotype57891 | TRA: CVVTDLGTYKYIF; (SEQ ID NO: 331) | CV | 8 |
| | TRB: CAISEGVWTGDTEAFF (SEQ ID NO: | | |
| | 332) | | |
| clonotype57892 | TRA: CAVFSGNTGKLIF; (SEQ ID NO: 333) | CV | 8 |
| | TRB: CASSFVENTEAFF(SEQ ID NO: 334) | | |
| clonotype57912 | TRA: CAAPFSSGSARQLTF; (SEQ ID NO: 335) | CV | 8 |
| | TRB: CASGGGTSNFRTYEQYF(SEQ ID NO: | | |
| | 336) | | |
| clonotype57918 | TRA: CASLTSGTYKYIF; (SEQ ID NO: 337) | CV | 8 |
| | TRA: CAVDILTGGGNKLTF; (SEQ ID NO: | | |
| | 338) | | |
| | TRB: CASSETDSVNEQFF(SEQ ID NO: 339) | | |
| clonotype57936 | TRA: CAPLRMGRLYF; (SEQ ID NO: 340) | CV | 8 |
| | TRB: CASSLMTLGNTEAFF(SEQ ID NO: | | |
| | 341) | | |
| clonotype57948 | TRA: CATDARNYQLIW; (SEQ ID NO: 342) | CV | 8 |
| | TRB: CASSDTGLAGELFF(SEQ ID NO: 343) | | |
| clonotype58058 | TRA: CALTDRGTNAGKSTF; (SEQ ID NO: 344) | CV | 8 |
| | TRB: CASSQDPQRGGGADTQYF(SEQ ID | | |
| | NO: 345) | | |
| clonotype58340 | TRA: CAEPSTGGFKTIF; (SEQ ID NO: 346) | CV | 8 |
| | TRA: CAESKTVTGGGNKLTF; (SEQ ID NO: | | |
| | 347) | | |
| | TRB: CASSSSGGERRAFF(SEQ ID NO: 348) | | |
| clonotype58428 | TRA: CALSDLGNEKLTF; (SEQ ID NO: 349) | CV | 8 |
| | TRA: CSYQKLVF; (SEQ ID NO: 350) | | |
| | TRB: CASSLGGLAGGEQFF(SEQ ID NO: | | |
| | 351) | | |
| clonotype62044 | TRA: CAMREGRDDKIIF; (SEQ ID NO: 352) | CV | 8 |
| | TRB: CASSLTLARTDTQYF(SEQ ID NO: | | |
| | 353) | | |
| clonotype73527 | TRA: CAENGPRVNTGFQKLVF; (SEQ ID | CV | 8 |
| | NO: 354) | | |
| | TRB: CASMKQTMNTEAFF(SEQ ID NO: | | |
| | 355) | | |
| clonotype73528 | TRA: CALRAPNARLMF; (SEQ ID NO: 356) | CV | 8 |
| | TRB: CASSFGQGSSEAFF(SEQ ID NO: 357) | | |
| clonotype57849 | TRA: CAPVGGTYKYIF; (SEQ ID NO: 358) | CV | 7 |
| | TRB: CASSPTGRGEQYF(SEQ ID NO: 359) | | |
| clonotype57864 | TRA: CACFGAGSYQLTF; (SEQ ID NO: 360) | CV | 7 |
| | TRB: CASSYTRTSNSPLHF(SEQ ID NO: 361) | | |
| clonotype57872 | TRA: CALRDNYGQNFVF; (SEQ ID NO: 362) | CV | 7 |
| | TRA: CAVRSYGGSQGNLIF; (SEQ ID NO: | | |
| | 363) | | |
| | TRB: CASSALGGGTDTQYF(SEQ ID NO: | | |
| | 364) | | |
| clonotype57876 | TRA: CALSSRAGGTSYGKLTF; (SEQ ID NO: 365) | CV | 7 |
| | TRA: CAVRINTGNQFYF; (SEQ ID NO: 366) | | |
| | TRB: CATSDSQVAGSSYNEQFF(SEQ ID | | |
| | NO: 367) | | |
| clonotype57881 | TRA: CAVPNQAGTALIF; (SEQ ID NO: 368) | CV | 7 |
| | TRB: CASSFRTGDQPQHF(SEQ ID NO: 369) | | |
| clonotype57883 | TRA: CAVQTSGTYKYIF; (SEQ ID NO: 370) | CV | 7 |
| | TRB: CASSLVGGAAEAFF(SEQ ID NO: 371) | | |
| clonotype57897 | TRA: CAVNFLSNNAGNMLTF; (SEQ ID NO: 372) | CV | 7 |
| | TRB: CASARYEETQYF(SEQ ID NO: 373) | | |
| clonotype57931 | TRA: CAVESSGGSNYKLTF; (SEQ ID NO: | CV | 7 |
| | 374) | | |
| | TRB: CSARDLSYTQYF(SEQ ID NO: 375) | | |
| clonotype57940 | TRA: CAFMKPVGTYKYIF; (SEQ ID NO: | CV | 7 |
| | 376) | | |
| | TRB: CSASGGDVDTQYF(SEQ ID NO: 377) | | |
| clonotype57966 | TRA: CVVSAGTGGFKTIF; (SEQ ID NO: 378) | CV | 7 |
| | TRB: CASSLGPEMGGHNEQFF(SEQ ID NO: | | |
| | 379) | | |
| clonotype57978 | TRA: CAVRGLSGTYKYIF; (SEQ ID NO: 380) | CV | 7 |
| | TRB: CASSLGTGHHEQFF(SEQ ID NO: 381) | | |
| clonotype58037 | TRA: CAFMTAFNNDMRF; (SEQ ID NO: 382) | CV | 7 |
| | TRB: CASSSGQGTSGGHNEQFF(SEQ ID | | |
| | NO: 383) | | |
| clonotype58052 | TRA: CGTEAAGNKLTF; (SEQ ID NO: 384) | CV | 7 |
| | TRB: CASSLLQGSSYNEQFF(SEQ ID NO: | | |
| | 385) | | |
| clonotype58065 | TRA: CAVNAPSSASKIIF; (SEQ ID NO: 386) | CV | 7 |
| | TRB: CASSPGHRGVNVAKNIQYF(SEQ ID NO: 387) | | |
| clonotype58147 | TRA: CATVETQGGSEKLVF; (SEQ ID NO: | CV | 7 |
| | 388) | | |
| | TRB: CASSLTPGYGEAFF(SEQ ID NO: 389) | | |
| clonotype58331 | TRA: CAGGFKTIF; (SEQ ID NO: 390) | CV | 7 |
| | TRA: CALSDENSGGSNYKLTF; (SEQ ID | | |
| | NO: 391) | | |
| | TRB: CSARGDSNEKLFF(SEQ ID NO: 392) | | |
| clonotype58367 | TRA: CARWSSARQLTF; (SEQ ID NO: 393) | CV | 7 |
| | TRA: CAVYSSASKIIF; (SEQ ID NO: 394) | | |
| | TRB: CASSLGLAGTYEQYF(SEQ ID NO: | | |
| | 395) | | |
| clonotype64911 | TRA: CALSGGYGQNFVF; (SEQ ID NO: 396) | CV | 7 |
| | TRB: CASSLAGTSTDTQYF(SEQ ID NO: | | |
| | 397) | | |
| clonotype57903 | TRA: CAVEAIQGAQKLVF; (SEQ ID NO: | CV | 7 |
| | 398) | | |
| | TRB: CASSEWGEQYF(SEQ ID NO: 399) | | |
| clonotype57858 | TRA: CAERDTGRRALTF(SEQ ID NO: 400) | CV | 6 |
| clonotype57860 | TRA: CVVSARNSGYALNF; (SEQ ID NO: 401) | CV | 6 |
| | TRB: CASSFGQGPYNEQFF(SEQ ID NO: | | |
| | 402) | | |
| clonotype57869 | TRA: CAKPRGRGTMEYGNKLVF; (SEQ ID NO: 403) | CV | 6 |
| | TRA: CAVNLRKTGNQFYF; (SEQ ID NO: | | |
| | 404) | | |
| | TRB: CASSLGETQYF (SEQ ID NO: 405) | | |
| clonotype57886 | TRA: CAVSDQGGSEKLVF; (SEQ ID NO: 406) | CV | 6 |
| | TRB: CASSEAPRFGNTIYF(SEQ ID NO: 407) | | |
| clonotype57889 | TRA: CAVRRYSGGGADGLTF; (SEQ ID NO: 408) | CV | 6 |
| | TRB: CSAGALQGATNEKLFF(SEQ ID NO: | | |
| | 409) | | |
| clonotype57890 | TRA: CAGGYQKVTF; (SEQ ID NO: 410) | CV | 6 |
| | TRB: CASSTLAGVSYNEQFF(SEQ ID NO: | | |
| | 411) | | |
| clonotype57898 | TRA: CAARISSGSARQLTF; (SEQ ID NO: 412) | CV | 6 |
| | TRB: CASSATYNEQFF (SEQ ID NO: 413) | | |
| clonotype57909 | TRA: CVVNQGGKLIF; (SEQ ID NO: 414) | CV | 6 |
| | TRB: CSGAAGGYEQYF(SEQ ID NO: 415) | | |
| clonotype57913 | TRA: CAMRARSNAGGTSYGKLTF; (SEQ ID | CV | 6 |
| | NO: 416) | | |
| | TRA: CIVRGRDQTGANNLFF; (SEQ ID NO: | | |
| | 417) | | |
| | TRB: CASSELGRDDEAFF(SEQ ID NO: 418) | | |
| clonotype57916 | TRA: CALRGNRDDKIIF; (SEQ ID NO: 419) | CV | 6 |
| | TRA: CAMKKDSNYQLIW; (SEQ ID NO: | | |
| | 420) | | |
| | TRB: CAISGAETQYF(SEQ ID NO: 421) | | |
| clonotype57925 | TRA: CAVRALTSGTYKYIF; (SEQ ID NO: | CV | 6 |
| | 422) | | |
| | TRB: CASSGGGGVSEQYF(SEQ ID NO: 423) | | |
| clonotype57944 | TRA: CAGATSGTYKYIF; (SEQ ID NO: 424) | CV | 6 |
| | TRB: CASSLSPGTFYEQYF(SEQ ID NO: 425) | | |
| clonotype57945 | TRA: CAVSPSGNTPLVF; (SEQ ID NO: 426) | CV | 6 |
| | TRB: CASSLTQGDGYTF(SEQ ID NO: 427) | | |
| clonotype57996 | TRA: CAVRHGDDKIIF; (SEQ ID NO: 428) | CV | 6 |
| | TRB: CASWTGTQETQYF(SEQ ID NO: 429) | | |
| clonotype58012 | TRA: CAASKGSDGQKLLF; (SEQ ID NO: 430) | CV | 6 |
| | TRB: CSARITLGELFF(SEQ ID NO: 431) | | |
| clonotype58051 | TRA: CAASISNAGGTSYGKLTF(SEQ ID | CV | 6 |
| | NO: 432) | | |
| clonotype58214 | TRA: CAVRGSGGSNYKLTF; (SEQ ID NO: 433) | CV | 6 |
| | TRB: CASSLVQSGELFF(SEQ ID NO: 434) | | |
| clonotype58253 | TRA: CAETGGGNKLTF; (SEQ ID NO: 435) | CV | 6 |
| | TRB: CASSSGTANEKLFF(SEQ ID NO: 436) | | |
| clonotype58374 | TRA: CAASSQAGTALIF; (SEQ ID NO: 437) | CV | 6 |
| | TRB: CASSIRSAGAGDTQYF(SEQ ID NO: | | |
| | 438) | | |
| clonotype58533 | TRA: CALSYLNQAGTALIF; (SEQ ID NO: 439) | CV | 6 |
| | TRB: CASSQDLVDREQYF(SEQ ID NO: 440) | | |
| clonotype58535 | TRA: CAAARDTGNQFYF; (SEQ ID NO: 441) | CV | 6 |
| | TRB: CASGGSWSKNIQYF(SEQ ID NO: 442) | | |
| clonotype58689 | TRA: CAANTGNQFYF; (SEQ ID NO: 443) | CV | 6 |
| | TRB: CASRWGLHQETQYF(SEQ ID NO: | | |
| | 444) | | |
| clonotype59145 | TRA: CAPRGLGGGKLIF; (SEQ ID NO: 445) | CV | 6 |
| | TRB: CASSTPHRGDGVNTEAFF(SEQ ID | | |
| | NO: 446) | | |
| clonotype60461 | TRA: CAAFLYF; (SEQ ID NO: 447) | CV | 6 |
| | TRB: CASSASTGGIGYTF (SEQ ID NO: 448) | | |
| clonotype61158 | TRA: CAVGVSGGGADGLTF; (SEQ ID NO: 449) | CV | 6 |
| | TRB: CASSLDRNEQFF(SEQ ID NO: 450) | | |
| clonotype62111 | TRA: CAVSNAGNNRKLIW; (SEQ ID NO: 451) | CV | 6 |
| | TRB: CASSYWGGGNQPQHF(SEQ ID NO: | | |
| | 452) | | |
| clonotype62791 | TRA: CAVGGRSGGYNKLIF; (SEQ ID NO: 453) | CV | 6 |
| | TRB: CASSLAQTGSGNTIYF(SEQ ID NO: | | |
| | 454) | | |
| clonotype72074 | TRA: CLVGDHSGNTPLVF; (SEQ ID NO: 455) | CV | 6 |
| | TRB: CSARAEGEGRYNEQFF(SEQ ID NO: | | |
| | 456) | | |
| clonotype73529 | TRA: CVVSSGSGSARQLTF; (SEQ ID NO: 457) | CV | 6 |
| | TRB: CASSLIGQGLRETQYF(SEQ ID NO: | | |
| | 458) | | |
| clonotype73530 | TRA: CAASRGNNRLAF; (SEQ ID NO: 459) | CV | 6 |
| | TRA: CAVSDGPGGYNKLIF; (SEQ ID NO: 460) | | |
| | TRB: CASSGGHNTEAFF(SEQ ID NO: 461) | | |
| clonotype73531 | TRA: CAVPGFGNEKLTF; (SEQ ID NO: 462) | CV | 6 |
| | TRB: CAISGGERGSYEQYF(SEQ ID NO: | | |
| | 463) | | |
| clonotype73533 | TRA: CAVGPGGYQKVTF; (SEQ ID NO: 464) | CV | 6 |
| | TRB: CASSLARRDREQFF(SEQ ID NO: 465) | | |
| clonotype73534 | TRA: CVVALLSGGFKTIF; (SEQ ID NO: 466) | CV | 6 |
| | TRB: CASSLWDSSYGYTF(SEQ ID NO: 467) | | |
| clonotype73535 | TRA: CAVDKVGSEKLVF; (SEQ ID NO: 468) | CV | 6 |
| | TRB: CSAGGGINEKLFF(SEQ ID NO: 469) | | |
| clonotype23651 | TRA: CAGPGNDMRF; (SEQ ID NO: 470) | CV | 310 |
| | TRB: CASSYSRSSGTNTEAFF(SEQ ID NO: | | |
| | 471) | | |
| clonotype57865 | TRA: CAVGRDKLIF; (SEQ ID NO: 472) | CV | 8 |
| | TRB: CAISENGGGGQGTEAFF(SEQ ID NO: | | |
| | 473) | | |
| clonotype58062 | TRA: CAVSDRGSTLGRLYF; (SEQ ID NO: 474) | CV | 6 |
| | TRB: CATSREEVLLRNQPQHF(SEQ ID NO: | | |
| | 475) | | |
| clonotype62630 | TRA: CALSGGVSNFGNEKLTF; (SEQ ID NO: 476) | CV | 6 |
| | TRA: CAVLEGRDKIIF; (SEQ ID NO: 477) | | |
| | TRB: CATAPGAGVGGYTF(SEQ ID NO: 478) | | |
| clonotype55171 | TRA: CAVPSISSGSARQLTF; (SEQ ID NO: 479) | CV | 3 |
| | TRB: CASRPSDRYNEQFF(SEQ ID NO: 480) | | |
| clonotype57875 | TRA: CAGDGSSNTGKLIF; (SEQ ID NO: 481) | CV | 3 |
| | TRB: CASSGTSRRQFF(SEQ ID NO: 482) | | |
| clonotype57878 | TRA: CAFREYGNKLVF; (SEQ ID NO: 483) | CV | 5 |
| | TRB: CASSTGTLFTGELFF(SEQ ID NO: 484) | | |
| clonotype57884 | TRA: CAVFNTDKLIF; (SEQ ID NO: 485) | CV | 5 |
| | TRB: CAWTGAGTYNEQFF(SEQ ID NO: | | |
| | 486) | | |
| clonotype57893 | TRA: CAARGFGAGNKLTF; (SEQ ID NO: 487) | CV | 5 |
| | TRA: CAGTSGTYKYIF; (SEQ ID NO: 488) | | |
| | TRB: CASSSGQSYEQYF(SEQ ID NO: 489) | | |
| clonotype57900 | TRA: CAVSVSGGGADGLTF; (SEQ ID NO: 490) | CV | 5 |
| | TRB: CASSLDRVGTEAFF(SEQ ID NO: 491) | | |
| clonotype57905 | TRA: CAMSGGYNKLIF; (SEQ ID NO: 492) | CV | 5 |
| | TRA: CVVSRSGGYQKVTF; (SEQ ID NO: | | |
| | 493) | | |
| | TRB: CSVAGLSGTDTQYF(SEQ ID NO: 494) | | |
| clonotype57906 | TRA: CALKALGSYIPTF; (SEQ ID NO: 495) | CV | 5 |
| | TRB: CASSPDSGANVLTF(SEQ ID NO: 496) | | |
| clonotype57907 | TRA: CALSAIGSGGSNYKLTF; (SEQ ID NO: 497) | CV | 5 |
| | TRB: CASSQGPVGTGGTDTQYF(SEQ ID | | |
| | NO: 498) | | |
| clonotype57917 | TRA: CALEVGSNTGKLIF; (SEQ ID NO: 499) | CV | 5 |
| | TRB: CASSYSATGVVYTGELFF(SEQ ID | | |
| | NO: 500) | | |
| clonotype57920 | TRA: CLVGGPDSGAGSYQLTF; (SEQ ID NO: 501) | CV | 5 |
| | TRB: CASSGRRVDTEAFF(SEQ ID NO: 502) | | |
| clonotype57923 | TRA: CAASIFGNEKLTF(SEQ ID NO: 503) | CV | 3 |
| clonotype57926 | TRA: CAVEVVSGGSYIPTF; (SEQ ID NO: 504) | CV | 5 |
| | TRB: CASSFGSGRVHEQFF(SEQ ID NO: | | |
| | 505) | | |
| clonotype57929 | TRA: CAVSSYLTDKLIF; (SEQ ID NO: 506) | CV | in |
| | TRB: CATSDQTGVRTF(SEQ ID NO: 507) | | |
| clonotype57935 | TRA: CAASIFGNEKLTF; (SEQ ID NO: 508) | CV | 5 |
| | TRB: CASSRQVRYEQYF(SEQ ID NO: 509) | | |
| clonotype57942 | TRA: CAMREGYQGAQKLVF; (SEQ ID NO: 510) | CV | 5 |
| | TRB: CASSFSSRQALMDEQFF(SEQ ID NO: | | |
| | 511) | | |
| clonotype57954 | TRA: CAYRSDNQGGKLIF(SEQ ID NO: 512); | CV | 5 |
| | TRB: CAISDRDRGRGFF(SEQ ID NO: 513) | | |
| clonotype57962 | TRA: CAPWGESSYKLIF; (SEQ ID NO: 514) | CV | 5 |
| | TRB: CAWSASWETQYF(SEQ ID NO: 515) | | |
| clonotype57973 | TRA: CAASGAGSYQLTF; (SEQ ID NO: 516) | CV | 5 |
| | TRB: CSARDRNSNEQFF(SEQ ID NO: 517) | | |
| clonotype57994 | TRA: CAVEQGGSEKLVF; (SEQ ID NO: 518) | CV | 5 |
| | TRB: CASSRDLFYSGANVLTF(SEQ ID NO: | | |
| | 519) | | |
| clonotype57999 | TRA: CAMREGLDNQGGKLIF; (SEQ ID NO: | CV | 5 |
| | 520) | | |
| | TRB: CSARESNRAAVGYTF(SEQ ID NO: | | |
| | 521) | | |
| clonotype58020 | TRA: CATVPYGNNRLAF; (SEQ ID NO: 522) | CV | 5 |
| | TRB: CASRSSNQPQHF(SEQ ID NO: 523) | | |
| clonotype58024 | TRA: CAASTGGGSNYKLTF; (SEQ ID NO: 524) | CV | 5 |
| | TRB: CASSLGSPLHF(SEQ ID NO: 525) | | |
| clonotype58027 | TRA: CAAAYSGGGADGLTF; (SEQ ID NO: 526) | CV | 5 |
| | TRB: CASSLDSTDTQYF(SEQ ID NO: 527) | | |
| clonotype58039 | TRA: CAVDTGNQFYF; (SEQ ID NO: 528) | CV | 3 |
| | TRB: CSARPAGRDEQYF(SEQ ID NO: 529) | | |
| clonotype58053 | TRA: CAFGLYAGGTSYGKLTF; (SEQ ID | CV | 5 |
| | NO: 530) | | |
| | TRB: CASSSRPGDEQYF(SEQ ID NO: 531) | | |
| clonotype58054 | TRA: CIVRFGSSNTGKLIF; (SEQ ID NO: 532) | CV | 3 |
| | TRB: CASSPGAPSGGETQYF(SEQ ID NO: | | |
| | 533) | | |
| clonotype58057 | TRA: CAGNSRDDKIIF; (SEQ ID NO: 534) | CV | 5 |
| | TRB: CSARKAGGYQPQHF(SEQ ID NO: 535) | | |
| clonotype58060 | TRA: CILISNFGNEKLTF; (SEQ ID NO: 536) | CV | 5 |
| | TRB: CASSQVMTHNTGELFF(SEQ ID NO: | | |
| | 537) | | |
| clonotype58071 | TRA: CATDGNNDMRF; (SEQ ID NO: 538) | CV | 5 |
| | TRB: CASSLGGVSLAQYF(SEQ ID NO: 539) | | |
| clonotype58115 | TRA: CAASPWGNARLMF; (SEQ ID NO: | CV | 5 |
| | 540) | | |
| | TRA: CAASREGNNARLMF; (SEQ ID NO: | | |
| | 541) | | |
| | TRB: CASSPFGENIQYF(SEQ ID NO: 542) | | |
| clonotype58169 | TRA: CAAAYARLMF; (SEQ ID NO: 543) | CV | 5 |
| | TRB: CASSPDGSSYNEQFF(SEQ ID NO: | | |
| | 544) | | |
| clonotype58218 | TRA: CVVRGGGYNKLIF; (SEQ ID NO: 545) | CV | 5 |
| | TRB: CASSPMAGSYNEQFF(SEQ ID NO: | | |
| | 546) | | |
| clonotype58251 | TRA: CALSGGDSSYKLIF; (SEQ ID NO: 547) | CV | 5 |
| | TRB: CASSFWFHEQYF(SEQ ID NO: 548) | | |
| clonotype58280 | TRB: CASSLPGGRSTDTQYF(SEQ ID NO: | CV | 3 |
| | 549) | | |
| clonotype58303 | TRA: CILNSGGGADGLTF; (SEQ ID NO: 550) | CV | 5 |
| | TRB: CASSKGQVLADTQYF(SEQ ID NO: | | |
| | 551) | | |
| clonotype58323 | TRA: CVVSDRSGGSYIPTF; (SEQ ID NO: | CV | 3 |
| | 552) | | |
| | TRB: CASSLGLAGAGELFF(SEQ ID NO: | | |
| | 553) | | |
| clonotype58349 | TRA: CTENRGSGGYQKVTF; (SEQ ID NO: 554) | CV | 5 |
| | TRB: CASSASQGLREKLFF(SEQ ID NO: | | |
| | 555) | | |
| clonotype58355 | TRA: CAFLERNTGKLIF; (SEQ ID NO: 556) | CV | 3 |
| | TRB: CASSLVTGAEQYF(SEQ ID NO: 557) | | |
| clonotype58377 | TRA: CVVNGGGTSYGKLTF; (SEQ ID NO: 558) | CV | 5 |
| | TRB: CATSRGQGRGTYEQYF(SEQ ID NO: | | |
| | 559) | | |
| clonotype58400 | TRA: CAATPNSGGSNYKLTF; (SEQ ID NO: | CV | 3 |
| | 560) | | |
| | TRA: CAFGGQGNLIF; (SEQ ID NO: 561) | | |
| | TRB: CASSLASTIAYEQYF(SEQ ID NO: 562) | | |
| clonotype58478 | TRA: CAVQELFSGGYNKLIF; (SEQ ID NO: 563) | CV | 5 |
| | TRB: CASSGPSGGAQETQYF(SEQ ID NO: | | |
| | 564) | | |
| clonotype58485 | TRA: CAGEPLGNTGKLIF; (SEQ ID NO: 565) | CV | 5 |
| | TRA: CVGGGTSYGKLTF; (SEQ ID NO: 566) | | |
| | TRB: CASSSPGKTSGDEQFF(SEQ ID NO: | | |
| | 567) | | |
| clonotype58487 | TRA: CGASAGGTSYGKLTF; (SEQ ID NO: 568) | CV | 5 |
| | TRB: CSARGKSGAFF(SEQ ID NO: 569) | | |
| clonotype58498 | TRA: CLYSGGYNKLIF; (SEQ ID NO: 570) | CV | 3 |
| | TRB: CASNWGRINSPLHF(SEQ ID NO: 571) | | |
| clonotype58847 | TRA: CAVPPYTGTASKLTF; (SEQ ID NO: 572) | CV | 5 |
| | TRB: CASSLGTGVGGSPLHF(SEQ ID NO: | | |
| | 573) | | |
| clonotype59208 | TRA: CVVNTGFQKLVF; (SEQ ID NO: 574) | CV | 3 |
| | TRB: CAISELQENTEAFF(SEQ ID NO: 575) | | |
| clonotype59374 | TRA: CAVQAGRNTDKLIF; (SEQ ID NO: | CV | 5 |
| | 576) | | |
| | TRB: CASSVGTYGGYTF(SEQ ID NO: 577) | | |
| clonotype60777 | TRA: CAGKGNQGGKLIF; (SEQ ID NO: 578) | CV | 3 |
| | TRB: CASSPQGHGYTF(SEQ ID NO: 579) | | |
| clonotype61418 | TRA: CAVISGYSTLTF(SEQ ID NO: 580) | CV | 3 |
| clonotype61484 | TRA: CAMRENTGGFKTIF; (SEQ ID NO: 581) | CV | 5 |
| | TRB: CSARDLHRGAGNQPQHF(SEQ ID NO: | | |
| | 582) | | |
| clonotype63292 | TRA: CVVSLNSGYSTLTF; (SEQ ID NO: 583) | CV | 3 |
| | TRB: CASSLPKNIQYF; (SEQ ID NO: 584) | | |
| | TRB: CASSSGGEQFF(SEQ ID NO: 585) | | |
| clonotype64366 | TRA: CAVEEGSNYQLIW; (SEQ ID NO: 586) | CV | 5 |
| | TRB: CASSEKGNYGYTF(SEQ ID NO: 587) | | |
| clonotype64660 | TRA: CAMSPKLGYALNF; (SEQ ID NO: 588) | CV | 3 |
| | TRB: CASSLGQGPSANEKLFF(SEQ ID NO: | | |
| | 589) | | |
| clonotype65111 | TRA: CARGVDTGNQFYF; (SEQ ID NO: 590) | CV | 3 |
| | TRA: CIVRAGSSNTGKLIF; (SEQ ID NO: | | |
| | 591) | | |
| | TRB: CASSYSRGRSPLHF(SEQ ID NO: 592) | | |
| clonotype65268 | TRA: CATDGWEGQNFVF; (SEQ ID NO: | CV | 3 |
| | 593) | | |
| | TRB: CASSLQGGTDTQYF(SEQ ID NO: 594) | | |
| clonotype65740 | TRA: CIVRPTGNQFYF; (SEQ ID NO: 595) | CV | 3 |
| | TRB: CASSNGGQDGYTF(SEQ ID NO: 596) | | |
| clonotype66085 | TRA: CAVSRRGFQKLVF; (SEQ ID NO: 597) | CV | 5 |
| | TRB: CAWVSDNTEAFF(SEQ ID NO: 598) | | |
| clonotype73532 | TRA: CAFMRNYGGATNKLIF; (SEQ ID NO: 599) | CV | 5 |
| | TRB: CAIRGGGTGSPLHF(SEQ ID NO: 600) | | |
| clonotype73536 | TRA: CATGPQGGSEKLVF; (SEQ ID NO: 601) | CV | 5 |
| | TRB: CSAAPGTGYQPQHF(SEQ ID NO: 602) | | |
| clonotype73538 | TRA: CALSEALTGGGNKLTF; (SEQ ID NO: 603) | CV | 3 |
| | TRB: CASSFGQASYEQYF(SEQ ID NO: 604) | | |
| clonotype73539 | TRA: CALPPRGSTLGRLYF; (SEQ ID NO: 605) | CV | 5 |
| | TRB: CASSMRRQPQHF(SEQ ID NO: 606) | | |
| clonotype73540 | TRA: CALSEGYSSASKIIF; (SEQ ID NO: 607) | CV | 5 |
| | TRB: CASRGVVGEQFF(SEQ ID NO: 608) | | |
| clonotype73541 | TRA: CAATGGSQGNLIF; (SEQ ID NO: 609) | CV | 5 |
| | TRB: CASSLAWGQSSYNEQFF(SEQ ID NO: | | |
| | 610) | | |
| clonotype73542 | TRA: CAVEDLGSGYSTLTF; (SEQ ID NO: 611) | CV | 3 |
| | TRB: CASSNTLGPGGYGYTF(SEQ ID NO: 612) | | |
| clonotype73544 | TRA: CAVMPGTSYGKLTF; (SEQ ID NO: 613) | CV | 5 |
| | TRB: CASGRTSGGAVTIEQFF(SEQ ID NO: | | |
| | 614) | | |
| clonotype73545 | TRA: CAGRRTGGGADGETF; (SEQ ID NO: | CV | 5 |
| | 615) | | |
| | TRB: CAITSGGSYNEQFF(SEQ ID NO: 616) | | |

The following Examples depict certain aspects and embodiments of the present disclosure.

### Example 1: CD4⁺ T Cell Responses in COVID-19 Illness

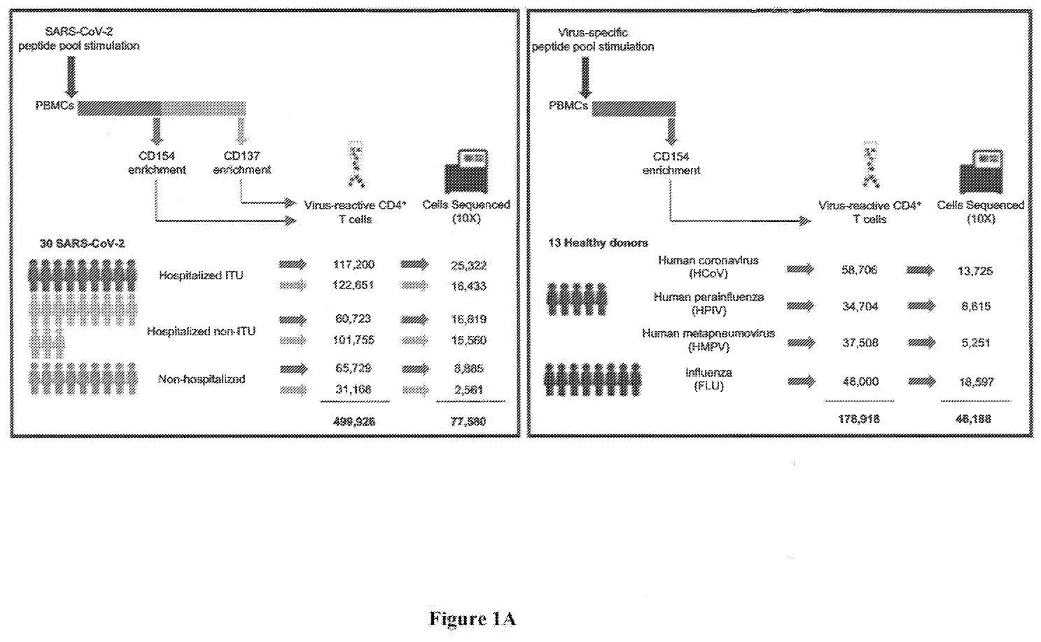

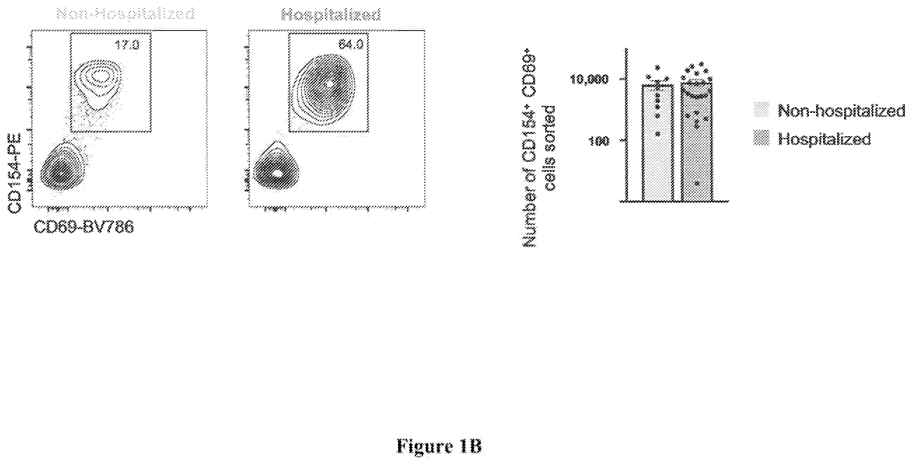

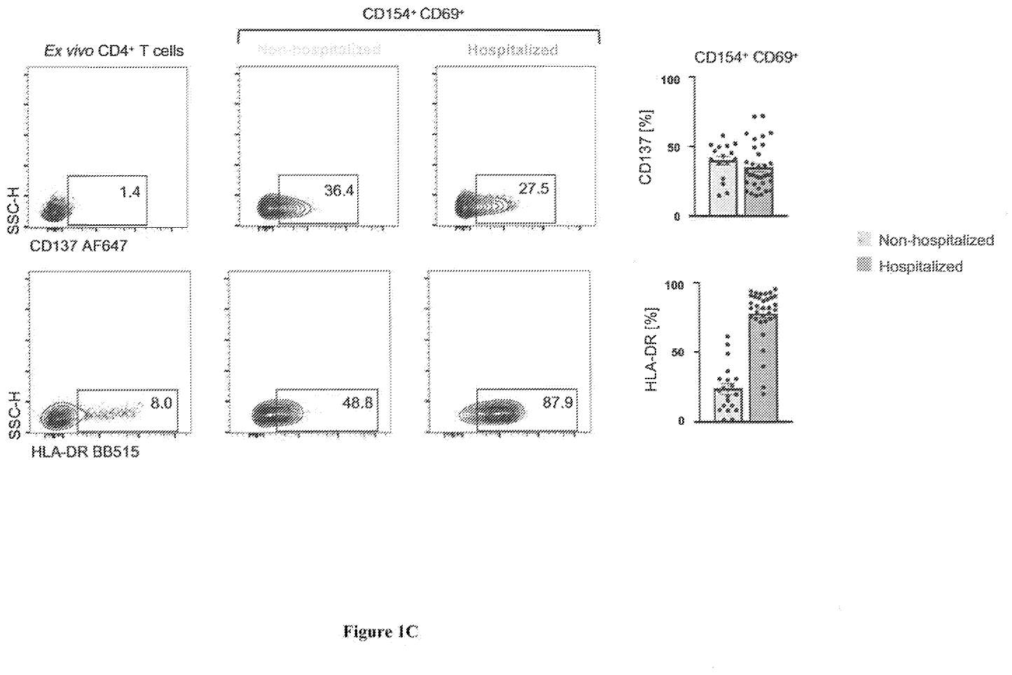

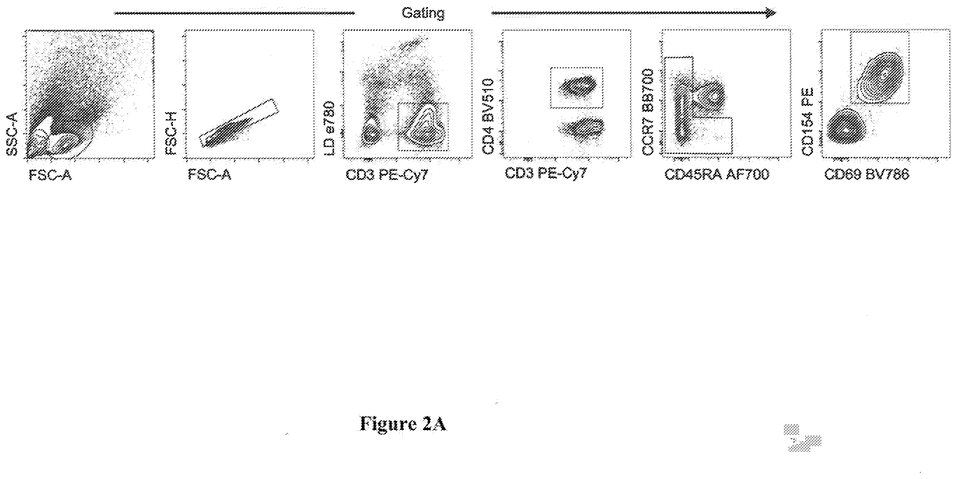

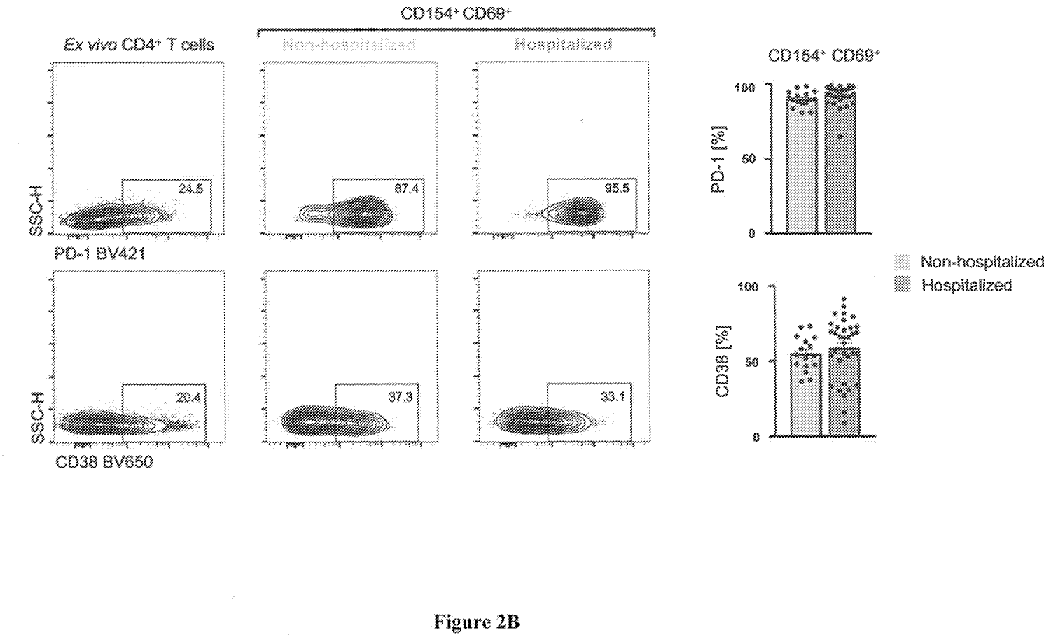

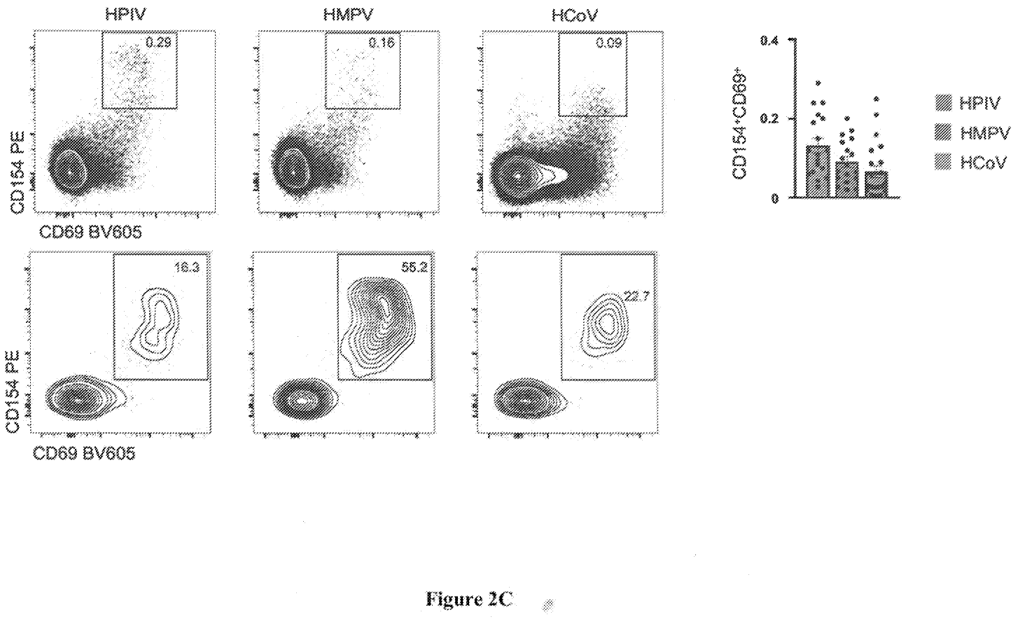

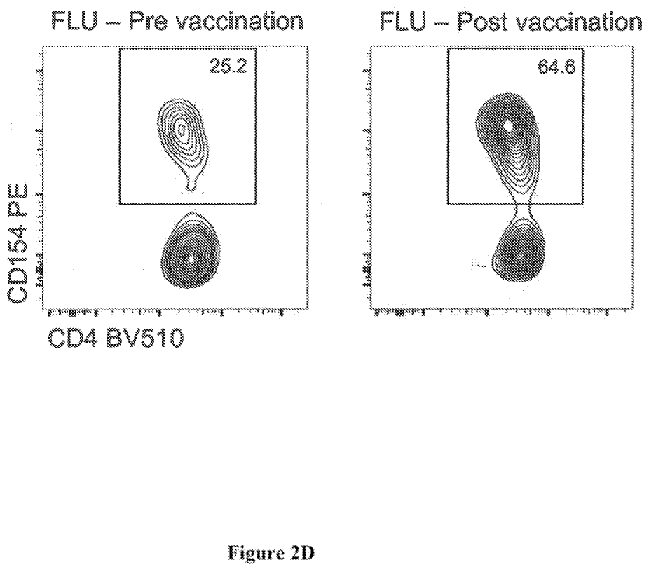

To capture CD4⁺ T cells responding to SARS-CoV-2 in patients with COVID-19 illness, the inventors employed the antigen-reactive T cell enrichment (ARTE) assay (Bacher et al., 2016; Bacher et al., 2019; Bacher et al., 2013) that relies on in vitro stimulation of peripheral blood mononuclear cells (PBMCs) for 6 hours with overlapping peptide pools targeting the immunogenic domains of the spike and membrane protein of SARS-CoV-2 (see Star Methods (Braun et al., 2020; Thieme et al., 2020)). Following in vitro stimulation, SARS-CoV-2-reactive CD4⁺ memory T cells were isolated based on the expression of cell surface markers (CD154 and CD69) that reflect recent engagement of the T cell receptor (TCR) by cognate MHC-peptide complexes (FIG. 2A). In the context of acute COVID-19 illness, CD4⁺ T cells expressing activation markers have been reported in the blood (Braun et al., 2020; Thevarajan et al., 2020); such CD4⁺ T cells, presumably activated in vivo by endogenous SARS-CoV-2 viral antigens, were also captured during the ARTE assay, thereby enabling us to study a comprehensive array of CD4⁺ T cell subsets responding to SARS-CoV-2. The inventors sorted >200,000 SARS-CoV-2-reactive CD4⁺ T cells from >1.3 billion PBMCs isolated from a total of 30 patients with COVID-19 illness (21 hospitalized patients with severe illness, 9 of whom required ICU treatment, and 9 non-hospitalized subjects with relatively milder disease, FIGS. 1A and 1B). In addition to expressing CD154 and CD69, sorted SARS-CoV-2-reactive CD4⁺ T cells co-expressed other activation-related cell surface markers like CD38, CD137 (4-1BB), CD279 (PD-1) and HLA-DR (FIGS. 1C and 2B).

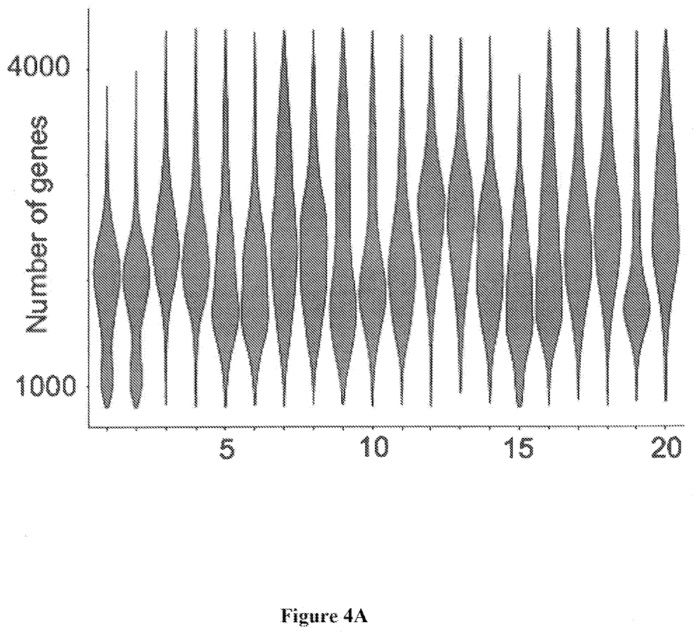

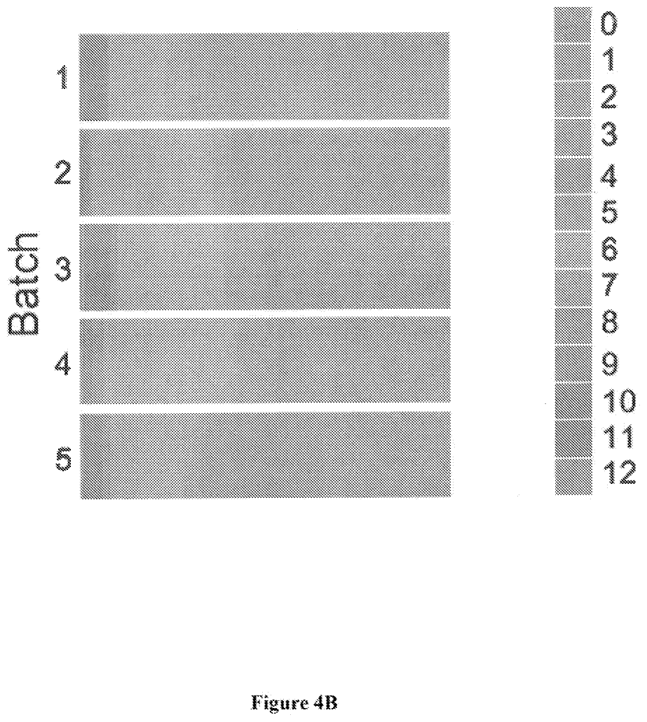

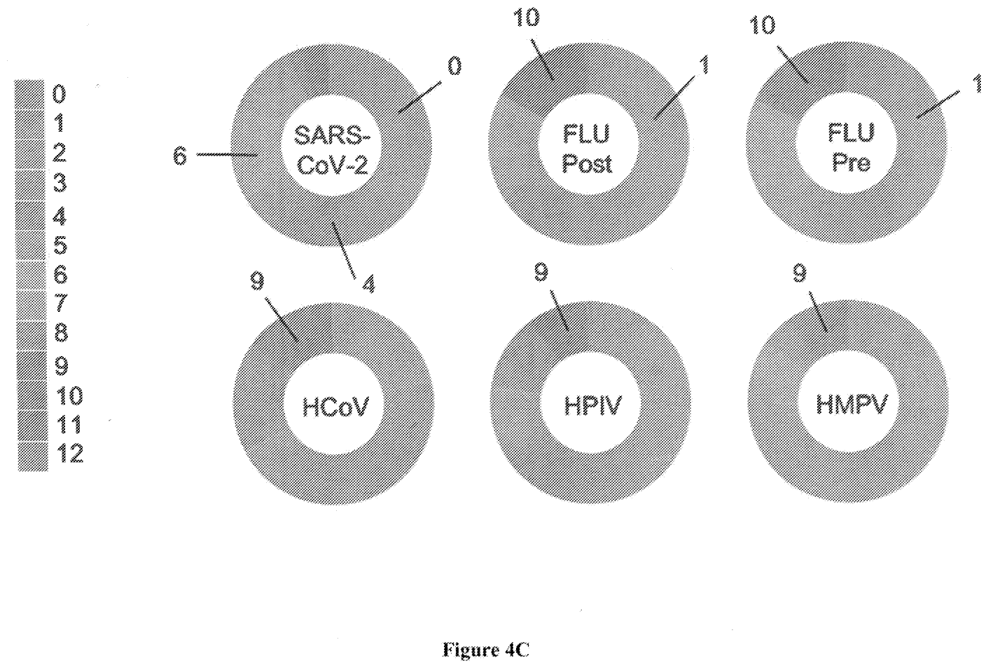

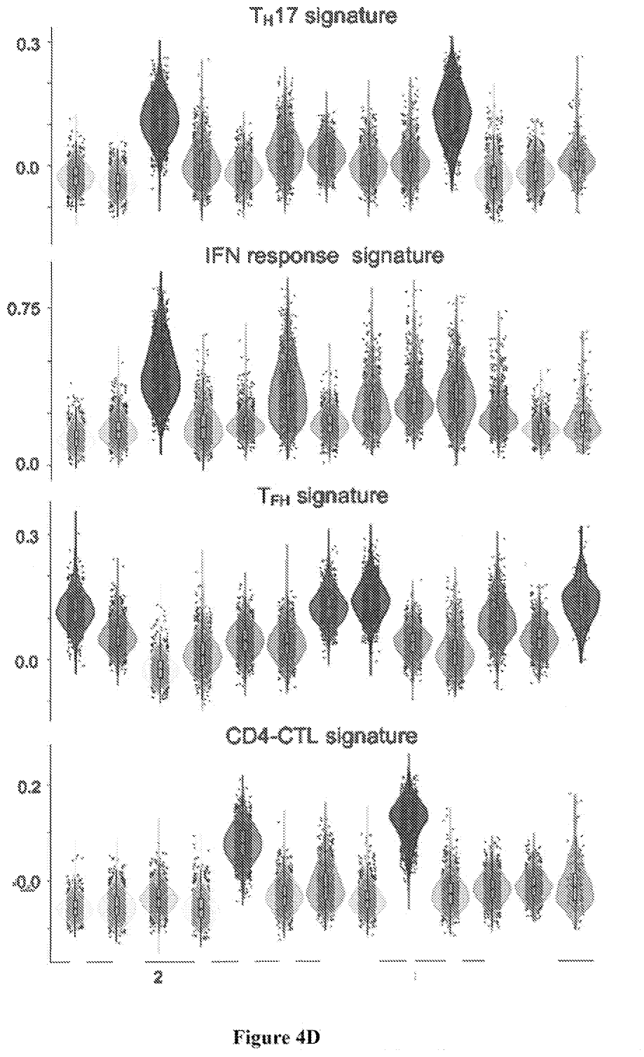

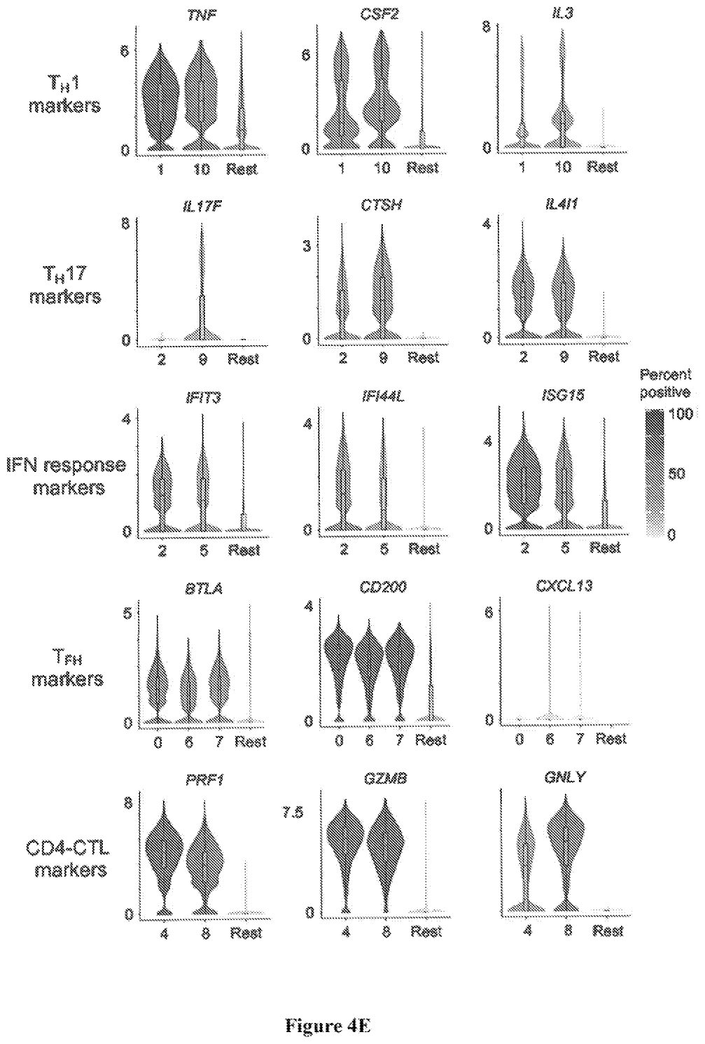

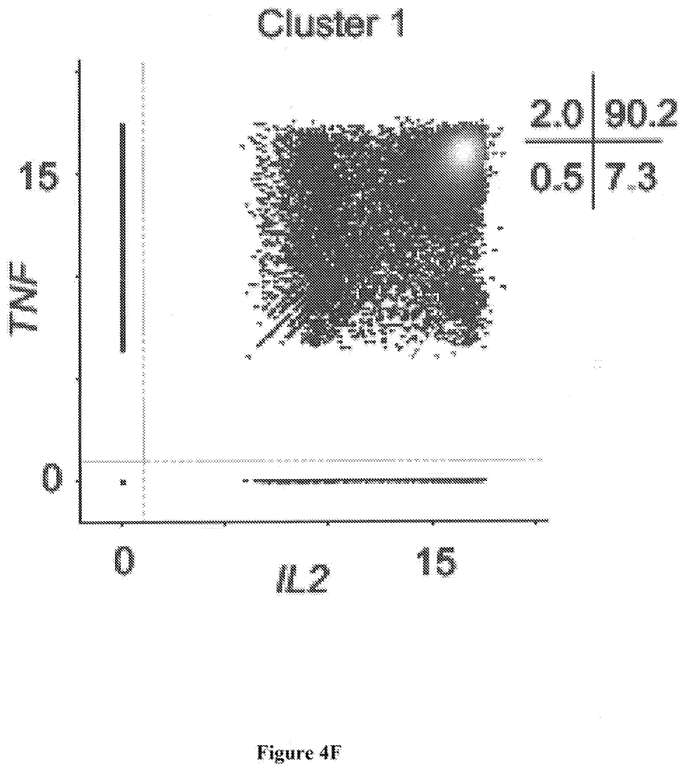

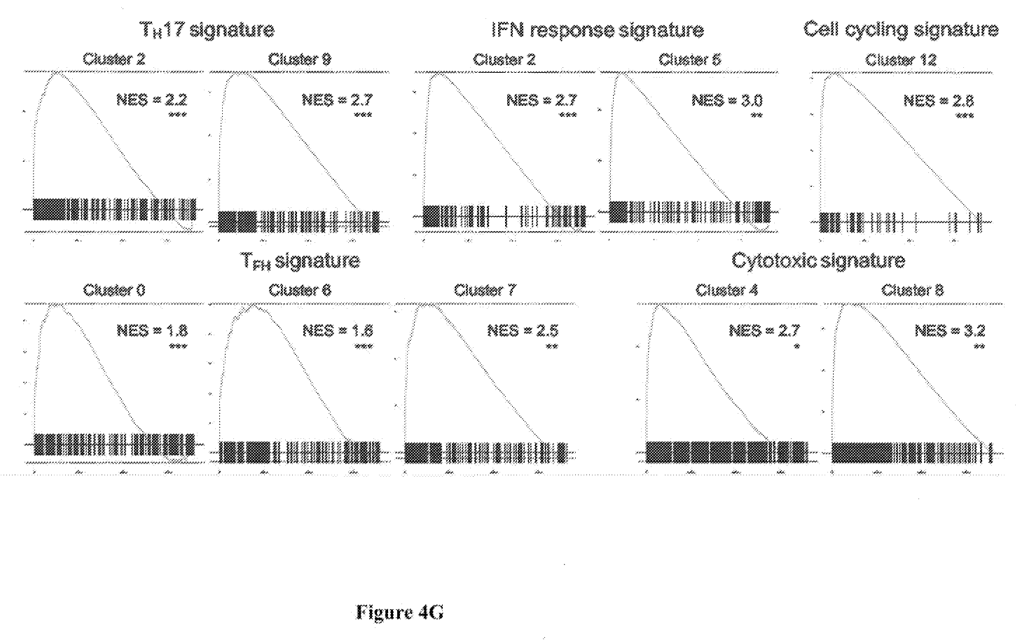

Recent evidence from studies in non-exposed individuals (blood sample obtained pre-COVID-19 pandemic) indicates that pre-existing human coronavirus (HCoV)-reactive CD4⁺ T cells can cross-react with SARS-CoV-2 antigens, and such cross-reactive cells are observed in up to 50% of the subjects studied (Braun et al., 2020; Grifoni et al., 2020). To capture such cross-reactive CD4⁺ T cells, likely to be human coronavirus (HCoV)-reactive, the inventors screened healthy non-exposed subjects and isolated CD4⁺ T cells responding to SARS-CoV-2 peptide pools from 4 subjects with highest responder frequency (FIGS. 1A and 2C). Next, for defining the CD4⁺ T cell subsets and their properties that distinguish SARS-CoV-2-reactive cells from other common respiratory virus-reactive CD4⁺ T cells, the inventors isolated CD4⁺ T cells responding to peptide pools specific to influenza (FLU) hemagglutinin protein (FLU-reactive cells, see Star Methods) from 8 additional healthy subjects who provided blood samples before and/or after influenza vaccination (FIGS. 1A and 2D). CD4⁺ T cells responding to peptide pools specific to other common respiratory viruses like human parainfluenza (HPIV) and human metapneumovirus (HMPV) were also isolated from healthy subjects (FIG. 2C). In total, the inventors interrogated the transcriptome and T cells receptor (TCR) sequence of >100,000 viral-reactive CD4⁺ T cells from 43 subjects (FIGS. 1A, 4A, and 4B).

### Example 2: SARS-CoV-2-Reactive CD4⁺ T Cells are Enriched for T_{FH} Cells and CD4-CTLs

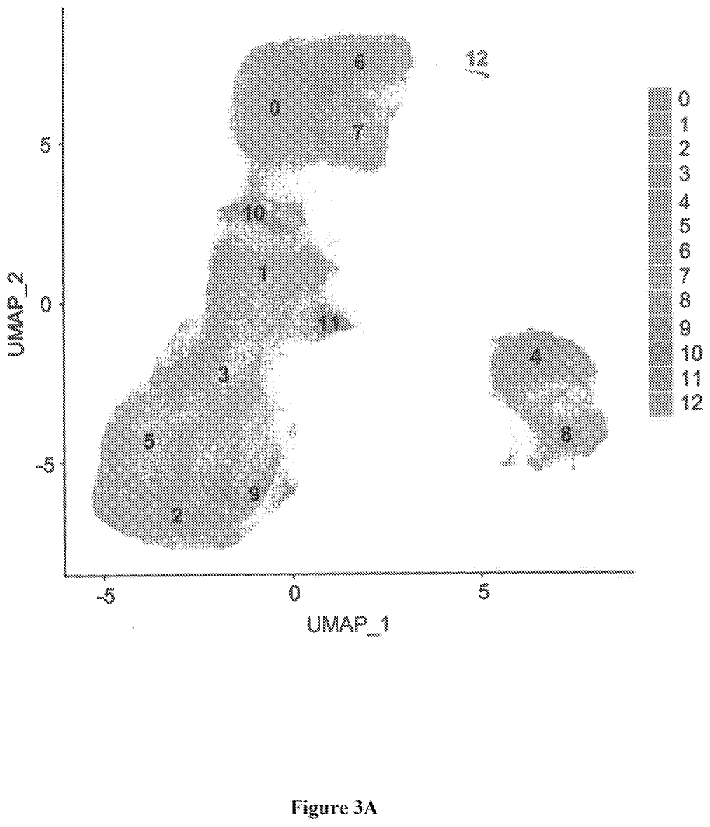

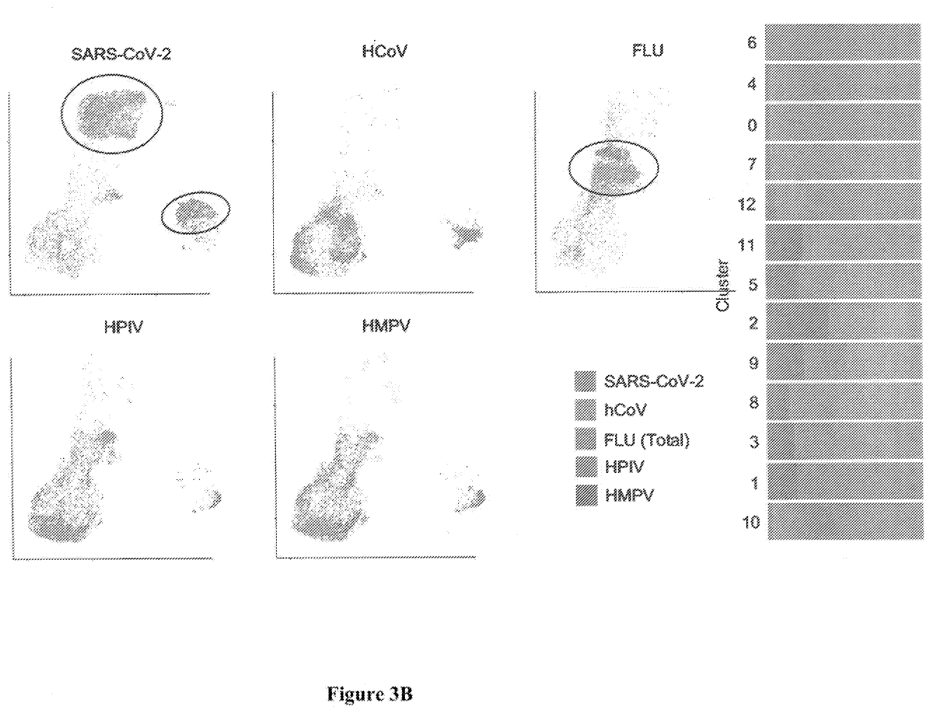

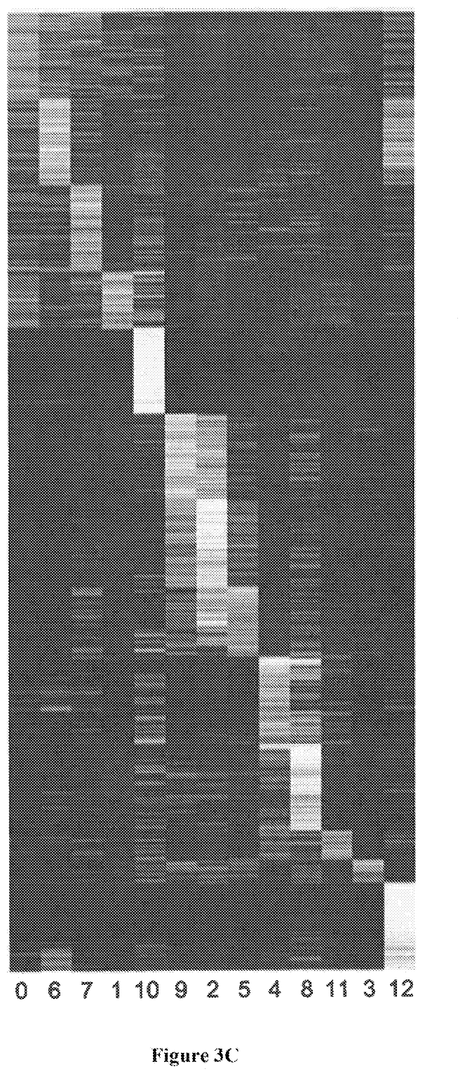

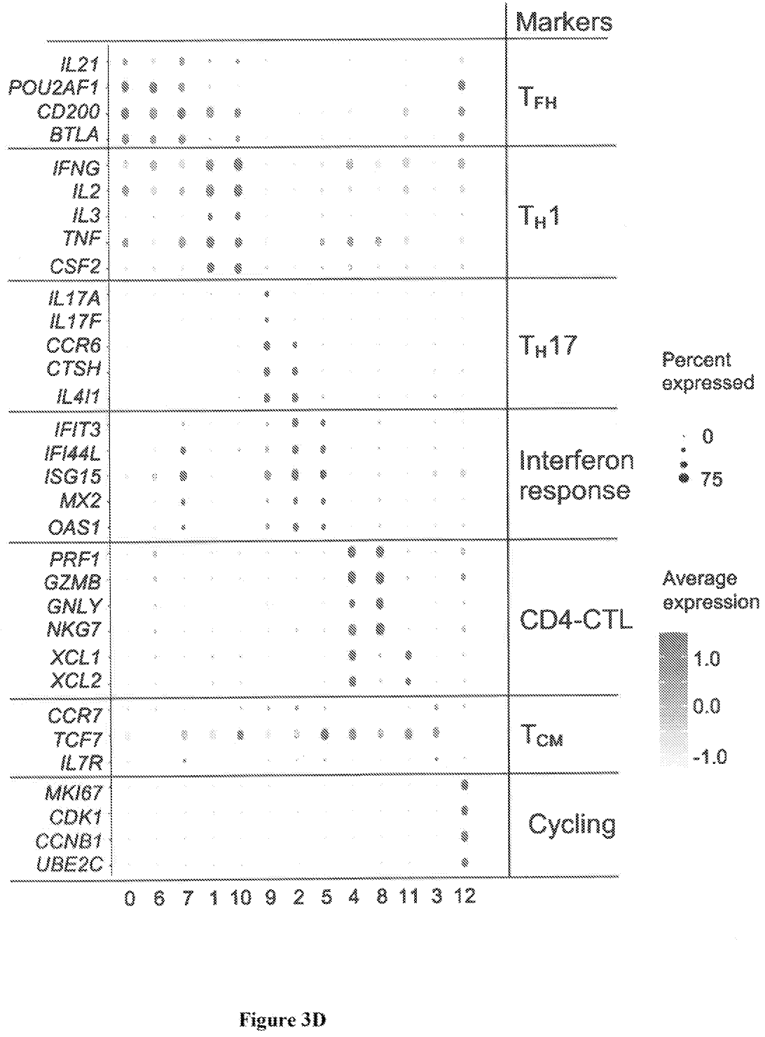

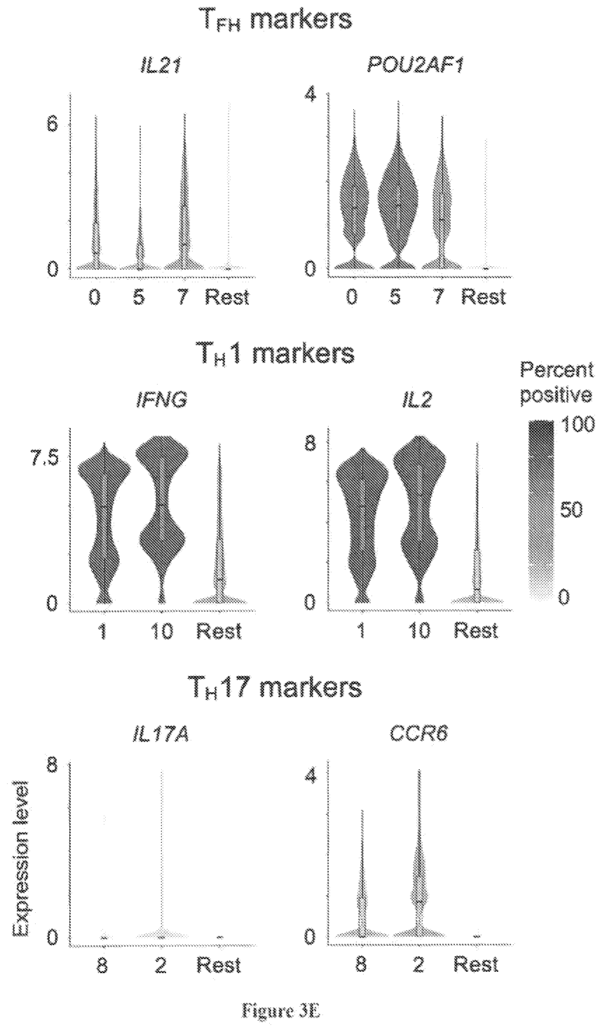

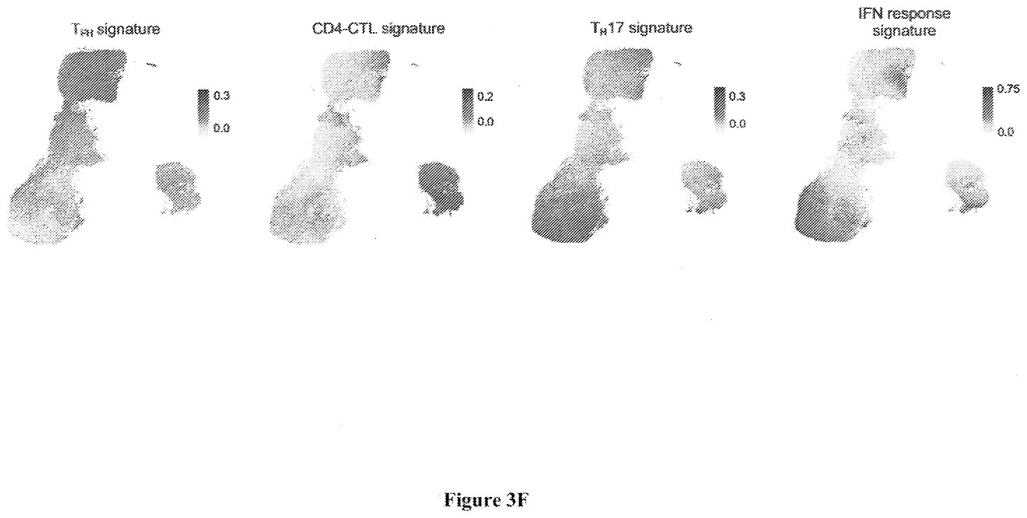

Analysis of the single-cell transcriptomes of all viral-reactive CD4⁺ T cells from all subjects revealed 13 CD4⁺ T cell subsets that clustered distinctly (each corresponding to the respective Tables 0-7), reflecting their unique transcriptional profiles (FIGS. 3A-D). Strikingly, a number of clusters were dominated by cells reactive to specific viruses (FIGS. 3B and 4C). For example, the vast majority of cells in clusters 1 and 10 were FLU-reactive (>75%), whereas cells in clusters 0,4,6,7 and 12 mainly consisted of SARS-CoV-2 reactive CD4⁺ T cells (>75%) from COVID-19 patients (FIGS. 3B and 4C). Conversely, cells in clusters (3, 5 and 11) were not preferentially enriched for any given virus (FIGS. 3B and 4C). These findings provide that distinct viral infections generate CD4⁺ T cell subsets with distinct transcriptional programs. This data highlights substantial heterogeneity in the nature of CD4⁺ T cells generated in response to different viral infections on the one hand and shared features on the other.

The clusters enriched for FLU-reactive CD4⁺ T cells (clusters 1 and 10) displayed features suggestive of polyfunctional T_{H}1 cells which have been associated with protective anti-viral immune responses (Seder et al., 2008). Such features include the expression of transcripts encoding for the canonical T_{H}1 transcription factor T-bet, cytokines linked to polyfunctionality, IFN-□□□IL-2 and TNF, and several other cytokines and chemokines like IL-3, CSF2, IL-23A and CCL20 (FIGS. 3D, 3E, 4E and 4F). SARS-CoV-2-reactive CD4⁺ T cells were under-represented in these clusters (cluster 1 and 10, <2%), when compared to FLU-reactive cells (>60%) or HMPV- and HPIV-reactive cells (˜15-20%) (FIG. 4C). Furthermore, SARS-CoV-2-reactive CD4⁺ T cells in cluster 1 expressed significantly lower levels of IFNG and IL2 transcripts when compared to FLU-reactive cells, which together suggested a failure to generate robust polyfunctional T_{H}1 cells in SARS-CoV-2 infection. A similar pattern was also observed in SARS-CoV2-peptide cross-reactive CD4⁺ T cells from healthy non-exposed subjects (FIGS. 3B and 4C) but not for HPIV- or HMPV-reactive CD4⁺ T cells, suggesting the defect in generating polyfunctional T_{H}1 cells may be a common feature for coronaviruses.

Other clusters that were relatively depleted of SARS-CoV-2-reactive CD4⁺ T cells included clusters 9 and 2, which were both enriched for T_{H}17 signature genes, with cluster 9 highly enriched for cells expressing IL17A and IL17F transcripts, thus representing bonafide T_{H}17 cells (FIGS. 3B-F and 4C-E). T_{H}17 cells have been associated with protective immune responses in certain models of viral infections (Acharya et al., 2017; Wang et al., 2011), however, in other contexts they have been shown to promote viral disease pathogenesis (Ma et al., 2019).

Clusters that were evenly distributed across all viral-specific CD4⁺ T cells include cluster 5 and 3. Cluster 5 displayed a transcriptional profile consistent with enrichment of interferon-response genes (IFIT3, IFI44L, ISG15, MX2, OAS1), and cluster 3 was enriched for CCR7, IL7R and TCF7 transcripts, likely representing central memory CD4⁺ T cell subset (FIGS. 3B-F and 4C-E).

Clusters 0, 6 and 7, which were colocalized in UMAP plots were dominated by SARS-CoV-2-reactive CD4⁺ T cells (FIG. 3B). Cells in these clusters were uniformly enriched for transcripts encoding for cytokines, surface markers and transcriptional coactivators associated with T follicular helper (T_{FH}) cell function (CXCL13, IL21, CD200, BTLA and POU2AF1) (Locci et al., 2013) (FIGS. 3B-F and 4C-E). Independent gene set enrichment analysis (GSEA) showed significant positive enrichment of T_{FH} Signature genes in these clusters, confirming that cells in these clusters represent circulating T_{FH} cells (FIG. 4G). Bonafide T_{FH} cell reside in the germinal center, however, T_{FH} cells have been described in the blood where increased numbers have been reported in viral infections and following vaccinations (Bentebibel et al., 2013; Koutsakos et al., 2018; Smits et al., 2020). Accordingly, the inventors found an increase in the proportions of cells in the T_{FH} clusters following flu-vaccination (FIG. 4C). The increase in circulating SARS-CoV-2-reactive T_{FH} subsets observed in patients with COVID-19 is therefore consistent with published reports in acute infections.

Cluster 12, which expressed high levels of transcripts linked to cell cycle genes MKI67 and CDK1, also contained a large proportion of SARS-CoV-2 reactive CD4⁺ T cells (FIGS. 3B-D), indicative of actively proliferating cells responsive to SARS-CoV-2 antigens. Cluster 4, also dominated by SARS-CoV-2-reactive CD4⁺ T cells, was characterized by high levels of PRF1, GZMB, GZMH, GNLY and NKG7 transcripts, which encode for molecules linked to cytotoxicity (Patil et al., 2018) (FIGS. 3B-F and 4C-E). GSEA analysis showed significant positive enrichment of cytotoxic signature genes in clusters 4 and 8 (FIG. 4G), confirming these clusters represent cytotoxic CD4⁺ T cells (CD4-CTLs). Overall, the single-cell transcriptomic analysis revealed substantial differences in the nature of CD4⁺ T cell responses to viral infections and highlight subsets that are specifically enriched or depleted in COVID-19 illness.

### Example 3: SARS-CoV-2-Reactive CD4⁺ T Cell Subsets Associated with Disease Severity

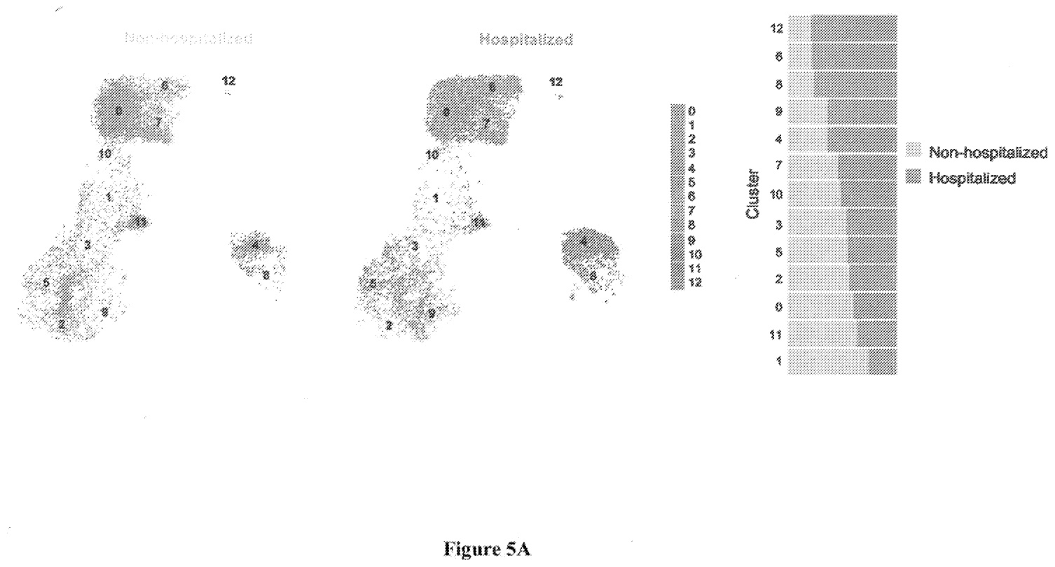

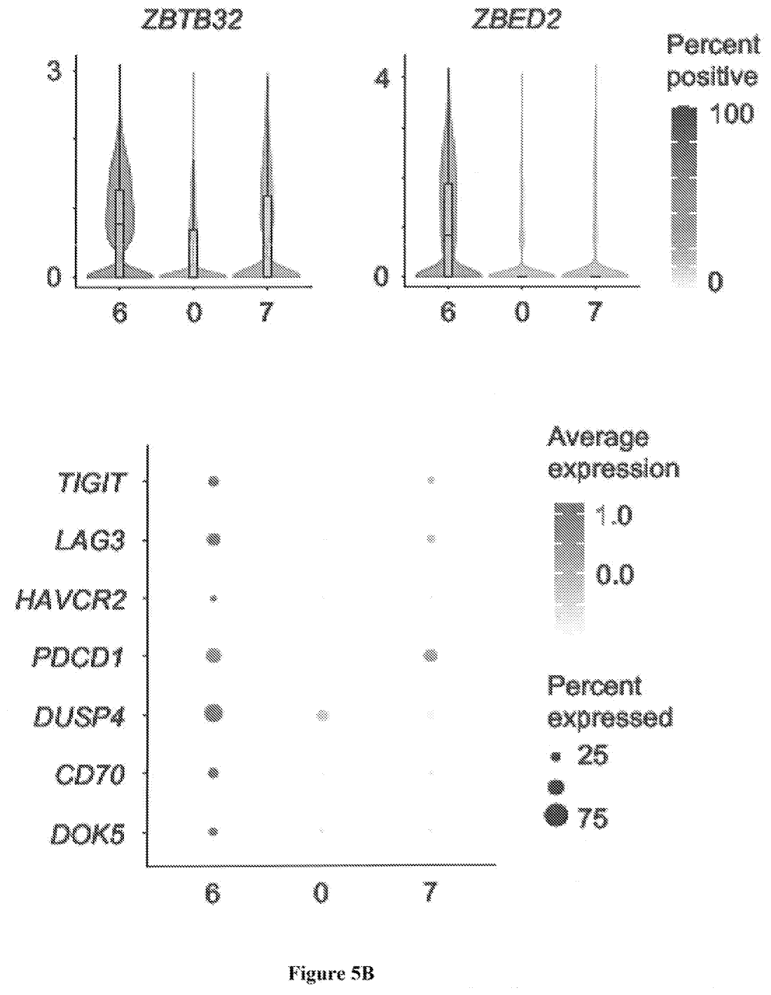

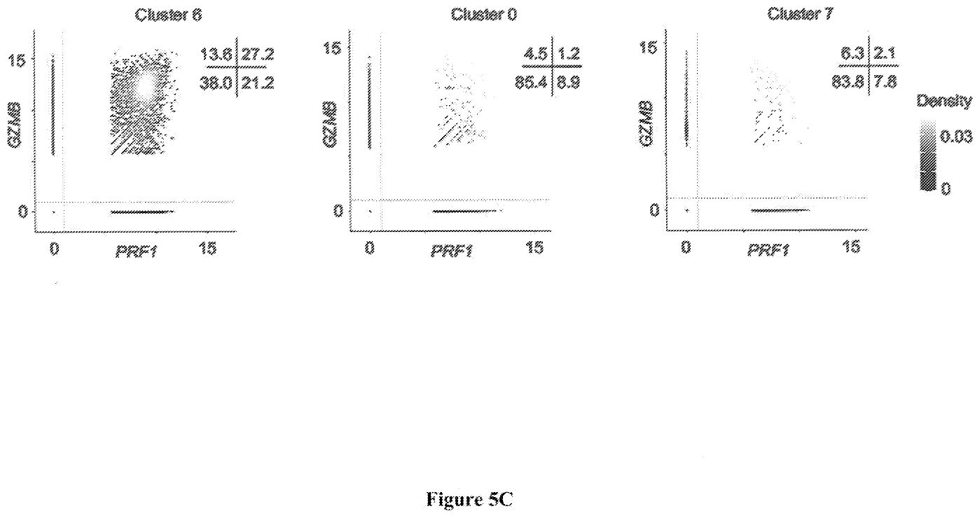

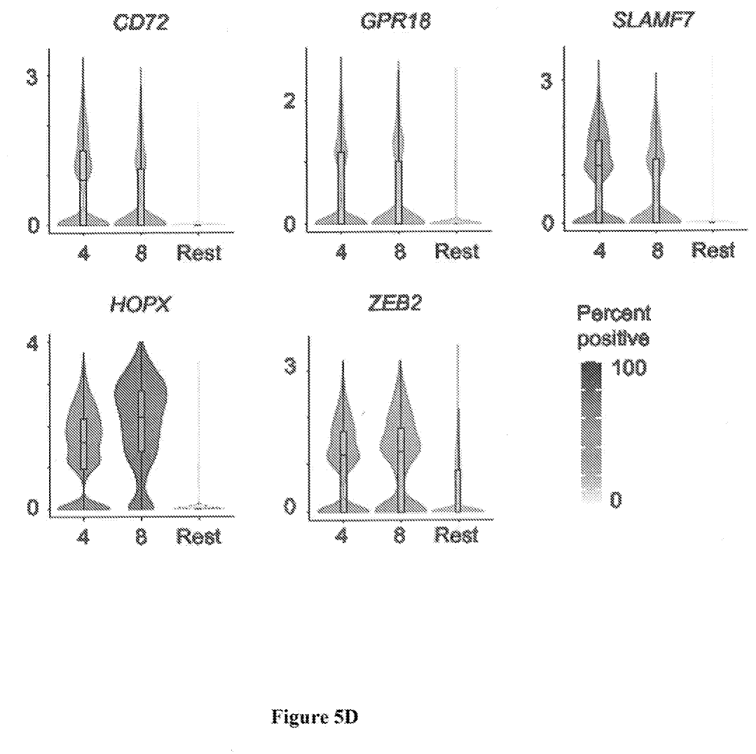

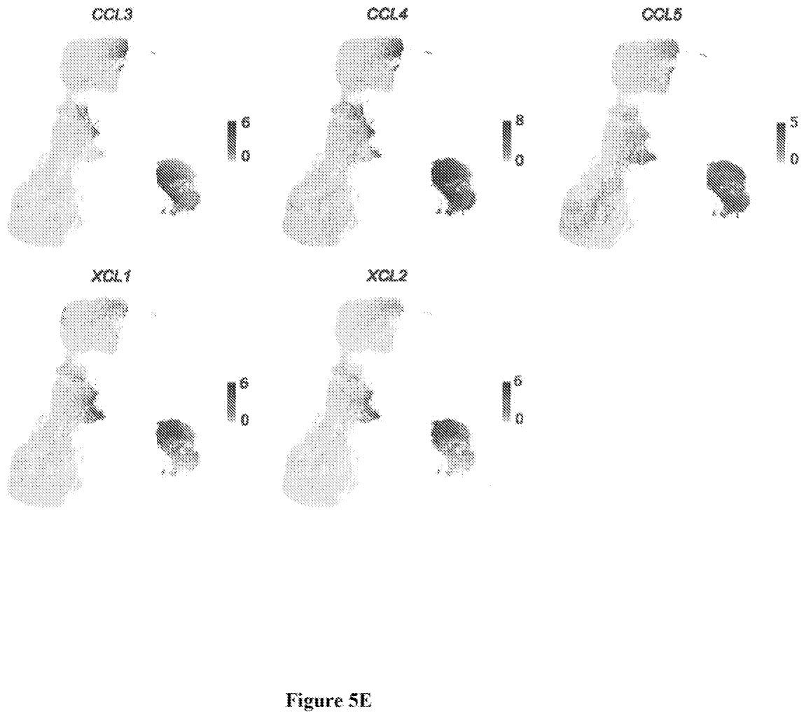

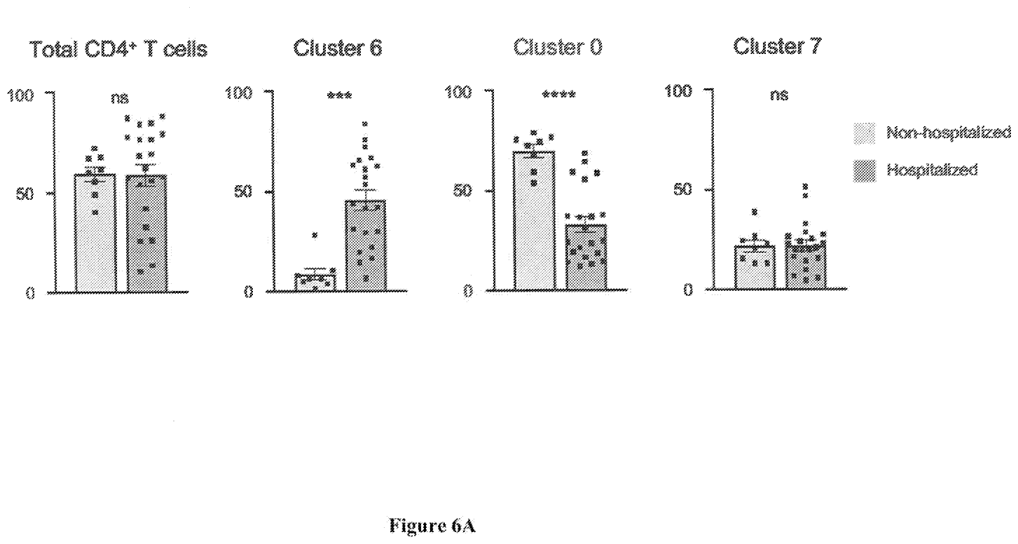

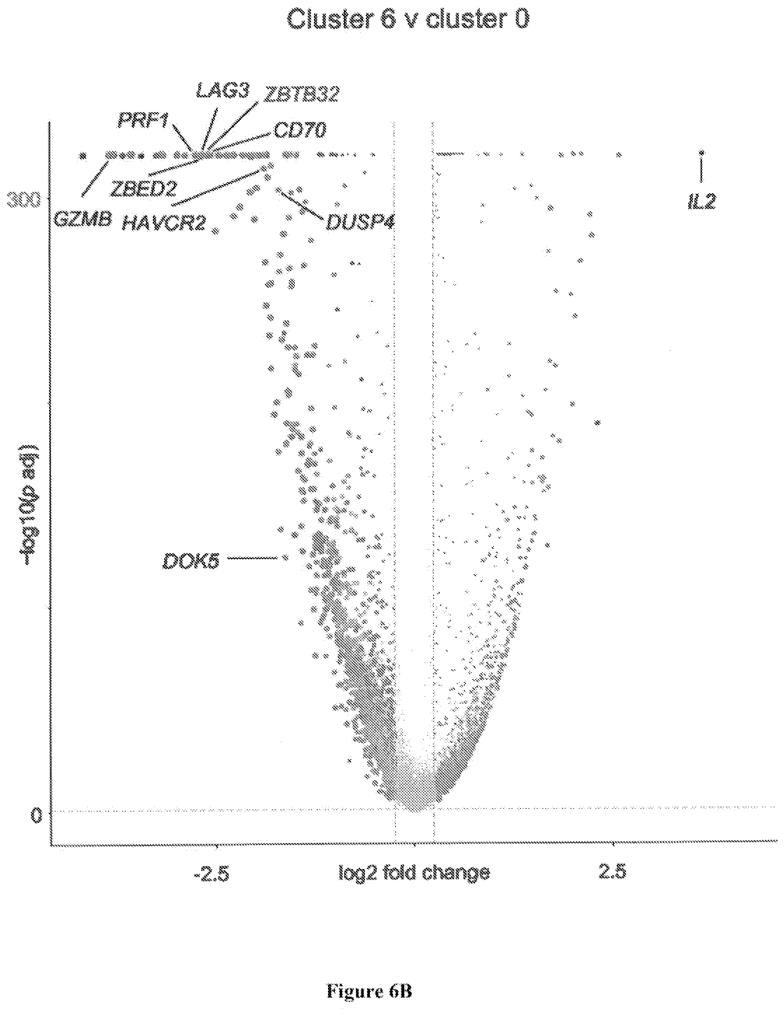

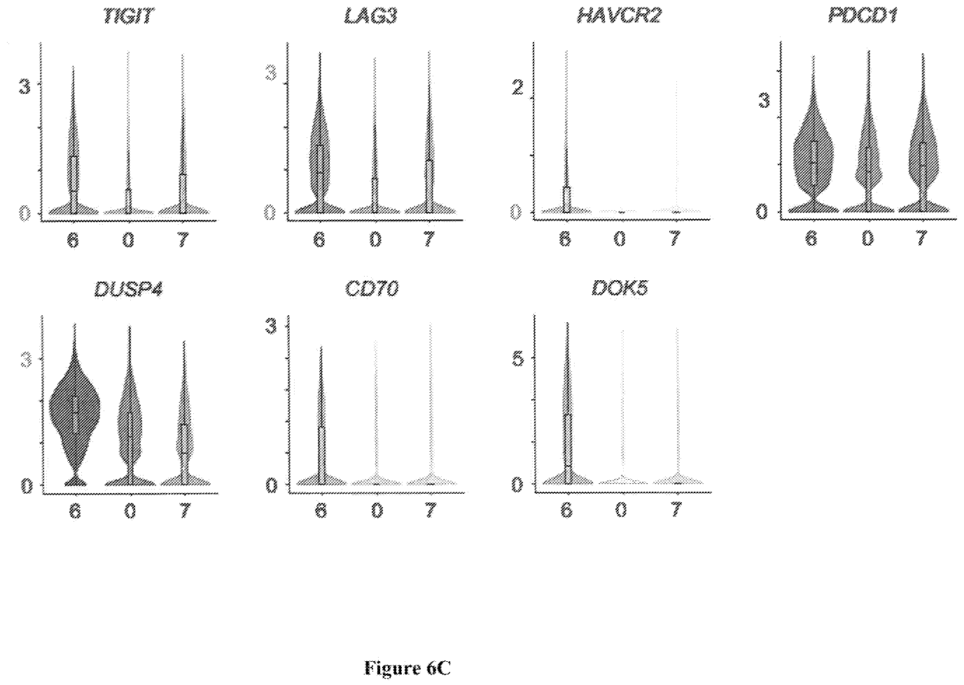

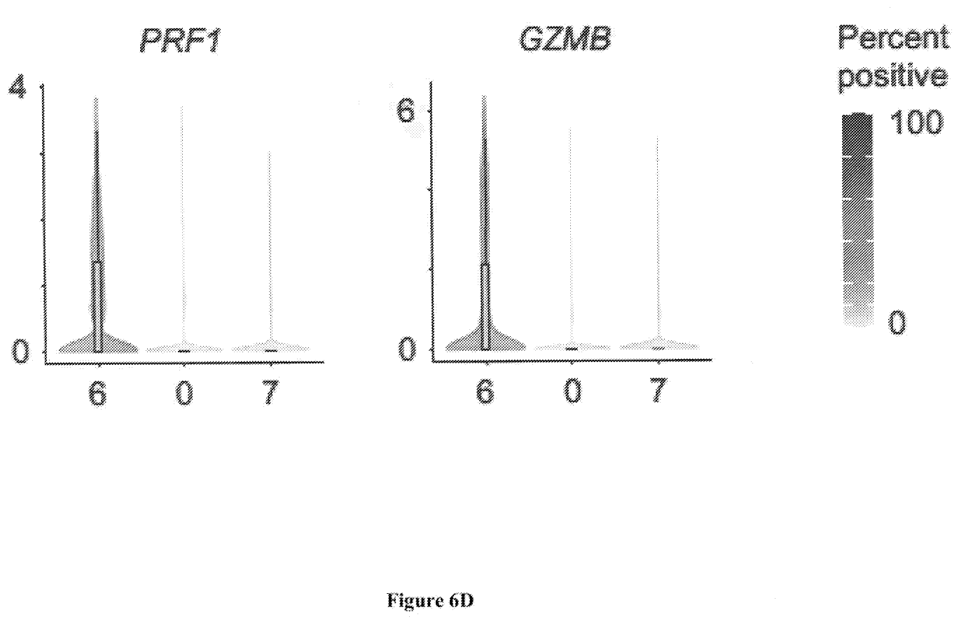

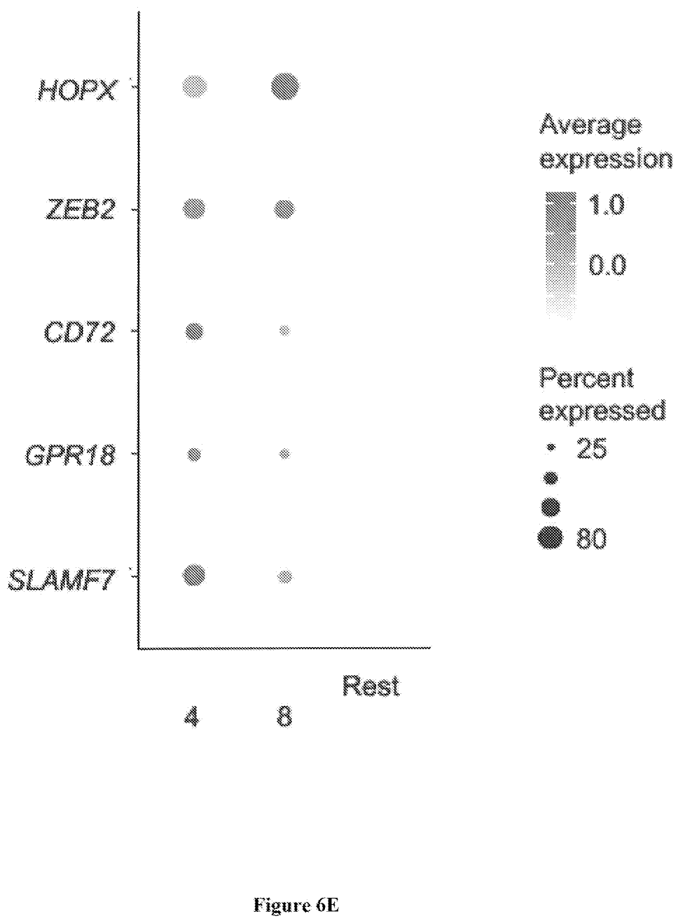

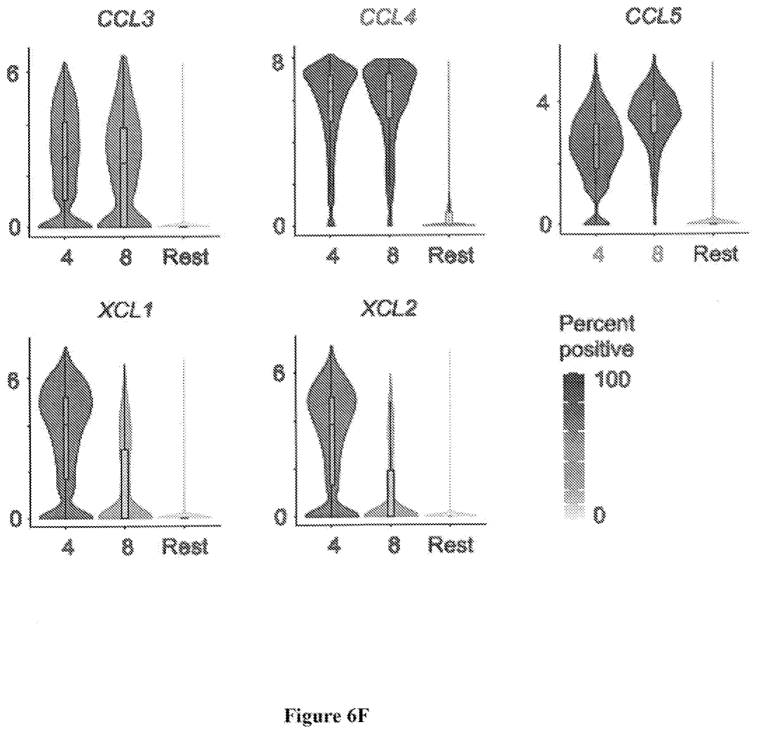

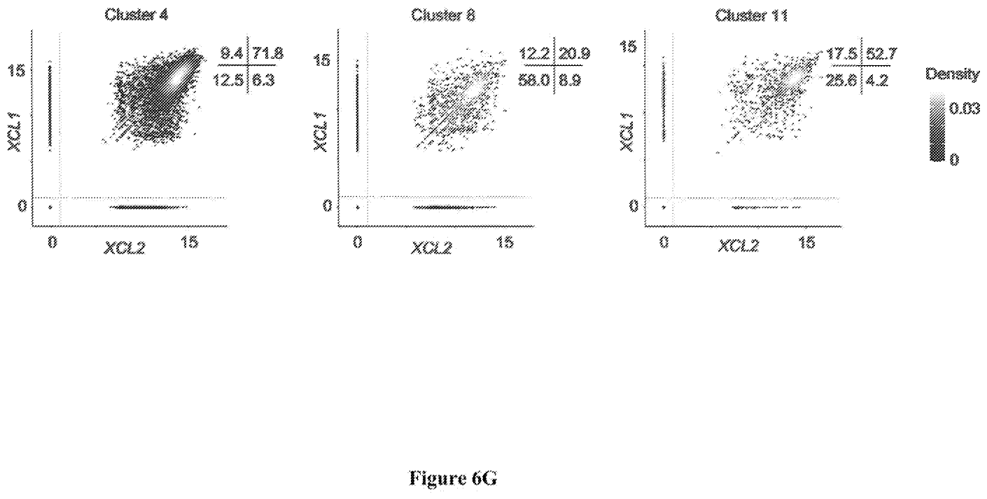

The inventors next assessed if the proportions of SARS-CoV-2 reactive CD4⁺ T cells in ay cluster were greater or lower in patients with severe COVID-19 (n=21, requiring hospitalization) when compared to those with milder disease (n=9, not needing hospitalization). Among the three T_{FH} clusters (clusters 0,6 and 7), which consisted almost exclusively of CD4⁺ T cells reactive to SARS-CoV-2, the relative proportion of cells in T_{FH} cluster 6 was greater in patients with severe disease compared to mild disease (FIGS. 5A and 6A). Transcripts encoding for transcription factors ZBED2 and ZBTB32 were enriched in the T_{FH} cluster 6 and were also expressed at significantly higher levels in patients with severe disease (FIGS. 5B and S3B). ZBTB32, also known as PLZP that belongs to a BTB-ZF family of transcriptional repressors like PLZF, BCL6 and ThPOK, has been shown to play a role in impairing anti-viral immune responses by negatively regulating T cell proliferation, cytokine production and development of long-term memory cells (Piazza et al., 2004; Shin et al., 2017). ZBED2, a novel zinc finger transcription factor without a mouse orthologue, has been linked to T cell dysfunction in the context of anti-tumor immune response (Li et al., 2019), and more recently shown to repress expression of interferon target genes (Somerville et al., 2020). In support of potential dysfunctional properties of the cells in the T_{FH} cluster 6, the inventors found increased expression of several transcripts linked to inhibitory function, like TIGIT, LAG3, TIM3 and PD1 (Thommen and Schumacher, 2018), and to negative regulation of T cell activation and proliferation, like DUSP4 and CD70 (Huang et al., 2012; O'Neill et al., 2017) (FIGS. 5B and 6C). Moreover, T_{FH} cells in cluster 6 also expressed high levels of cytotoxicity-associated transcripts (PRF1, GZMB) (FIGS. 5C and 6D), reminiscent of the recently described cytotoxic T_{FH} cells, which were shown to directly kill B cells and associated with the pathogenesis of recurrent tonsillitis in children (Dan et al., 2019). Together, these findings show that T_{FH} cells in cluster 6, which are increased in severe COVID-19 illness, displayed cytotoxicity features that may impair humoral (B cell) immune responses.

While T cells with cytotoxic function predominantly consist of conventional MHC class I-restricted CD8⁺ T cells, MHC class II-restricted CD4⁺ T cells with cytotoxic potential (CD4-CTLs) have been reported in several viral infections in humans and are associated with better clinical outcomes (Cheroutre and Husain, 2013; Weiskopf et al., 2015a). Paradoxically, in SARS-CoV-2 infection, the inventors find that cells in the CD4-CTL clusters (cluster 4 and 8) were present at higher frequencies in hospitalized patients with severe disease compared to those with milder disease, potentially contributing to disease severity, although the inventors observed substantial heterogeneity in responses among patients (FIG. 5A). Interrogation of the transcripts enriched in the CD4-CTL subsets pointed to several interesting molecules and transcription factors that are likely to play an important role in their maintenance and effector function. These include molecules like CD72 and GPR18 that are known to enhance T cell proliferation and maintenance of mucosal T cell subsets, respectively (Jiang et al., 2017; Wang et al., 2014) (FIGS. 5D and 6E). Additional examples include transcription factors HOPX and ZEB2 (FIGS. 5D and S3E) that have been shown to positively regulate effector differentiation, function, persistence and survival of T cells (Albrecht et al., 2010; Omilusik et al., 2015). Besides cytotoxicity-associated transcripts, the CD4-CTL subsets (cluster 4 and 8) were highly enriched for transcripts encoding for a number of chemokines like CCL3 (also known as macrophage inflammatory protein (MIP)-1α), CCL4 (MIP-1β) and CCL5 (FIGS. 5E and 6F); these chemokines play an important role in the recruitment of myeloid cells (neutrophils, monocytes, macrophages), NK cells and T cells expressing chemokine receptors CCR1, CCR3 and CCR5 (Hughes and Nibbs, 2018). The CD4-CTL subset in cluster 4 also expressed high levels of transcripts encoding for chemokines XCL1 and XCL2 (FIGS. 5E and 6G) that specifically recruit XCR1-expressing conventional type 1 dendritic cells (cDC1) to sites of immune responses where they play a key role in promoting the CD8⁺ T cell responses by antigen cross-presentation (Lei and Takahama, 2012). Overall, the transcriptomic features of SARS-CoV-2-reactive CD4-CTLs show that they are likely to be more persistent and play an important role in orchestrating immune responses by recruiting innate immune cells to enhance CD8⁺ T cell responses, while also directly mediating cytotoxic death of MHC class II-expressing virally-infected cells.

### Example 4: Massive Clonal Expansion of CD4-CTLs

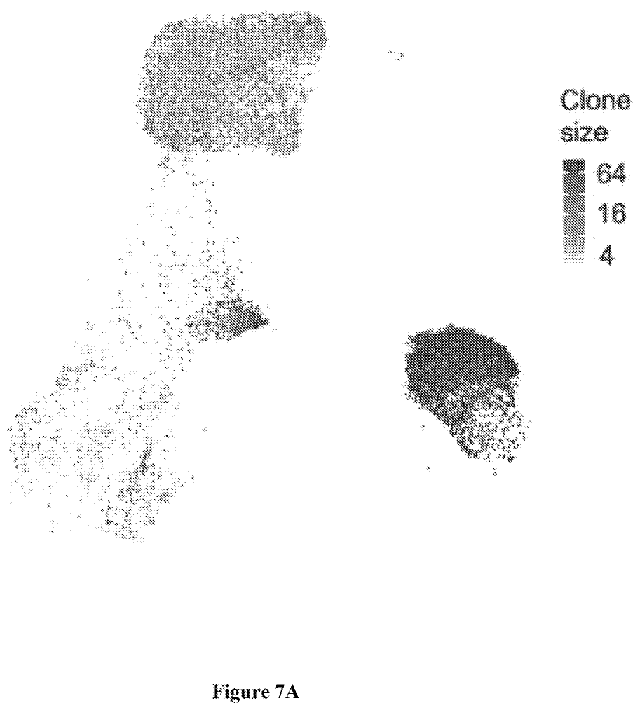

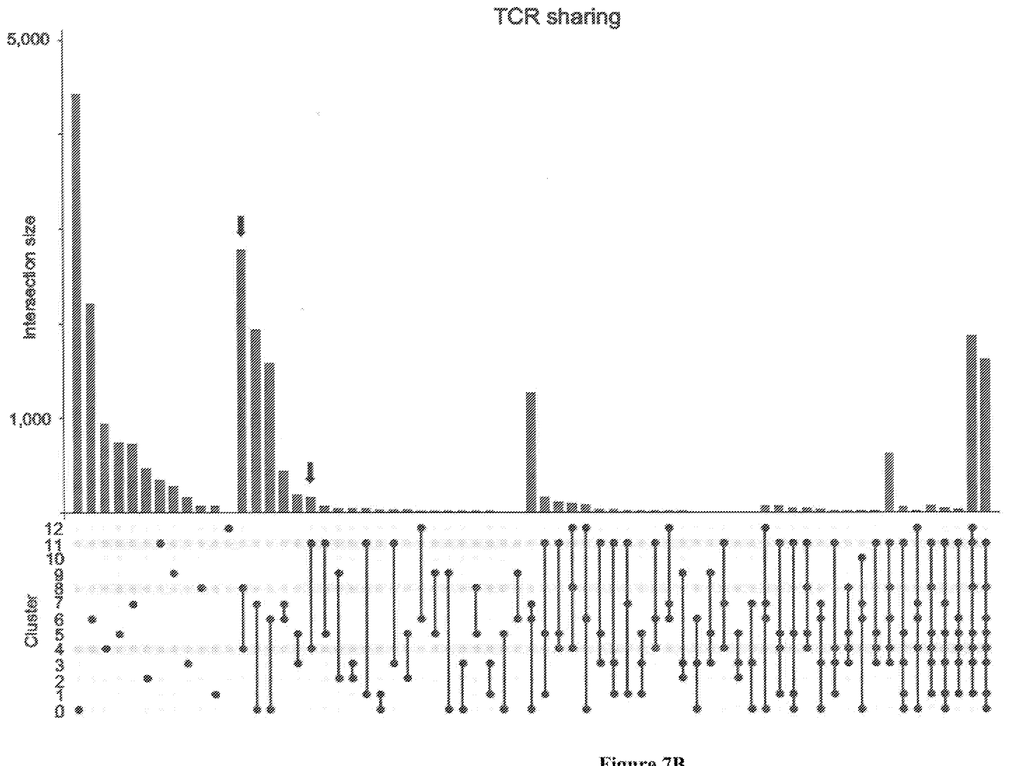

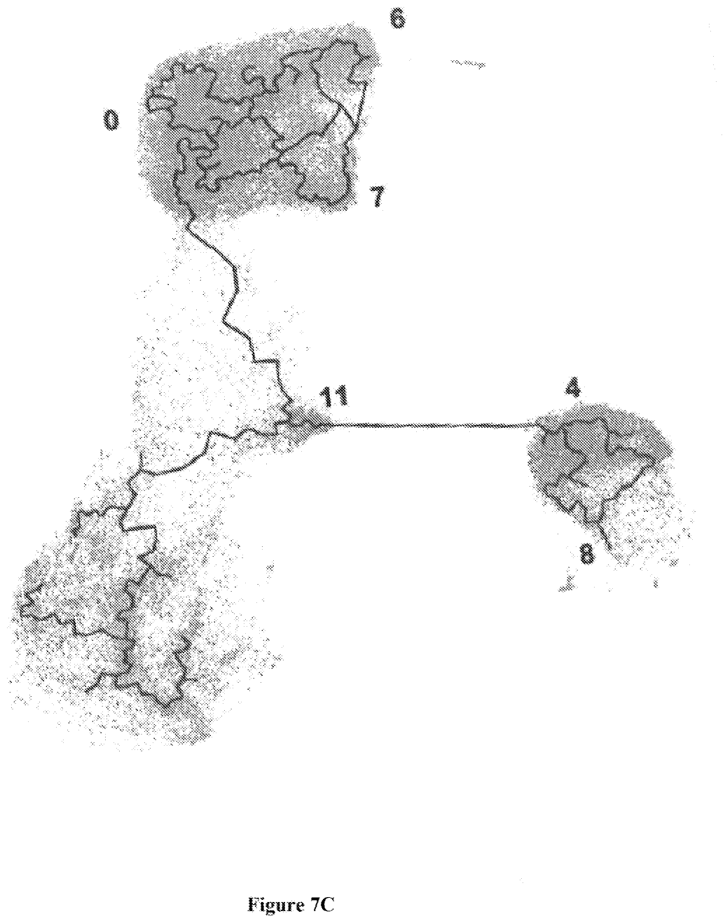

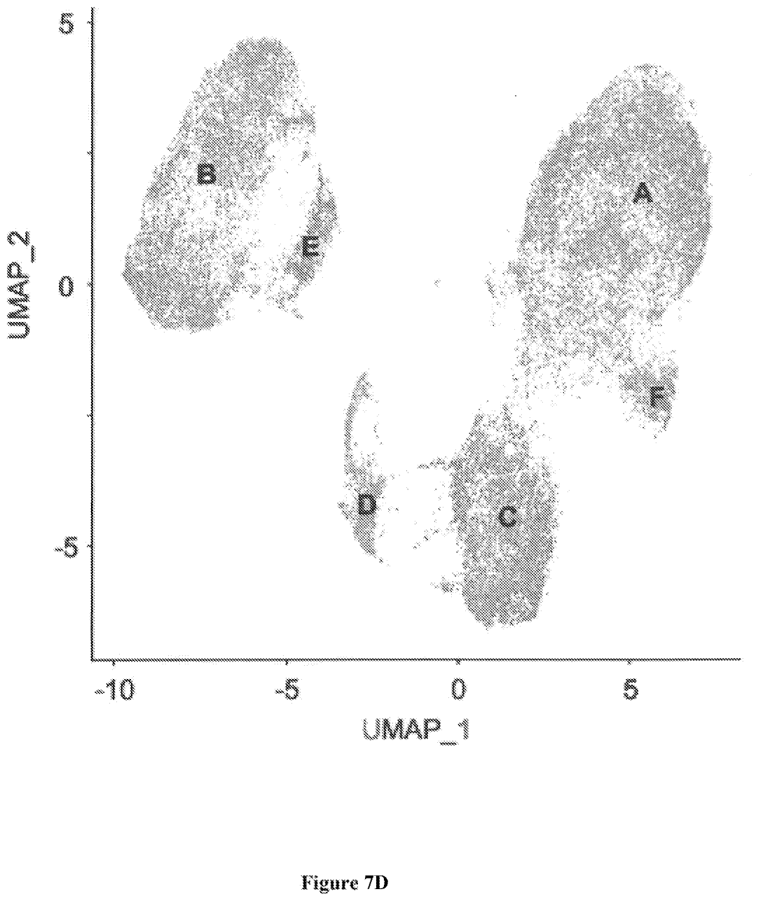

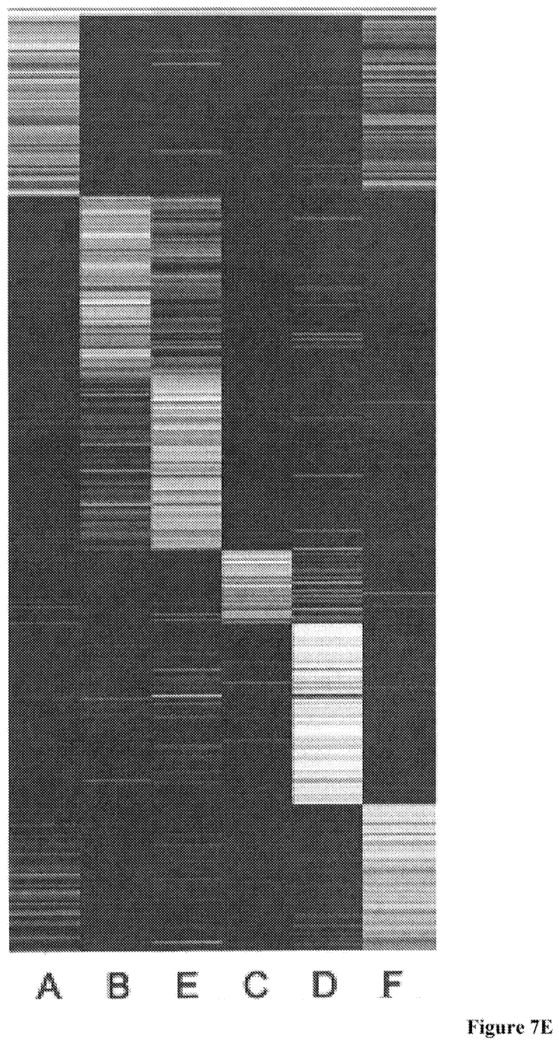

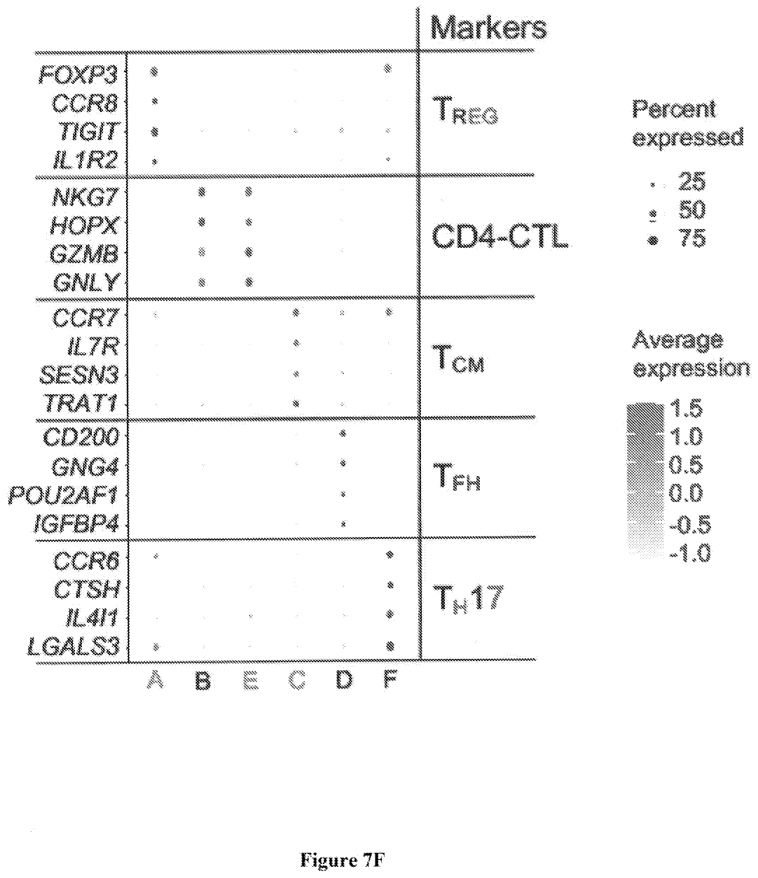

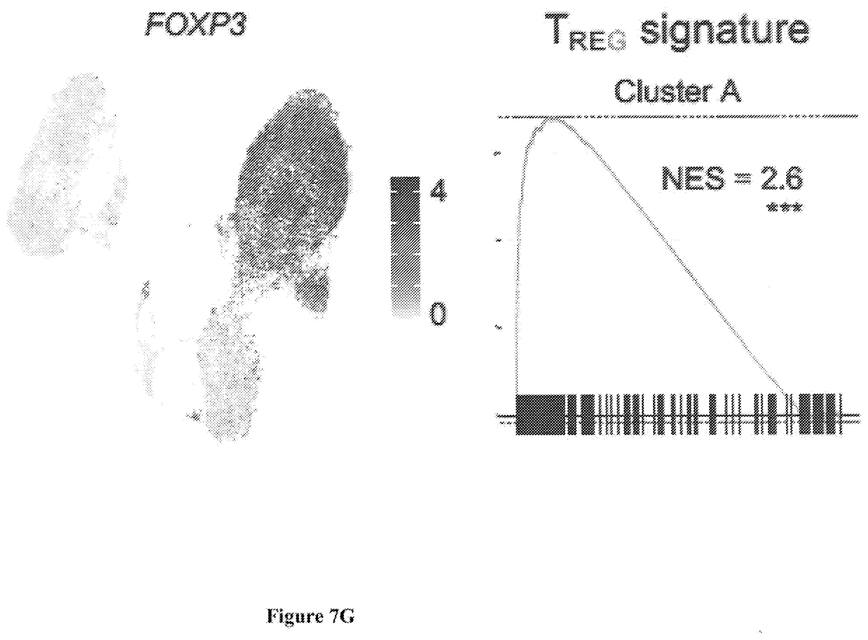

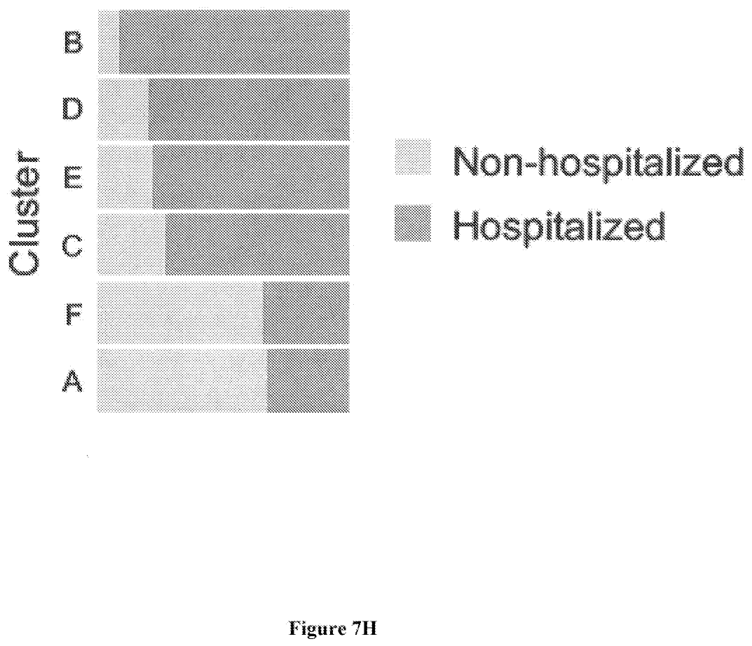

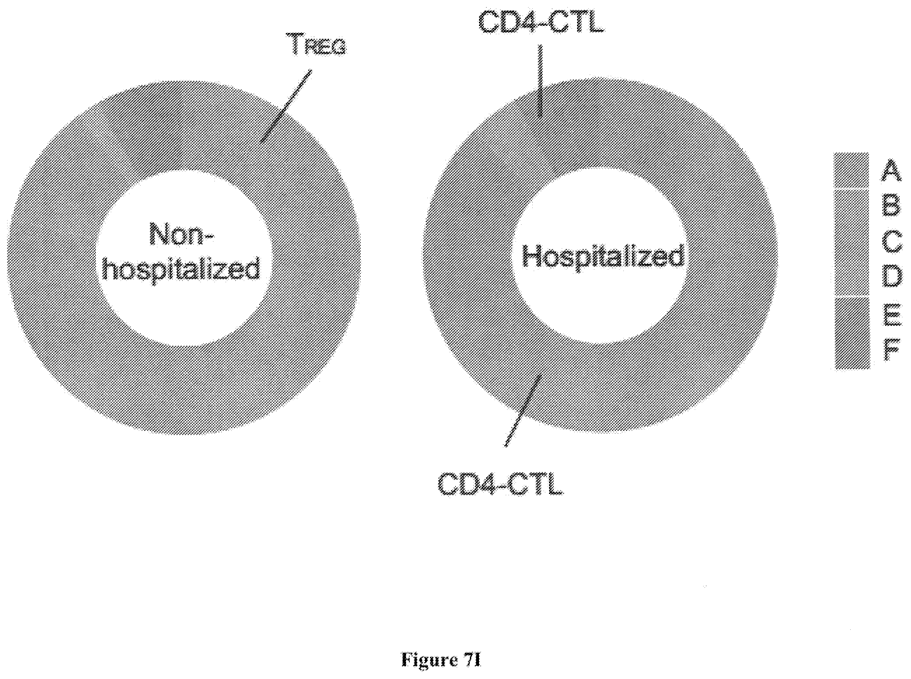

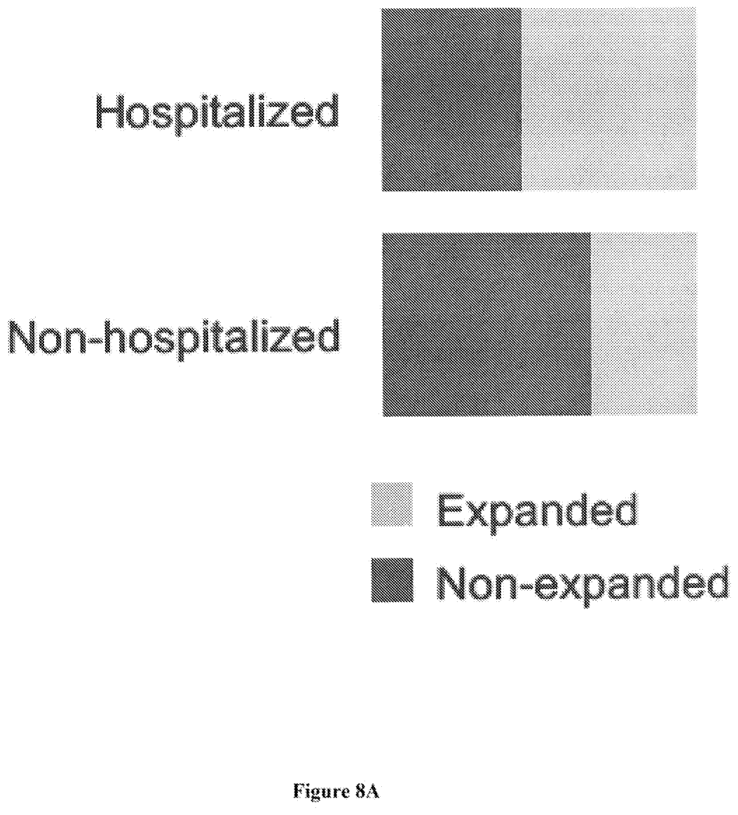

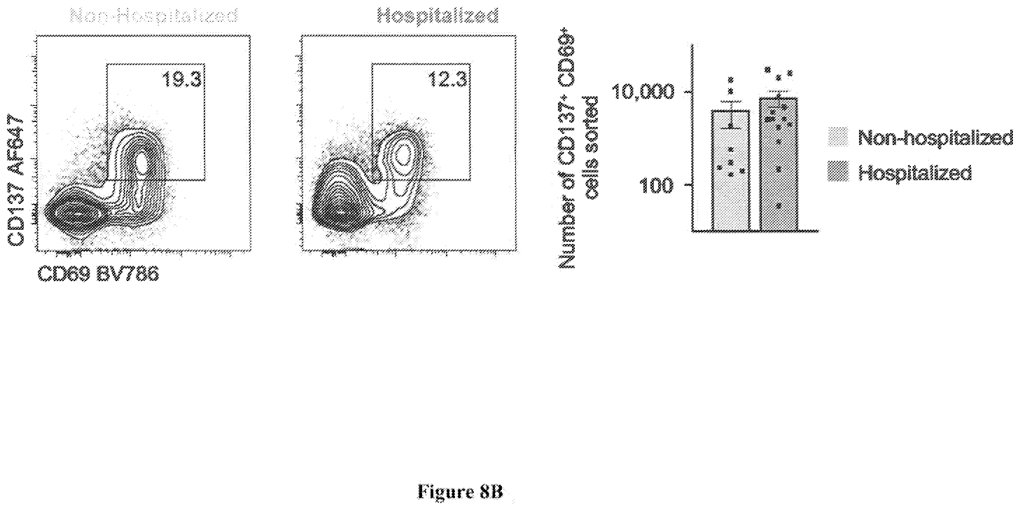

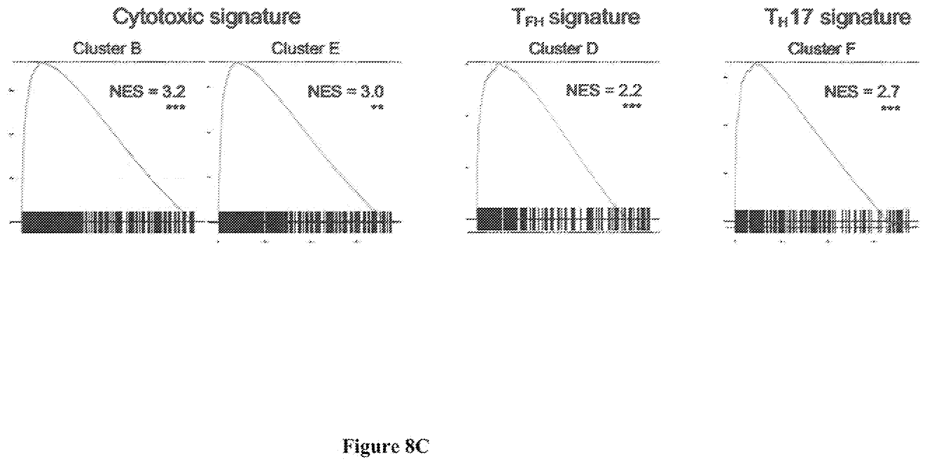

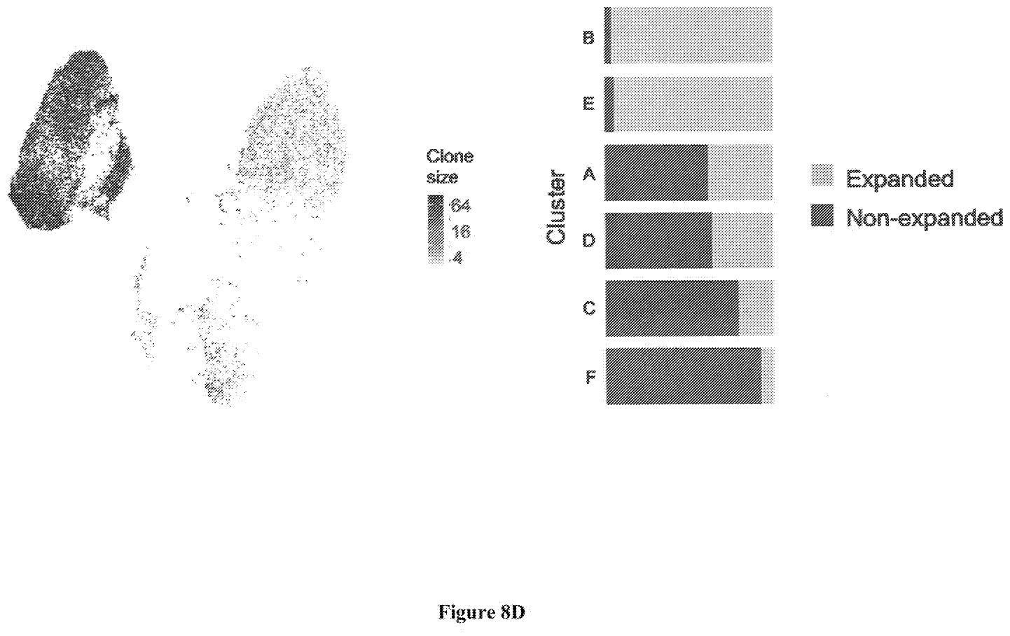

The recovery of paired T cell receptor (TCR) sequences from individual single cells enabled us to link transcriptome data to clonotype information and evaluate the clonal relationship between different CD4⁺ T cell subsets as well as determine the nature of subsets that display greatest clonal expansion. In SARS-CoV-2 infection, hospitalized patients were characterized by large clonal expansion of the virus-reactive CD4⁺ T cells; in contrast, in non-hospitalized patients, less than 45% of TCRs recovered were clonally expanded (FIG. 8A). Among SARS-CoV-2-reactive CD4⁺ T cells, CD4-CTL subsets (cluster 4 and 8) displayed the greatest clonal expansion (>75% of cells were clonally-expanded), indicating preferential expansion and persistence of CD4-CTLs in COVID-19 illness (FIG. 7A). Analysis of clonally-expanded SARS-CoV-2-reactive CD4⁺ T cells from COVID-19 patients showed extensive sharing of TCRs between cells in clusters 4 and 8, as well as those in cluster 11 (FIG. 7B), which, notably, was enriched for the expression of XCL1 and XCL2 transcripts and also for cytotoxicity-associated transcripts, albeit at lower levels compared to the established CD4-CTL clusters (FIGS. 5E and 6G). Thus, cells in cluster 11 are likely to be an intermediate transition population, a hypothesis supported by single-cell trajectory analysis that showed potential temporal connection and transcriptional similarity between these subsets (FIG. 7C).

### Example 5: SARS-CoV2-Reactive T_{REG} are Reduced in Severe COVID-19 Illness

In order to capture SARS-CoV-2-reactive CD4⁺ T cells that may not upregulate the activation markers (CD154 and CD69) after 6 hours of in vitro stimulation with SARS-CoV-2 peptide pools, the inventors stimulated PMBCs from the same cultures for a total of 24 hours (see STAR Methods) and captured cells based on co-expression of activation markers CD137 (4-1BB) and CD69, a strategy that allowed us to additionally capture antigen-specific regulatory T cells (T_{REG}) (Bacher et al., 2016)(FIGS. 7D-G and 8B). The analysis of a total of 31,278 single-cell CD4⁺ T cell transcriptomes revealed 6 distinct clusters (FIGS. 7D-F). The T_{H} subset (cluster E) was detectable at relatively lower frequencies in the 24-hour condition, though they represented the major CD4⁺ T cell subsets in the 6-hour stimulation condition (FIGS. 7D and 3A). Consistent with delayed kinetics of activation of central memory T cells (T_{CM} cells), the inventors identified a higher proportion of CD4⁺ T cells expressing transcripts linked to central memory cells (CCR7, IL7R and TCF7) (cluster C) (FIGS. 7D and 3A). The largest cluster (cluster A) was characterized by high expression of FOXP3 transcripts, which encodes for the T_{REG} master transcription factor FOXP3 (FIGS. 7D-G). Independent GSEA analysis showed significant positive enrichment of T_{REG} Signature genes in these clusters, providing that cells in these clusters represented SARS-CoV-2-reactive T_{REG} cells (FIG. 7G, right). Notably, the T_{REG} cluster contained a relatively lesser proportion of cells from hospitalized COVID-19 patients with severe illness compared to non-hospitalized subjects with milder disease (FIGS. 7H and 7I), providing a potential defect in the generation of immunosuppressive SARS-CoV-2-reactive T_{REG} cells in severe illness. Consistent with the data from the 6-hour stimulation conditions, the inventors found that cells in the CD4-CTL clusters (cluster B and D) were present at higher frequencies in patients with severe disease (FIGS. 7H and 7I). They also showed the greatest clonal expansion compared to other clusters (FIG. 8E), showing importance of the CD4-CTL subset in immune responses to SARS-CoV-2 infection.

CD4⁺ T cell subsets that are reactive to SARS-CoV-2 and other respiratory viruses show remarkable heterogeneity, and across patients with differing severity of COVID-19. Polyfunctional T_{H}1 cells, which are abundant among FLU-reactive CD4⁺ T cells and are considered to be protective (Seder et al., 2008), were present in lower frequencies among SARS-CoV-2-reactive CD4⁺ T cells from patients with severe COVID-19. Lower frequencies of T_{H}17 cells were also observed among SARS-CoV-2-reactive CD4⁺ T cells. In contrast, the inventors find increased proportions of SARS-CoV-2-reactive T_{FH} cells with dysfunctional and cytotoxicity features in hospitalized patients with severe COVID-19 illness. These findings raise the possibility that certain aspects of antigen-specific CD4⁺ T cell responses required for immune-protection are not optimally generated in COVID-19. Another striking observation is the abundance of CD4-CTLs that express high levels of transcripts encoding for multiple chemokines (XCL1, XCL2, CCL3, CCL4, CCL5) in SARS-CoV-2-reactive CD4⁺ T cells, particularly, from patients with severe COVID-19 illness. The magnitude of CD4-CTL response has been associated with better clinical outcomes in viral infections and following vaccination (Juno et al., 2017), providing that the CD4-CTL responses in COVID-19 illness may also be linked to protection.

### Example 6: Experimental Model and Subject Details (Used in Examples 1-5; and Also Referred to Herein as STAR Methods)

### COVID-19 Patients and Samples.

Ethical approval for this study from the Berkshire Research Ethics Committee 20/SC/0155 and the Ethics Committee of La Jolla Institute was in place. Written consent was obtained from all subjects. 21 hospitalized patients in a large teaching hospital in the south of England with SARS-CoV-2 infection, confirmed by reverse transcriptase polymerase chain reaction (RT-PCR) assay for detecting SARS-CoV2, between April-May 2020 were recruited to the study. A further cohort of 9 participants consisting of healthcare workers who were not hospitalized with COVID-19 illness, confirmed based on RT-PCR assay or serological evidence of SARS-CoV-2 antibodies, were also recruited over the same period. All subjects provided up to 80 mls of blood for research studies. Clinical and demographic data were collected from patient records for hospitalized patients including comorbidities, blood results, drug intervention, radiological involvement, thrombotic events, microbiology and virology results. The median age of patients with COVID-19 illness was 53 (26-82) and 67% were male. This cohort consisted of 24 (81%) White British/White Other, 4 (13%) Indian and 2 (7%) Black British participants. Of the 30 participants, 9 (30%) had mild disease and were not hospitalized, 21 (70%) had moderate/severe disease and were hospitalized. The median age of the non-hospitalized group was 40 (26-50) and 44% were male. The median age of the hospitalized patients was 60 (33-82) and 76% were male. All hospitalized patients survived to discharge from hospital.

### Healthy Controls

To study HPIV, HMPV and SARS-CoV-2 reactive CD4⁺ T cells, the inventors utilized de-identified buffy coat samples from healthy adult donors who donated blood at the San Diego Blood Bank before 2019, prior to the Covid-19 pandemic. Donors were considered to be in good health, free of cold or flu-like symptoms and with no history of Hepatitis B or Hepatitis C infection. To study FLU-reactive cells, the inventors obtained de-identified blood samples from 8 donors enrolled in the La LJI's Normal Blood Donor Program before and/or after (12-14 days) receiving the FLUCELVAX vaccine. Approval for the use of this material was obtained from the Ethics Committee of La Jolla Institute.

### Method Details

### PBMC Processing

Peripheral blood mononuclear cells (PBMCs) were isolated from up to 80 ml of anti-coagulated blood by density centrifugation over Lymphoprep (Axis-Shield PoC AS, Oslo, Norway) and cryopreserved in 50% decomplemented human antibody serum, 40% complete RMPI 1640 medium and 10% DMSO.

### SARS-CoV-2 Peptide Pools

Pools of lyophilized peptides covering the immunodominant sequence of the spike glycoprotein ad the complete sequence of the membrane glycoprotein of SARS-CoV-2 (15-mer sequences with 11 amino acids overlap) were obtained from Miltenyi Biotec (Constantin J Thieme, 2020), resuspended and stored according to the manufacturer's instructions.

### Epitope MegaPool (MP) Design

The Human Parainfluenza (HPIV), Metapneumovirus (HMPV) CD4⁺ T cell megapools (MPs) were produced by sequential lyophilization of viral-specific epitopes as previously described (Carrasco Pro et al., 2015; Weiskopf et al., 2015b). T cell prediction was performed using TepiTool tool, available in IEDB analysis resources (IEDB-AR), applying the 7-allele prediction method and a median cutoff≤20 (Dhanda et al., 2019; Paul et al., 2015; Paul et al., 2016). For the HA-influenza MP, the inventors selected 177 experimentally defined epitopes, retrieved by querying the IEDB database on 07/12/19 with search parameters “positive assay only, No B cell assays, No MHC ligand assay, Host: Homo Sapiens and MHC restriction class II”. The list of epitopes was enriched with predicted peptides derived from the HA sequences of the vaccine strains available in 2017-2018 and 2018-2019 (A/Michigan/45/2015(H1N1), B/Brisbane/60/2008,A/Hong_Kong/4801/2014_H3N2, A/Michigan/45/2015(H1N1), A/Alaska/06/2016(H3N2), B/Iowa/06/2017, B/Phuket/3073/2013). The resulting peptides were then clustered using the IEDB cluster 2.0 tool and the IEDB recommended method (cluster-break method) with a 70% cut off for sequence identity applied (Dhanda et al., 2019; Dhanda et al., 2018). Peptides were synthesized as crude material (A&A, San Diego, CA), resuspended in DMSO, pooled according to each MP composition and finally sequentially lyophilized (Carrasco Pro et al., 2015). For screening healthy non-exposed subjects (samples provided before the current pandemic) who cross-react to SARS-CoV-2, the inventors screened 20 healthy non-exposed subjects using SARS-CoV-2 peptide CD4-R and CD4-S pools, as described (Grifoni et al., 2020).

### Antigen-Reactive T Cell Enrichment (ARTE) Assay

Enrichment and FACS sorting of virus-reactive CD154⁺ or CD137⁺ CD4⁺ memory T cells following peptide pool stimulation was adapted from Bacher et al. 2016 (Bacher et al., 2016). Briefly, PBMCs from each donor, were thawed, washed, plated in 6-well culture plates at a concentration of 5×10⁶ cells/ml in 1 ml of serum-free TexMACS medium (Miltenyi Biotec) and left overnight (5% CO₂, 37° C.). Cells were stimulated by the addition of individual virus-specific peptide pools (1 μg/ml) for 6 h in the presence of a blocking CD40 antibody (1 μg/ml; Miltenyi Biotec). For subsequent MACS-based enrichment of CD154⁺, cells were sequentially stained with fluorescence-labeled surface antibodies, Cell-hashtag TotalSeq™-C antibody (0.5 μg/condition), and a biotin-conjugated CD154 antibody (clone 5C8; Miltenyi Biotec) followed by anti-biotin microbeads (Miltenyi Biotec). Labelled cells were added to MS columns (Miltenyi Biotec) and positively selected cells (CD154⁺) were eluted and used for FACS sorting of CD154⁺ memory CD4⁺ T cells. The flow-through from the column was collected and re-plated to harvest cells responding 24 h after peptide stimulation. Analogous to enrichment for CD154⁺, CD137-expressing CD4⁺ memory T cells were positively selected by staining with biotin-conjugated CD137 antibody (clone REA765; Miltenyi Biotec) followed by anti-biotin MicroBeads and applied to a new MS column. Following elution, enriched populations were immediately sorted using a FACSAria Fusion Cell Sorter (Becton Dickinson) based on dual expression of CD154 and CD69 for 6-hour stimulation condition, and CD137 and CD69 for 24-hour stimulation condition. The gating strategy used for sorting is shown in FIGS. 2A and 8B. All flow cytometry data were analyzed using FlowJo software (version 10).

### Cell Isolation and Single-Cell RNA-Seq Assay (10× Platform).

For combined single-cell RNA-seq and TCR-seq assays (10× Genomics), a maximum of 60,000 virus-reactive memory CD4⁺ T cells from up to 8 donors were pooled by sorting into low retention 1.5 mL collection tubes, containing 500 μL of a 1:1 solution of PBS:FBS supplemented with RNAse inhibitor (1:100). Following sorting, ice-cold PBS was added to make up to a volume of 1400 μl. Cells were then centrifuged for 5 minutes (600 g at 4° C.) and the supernatant was carefully removed leaving 5 to 10 μl. 25 μl of resuspension buffer (0.22 μm filtered ice-cold PBS supplemented with ultra-pure bovine serum albumin; 0.04%, Sigma-Aldrich) was added to the tube and the pellet was gently but thoroughly resuspended. Following careful mixing, 33 μl of the cell suspension was transferred to a PCR-tube for processing as per the manufacturer's instructions (10× Genomics).

Briefly, single-cell RNA-sequencing library preparation was performed as per the manufacturer's recommendations for the 10× Genomics 5′TAG v1.0 chemistry with immune profiling and cell surface protein technology. Both initial amplification of cDNA and library preparation were carried out with 13 cycles of amplification; V(D)J and cell surface protein libraries were generated corresponding to each 5′TAG gene expression library using 9 cycles and 8 cycles of amplification, respectively. Libraries were quantified and pooled according to equivalent molar concentrations and sequenced on Illumina's NovaSeq6000 sequencing platform with the following read lengths: read 1—101 cycles; read 2—101 cycles; and i7 index—8 cycles.

### Single-Cell Transcriptome Analysis

Reads from single-cell RNA-seq were aligned and collapsed into Unique Molecular Identifiers (UMI) counts using 10× genomics' Cell Ranger software (v3.1.0) and mapping to GRCh37 reference (v3.0.0) genome. Hashtag UMI counts for each TotalSeq™-C antibody capture library were generated with the Feature Barcoding Analysis pipeline from Cell Ranger. To demultiplex donors, UMI counts of cell barcodes were first obtained from the raw data output, and only cells with at least 100 UMI were considered for donor assignment. Donor identities were inferred by MULTIseqDemux (autoThresh=TRUE and maxiter=10) from Seurat (v3.1.5) using the UMI counts. Each cell barcode was assigned a donor ID, marked as a Doublet, or having a Negative enrichment. Cells with multiple barcodes were re-classified as doublets if the ratio of UMI counts between the top 2 barcodes was less than 3. Cells labeled as Doublet or Negative were removed from downstream analyses. Raw 10× data, from four libraries, was aggregated using Cell Ranger's aggr function (v3.1.0). The merged data was transferred to the R statistical environment for analysis using the package Seurat (v3.1.5) (Stuart et al., 2019). To further minimize doublets and to eliminate cells with low quality transcriptomes, cells expressing <800 and >4400 unique genes, <1500 and >20,000 total UMI content, and >10% of mitochondrial reads were excluded. The summary statistics for all the single-cell transcriptome libraries indicate good quality data with no major differences in quality control metrices across multiple batches (FIG. 4A). This procedure was independently applied for data from CD4⁺ T cells stimulated for 6 hours and 24 hours.

For single-cell transcriptome analysis only genes expressed in at least 0.1% of the cells were included. The transcriptome data was then log-transformed and normalized (by a factor of 10,000) per cell, using default settings in Seurat software. Variable genes with a mean expression greater than 0.01 and explaining 25% of the total variance were selected using the Variance Stabilizing Transformation method, as described (Stuart et al., 2019). Transcriptomic data from each cell was then further scaled by regressing the number of UMI-detected and percentage of mitochondrial counts. For data from CD4+ T cells stimulated for 6 hours, principal component analysis was performed using the variable genes, and based on the standard deviation of PCs in the “elbow plot”, the first 38 principal components (PCs) were selected for further analyses. Cells were clustered using the FindNeighbors and FindClusters functions in Seurat with a resolution of 0.6. The robustness of clustering was independently verified by other clustering methods and by modifying the number of PCs and variable genes utilized for clustering. Analysis of clustering patterns across multiple batches revealed no evidence of strong batch effects (FIG. 2A, right panel). For data from CD4⁺ T cells stimulated for 24 hours, principal component analysis was performed using the genes explaining 25% of the variance, and the first 16 principal components (PCs) were selected for further analyses. Cells were clustered using the FindNeighbors and FindClusters functions in Seurat with a resolution of 0.2. Further visualizations of exported normalized data such has “violin” plots were generated using the Seurat package and custom R scripts. Violin shape represents the distribution of cell expressing transcript of interest (based on a Gaussian Kernel density estimation model) and are colored according to the percentage of cells expressing the transcript of interest.

### Single-Cell Differential Gene Expression Analysis

Pair-wise single-cell differential gene expression analysis was performed using the MAST package in R (v1.8.2) (Finak et al., 2015) after conversion of data to counts per million (CPM+1). A gene was considered differentially expressed when Benjamini-Hochberg-adjusted P-value was <0.05 and a log 2 fold change was more than 0.25. For finding cluster markers (transcripts enriched in a given cluster) the function FindAllMarkers from Seurat was used.

### Gene Set Enrichment Analysis and Signature Module Scores

GSEA scores were calculated with the package fgsea in R using the signal-to-noise ratio as a metric. Gene sets were limited by minSize=3 and maxSize=500. Normalized enrichment scores were presented as * plots. Signature module scores were calculated with AddModuleScore function, using default settings in Seurat. Briefly, for each cell, the score is defined by the mean of the signature gene list after the mean expression of an aggregate of control gene lists is subtracted. Control gene lists were sampled (same size as the signature list) from bins created based on the level of expression of the signature gene list.

### Single-Cell Trajectory Analysis

The “branched” trajectory was constructed using Monocle 3 (v0.2.1, default settings) with the number of UMI and percentage of mitochondrial UMI as the model formula, and including the highly variable genes from Seurat for consistency. After setting a single partition for all cells, the cell-trajectory was projected on the PCA and UMAP generated from Seurat analysis. The ‘root’ was selected by the get_earliest_principal_node function provided in the package.

### T Cell Receptor (TCR) Sequence Analysis

Reads from single-cell V(D)J TCR sequence enriched libraries were 5 processed with the vdj pipeline from Cell Ranger (v3.1.0 and human annotations reference GRCh38, v3.1.0, as recommended). In brief, the V(D)J transcripts were assembled and their annotations were obtained for each independent library. In order to perform combined analysis of single-cell transcriptome and TCR sequence from the same cells V(D)J libraries were first aggregated using a custom script. Then cell barcode suffixes from these libraries were revised according to the order of their gene expression libraries. Unique clonotypes, as defined by 10× Genomics as a set of productive Complementarity-Determining Region 3 (CDR3) sequences, were identified across all library files and their frequency and proportion (clone statistics) were calculated based on the aggregation result. This procedure was independently applied for data from CD4⁺ T cells stimulated for 6 hours and 24 hours. Based on the vdj aggregation files, barcodes captured by the gene expression data and previously filtered to keep only good quality cells, were annotated with a specific clonotype ID alongside their clone size (number of cells with the same clonotypes in both the TCR alpha and beta chains) statistics. Cells that share clonotype with more than 1 cell were called as clonally expanded (clone size 2). Clone size for each cell was visualized on UMAP. Sharing of clonotype between cells in different clusters was depicted using the tool UpSetR.

### Quantification and Statistical Analysis

Processing of data, applied methods and codes are described in the respective section in the STAR Methods. The number of subjects, samples and replicates analyzed, and the statistical test performed are indicated in the figure legends. Statistical analysis for comparison between two groups was assessed with Student's unpaired two-tailed t-test using GraphPad Prism 7.0d.

### Example 7: CD4+ T Cell Responses in COVID-19 Illness

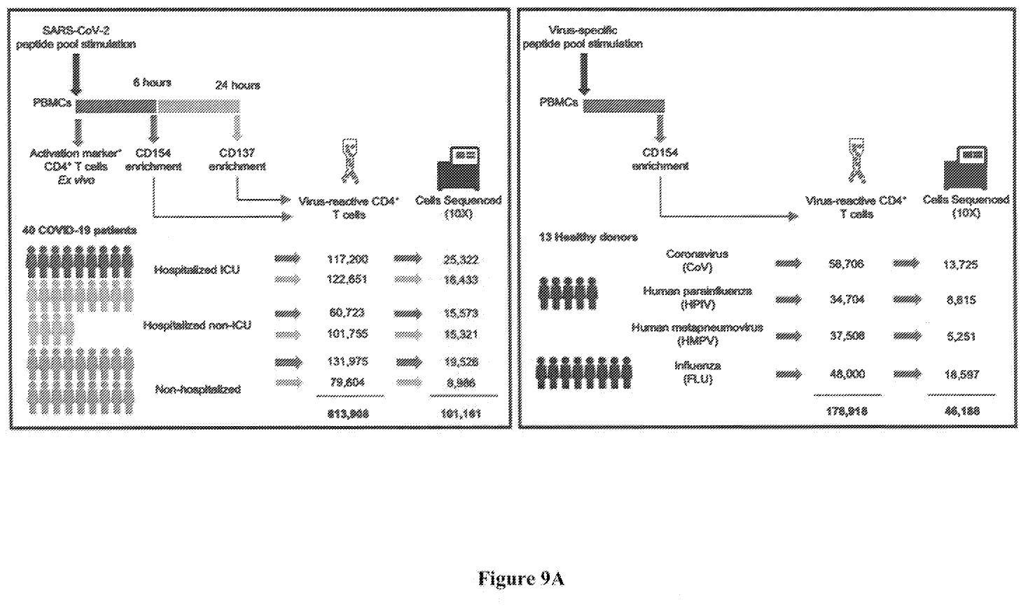

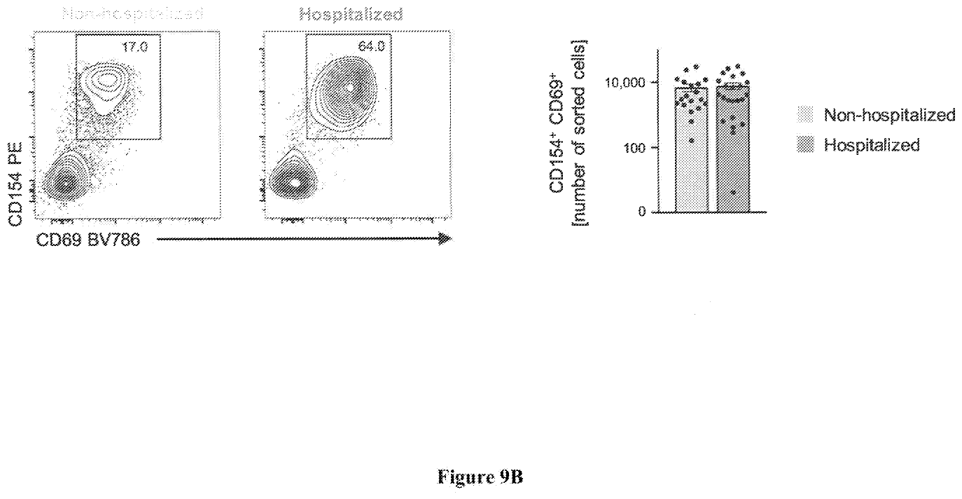

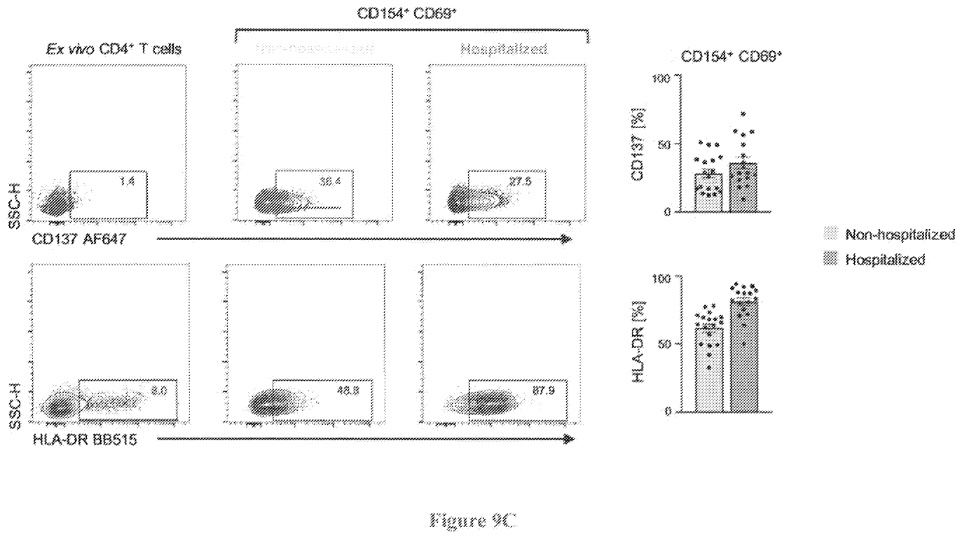

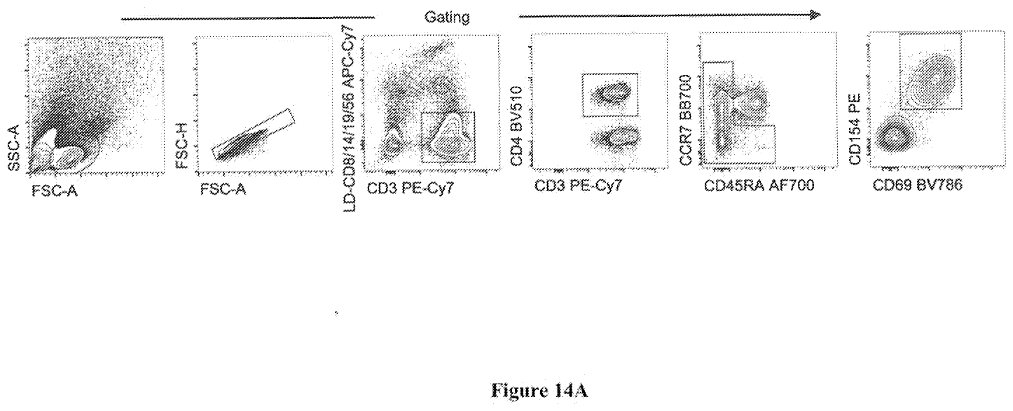

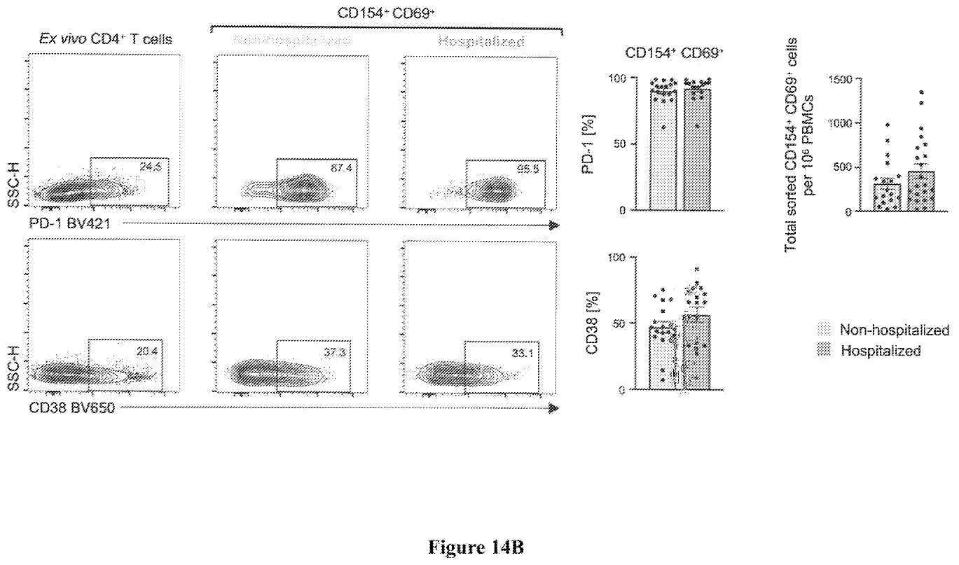

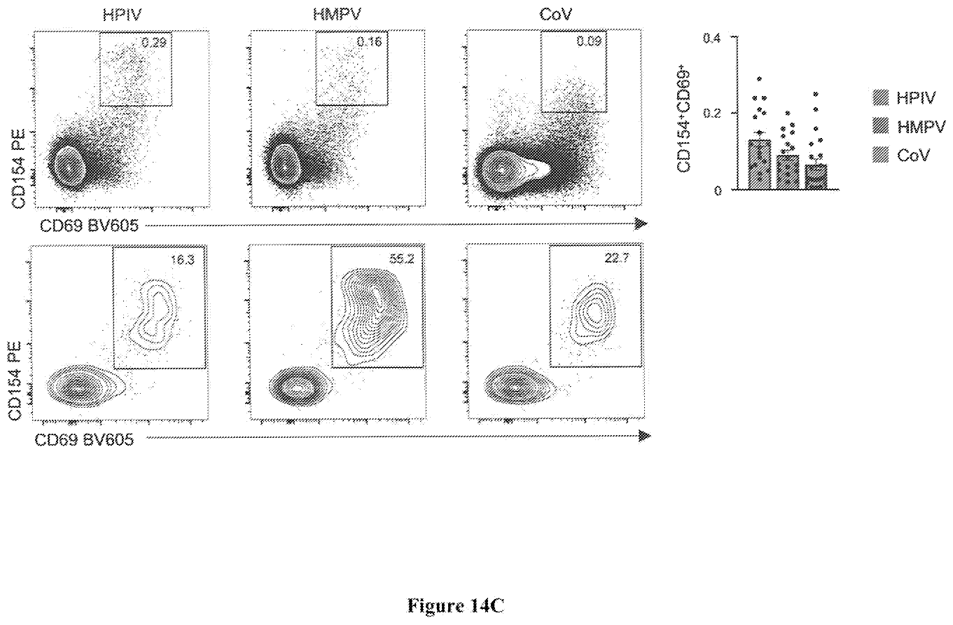

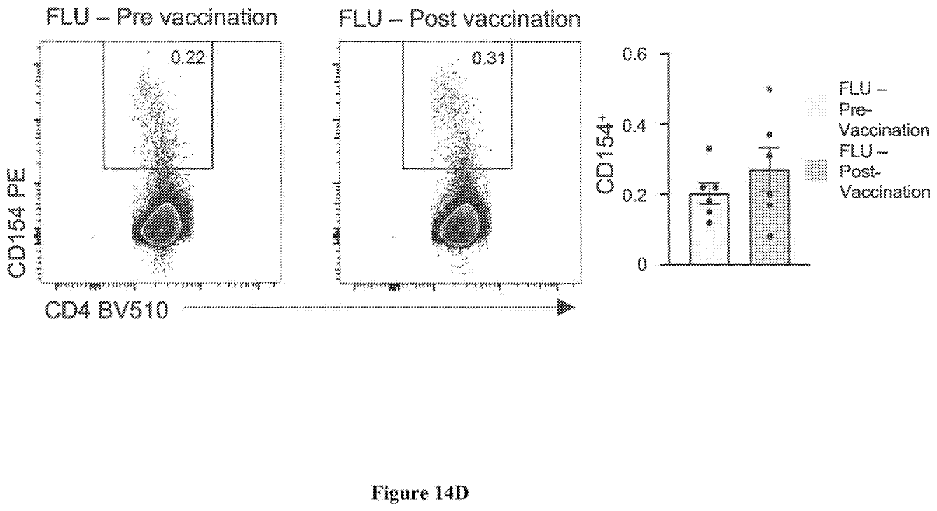

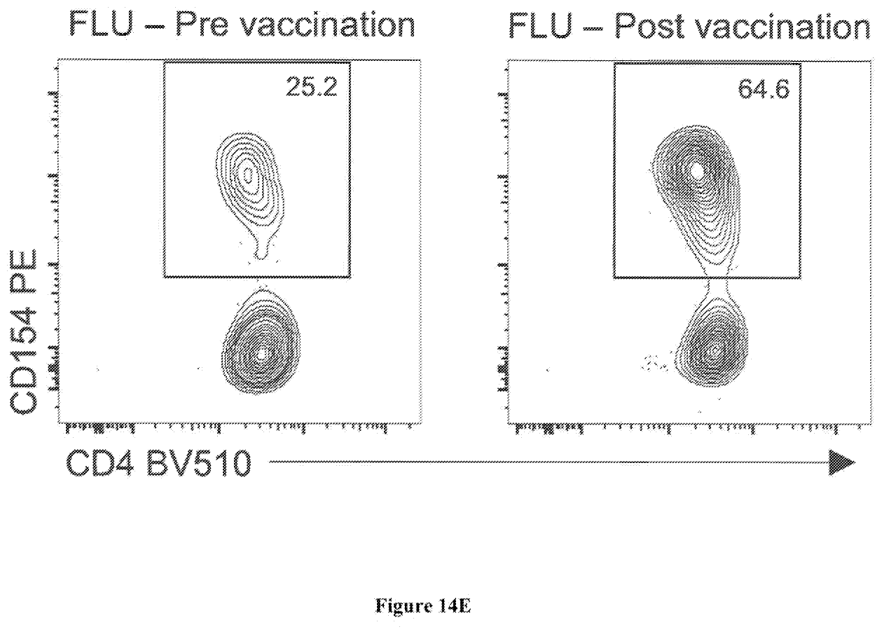

To capture CD4+^{T} cells responding to SARS-CoV-2 in patients with COVID-19 illness, we employed the antigen-reactive T cell enrichment (ARTE) assay (Bacher et al., 2013, 2016, 2019; Schmiedel et al., 2018) that relies on in vitro stimulation of peripheral blood mononuclear cells (PBMCs) for 6 h with overlapping peptide pools targeting the immunogenic domains of the spike and membrane proteins of SARS-CoV-2 (see STAR Methods; Thieme et al., 2020). Following in vitro stimulation, SARS-CoV-2-reactive CD4+ memory T cells were isolated based on the expression of cell surface markers (CD154 and CD69) that reflect recent engagement of the T cell receptor (TCR) by cognate major histocompatibility complex (MHC)-peptide complexes (FIG. 14A). In the context of acute COVID-19 illness, CD4+ T cells expressing activation markers have been reported in the blood (Braun et al., 2020; Thevarajan et al., 2020); such CD4+ T cells, presumably activated in vivo by endogenous SARS-CoV-2 viral antigens, were also captured during the ARTE assay, thereby enabling us to study a comprehensive array of CD4+ T cell subsets responding to SARS-CoV-2. We sorted >300,000 SARS-CoV-2-reactive CD4+ T cells from >1.3 billion PBMCs isolated from a total of 40 patients with COVID-19 illness (22 hospitalized patients with severe illness, 9 of whom required intensive care unit [ICU] treatment, and 18 non-hospitalized subjects with relatively milder disease; FIGS. 9A and 9B). In addition to expressing CD154 and CD69, sorted SARS-CoV-2-reactive CD4+ T cells co-expressed other activation-related cell surface markers like CD38, CD137(4-1BB), CD279 (PD-1), and HLA-DR (FIGS. 9C and 14B).

Recent evidence from studies in non-exposed individuals (blood sample obtained pre-COVID-19 pandemic) indicates pre-existing SARS-CoV-2-reactive CD4+ T cells, possibly indicative of human coronavirus (HCoV) cross-reactivity. Such cells are observed in up to 50% of the subjects studied (Braun et al., 2020; Grifoni et al., 2020; Le Bert et al., 2020). To capture such SARS-CoV-2-reactive CD4+ T cells, likely to be coronavirus (CoV)-reactive, we screened healthy non-exposed subjects and isolated CD4+ T cells responding to SARS-CoV-2 peptide pools from 4 subjects with highest responder frequency (FIGS. 9A and 14C). Next, for defining the CD4+ T cell subsets and their properties that distinguish SARS-CoV-2-reactive cells from other common respiratory virus-reactive CD4+ T cells, we isolated CD4+ T cells responding to peptide pools specific to influenza hemagglutinin protein (FLU-reactive cells, see STAR Methods) from 8 additional healthy subjects who provided blood samples before and/or after influenza vaccination (FIGS. 9A, 14D, and 14E). CD4+ T cells responding to peptide pools specific to other common respiratory viruses like human parainfluenza (HPIV) and human metapneumovirus (HMPV) were also isolated from healthy subjects (FIG. 14C). In total, we interrogated the transcriptome and TCR sequence of >100,000 viral reactive CD4+ T cells from 53 subjects (FIGS. 9A, 14A, and 14B).

### Example 8: SARS-CoV-2-Reactive CD4+ T Cells are Enriched for TFH Cells and CD4-CTLs

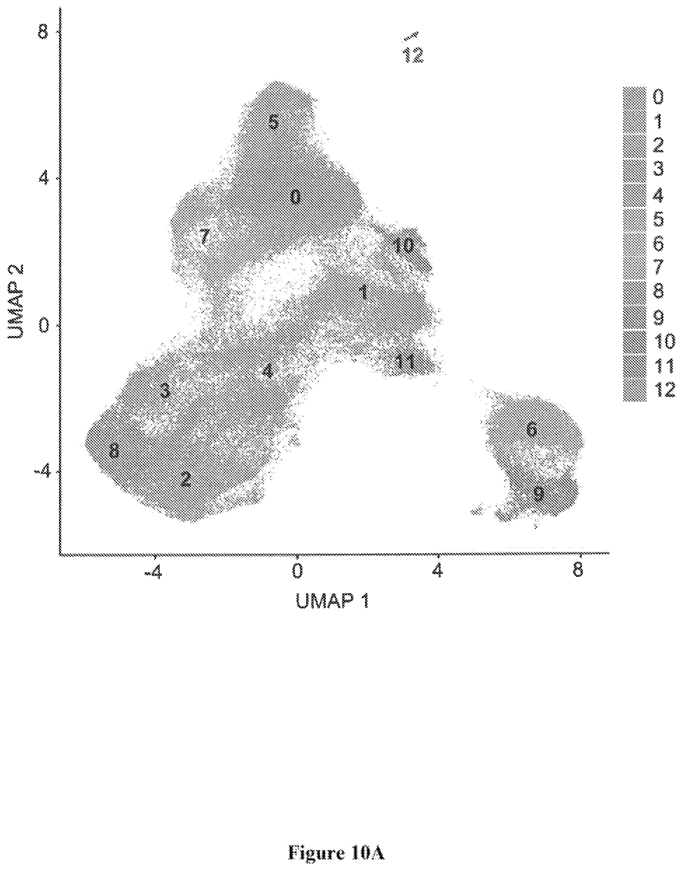

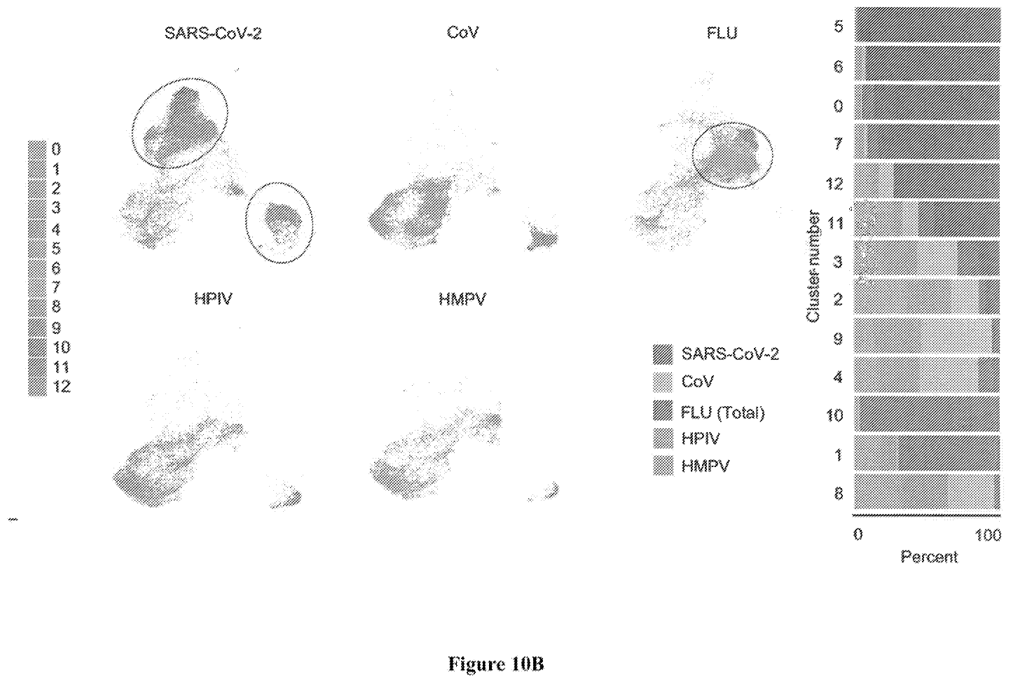

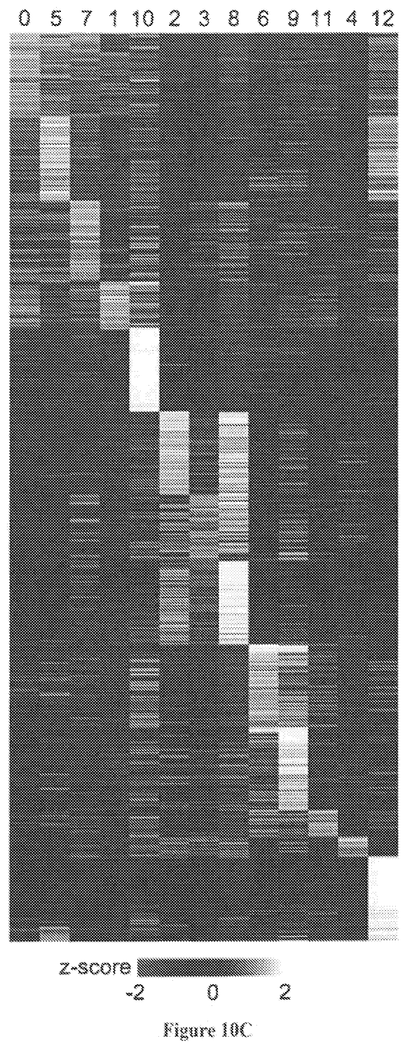

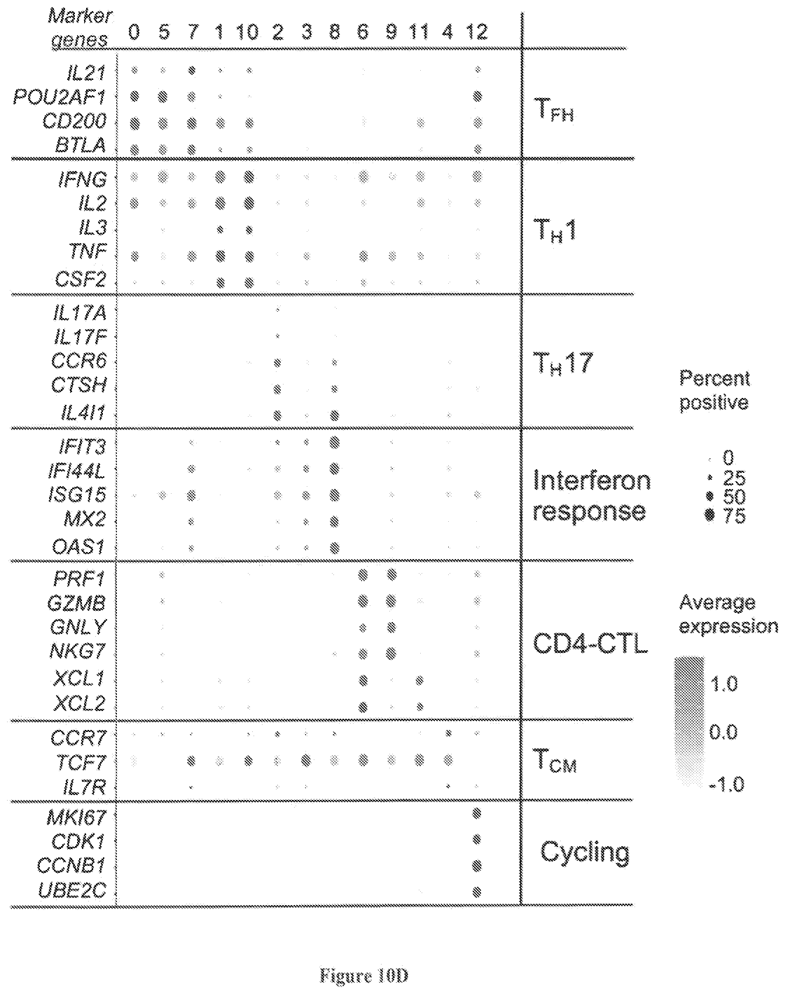

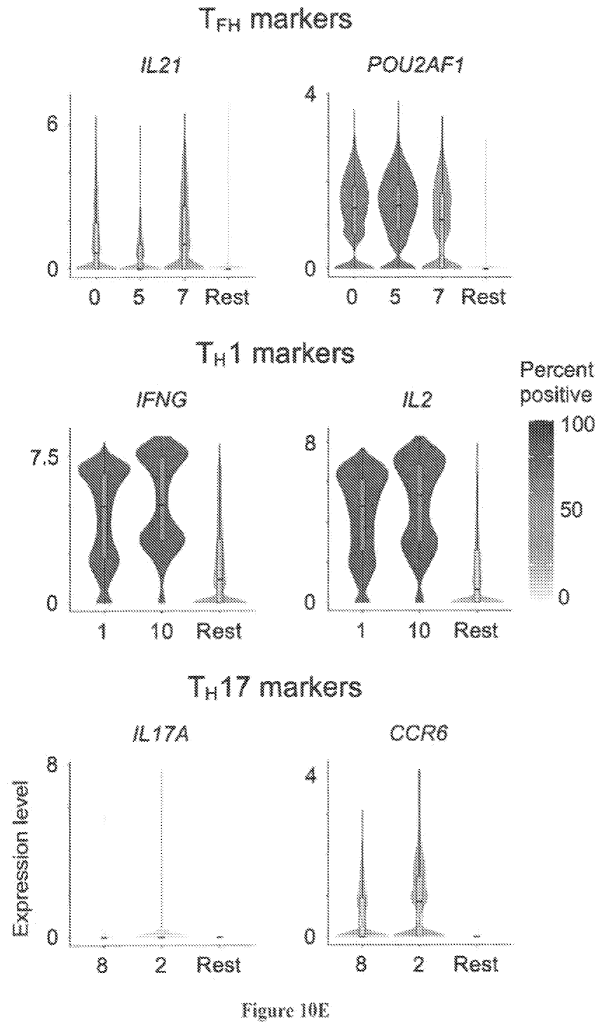

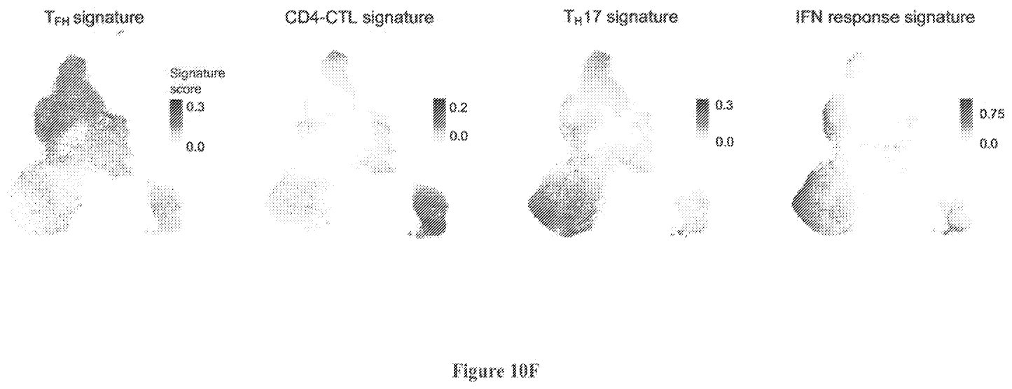

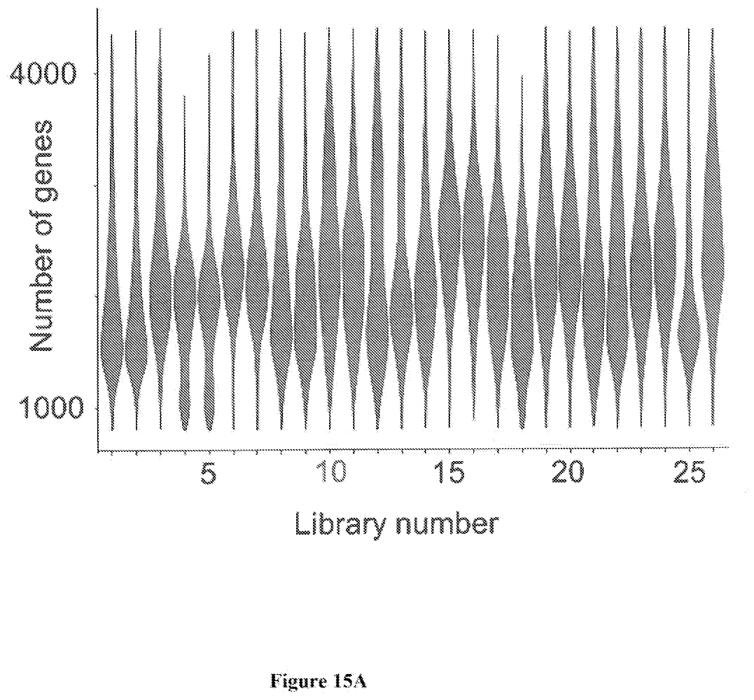

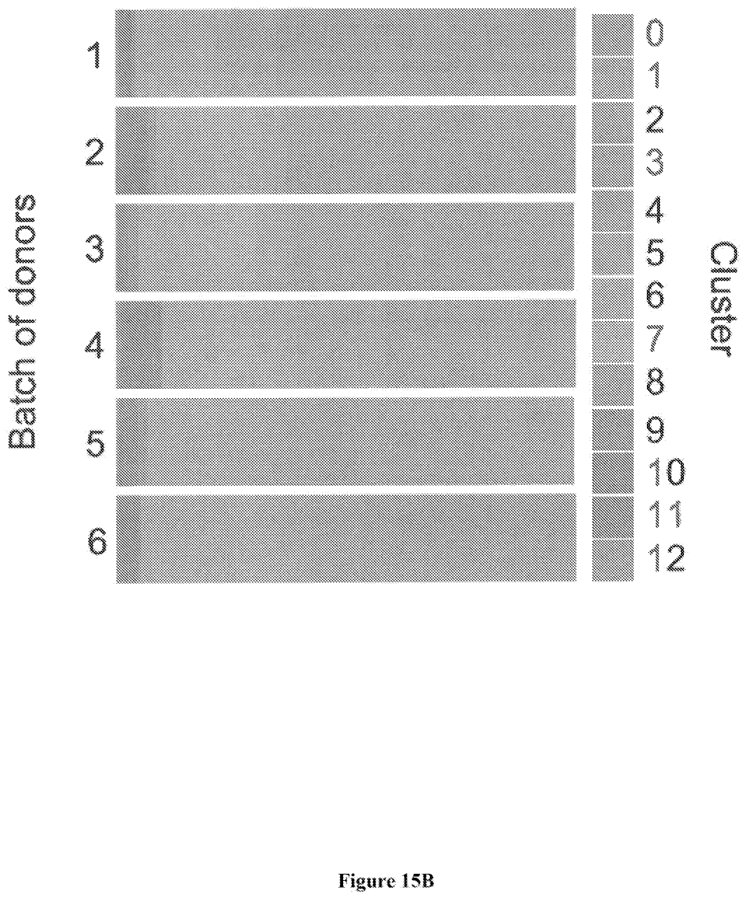

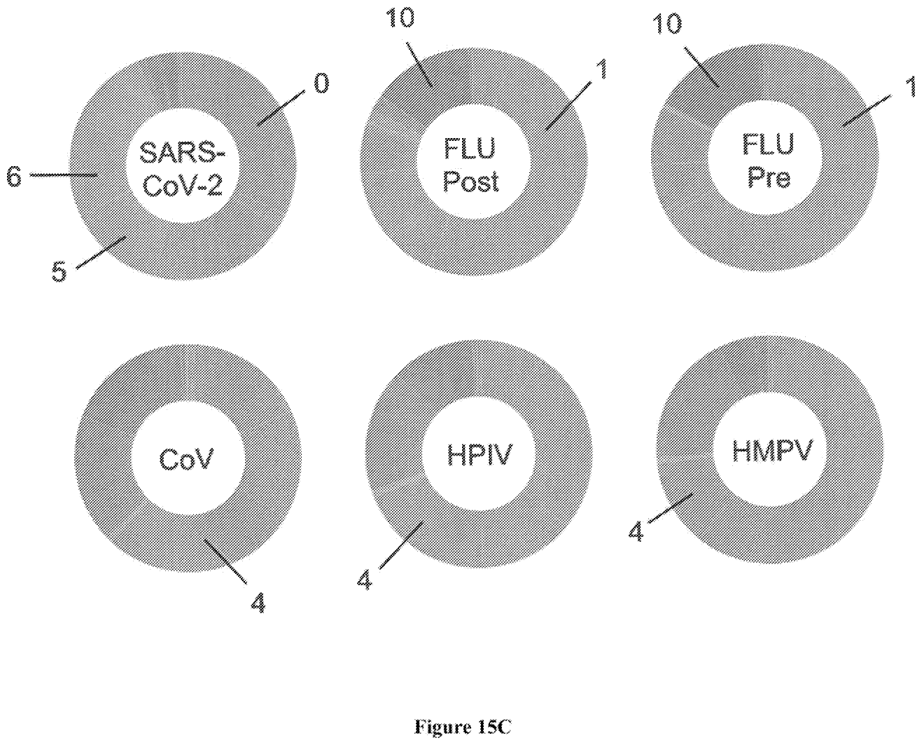

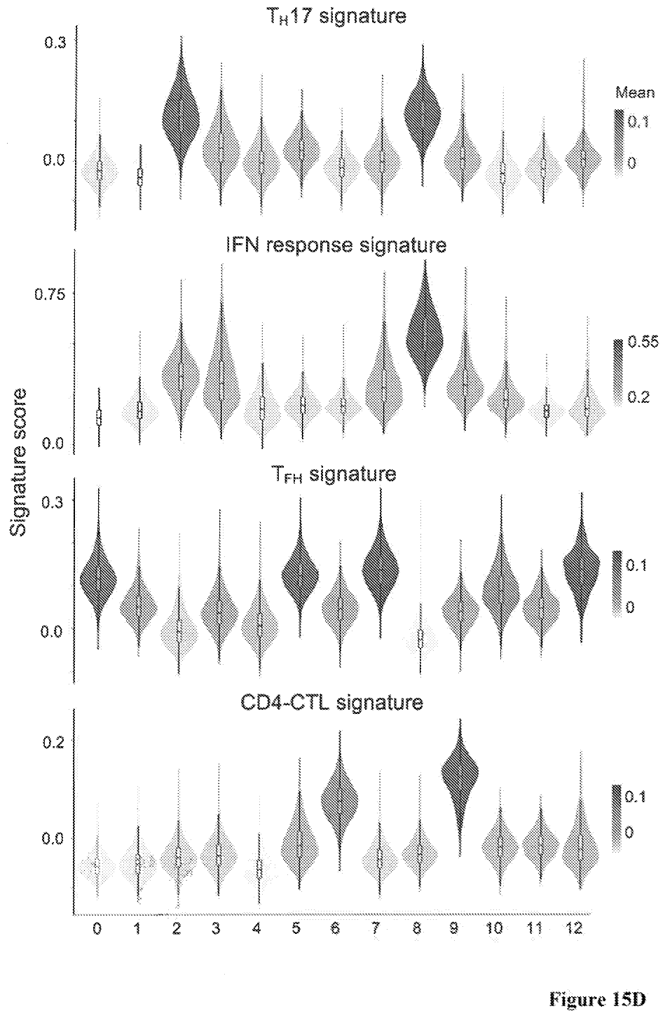

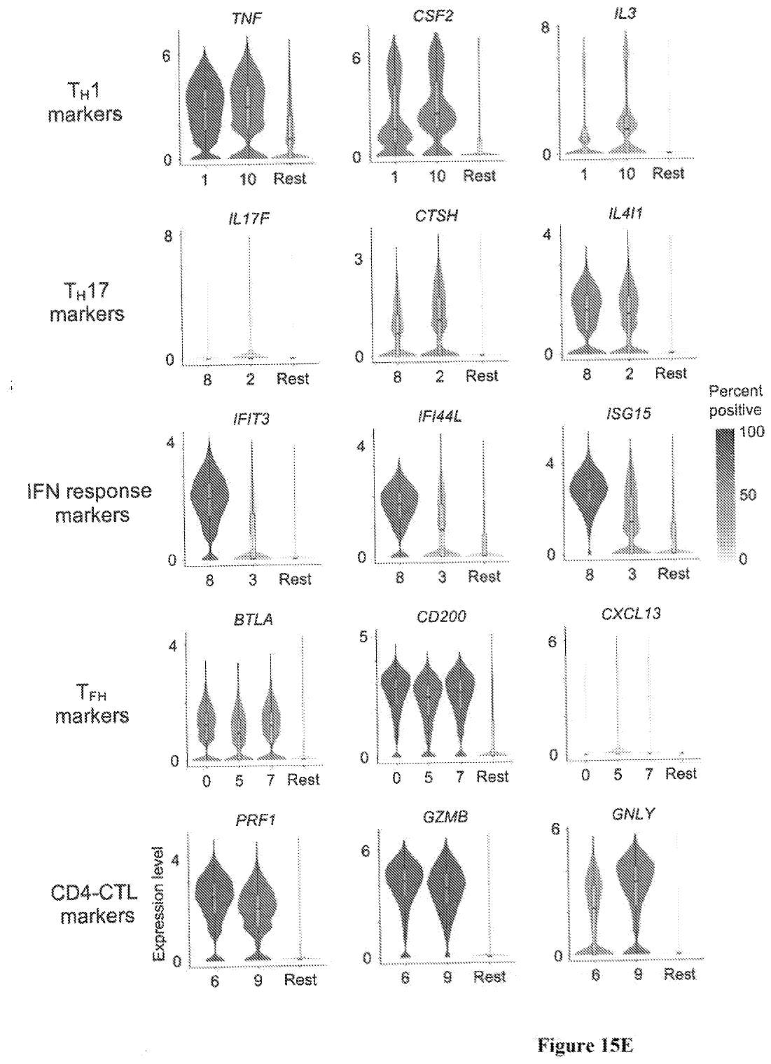

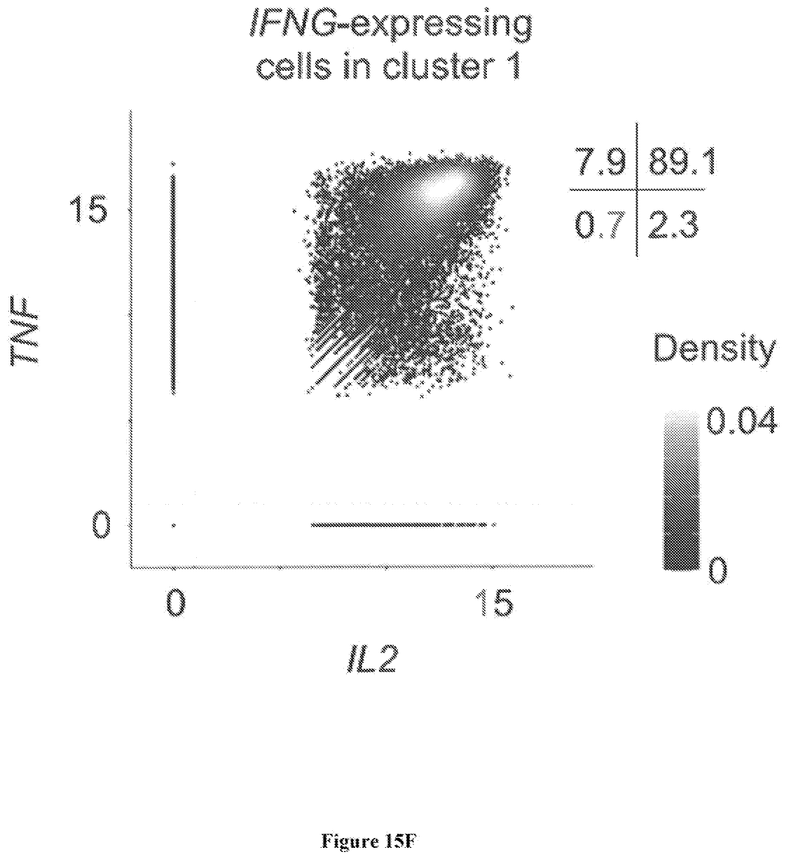

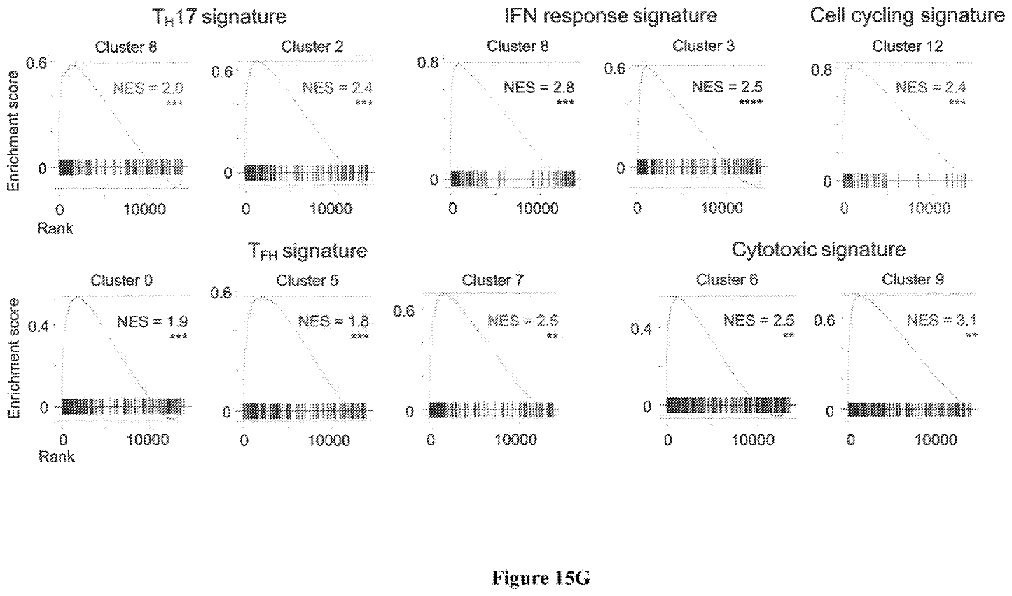

Analysis of the single-cell transcriptomes of all viral-reactive CD4+ T cells from all subjects revealed 13 CD4+ T cell subsets that clustered distinctly, reflecting their unique transcriptional profiles (FIGS. 10A-10D). Strikingly, a number of clusters were dominated by cells reactive to particular viruses (FIGS. 2B and S2C). For example, the vast majority of cells in clusters 1 and 10 were FLU-reactive (>65%), whereas cells in clusters 0, 5, 6, 7, and 12 mainly consisted of SARS-CoV-2-reactive CD4+ T cells (>70%) from COVID-19 patients (FIGS. 10B and 15C). Conversely, cells in clusters 2, 3, 4, 8, and 9 were not preferentially enriched for reactivity to any given virus (FIGS. 10B and 15C). These findings suggest that distinct viral infections generate CD4+ T cell subsets with distinct transcriptional programs, although the timing of survey (acute illness versus past infection) will also contribute to their cellular states. Our data highlight substantial heterogeneity in the nature of CD4+T cells generated in response to different viral infections on the one hand and shared features on the other.

The clusters enriched for FLU-reactive CD4+ T cells (clusters 1 and 10) displayed features suggestive of polyfunctional T helper (TH)1 cells which have been associated with protective anti-viral immune responses (Seder et al., 2008). Such features include the expression of transcripts encoding for the cytokines linked to polyfunctionality such as IFN-g, IL-2, and TNFa, and several other cytokines and chemokines like IL-3, CSF2, IL-23A, and CCL20 (FIGS. 10D, 10E, 15E, and 15F). SARS-CoV-2-reactive CD4+ T cells were underrepresented in these clusters (cluster 1 and 10, <2%) when compared to FLU-reactive cells (>70%) or HMPV- and HPIV-reactive cells (˜5%-20%) (FIG. 15C). Furthermore, SARS-CoV-2-reactive CD4+ T cells in cluster 1 expressed significantly lower levels of IFNG and IL2 transcripts when compared to FLU-reactive cells. Together, these data suggested a failure to generate robust polyfunctional T_{H}1 cells in SARS-CoV-2 infection. A similar pattern was also observed in SARS-CoV-2-reactive CD4+ T cells from healthy non-exposed subjects (FIGS. 10B and 15C) but not for HPIV or HMPV-reactive CD4+ T cells, suggesting the defect in generating polyfunctional TH1 cells may be a common feature for coronaviruses, although further studies specifically analyzing HCoV-reactive CD4+ T cells in healthy individuals will be required to verify this.

Other clusters that were relatively underrepresented for SARS-CoV-2-reactive CD4+ T cells included clusters 2 and 8, which were both enriched for TH17 signature genes, with cluster 2 highly enriched for cells expressing IL17A and IL17F transcripts, thus representing bona fide TH17 cells (FIGS. 10B-10F and 15C-15E). TH17 cells have been associated with protective immune responses in certain models of viral infections (Acharya et al., 2016; Wang et al., 2011); however, in other contexts they have been shown to promote viral disease pathogenesis (Acharya et al., 2016; Ma et al., 2019). Therefore, the functional relevance of an impaired T_{H}17 response in COVID-19 is not clear and requires further investigation.

Clusters that were evenly distributed across all viral-specific CD4+ T cells include clusters 3 and 4. Cluster 3 displayed a transcriptional profile consistent with enrichment of interferon (IFN)-response genes (IFIT3, IFI44L, ISG15, MX2, OAS1), and cluster 4 was enriched for CCR7, IL7R, and TCF7 transcripts, likely representing central memory CD4+ T cell subset (FIGS. 10B-10F and 15C-15E). Cluster 12, which expressed high levels of transcripts linked to cell cycle genes MK167 and CDK1, also contained a large proportion of SARS-CoV-2-reactive CD4+ T cells (FIGS. 10B-10D), indicative of actively proliferating cells responsive to SARS-CoV-2 antigens. Cluster 6, also dominated by SARS-CoV-2-reactive CD4+ T cells, was characterized by high levels of PRF1, GZMB, GZMH, GNLY, and NKG7 transcripts, which encode for molecules linked to cytotoxicity (Patil et al., 2018) (FIGS. 10B-10F and 15C-15E). Gene set enrichment analysis (GSEA) showed significant positive enrichment of signature genes for cytotoxicity in clusters 6 and 9 (FIG. 15G), confirming these clusters represent cytotoxic CD4+ T cells (CD4-CTLs).

Clusters 0, 5, and 7, which were colocalized in the uniform manifold approximation and projection (UMAP) plot, were dominated by SARS-CoV-2-reactive CD4+ T cells (FIGS. 10A and 10B). Cells in these clusters were uniformly enriched for transcripts encoding for cytokines, surface markers, and transcriptional coactivators associated with T follicular helper (TFH) cell function (CXCL13, IL21, CD200, BTLA, and POU2AF1) (Locci et al., 2013) (FIGS. 10B-10F and 15C-15E). Independent GSEA showed significant positive enrichment of TFH signature genes in these clusters, confirming that cells in these clusters represent circulating TFH cells (FIG. 15G). Bona fide TFH cells reside in the germinal center; however, TFH cells have been described in the blood where increased numbers have been reported during viral infections and following vaccinations (Bentebibel et al., 2013; Koutsakos et al., 2018; Smits et al., 2020). Thus, the increase in circulating SARSCoV-2-reactive TFH subsets observed in patients with COVID-19 is consistent with published reports in acute infections. Overall, our single-cell transcriptomic analysis revealed substantial differences in the nature of CD4+ T cell responses to viral infections and highlight subsets that are specifically enriched or depleted in COVID-19 illness.

### Example 9: SARS-CoV-2-Reactive CD4+ T Cell Subsets Associated with Disease Severity

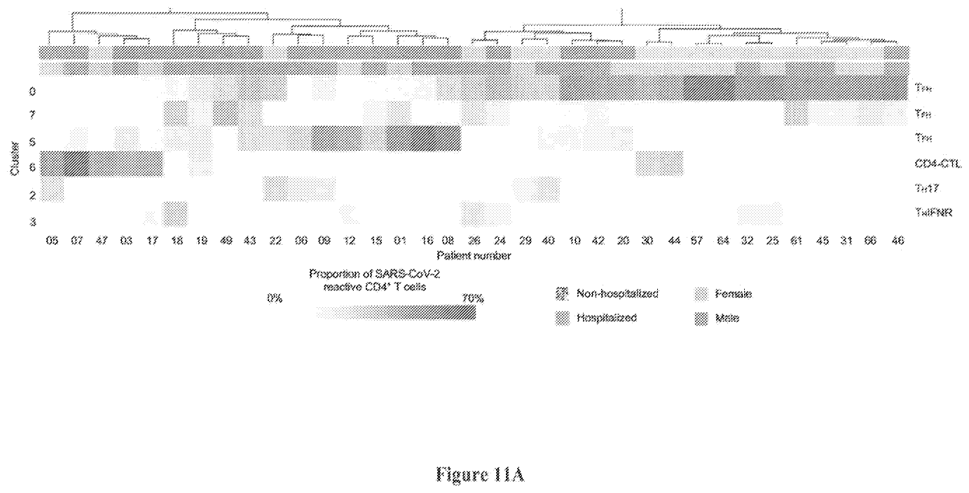

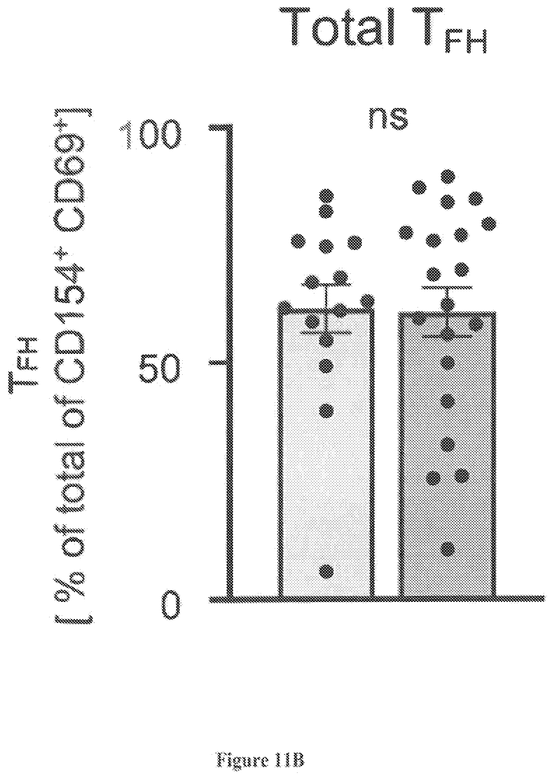

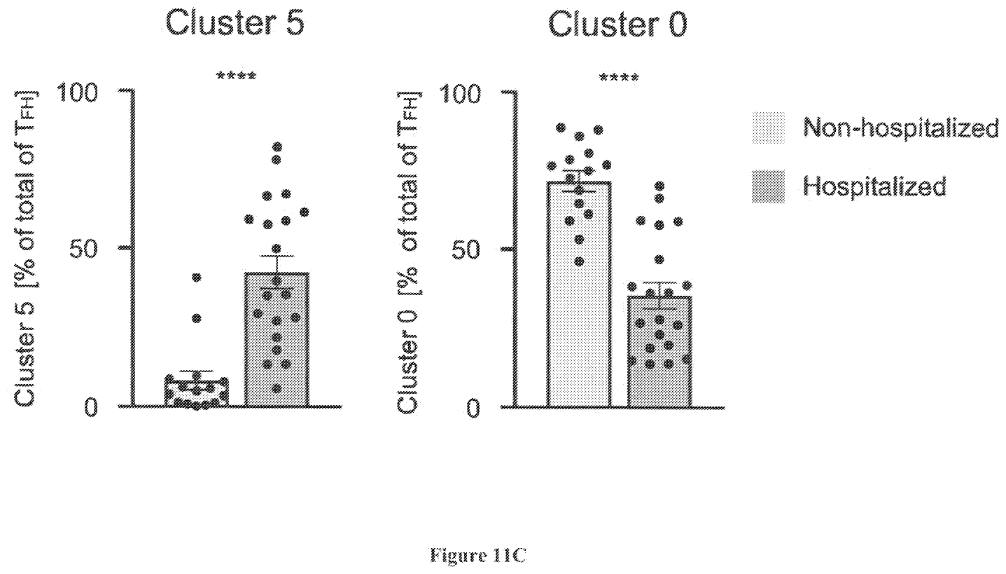

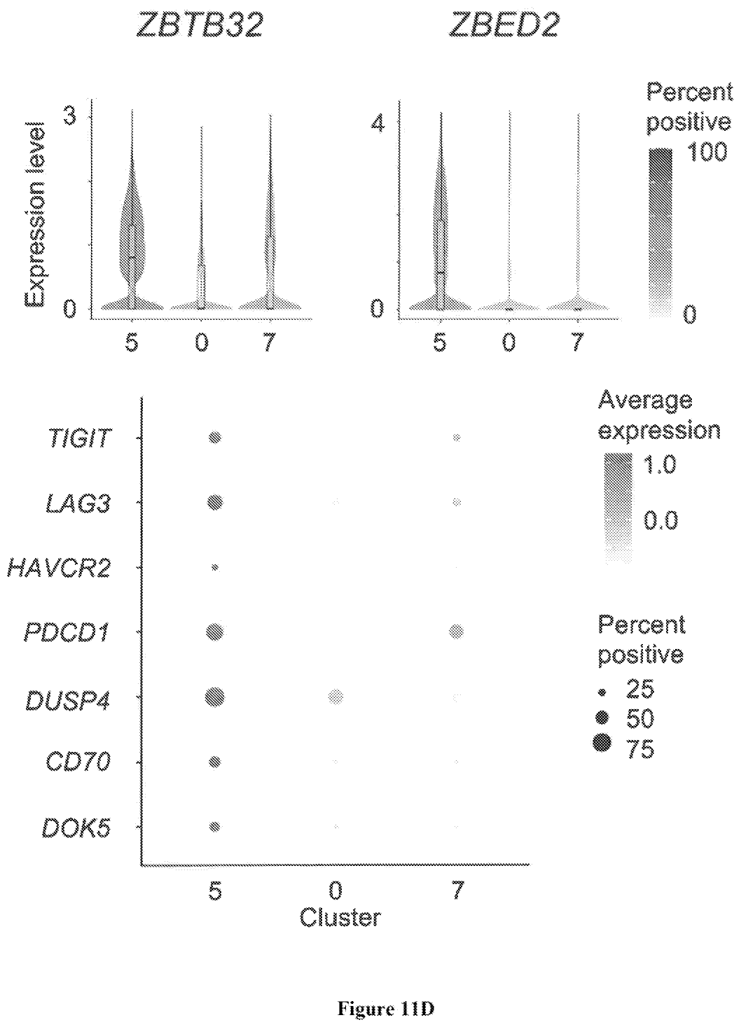

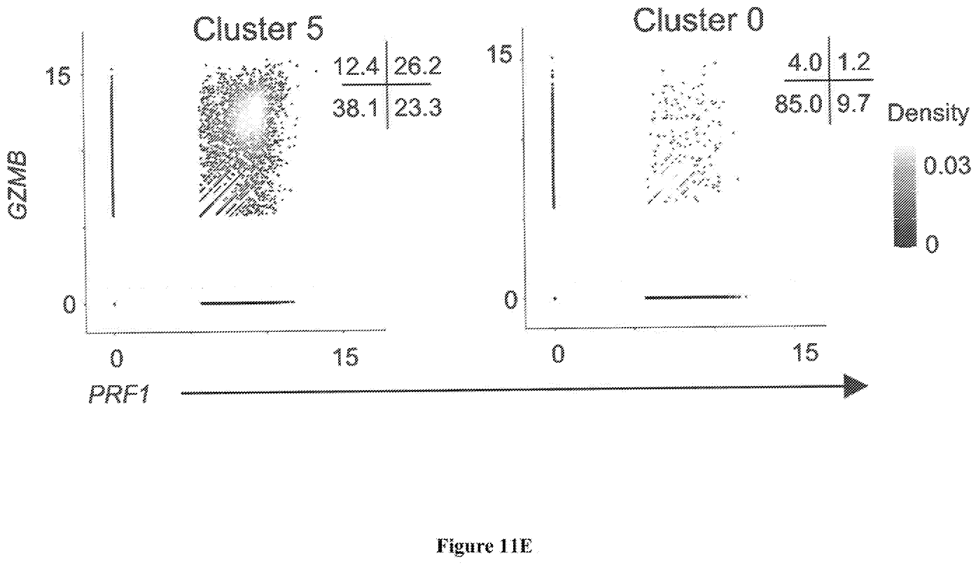

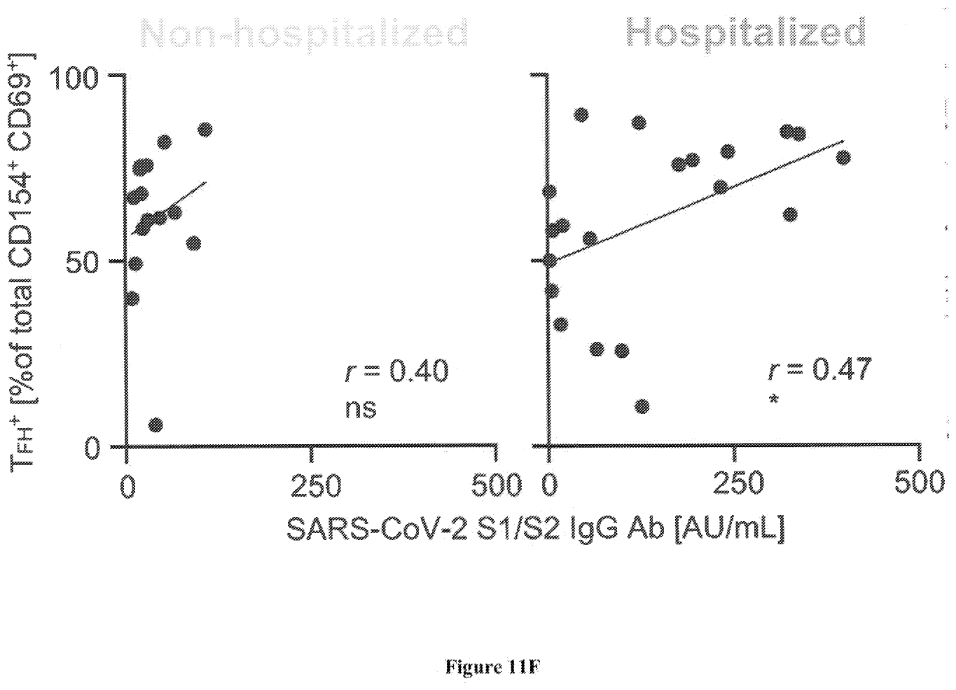

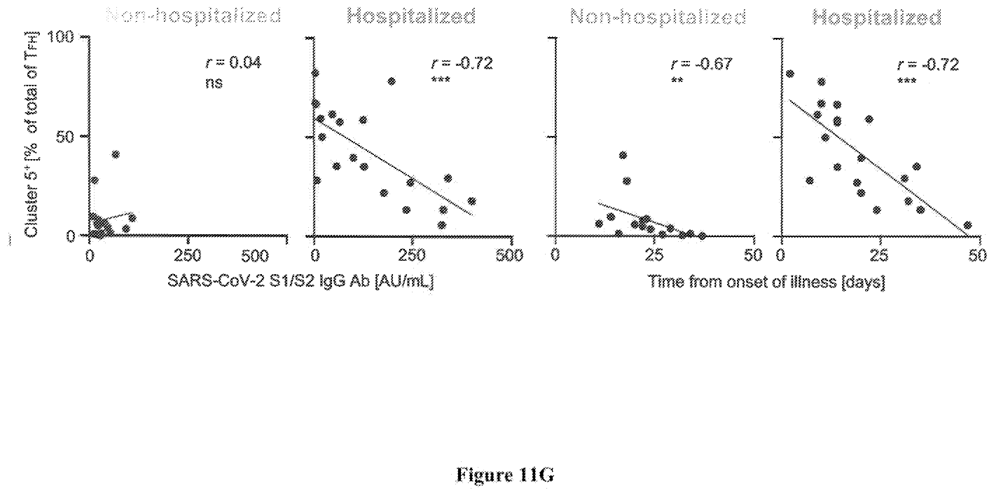

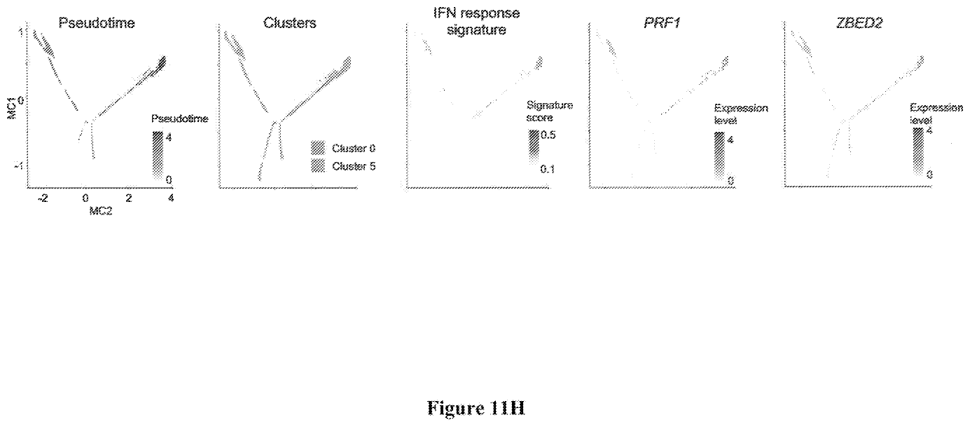

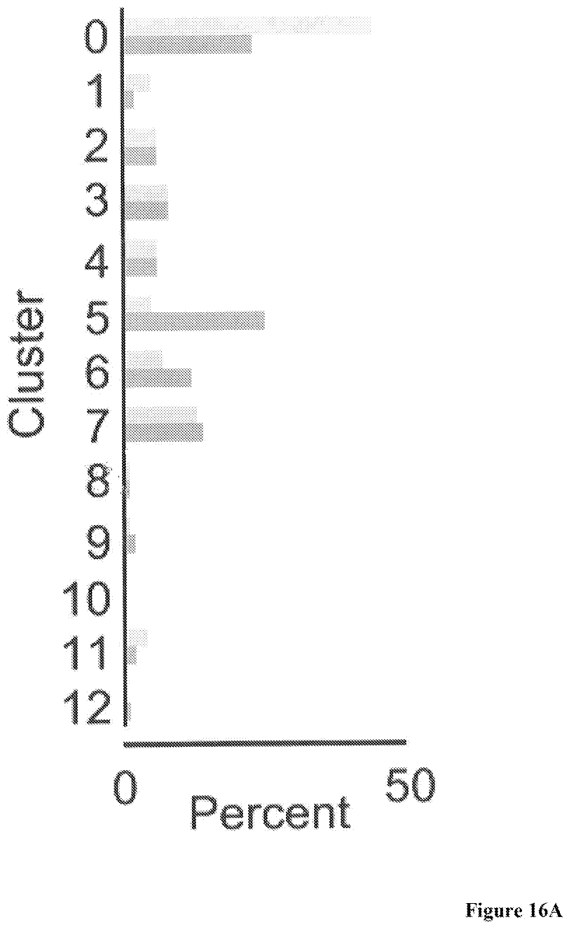

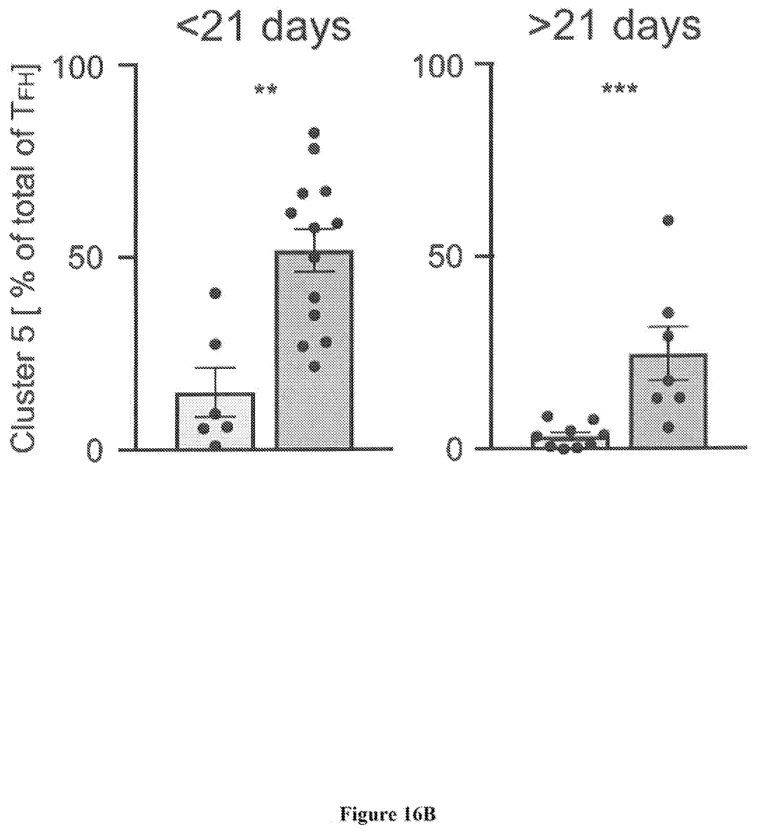

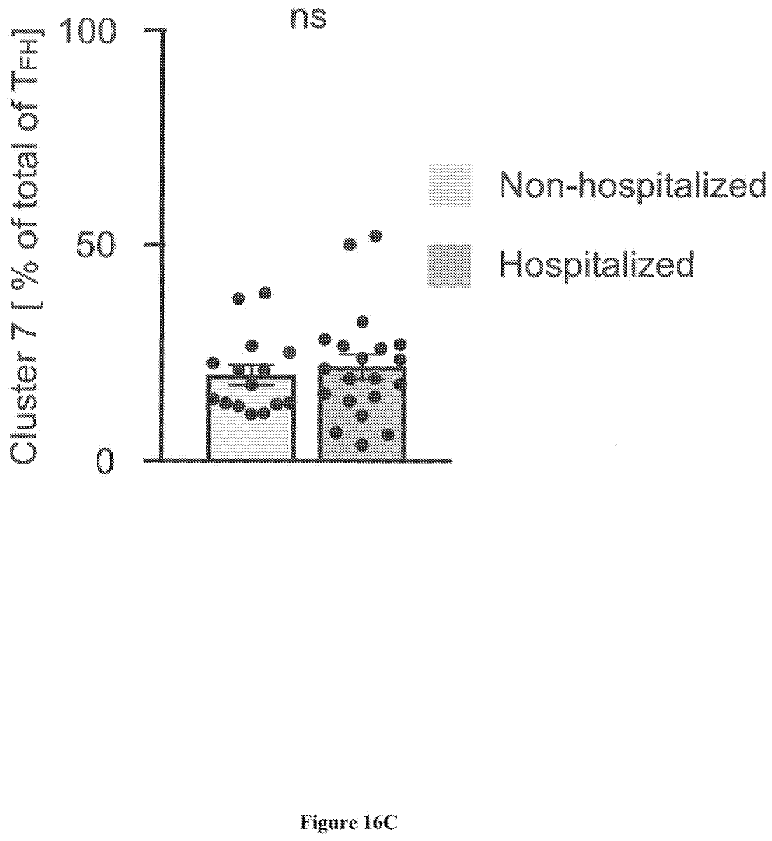

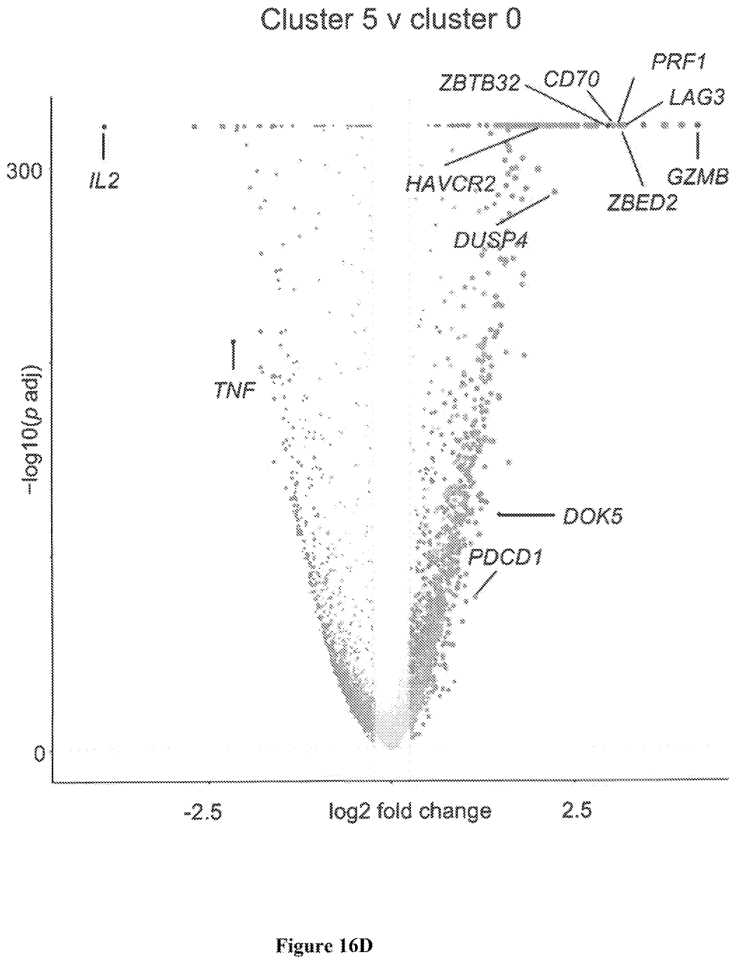

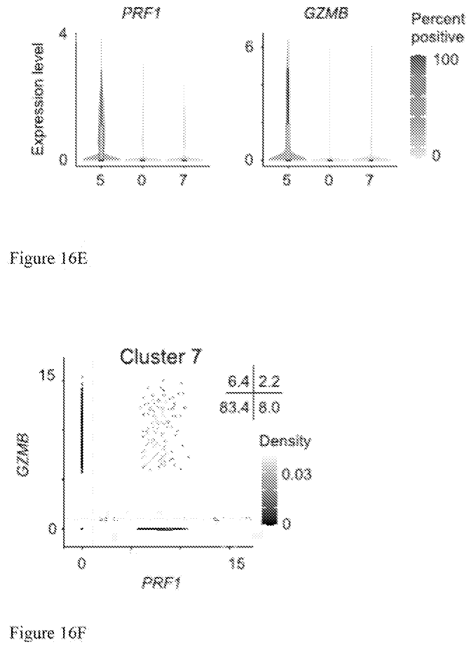

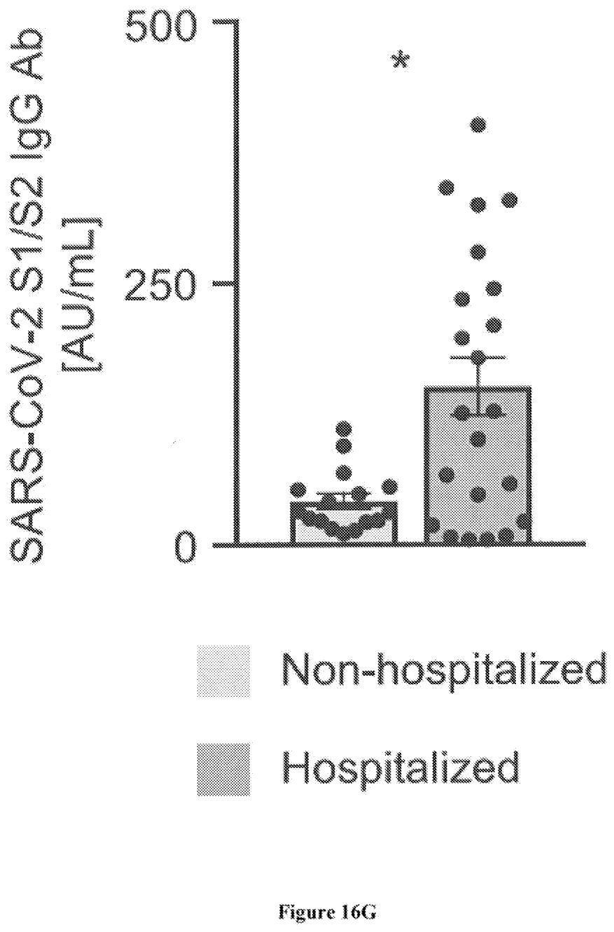

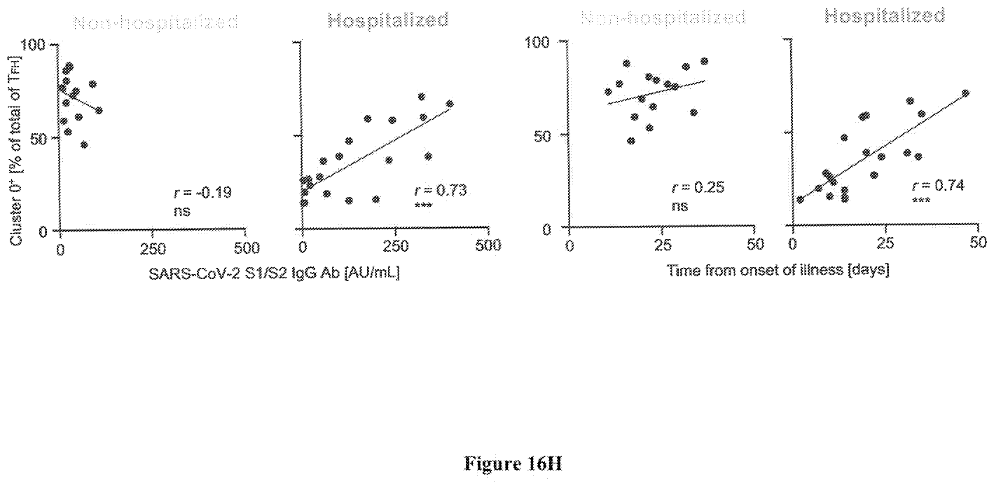

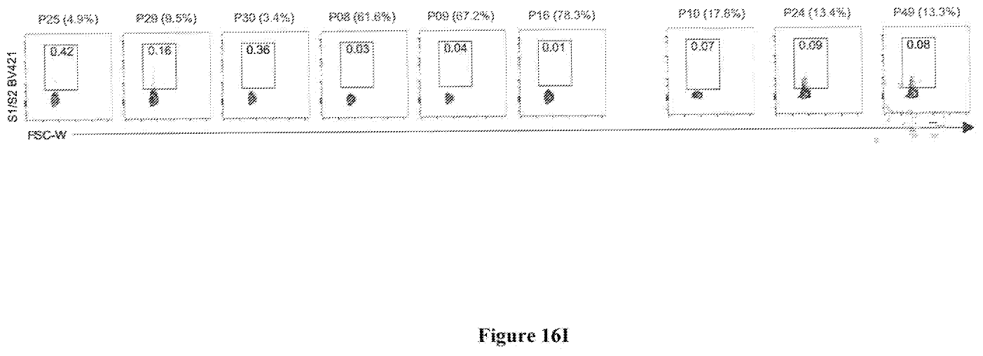

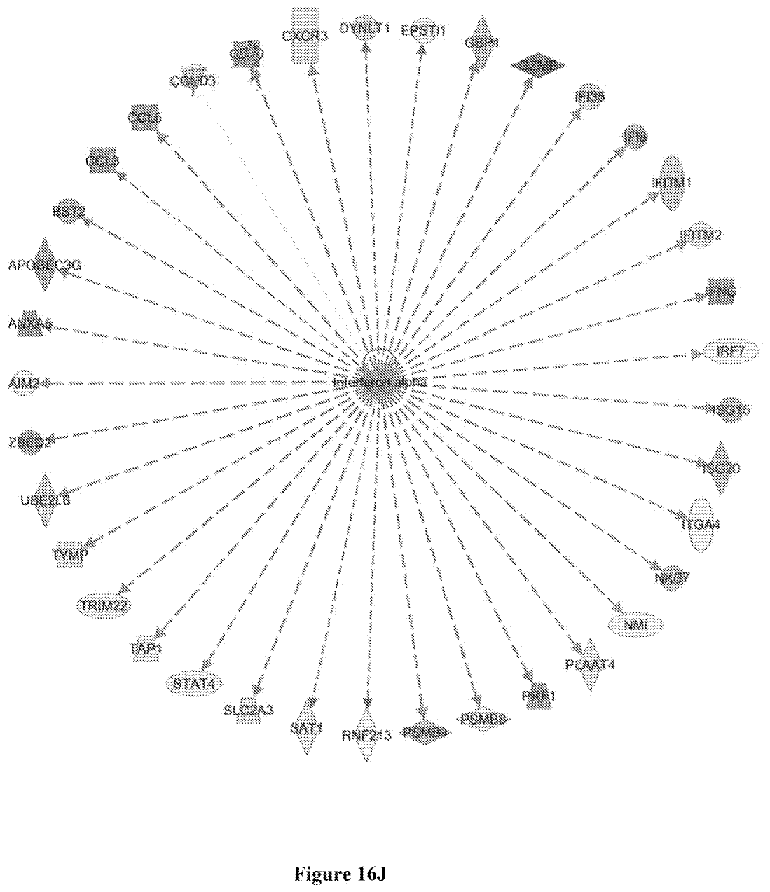

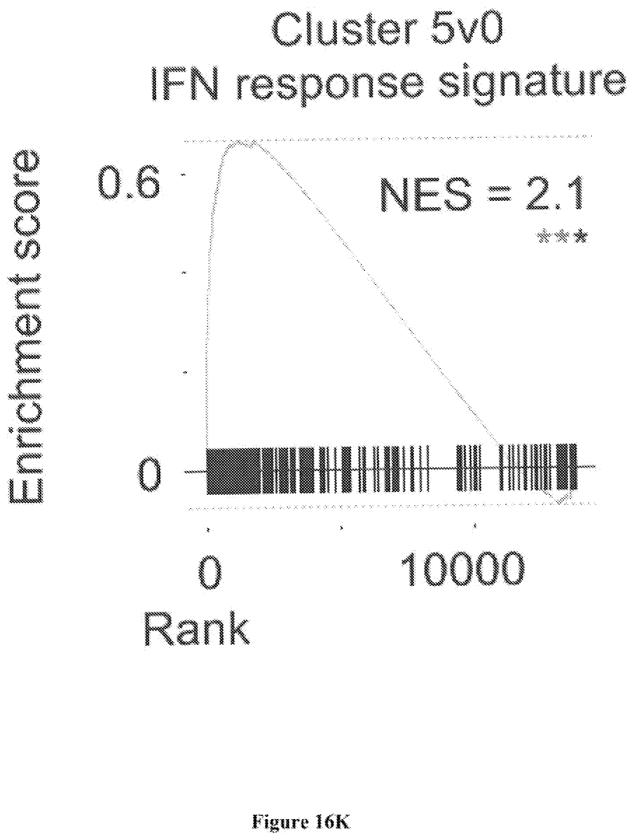

We next assessed if the proportions of SARS-CoV-2-reactive CD4+ T cells in any cluster were greater or lower in hospitalized COVID-19 patients when compared to non-hospitalized patients. Unsupervised clustering of patients, based on the proportions of SARS-CoV-2-reactive CD4+ T cells in different clusters, showed that patients with an increased proportion of TFH cells in cluster 0 clustered distinctly from those with increased proportions of TFH cells in cluster 5 or CD4-CTL cells (cluster 6) (FIG. 11A). The total frequency of SARS-CoV-2-reactive CD4+T cells with a TFH profile (cluster 0, 5, and 7) was not significantly different between hospitalized and non-hospitalized COVID-19 patients (FIG. 11B). However, the relative proportion of TFH cells in cluster 5 was significantly greater in hospitalized patients (severe disease) compared to non-hospitalized patients (mild disease), and the inverse was observed for the proportion of TFH cells in cluster 0 (FIGS. 11C and 16A). This pattern was maintained irrespective of whether the patients' samples were analyzed early (<3 weeks from symptom onset) or later (>3 weeks) in the course of illness (FIG. S3B). Notably, the proportion of TFH cells in cluster 7 was not significantly different between hospitalized and non-hospitalized COVID-19 patients (FIG. 16C).

To determine the transcriptional features that differentiated SARS-CoV-2-reactive TFH cells present in cluster 5 from those in cluster 0, we performed single-cell differential gene expression analysis (FIG. 16D). Transcripts encoding for transcription factors zinc finger BED-type-containing 2 (ZBED2) and zinc finger and BTB domain-containing protein 32(ZBTB32) were enriched in TFH cells in cluster 5 and were also expressed at significantly higher levels in hospitalized COVID-19 patients (FIGS. 11D and 16D). ZBTB32, also known as PLZP, belongs to a broad-complex, tramtrack and bric-a'-brac zinc finger (BTB-ZF) family of transcriptional repressors like PLZF, B-cell lymphoma 6 (BCL6), and T-helper-inducing POZ-Kruppel-like factor (ThPOK) and has been shown to play a role in impairing anti-viral immune responses by negatively regulating T cell proliferation, cytokine production, and development of long-term memory cells (Piazza et al., 2004; Shin et al., 2017). ZBED2, a novel zinc finger transcription factor without a mouse ortholog, has been linked to T cell dysfunction in the context of anti-tumor immune response (Li et al., 2019) and more recently shown to repress expression of IFN target genes (Somerville et al., 2020). In support of potential dysfunctional properties of the cells in the TFH cluster 5, we found increased expression of several transcripts encoding for molecules linked to inhibitory function, like TIGIT, LAG3, TIM3, and PD1 (Thommen and Schumacher, 2018), and to negative regulation of T cell activation and proliferation, like DUSP4 and CD70 (Huang et al., 2012; O'Neill et al., 2017) (FIGS. 11D and 16D).

Most strikingly, TFH cells in cluster 5 expressed high levels of cytotoxicity-associated transcripts (PRF1, GZMB) (FIGS. 11E, 16D, and 16E), reminiscent of the recently described cytotoxic TFH cells, which were shown to directly kill B cells and associated with the pathogenesis of recurrent tonsillitis in children (Dan et al., 2019). Of relevance, recent studies reported a striking loss of germinal center B cells in the thoracic lymph nodes and spleen of patients who died of SARS-CoV-2 infection (Kaneko et al., 2020), as well as slightly lower SARS-CoV-2 spike protein (S)-specific immunoglobulin M (IgM) antibodies in deceased COVID-19 patients (Atyeo et al., 2020). On the basis of these findings, we hypothesized that the cytotoxic TFH cells (cluster 5) observed in hospitalized COVID-19 patients may impair humoral (B cell) immune responses to SARS-CoV-2. To test this association, we assessed the correlation between the proportions of SARS-CoV-2-reactive TFH cell subsets and immunoglobulin G (IgG) antibody titers against the SARS-CoV-2 S1/S2 (S1 and S2 subunits), which was higher in hospitalized patients (FIGS. 11F, 11G, and 16G). Although the total frequency of SARS-CoV-2-reactive TFH cells (clusters 0, 5, and 7) showed a positive correlation with antibody levels in hospitalized COVID-19 patients, but not in non-hospitalized COVID-19 patients (FIG. 11F), the relative proportions of cytotoxic TFH cells (TFH cells in cluster 5) showed a strong negative correlation with anti-S1/S2 antibody levels in hospitalized COVID-19 patients (FIG. 11G). Conversely, the proportions of TFH cells in cluster 0 (noncytotoxic) were positively correlated with antibody concentrations in hospitalized COVID-19 patients (FIG. 16H). We noted that the magnitude of cytotoxic TFH response (cluster 5) also showed a significant negative correlation with the time interval between onset of illness and sample collection, suggesting that their association with antibody levels could be confounded by the timing of analysis of patients' samples (FIG. 11G). Furthermore, we did not observe this negative association between cytotoxic TFH cells and anti-S1/S2 antibody levels in non-hospitalized patients, which suggested that other mechanisms such as lower viral titers may explain the low levels of anti-S1/S2 antibodies in non-hospitalized patients. To further assess effects on B cell function, we analyzed B cells specific for SARS-CoV-2 spike protein (S1 and S2 subunits) from nine patients with varying proportion of cytotoxic TFH cells. Notably, in the hospitalized patients with high proportions of cytotoxic TFH cells (patients 08, 09, and 16), we observed a much smaller number of S1/S2-specific B cells compared to those with lower proportions of these cytotoxic TFH cells (FIG. 16I). Future longitudinal studies that examine the kinetics of T and B cell responses to SARS-CoV-2 are likely to provide more definitive and time resolved associations between cytotoxic TFH cell and antibody responses.

Next, to characterize upstream regulators that may induce the differentiation and maintenance of the cytotoxic TFH cells, we performed Ingenuity Pathway analysis (IPA) of the transcripts increased in SARS-CoV-2-reactive TFH cells in cluster 5 (cytotoxic) when compared to those in cluster 0 (Tables S3D and S3E). Surprisingly, we found that type 1 and 2 IFNs emerged as the top upstream activators of genes enriched in the cytotoxic TFH cluster (FIG. 16J). GSEA confirmed that IFN response signatures were also significantly enriched in the cytotoxic TFH cluster (cluster 5) (FIG. S3K). Single-cell trajectory analysis showed that a large fraction of cytotoxic TFH cells (cluster 5) followed a separate trajectory from cluster 0 cells (FIG. 11H), and cells in this track were enriched for the IFN response signature. In addition, we found that transcripts encoding perforin (PRF1) and the transcription factor ZBED2 were also enriched in the cytotoxic TFH cell trajectory, which suggested the hypothesis that ZBED2 may contribute to the differentiation or function of cytotoxic TFH cells, although further studies will be needed to verify this.

### Example 10: Massive Clonal Expansion of CD4-CTLs

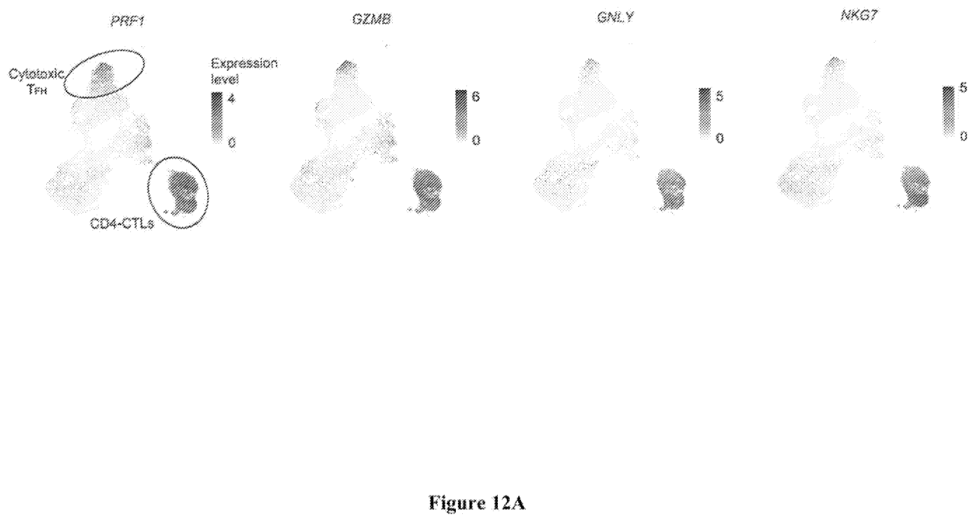

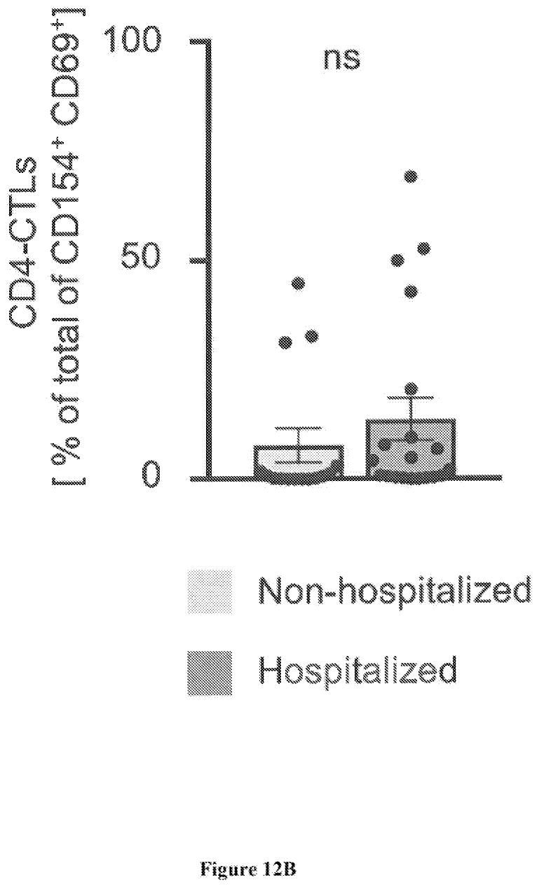

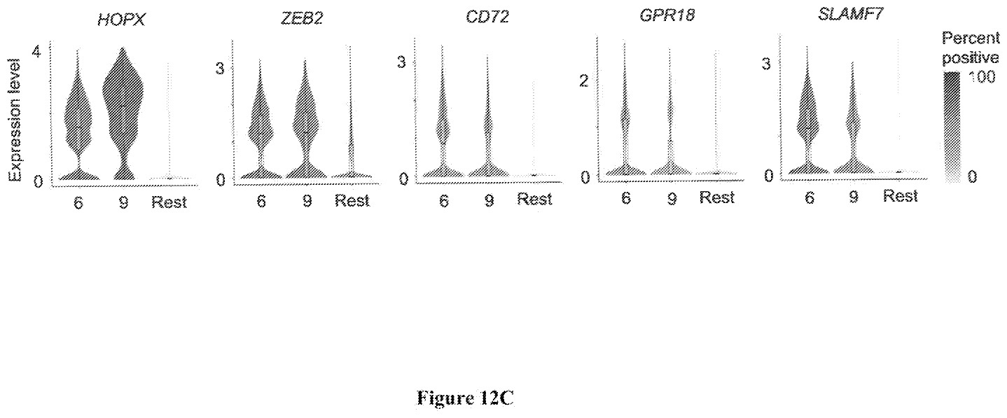

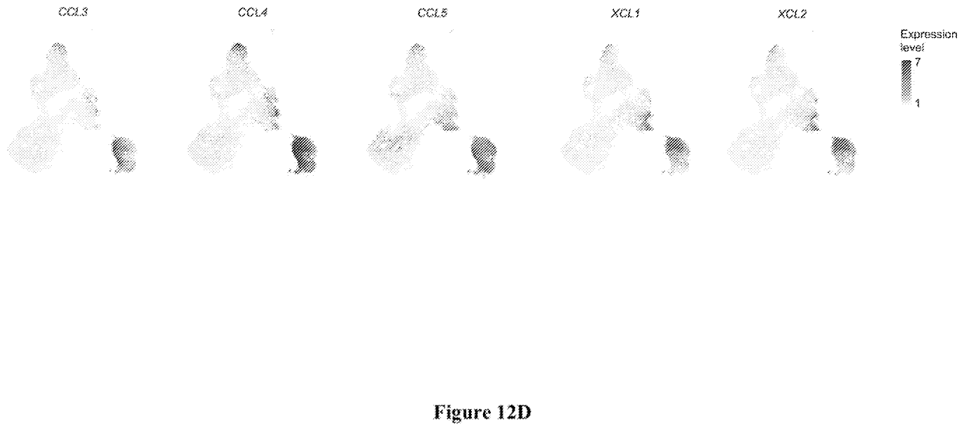

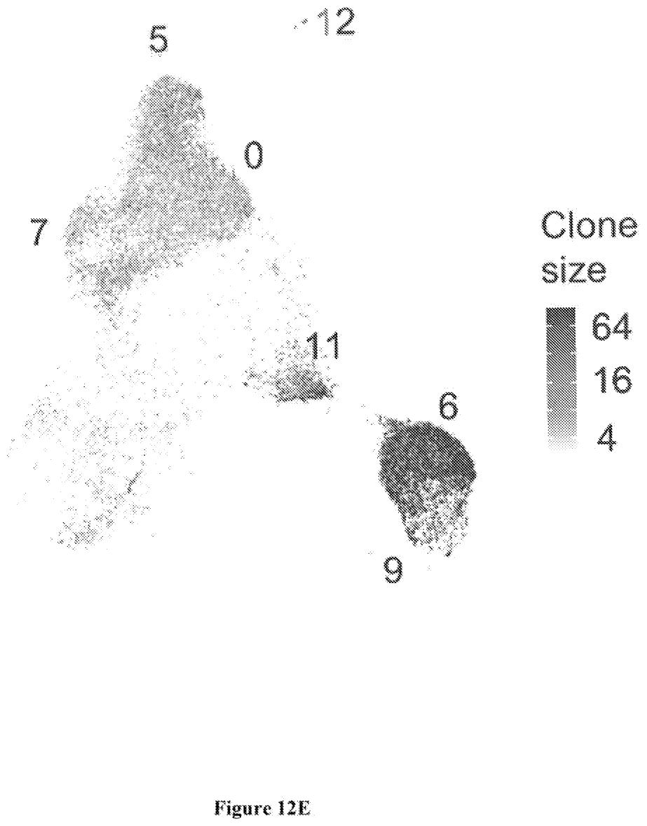

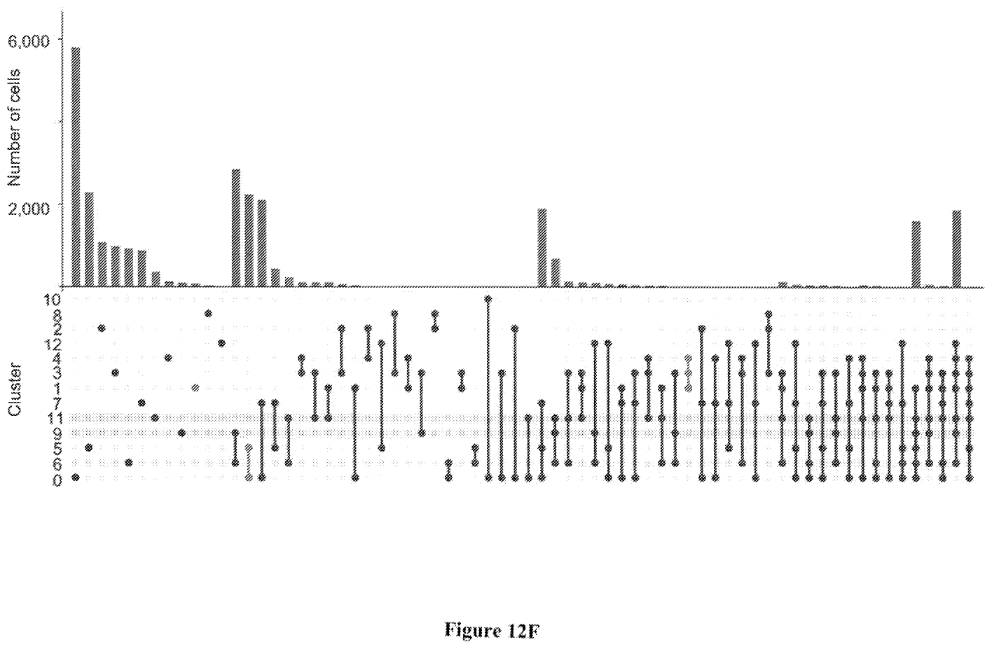

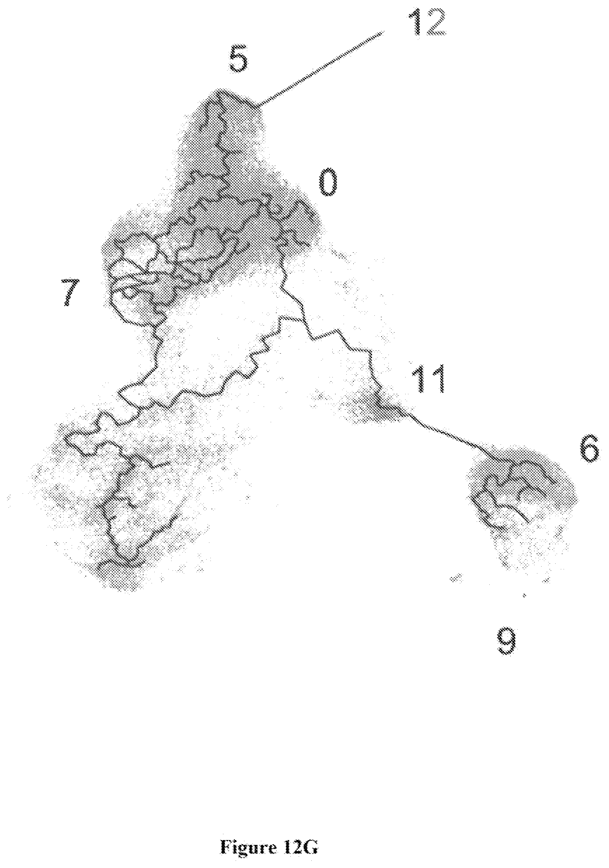

While T cells with cytotoxic function are thought to predominantly consist of conventional MHC class I-restricted CD8+ T cells, MHC class II-restricted CD4+ T cells with cytotoxic potential (CD4-CTLs) have also been reported in several viral infections in humans and are associated with better clinical outcomes (Cheroutre and Husain, 2013; Juno et al., 2017; Meckiff et al., 2019; Weiskopf et al., 2015a). Paradoxically, in SARSCoV-2 infection, we find that cells in the CD4-CTL clusters (FIG. 12A; cluster 6 and 9) were present at higher frequencies in some hospitalized COVID-19 patients compared to non-hospitalized patients, potentially contributing to disease severity, although we observed substantial heterogeneity in responses among patients (FIGS. 12B and 11A).

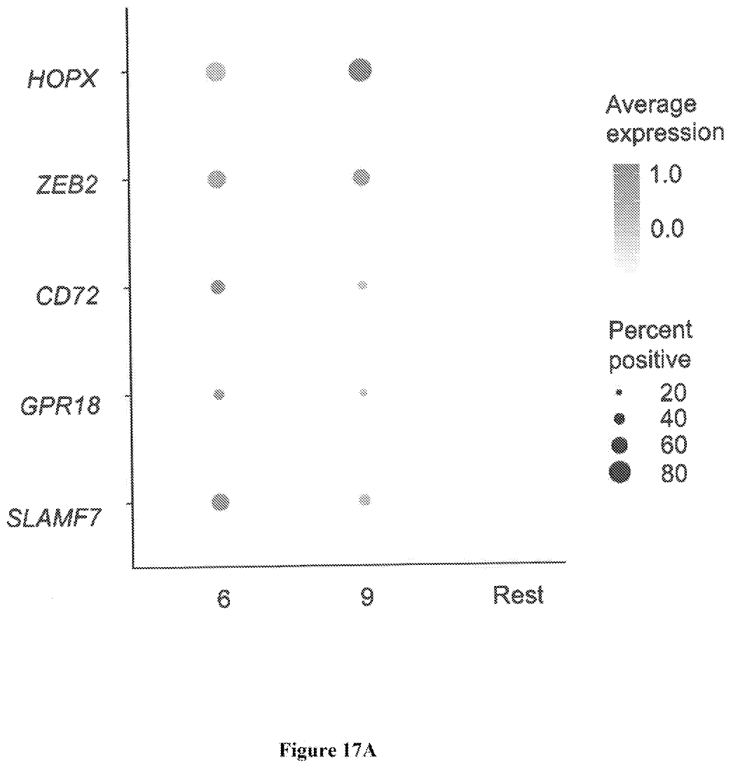

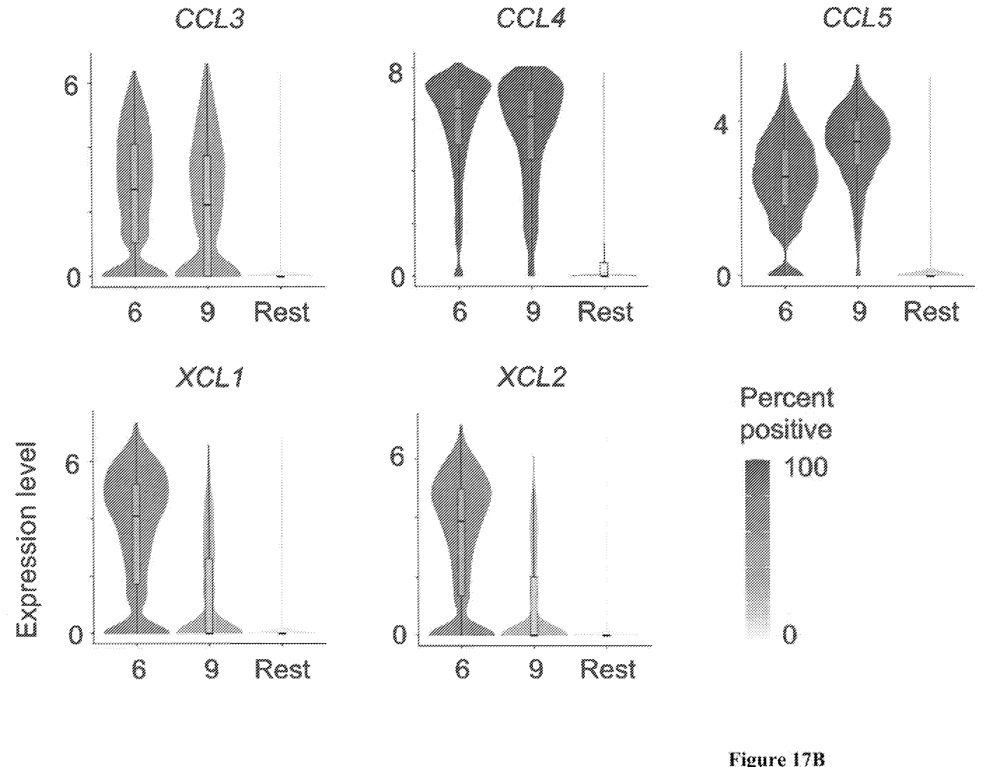

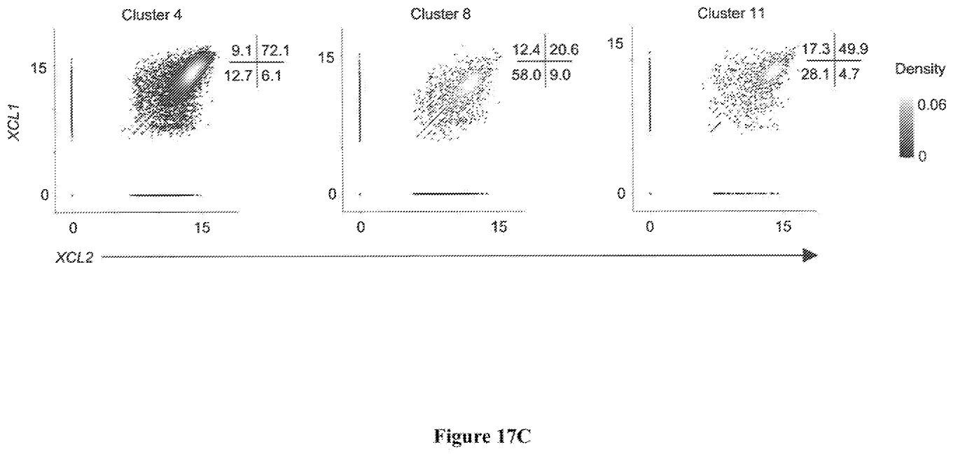

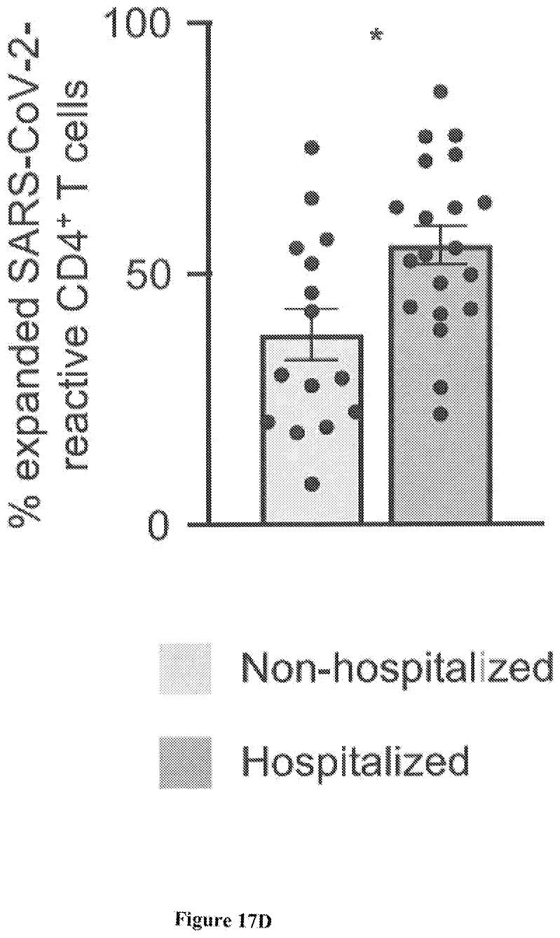

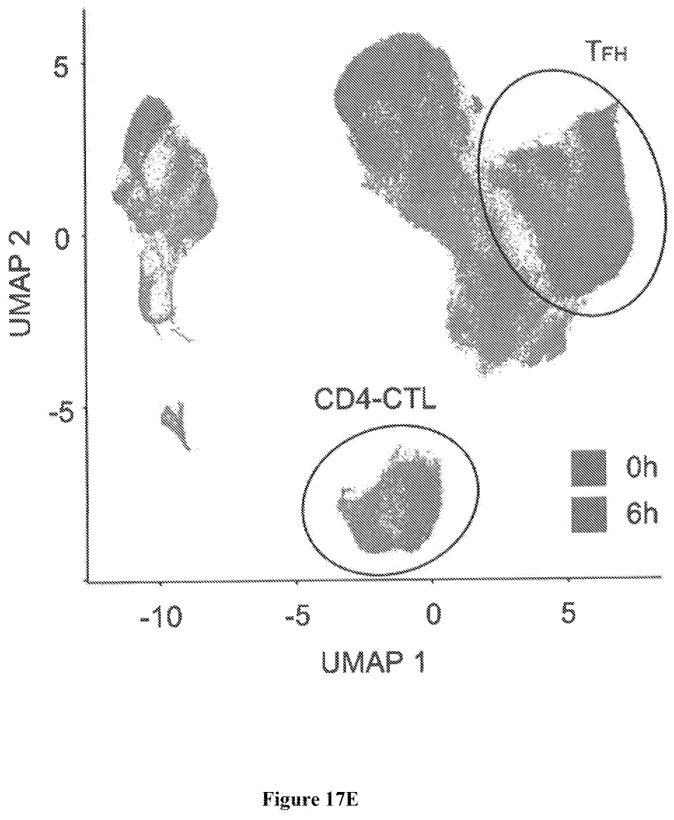

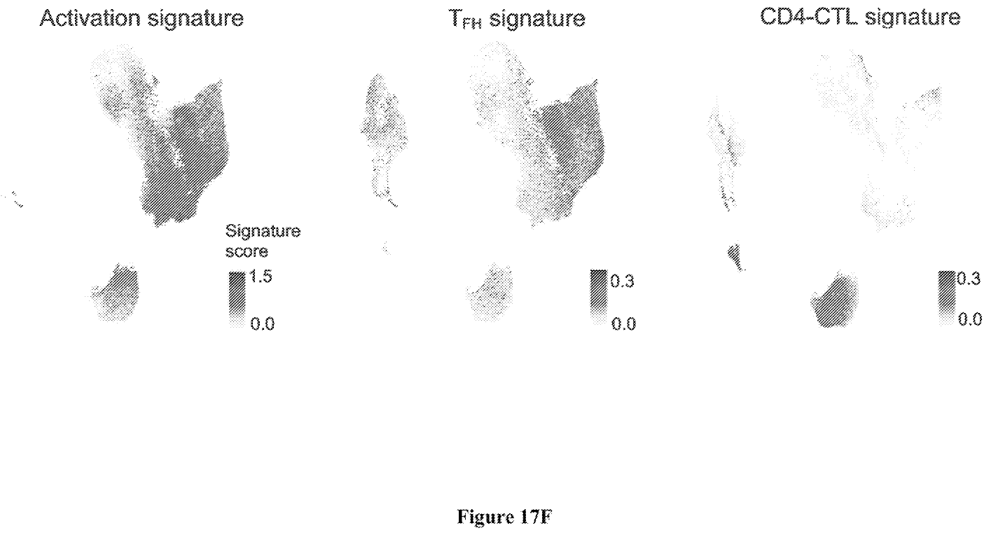

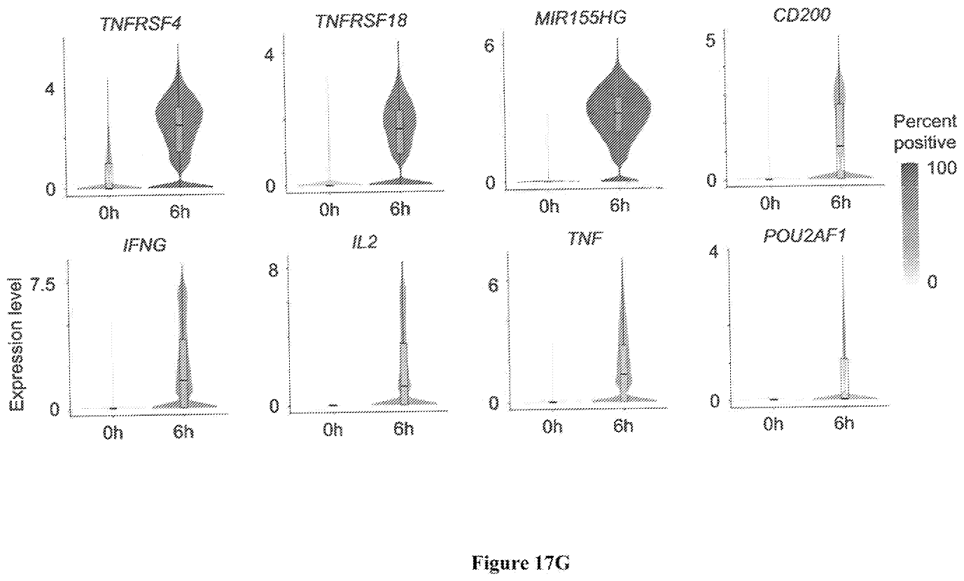

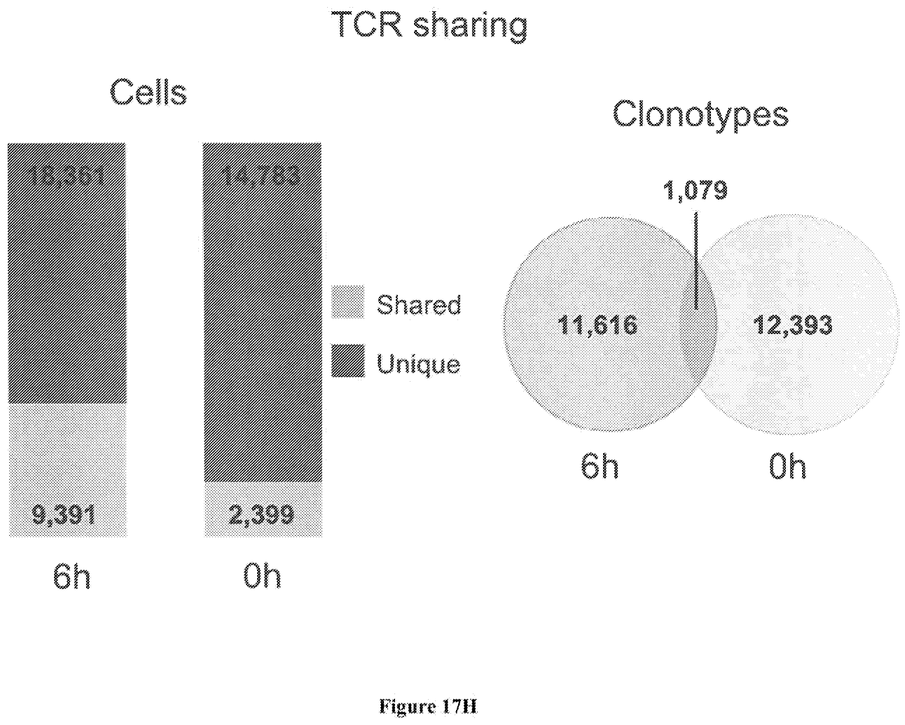

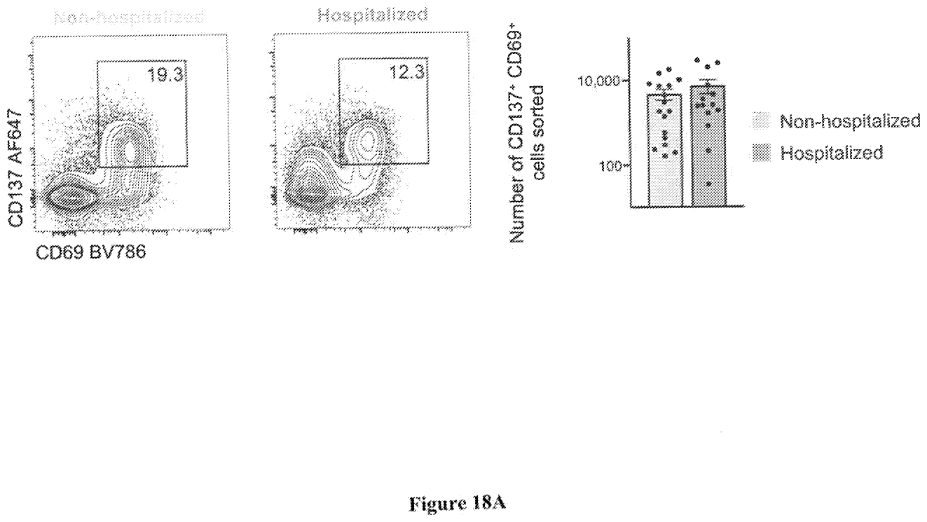

Interrogation of the transcripts enriched in the CD4-CTL subsets pointed to several interesting molecules and transcription factors that are likely to play an important role in their maintenance and effector function. These include molecules like CD72 and GPR18 that are known to enhance T cell proliferation and maintenance of mucosal T cell subsets, respectively (Jiang et al., 2017; Wang et al., 2014) (FIGS. 4C and S4A). Additional examples include transcription factors HOPX and ZEB2 (FIGS. 12C and 17A) that have been shown to positively regulate effector differentiation, function, persistence, and survival of T cells (Albrecht et al., 2010; Omilusik et al., 2015). Besides cytotoxicity associated transcripts, the CD4-CTL subsets (clusters 6 and 9) and cytotoxic TFH cells (cluster 5) were highly enriched for transcripts encoding for a number of chemokines like CCL3 (also known as macrophage inflammatory protein [MIP]-1a), CCL4 (MIP-1b), and CCL5 (FIGS. 12D and 15F); these chemokines play an important role in the recruitment of myeloid cells (neutrophils, monocytes, macrophages), NK cells, and T cells expressing C—C type chemokine receptors (CCR)1, CCR3, and CCR5 (Hughes and Nibbs, 2018). The CD4-CTL subset in cluster 6 and cytotoxic TFH cells (cluster 5) also expressed high levels of transcripts encoding for chemokines XCL1 and XCL2 (FIGS. 12D, 17B, and 17C) that specifically recruit XCR1-expressing conventional type 1 dendritic cells (cDC1) to sites of immune responses where they play a key role in promoting the CD8+ T cell responses by antigen cross-presentation (Lei and Takahama, 2012). Overall, the transcriptomic features of SARS-CoV-2-reactive CD4-CTLs and cytotoxic TFH cells suggest that they are likely to play an important role in orchestrating immune responses by recruiting innate immune cells to enhance CD8+T cell responses, while also directly mediating cytotoxic death of MHC class II-expressing virally infected cells.

The recovery of paired TCR sequences from individual single cells enabled us to link transcriptome data to clonotype information and evaluate the clonal relationship between different CD4+ T cell subsets as well as determine the nature of subsets that display greatest clonal expansion. In SARS-CoV-2 infection, hospitalized patients were characterized by large clonal expansion of the virus-reactive CD4+ T cells (mean of 55.8%); in contrast, in non-hospitalized patients, recovered TCRs were less clonally expanded (mean of 38.0%) (FIG. S4D). Among SARS-CoV-2-reactive CD4+ T cells, CD4− CTL subsets (clusters 6 and 9) displayed the greatest clonal expansion (>75% of cells were clonally expanded), indicating preferential expansion and persistence of CD4-CTLs in some patients with COVID-19 illness (FIG. 12E and. Analysis of clonally expanded SARS-CoV-2-reactive CD4+ T cells from COVID-19 patients showed extensive sharing of TCRs between cells in clusters 6 and 9, as well as those in cluster 11 (FIG. 12F), which, notably, was enriched for the expression of XCL1 and XCL2 transcripts and also for cytotoxicity-associated transcripts, albeit at lower levels compared to the established CD4-CTL clusters (FIGS. 12D and 17C and. Thus, cells in cluster 11 are likely to be an intermediate transition population, a hypothesis supported by single-cell trajectory analysis that showed potential temporal connection and transcriptional similarity between these subsets (FIG. 12G).

Initial reports in patients with acute COVID-19 have suggested that circulating T cells that express activation markers such as CD38, HLA-DR, and PD-1 ex vivo (without in vitro peptide stimulation) are enriched for SARS-CoV-2-reactive T cells (Braun et al., 2020; Thevarajan et al., 2020). However, a recent study indicated that bystander T cells reactive to other antigens (e.g., CMV and EBV) can also express these activation markers, likely to be non-specifically activated without TCR engagement (Sekine et al., 2020). Thus, studies in active SARS-CoV-2 infection that just examine T cells expressing activation markers are not likely to reveal the full potential effector function of SARS-CoV-2-reactive T cells. To determine the specificity and molecular features of such T cells expressing activation markers ex vivo, we isolated CD38high HLA-DRhigh PD-1+ memory CD4+ T cells from hospitalized COVID-19 patients and performed single-cell transcriptome and TCR sequence analysis of >20,000 cells. CD4+ T cells expressing activation markers ex vivo clustered distinctly from the SARS-CoV-2-reactive CD4+ T cells, which were isolated following in vitro stimulation with SARS-CoV-2 peptides for 6 h (FIG. 17E). The CD4+ T cells expressing activation markers ex vivo displayed reduced activation and TFH signature scores and had lower expression of transcripts encoding effector cytokines (IFN-g, IL-2, TNFa), activation markers (OX40), and TFH associated genes (CD200, POU2AF1) (FIGS. 17F and 17G). Furthermore, by comparison of single-cell TCR sequences, we found that 33.8% of SARS-CoV-2-reactive CD4+ T cells shared clonotypes with CD4+ T cells expressing activation markers ex vivo, and 12.2% of CD4+ T cells expressing activation markers ex vivo shared their TCRs with SARS-CoV-2-reactive CD4+ T cells (FIG. 17H). Our findings indicate that using surface activation markers as a strategy to enrich for SARS-CoV-2-reactive T cells without SARS-CoV-2 peptide stimulation (ARTE assay) may not capture the full spectrum of SARS-CoV-2-reactive T cells, like TFH biology and their cytokine profiles, although the transcriptomic features of such in vitro activated cells may be affected by antigen-presenting cells present in the cultures.

### Example 11: SARS-CoV-2-Reactive T_{REG} Cells are Reduced in Hospitalized COVID-19 Patients

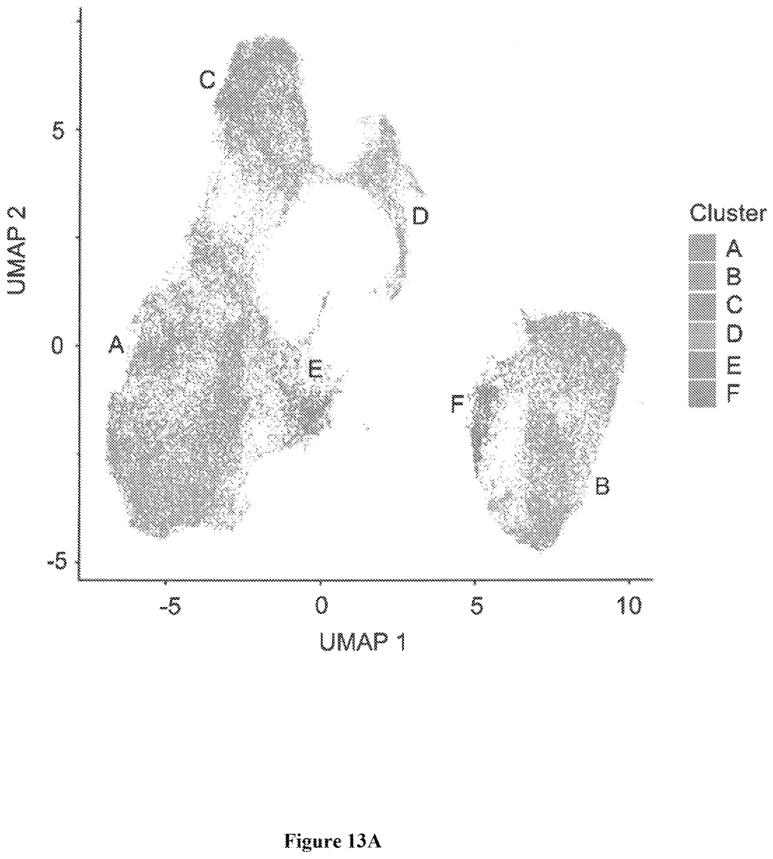

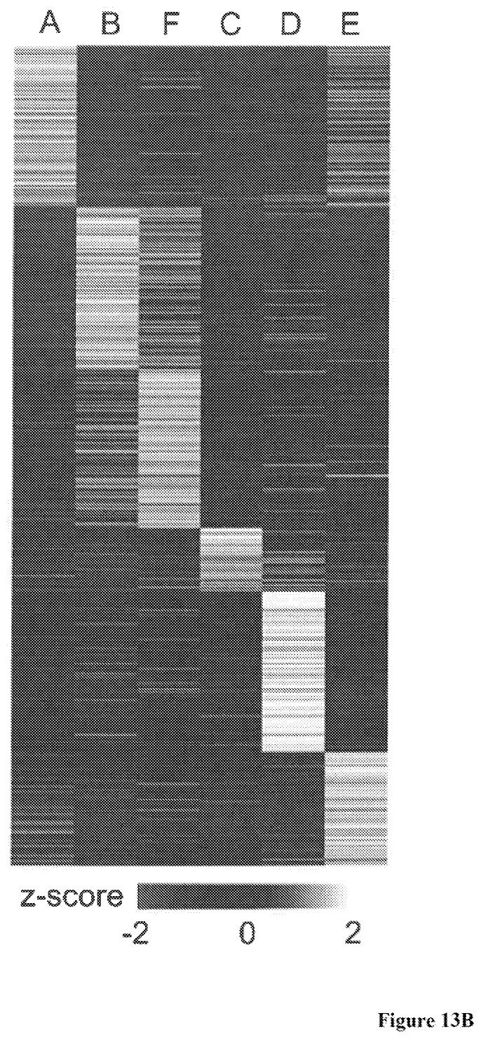

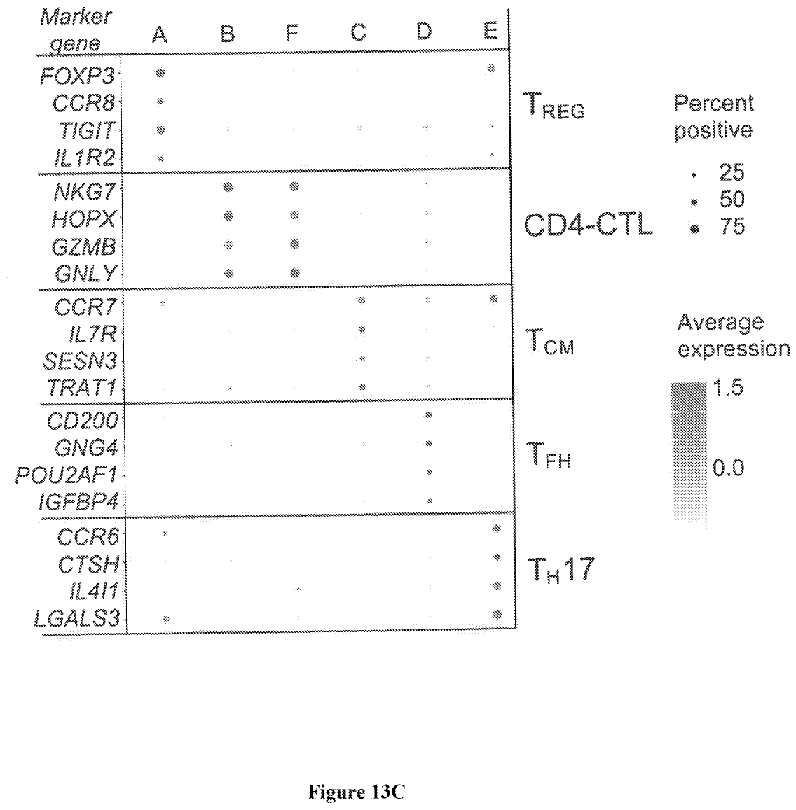

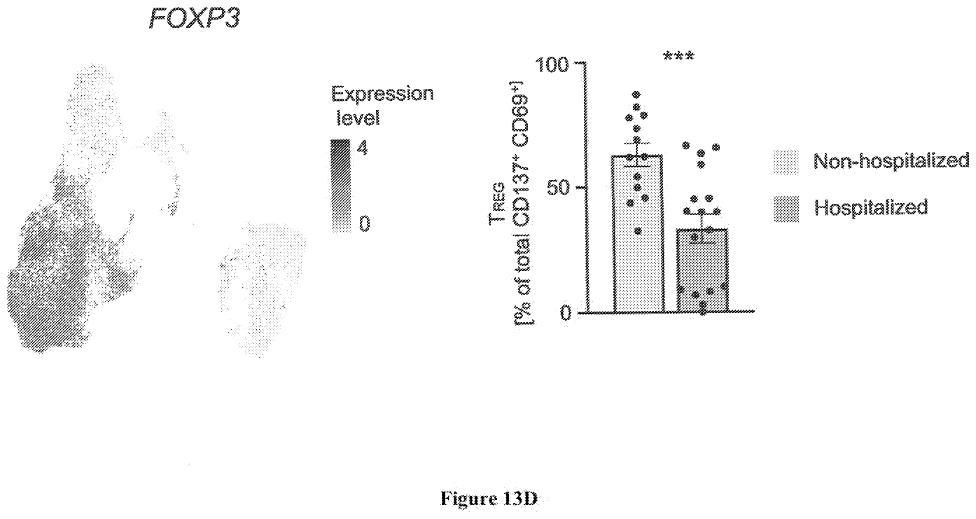

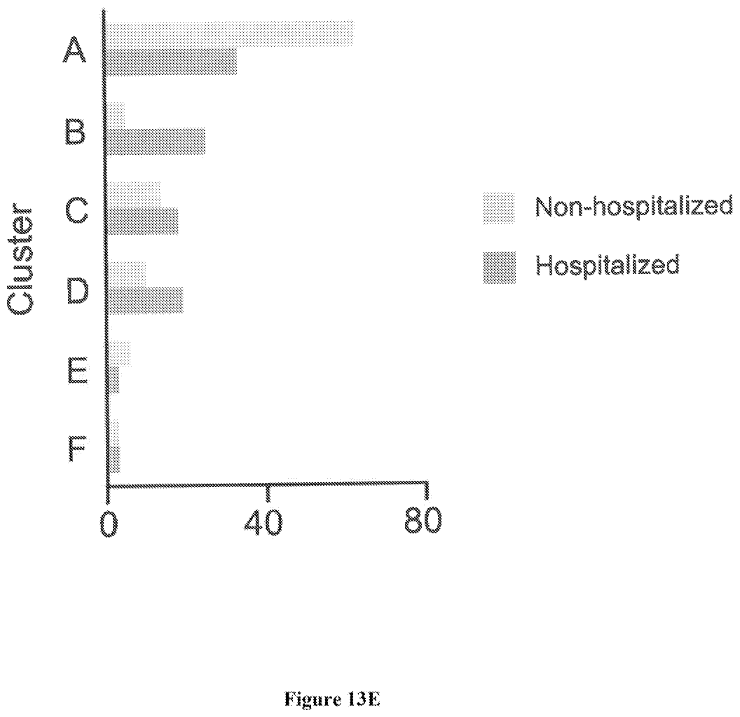

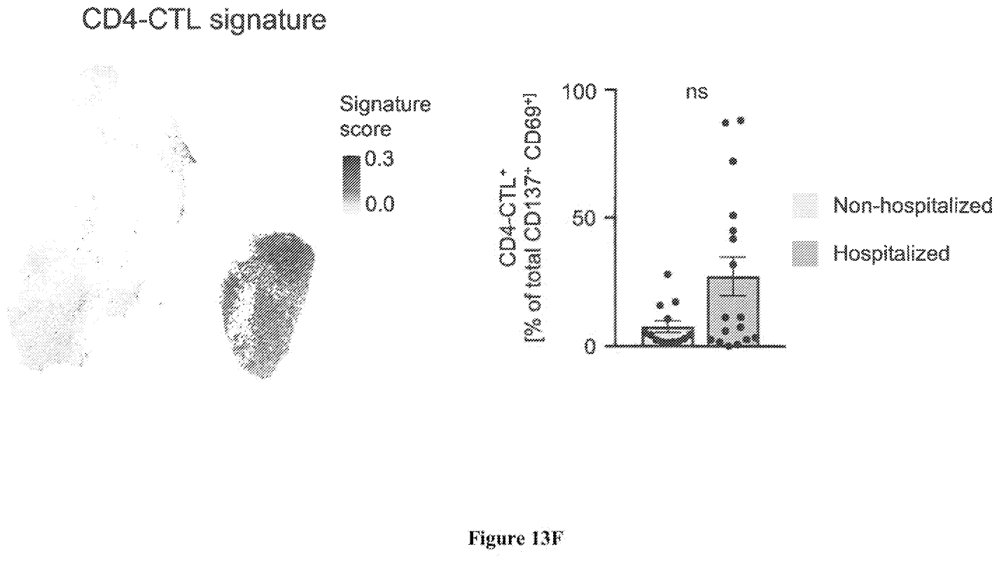

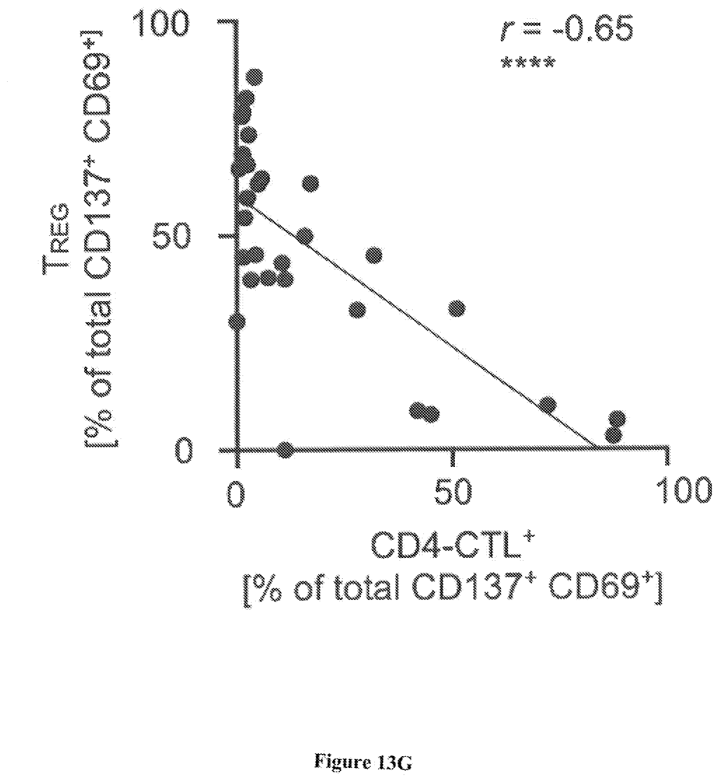

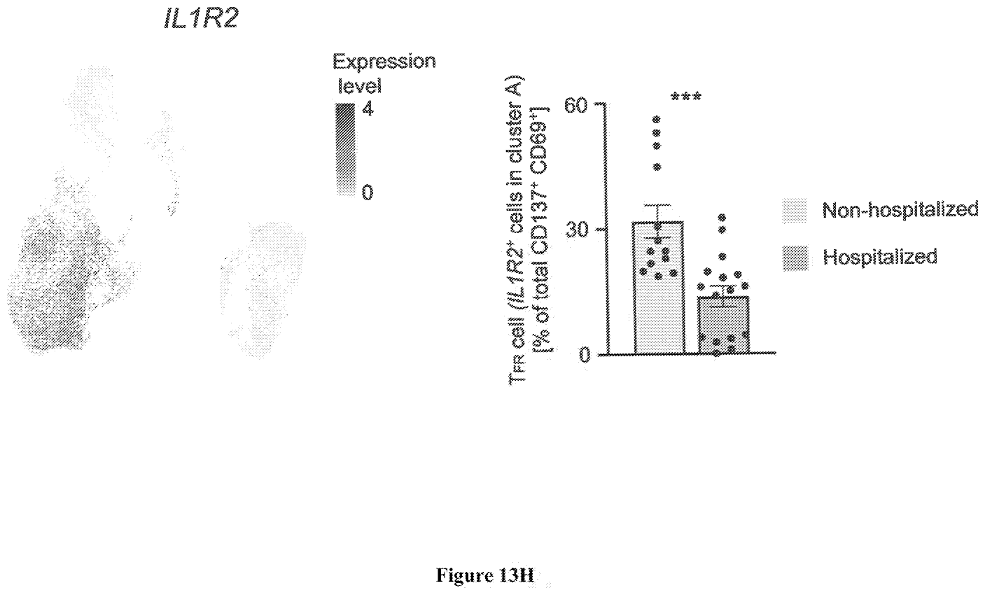

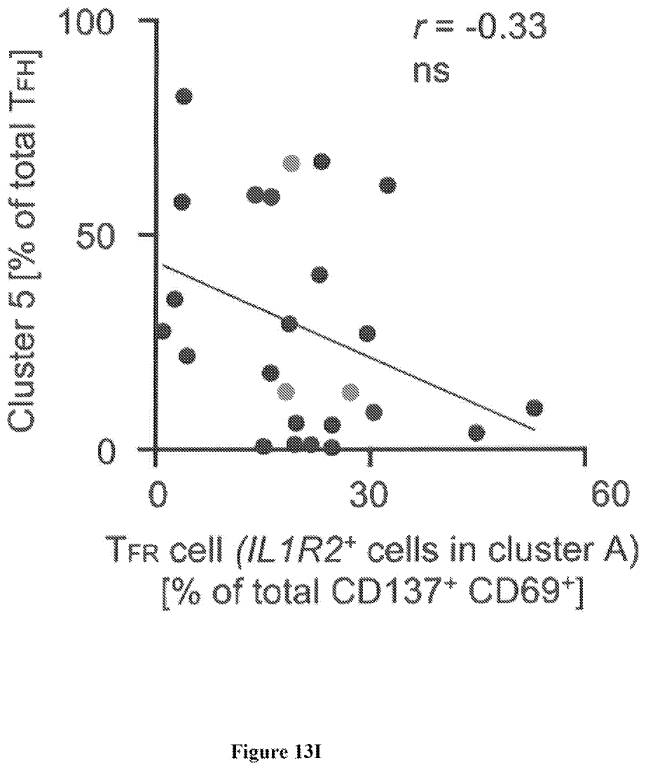

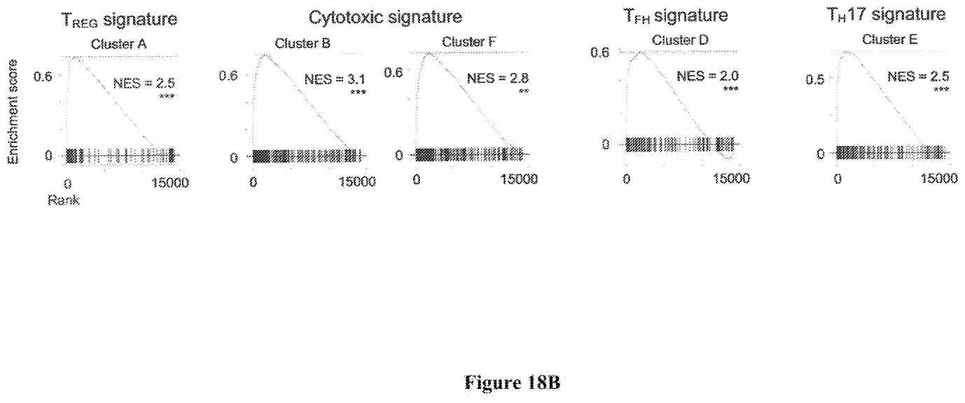

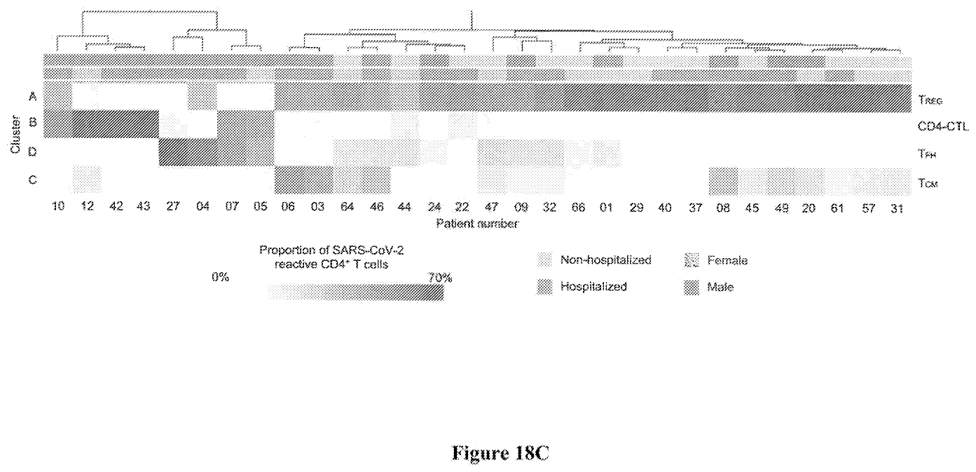

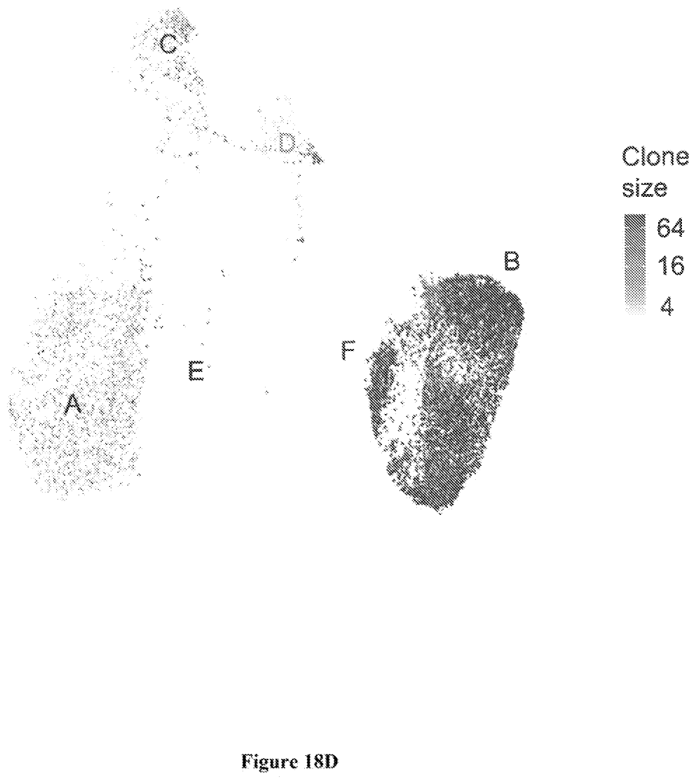

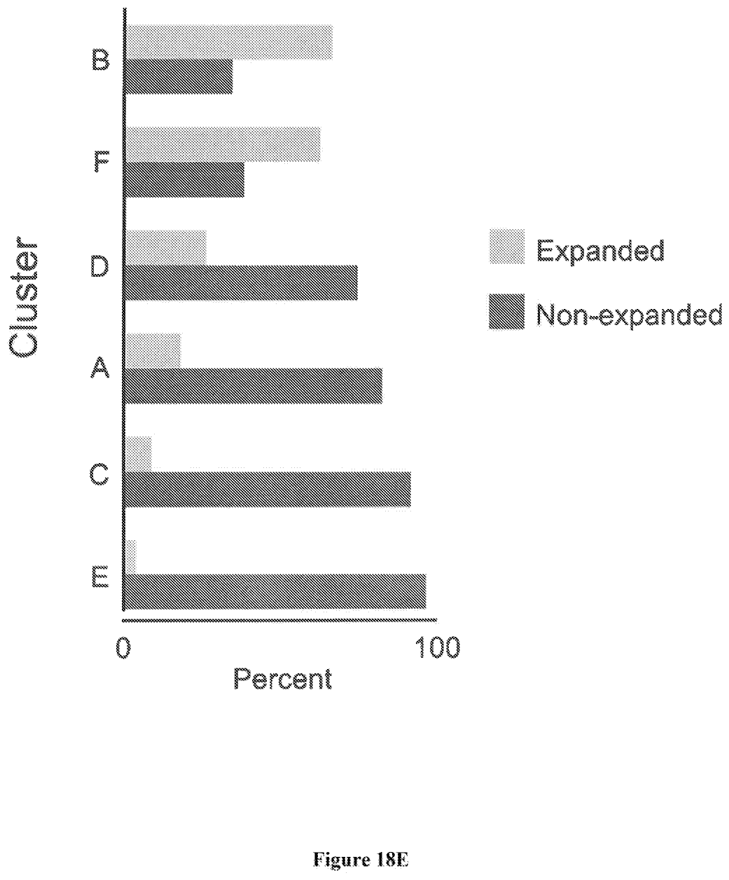

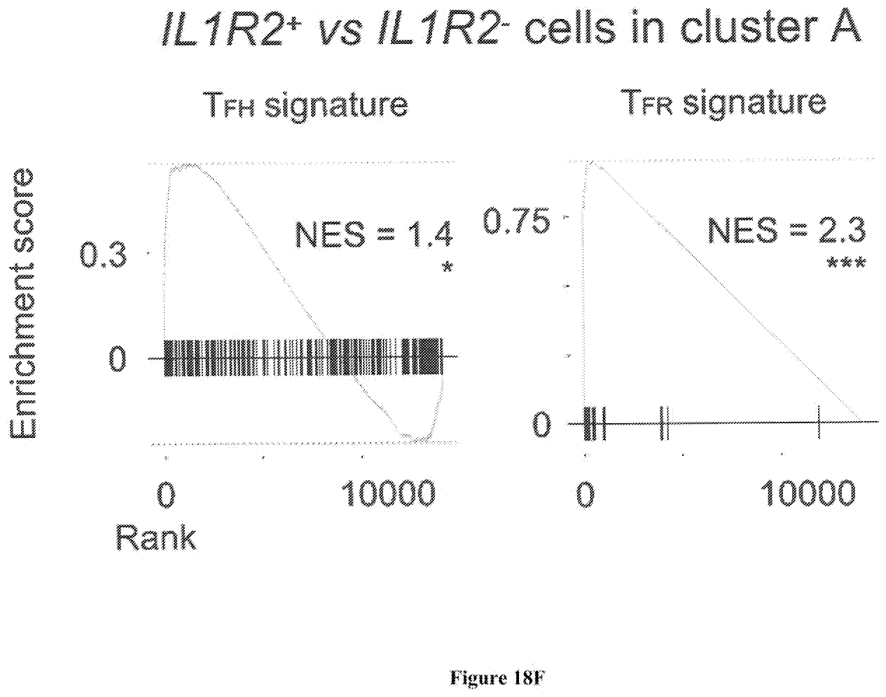

In order to capture SARS-CoV-2-reactive CD4+ T cells that may not upregulate the activation markers (CD154 and CD69) after 6 h of in vitro stimulation with SARS-CoV-2 peptide pools, we stimulated PMBCs from the same cultures for a total of 24 h (see STAR Methods) and captured cells based on co-expression of activation markers CD137 (4-1BB) and CD69, a strategy that allowed us to additionally capture antigen-specific regulatory T cells (T_{REG}) (Bacher et al., 2016) (FIGS. 13A and 18A). Our analysis of a total of 38,519 single-cell CD4+ T cell transcriptomes revealed 6 distinct clusters (FIGS. 13A-13C). The TFH subset (cluster D) was detectable at relatively lower frequencies in the 24 h condition, though they represented the major CD4+ T cell subsets in the 6 h stimulation condition (FIGS. 10A and 13A). Consistent with delayed kinetics of activation of central memory T (TCM) cells, we identified a higher proportion of CD4+ T cells expressing transcripts linked to central memory cells (CCR7, IL7R, and TCF7) (cluster C) (FIGS. 10A, 13A, and 13C).

The largest cluster (cluster A) was characterized by high expression of FOXP3 transcripts, which encodes for the T_{REG} master transcription factor forkhead box P3 (FOXP3) (Rudensky, 2011) (FIGS. 13A-13D). Independent GSEA analysis showed significant positive enrichment of T_{REG} signature genes in this cluster, suggesting that cells in this cluster represented SARS-CoV-2-reactive T_{REG} cells (FIG. 18B). Notably, the proportion of cells in the T_{REG} cluster was significantly lower in hospitalized COVID-19 patients compared to non-hospitalized patients (FIGS. 13D, 13E, and 18C), suggesting a potential defect in the generation of immunosuppressive SARS-CoV-2-reactive T_{REG} cells in hospitalized patients. Consistent with our data from 6 h stimulation condition, we found that cells in the CD4-CTL clusters (clusters B and F) were present at higher frequencies in some hospitalized COVID-19 patients (FIGS. 13E, 13F, and 18C). They also showed the greatest clonal expansion compared to other clusters (FIGS. 18D an 18E), suggesting potential importance of the CD4-CTL subset in driving immune responses to SARS-CoV-2 infection.

Correlation analysis of the proportion of CD4-CTLs and T_{REG} in our 24 h dataset revealed a significant negative correlation, which indicated that patients with an impaired T_{REG} response to SARS-CoV-2 mounted a stronger CD4-CTL response (FIG. 13G). A recent study in a murine model showed that cytotoxic TFH responses are curtailed by a subset of T_{REG} cells called follicular regulatory T (TFR) cells (Xie et al., 2019). To determine if such association is observed in our datasets, we first quantified TFR cells based on the expression of IL1R2 (Eschweiler et al., 2020) from cells in the T_{REG} cluster A (FIG. 13H). Independent GSEA confirmed that IL1R2-expressing cells were significantly enriched for follicular and TFR signature genes (FIG. 18F), which indicated they represent TFR cells. Over 40% of the cells in the T_{REG} cluster expressed IL1R2; this indicates that a strong circulating TFR response is generated in SARS-CoV-2 infection. Importantly, the proportion of TFR cells was significantly lower in hospitalized COVID-19 patients (FIG. 13H) and showed a modest negative correlation with the proportion of cytotoxic TFH cells (FIG. 13I). On the basis of these findings and the known function of these T_{REG} subsets, we hypothesize that the magnitude of T_{REG} and TFR responses to SARS-CoV-2 are likely to modulate cytotoxic CD4+ T and B cell responses in COVID-19 illness, although further studies are required to confirm this hypothesis.

### Example 12: Experimental Model and Subject Details (Used in Examples 7-11) COVID-19 Patients and Samples

Ethical approval for this study from the Berkshire Research Ethics Committee 20/SC/0155 and the Ethics Committee of La Jolla Institute for Immunology (LJI) was in place. Written consent was obtained from all subjects. 22 hospitalized patients in a large teaching hospital in the south of England with SARS-CoV-2 infection, confirmed by reverse transcriptase polymerase chain reaction (RT-PCR) assay for detecting SARS-CoV-2, between April-May 2020 were recruited to the study. A further cohort of 18 participants consisting of healthcare workers who were not hospitalized with COVID-19 illness, confirmed based on RT-PCR assay or serological evidence of SARS-CoV-2 antibodies, were also recruited over the same period. All subjects provided up to 80 mL of blood for research studies. Clinical and demographic data were collected from patient records for hospitalized patients including comorbidities, blood results, drug intervention, radiological involvement, thrombotic events, microbiology, and virology results. The 22 hospitalized patients had a median age of 60 (33-82), 17 of these patients (77%) were men and this cohort consisted of 16 (73%) White British/White Other, 4 (18%) Indian, and 2 (9%) Black British patients. All hospitalized patients survived to discharge from hospital. All hospitalized patients were still symptomatic at time of blood collection, whereas some of the non-hospitalized patients (4/18) were symptom free. The 18 non-hospitalized participants had a median age of 39 (22-50), 8 (44%) of these participants were men and this cohort consisted of 15 (83%) White British/White Other, 2 (11%) Arab, and 1 (6%) Chinese participant. We noted that the median age of the non-hospitalized patients was lower than the hospitalized COVID-19 patients.

### Healthy Controls

To study HPIV, HMPV, and SARS-CoV-2-reactive CD4+ T cells from healthy non-exposed subjects (pre-COVID-19 pandemic), we utilized de-identified buffy coat samples from 5 healthy adult donors who donated blood at the San Diego Blood Bank before 2019, prior to the Covid-19 pandemic. Donors were considered to be in good health, free of cold or flu-like symptoms and with no history of Hepatitis B or Hepatitis C infection. The median age was 50 (32-71) and 4 of these patients (80%) were men. To study FLU-reactive cells, we obtained de-identified blood samples from 8 donors enrolled in the LJI Normal Blood Donor Program before and/or after (12-14 days) receiving the FLUCELVAX vaccine (September and October 2019). The median age was 37 (26-57) and 5 of these patients (63%) were women. Approval for the use of this material was obtained from the LJI Ethics Committee.

### Method Details

### PBMC Processing

Peripheral blood mononuclear cells (PBMCs) were isolated from up to 80 ml of anti-coagulated blood by density centrifugation over Lymphoprep (Axis-Shield PoC AS, Oslo, Norway) and cryopreserved in 50% decomplemented human antibody serum, 40% complete RMPI 1640 medium and 10% DMSO.

### SARS-CoV-2 Peptide Pools

Pools of lyophilized peptides covering the immunodominant sequence of the spike glycoprotein and the complete sequence of the membrane glycoprotein of SARS-CoV-2 (15-mer sequences with 11 amino acids overlap) were obtained from Miltenyi Biotec (Thieme et al., 2020) resuspended and stored according to the manufacturer's instructions.

### SARS-CoV-2 Antibody Testing

The LIAISON SARS-CoV-2 S1/S2 IgG (DiaSorin S.p.A., Saluggia, Italy) was utilized as per the manufacturer's instructions to obtain quantitative antibody results from plasma samples via an indirect chemiluminescence immunoassay (CLIA) in a United Kingdom Accreditation Service (UKAS) diagnostic laboratory at University Hospital Southampton. Sample results were interpreted as positive (R 15 AU/mL), Equivocal (R 12.0 and <15.0 AU/mL) and negative (<12 AU/mL).

### SARS-CoV-2 Spike Protein-Specific B Cell Responses

To assess the level of SARS-CoV-2 S1/S2-specific B cells, cells were prepared in staining buffer (PBS with 2% FBS and 2 mMEDTA), FcgR blocked (clone 2.4G2, BD Biosciences), stained with indicated primary antibodies and biotinylated S1/S2 proteins (Sino Biological) for 30 min at 4_C; washed, and subsequently stained with streptavidin-BV421. Patients 10, 24 and 49 were analyzed on a different day with a lower intensity violet laser and required different gating.

### Epitope Megapool to Peptide (MP) Design

The Human Parainfluenza (HPIV) and Metapneumovirus (HMPV) CD4+ T cell peptide megapools (MPs) were produced by sequential lyophilization of viral-specific epitopes as previously described (Carrasco Pro et al., 2015, Weiskopf et al., 2015b). T cell prediction was performed using TepiTool tool, available in identification epitope database analysis resources (IEDB-AR, LI), applying the 7-allele prediction method and a median cutoff %20 (Dhanda et al., 2019, Paul et al., 2015, Paul et al., 2016). For the HA-influenza MP, we selected 177 experimentally defined epitopes, retrieved by querying the IEDB database (www.IEDB.org) on 07/12/19 with search parameters “positive assay only, No B cell assays, No MHC ligand assay, Host: Homo sapiens and MHC restriction class II.” The list of epitopes was enriched with predicted peptides derived from the HA sequences of the vaccine strains available in 2017-2018 and 2018-2019 (A/Michigan/45/2015(H1N1), B/Brisbane/60/2008, A/Hong_Kong/4801/2014(H3N2), A/Michigan/45/2015(H1N1), A/Alaska/06/2016(H3N2), B/Iowa/06/2017, and B/Phuket/3073/2013). The resulting peptides were then clustered using the IEDB cluster 2.0 tool and the IEDB recommended method (cluster-break method) with a 70% cut off for sequence identity applied (Dhanda et al., 2019, Dhanda et al., 2018) (Table SlE). Peptides were synthesized as crude material (A&A, San Diego, CA), resuspended in DMSO, pooled according to each MP composition and finally sequentially lyophilized (Carrasco Pro et al., 2015). For screening healthy non-exposed subjects (samples provided before the current pandemic) who cross-react to SARS-CoV-2, we screened 20 healthy non-exposed subjects using SARS-CoV-2 peptide CD4-R and CD4-S pools, as described (Grifoni et al., 2020).

### Antigen-Reactive T Cell Enrichment (ARTE) Assay

Enrichment and FACS sorting of virus-reactive CD154+ CD4+ memory T cells following peptide pool stimulation was adapted from Bacher et al. 2016 (Bacher et al., 2016). Briefly, PBMCs from each donor, were thawed, washed, plated in 24-well culture plates at a concentration of 5 3 106 cells/mL in 1 mL of serum-free TexMACS medium (Miltenyi Biotec) and left overnight (5% CO2, 37_C). Cells were stimulated by the addition of individual virus-specific peptide pools (1 mg/mL) for 6 h in the presence of a blocking CD40 antibody (1 mg/mL; Miltenyi Biotec). For subsequent MACS-based enrichment of CD154+, cells were sequentially stained with fluorescence-labeled surface antibodies (antibody list in Table SIG), Cell-hashtag TotalSeq-C antibody (0.5 mg/condition), and a biotin conjugated CD154 antibody (clone 5C8; Miltenyi Biotec) followed by anti-biotin microbeads (Miltenyi Biotec). Labeled cells were added to MS columns (Miltenyi Biotec) and positively selected cells (CD154+) were eluted and used for FACS sorting of CD154+ memory CD4+ T cells. The flow-through from the column was collected and re-plated to harvest cells responding 24 h after peptide stimulation. Analogous to enrichment for CD154+, CD137-expressing CD4+ memory T cells were positively selected by staining with biotin-conjugated CD137 antibody (clone REA765; Miltenyi Biotec) followed by anti-biotin MicroBeads and applied to a new MS column. Following elution, enriched populations were immediately sorted using a FACSAria Fusion Cell Sorter (Becton Dickinson) based on dual expression of CD154 and CD69 for the 6 h stimulation condition, and CD137 and CD69 for the 24 h stimulation condition. The gating strategy used for sorting is shown in FIGS. S1A and S4B. All flow cytometry data were analyzed using FlowJo software (version 10).

### Cell Isolation and Single-Cell RNA-Seq Assay (10× Platform)

For combined single-cell RNA-seq and TCR-seq assays (10× Genomics), a maximum of 60,000 virus-reactive memory CD4+ T cells from up to 8 donors were pooled by sorting into low retention 1.5 mL collection tubes, containing 500 ml of a 1:1 solution of PBS:FBS supplemented with recombinant RNase inhibitor (1:100, Takara). For healthy donors, when possible, equal numbers of cells were isolated from each donor and pooled before 10× Genomics single-cell RNA-seq experiments. For analysis of FLU-reactive CD4+ T cell responses, we sequenced paired pre- and post-vaccination samples from 4 donors and supplemented this with 2 non-paired samples for both pre- and post-vaccination. Samples from both pre- and post-vaccination were pooled for analysis of FLU-reactive CD4+ T cells. Following sorting, ice-cold PBS was added to make up to a volume of 1400 ml. Cells were then centrifuged for 5 min (600 g at 4_C) and the supernatant was carefully removed leaving 5 to 10 ml. 25 ml of resuspension buffer (0.22 mm filtered ice-cold PBS supplemented with ultra-pure bovine serum albumin; 0.04%, Sigma-Aldrich) was added to the tube and the pellet was gently but thoroughly resuspended. Following careful mixing, 33 ml of the cell suspension was transferred to a PCR-tube for processing as per the manufacturer's instructions (10× Genomics). Briefly, single-cell RNA-sequencing library preparation was performed as per the manufacturer's recommendations for the 10× Genomics 5′ TAG v1.0 chemistry with immune profiling and cell surface protein technology. Both initial amplification of cDNA and library preparation were carried out with 13 cycles of amplification; V(D)J and cell surface protein libraries were generated corresponding to each 5″ TAG gene expression library using 9 cycles and 8 cycles of amplification, respectively. Libraries were quantified and pooled according to equivalent molar concentrations and sequenced on Illumina NovaSeq6000 sequencing platform with the following read lengths: read 1-101 cycles; read 2-101 cycles; and i7 index—8 cycles.

### Single-Cell Transcriptome Analysis

Reads from single-cell RNA-seq were aligned and collapsed into Unique Molecular Identifiers (UMI) counts using 10× Genomics' Cell Ranger software (v3.1.0) and mapped to GRCh37 reference (v3.0.0) genome. Hashtag UMI counts for each TotalSeq-C antibody capture library were generated with the Feature Barcoding Analysis pipeline from Cell Ranger. To demultiplex donors, UMI counts of cell barcodes were first obtained from the raw data output, and only cells with at least 100 UMI for the hashtag with the highest UMI counts were considered for donor assignment. Donor identities were inferred by MULTIseqDemux (autoThresh=TRUE and maxiter=10) from Seurat (v3.1.5) using the UMI counts. Each cell barcode was assigned a donor ID, marked as a Doublet or having a Negative enrichment. Cells were re-classified as doublets if the ratio of UMI counts between the top 2 barcodes was less than 3. Cells labeled as Doublet or Negative were removed from downstream analyses. Raw 10× data were independently aggregated using Cell Ranger's aggr function (v3.1.0). Donors P28 and P48 were not stained with hashtag antibodies and therefore did not contribute to any donor specific data. The merged data was transferred to the R statistical environment for analysis using the package Seurat (v3.1.5) (Stuart et al., 2019). To further minimize doublets and to eliminate cells with low quality transcriptomes, cells expressing <800 and >4400 unique genes, <1500 and >20,000 total UMI content, and >10% of mitochondrial UMIs were excluded. The summary statistics for all the single-cell transcriptome libraries are provided in Table S2C-E and indicate good quality data with no major differences in quality control metrics across multiple batches, where batches are groups of donors whose libraries were sequenced together (FIG. S2A). This procedure was independently applied for data from CD4+ T cells stimulated for 0 and 6 h, 6 and 24 h.

For single-cell transcriptome analysis only genes expressed in at least 0.1% of the cells were included. The transcriptome data was then log-transformed and normalized (by a factor of 10,000) per cell, using default settings in Seurat software (Stuart et al., 2019). Variable genes with a mean UMI expression greater than 0.01 and explaining 25% of the total variance were selected using the Variance Stabilizing Transformation method, as described (Stuart et al., 2019). Transcriptomic data from each cell was then further scaled by regressing the number of UMI-detected and percentage of mitochondrial counts. For data from CD4+ T cells stimulated for 6 h, principal component analysis was performed using the variable genes, and based on the standard deviation of PCs in the “elbow plot,” the first 38 principal components (PCs) were selected for further analyses. Cells were clustered using the Find Neighbors and Find Clusters functions in Seurat with a resolution of 0.6. The robustness of clustering was independently verified by other clustering methods and by modifying the number of PCs and variable genes utilized for clustering. Analysis of clustering patterns across multiple batches revealed no evidence of strong batch effects (FIG. S2A). For data from CD4+ T cells stimulated for 24 h, the first 16 PCs were selected for further analyses. Cluster 6 (G) in the 24 h dataset was merged with cluster 0 (A) after being identified as T_{REG}. For 0 and 6 h aggregation analysis, 30 PCs were taken. Finally, cells were clustered using the FindNeighbors and FindClusters functions in Seurat with a resolution of 0.6 and 0.2 for 6 and 0 h aggregation and 24 h, respectively. Further visualizations of exported normalized data such as UMAP or “violin” plots were generated using the Seurat package and custom R scripts. Violin shape represents the distribution of cell expressing transcript of interest (based on a Gaussian Kernel density estimation model) and are colored according to the percentage of cells expressing the transcript of interest.

### Single-Cell Differential Gene Expression Analysis

Pairwise single-cell differential gene expression analysis was performed using the MAST package in R (v1.8.2) (Finak et al., 2015) after conversion of data to log 2 counts per million (log 2(CPM+1)). A gene was considered differentially expressed when Benjamini-Hochberg adjusted P-value was <0.05 and a log 2 fold change was more than 0.25. For finding cluster markers (transcripts enriched in a given cluster) the function FindAllMarkers from Seurat was used.

### Gene Set Enrichment Analysis and Signature Module Scores

GSEA scores were calculated with the package fgsea in R using the signal-to-noise ratio (or the log 2 fold change for cluster 5 versus cluster 0 comparison) as a metric. Gene sets were limited by minSize=3 and maxSize=500. Normalized enrichment scores were presented as GSEA plots. Signature module scores were calculated with AddModuleScore function, using default settings in Seurat. Briefly, for each cell, the score is defined by the mean of the signature gene list after the mean expression of an aggregate of control gene lists is subtracted. Control gene lists were sampled (same size as the signature list) from bins created based on the level of expression of the signature gene list. Gene lists used for analysis are provided in Table S2H

### Single-Cell Trajectory Analysis

The “branched” trajectory was constructed using Monocle 3 (v0.2.1, default settings) (Trapnell et al., 2014) with the number of UMI, percentage of mitochondrial UMI as the model formula and including the highly variable genes from Seurat for consistency. After setting a single partition for all cells, the cell-trajectory was projected on the PCA and UMAP generated from Seurat analysis. The ‘root’ was selected by the get_earliest_principal_node function provided in the package. Monocle 3 alpha was used to analyze cluster 0 and 5 using the DDRTree algorithm for dimensional reduction after selecting the top 500 highly variable genes with Seurat.

### T Cell Receptor (TCR) Sequence Analysis

Reads from single-cell V(D)J TCR sequence enriched libraries (Table S2D) were processed with the vdj pipeline from Cell Ranger (v3.1.0 and human annotations reference GRCh38, v3.1.0, as recommended). In brief, the V(D)J transcripts were assembled and their annotations were obtained for each independent library. In order to perform combined analysis of single-cell transcriptome and TCR sequence from the same cells, V(D)J libraries were first aggregated using a custom script. Then cell barcode suffixes from these libraries were revised according to the order of their gene expression libraries. Unique clonotypes, as defined by 10× Genomics as a set of productive Complementarity-Determining Region 3 (CDR3) sequences, were identified across all library files and their frequency and proportion (clone statistics) were calculated based on the aggregation result considering only the cells present in the gene expression libraries. This procedure was independently applied for data from CD4+ T cells stimulated for 6 and 24 h. Based on the vdj aggregation files, barcodes captured by our gene expression data and previously filtered to keep only good-quality cells, were annotated with a specific clonotype ID alongside their clone size (number of cells with the same clonotypes in either one or both the TCR alpha and beta chains) and other statistics (Table S4A,B,E and F). Cells that share clonotype with more than 1 cell were called as clonally expanded (clone size >2). Clone size for each cell was visualized on UMAP, depicting only SARS-CoV-2-reactive CD4+ T cells. Sharing of clonotype between cells in different clusters was depicted using the tool UpSetR (Conway et al., 2017). Finally, in order to assess the sharing between the 0- and 6 h datasets, the same aggregation process was applied for all of the vdj libraries from these data and only SARS-CoV-2-reactive CD4+ T cells specifically isolated from matched patients between sets were considered.

### Quantification and Statistical Analysis

Processing of data, applied methods and codes are described in the respective section in the STAR Methods. The number of subjects, samples, replicates analyzed, and the statistical test performed are indicated in the figure legends or STAR methods. Statistical analysis for comparison between two groups were assessed with Mann Whitney U test and correlation assessed with spearman test with using GraphPad Prism.

1. A method comprising:
(a) obtaining a biological sample;
(b) quantifying a level of a biological feature associated with the number or activity of cytotoxic follicular helper (TFH) or CD4-CTL cells from the biological sample; and
(c) comparing the level of the biological feature associated with the TFH or CD4-CTL cells against a quantifiable reference value, wherein when the level of the biological feature is higher than the quantifiable reference value, the viral infection is associated with SARS-CoV-2.

2. The method of claim 1, wherein the quantifiable reference value comprises a biological feature associated with the activity or number of TFH or CD4-CTL cells isolated from a source infected with a non-SARS-CoV-2 virus.

3. The method of claim 1, wherein the quantifiable reference value comprises a biological feature associated with TFH or CD4-CTL cells isolated from a source infected with an influenza virus.

4. The method of claim 1, wherein the biological feature comprises the expression or activity of one or more genes set forth in Table 2 and/or Table 3, or one or more of the T-cell receptor (TCR) sequences set forth in Table 6, or a homolog, variant, subsequence, or derivative thereof.

5. The method of claim 4, wherein the biological feature comprises expression or activity of one or more of CXCL13, IL21, CD200, BTLA, POU2AF1, PRF1, GZMB, GZMH, GNLY, or NKG7.

6. A method comprising:
(a) obtaining a biological;
(b) quantifying a level of a biological feature associated with the number or activity of cytotoxic follicular helper (TFH) or cells from the biological sample; and
(c) comparing the level of the biological feature against a quantifiable reference value, wherein when the level of the biological feature is above the quantifiable reference value, the virally-induced disease is severe.

7. The method of claim 6, wherein the quantifiable reference value comprises a biological feature associated with the number or activity of TFH cells isolated from a second subject suffering from a non-severe case of the virally-induced disease.

8. The method of claim 6, wherein the biological feature comprises expression or activity of one or more genes set forth in Table 3, or one or more of the TCR sequences set forth in Table 6, or a homolog, variant, subsequence, or derivative thereof.

9. The method of claim 6, wherein the biological feature comprises expression or activity of one or more of ZBED2, ZBTB32, TIGIT, LAG3, TIM3, PD1, DUSP4, CD70, PRF1, or GZMB.

10. The method of claim 6, wherein the virally-induced disease is COVID-19 or is associated with SARS-CoV-2.

11. A method comprising:
(a) obtaining a biological sample;
(b) quantifying a level of a biological feature associated with the number or activity of CD4-CTL cells from the biological sample; and
(c) comparing the level of the biological feature against a quantifiable reference value, wherein when the level of the biological feature is above the quantifiable reference value, the virally-induced disease is severe.

12. The method of claim 11, wherein the quantifiable reference value comprises a biological feature associated with the number or activity of CD4-CTL cells isolated from a second subject suffering from a non-severe case of the virally-induced disease.

13. The method of claim 11, wherein the biological feature comprises expression or activity of one or more genes set forth in Table 2 or Table 4, or one or more of the TCR sequences set forth in Table 6, or a homolog, variant, subsequence, or derivative thereof.

14. The method of claim 11, wherein the biological feature comprises expression or activity of one or more of CD72, GPR18, HOPX, ZEB2, CCL3, CCL4, CCL5, CCR1, CCR3, CCR5, XCL1, or XCL2.

15. The method of claim 11, wherein the virally-induced disease is COVID-19 or is associated with SARS-CoV-2.

16. A method comprising:
(a) obtaining a biological sample;
(b) quantifying a level of a biological feature associated with the number or activity of TREG cells from the biological sample; and
(c) comparing the level of the biological feature associated with T_{REG} against a quantifiable reference value, wherein when the level of the biological feature is below the quantifiable reference value, the virally-induced disease is severe.

17. The method of claim 16, wherein the quantifiable reference value comprises a biological feature associated with the number or activity of TREG cells isolated from a second subject suffering from a mild form of the virally-induced disease.

18. The method of claim 16, wherein the biological feature comprises expression or activity of FOXP3, or one or more of the TCR sequences set forth in Table 7, or a homolog, variant, subsequence, or derivative thereof.

19. (canceled)

20. The method of claim 16, wherein the biological feature comprises the expression or activity of T-bet, IFN-γ, IL-2, TNF, IL-3, CSF2, IL-23A, or CCL20.

21. The method of claim 16, wherein the virally-induced disease is COVID-19 or is associated with SARS-CoV-2.

22.-121. (canceled)
